(19) United States
(12) Patent Application Publication
(10) Pub. No.: US 2014/0186905 A1
(43) Pub. Date: Jul. 3, 2014

(54) BIOTECHNOLOGICAL SYNTHESIS PROCESS OF ORGANIC COMPOUNDS WITH THE AID OF AN ALKL GENE PRODUCT

(71) Applicants: Steffen Schaffer, Herten (DE); Jasmin Gielen, Bochum (DE); Nicole Decker, Recklinghausen (DE); Nicole Kirchner, Recklinghausen (DE); Thomas Haas, Muenster (DE); Markus Poetter, Shanghai (CN); Harald Haeger, Luedinghausen (DE)

(72) Inventors: Steffen Schaffer, Herten (DE); Jasmin Gielen, Bochum (DE); Nicole Decker, Recklinghausen (DE); Nicole Kirchner, Recklinghausen (DE); Thomas Haas, Muenster (DE); Markus Poetter, Shanghai (CN); Harald Haeger, Luedinghausen (DE)

(73) Assignee: Evonik Degussa GmbH, Essen (DE)

(21) Appl. No.: 14/238,576

(22) PCT Filed: Aug. 15, 2012
(86) PCT No.: PCT/EP2012/065933

§ 371 Date: Feb. 12, 2014

(30) Foreign Application Priority Data
Aug. 15, 2011 (DE) 10 2011 110 945.9

(51) Int. Cl. C12P 7/64; C12P 5/02; C12P 13/00

(52) U.S. Cl. CPC C12P 7/649; C12P 7/6409; C12P 13/001; C12P 5/026
USPC 435/128; 435/252.33; 435/134; 435/167

(57) ABSTRACT

Subject matter of the invention is a biotechnological process for the production of organic compounds with the aid of at least one alkL gene product.

### FIELD OF THE INVENTION

Subject matter of the invention is a biotechnological process for the production of organic compounds with the aid of at least one alkL gene product.

### PRIOR ART

Fatty acids and their derivatives are currently obtained exclusively from vegetable and animal oils or fats. This has a number of disadvantages:

As a consequence of the BSE crisis, animal fats in particular are virtually no longer accepted by the customer as raw materials. Vegetable oils which contain short- and medium-chain fatty acids are either not readily available or are produced in tropical regions. Here, the sustainability of the production is open to question in many cases because it may be the case that rainforest is destroyed so as to make the cropping areas available.

Furthermore, vegetable and animal oils and fats have fatty acid spectra which are specific for the raw material in question, but fixed. The consequence is a coupled production, which may determine the price of a particular fatty acid species. Finally, many of the vegetable oils are simultaneously also foodstuffs so that, under certain circumstances, competition may emerge between the use as a feed stock substance and the use as a foodstuff.

This is why there is a search for alternative sources and production pathways for fatty acids, and, as a result, a great deal of research effort is currently being invested into the production of fatty acids in, for example, algae, but also in particular in recombinant microorganisms such as, for example, yeasts and bacteria.

Although a series of technologies are being developed for the production of fatty-acid-based fuels and chemicals from renewable raw materials, in particular carbohydrates, the yields achieved are too low for a meaningful commercial utilization.

The problem of the invention was to provide a more productive biological process for the production of organic compounds.

### DESCRIPTION OF THE INVENTION

Surprisingly, it has been found that the coexpression of an alkL gene product in the producing microorganism, which coexpression is described hereinbelow, is capable of solving the problem of the invention.

Subject matter of the present invention, therefore, are microorganisms which synthesize organic substances and which express alkL at a higher level.

A further subject matter of the invention is the use of the abovementioned microorganisms for the production of organic substances, and a process for producing organic substances using the microorganisms.

An advantage of the present invention is that the product inhibition in the production process can be reduced greatly.

A further advantage is that the space-time yield and the carbon yield of the process are increased in comparison with microorganisms which express no, or less, alkL.

Yet another advantage of the present invention is that the product concentration in the culture supernatant is increased so as to facilitate efficient work-up.

Unless otherwise specified, all percentages stated (%) are percent by mass.

The invention comprises methods for generating recombinant microbial cells which are capable of producing organic substances, such as carboxylic acids and carboxylic acid derivatives, such as, for example, carboxylic acid esters, alkanes, alkan-1-ols, alkan-1-als, alkan-1-amines and 1-alkenes, from unrelated carbon sources.

The present invention therefore comprises a microorganism which includes a first genetic modification so that it is capable of forming more organic substance from at least one simple carbon source in comparison with its wild type, characterized in that the microorganism includes a second genetic modification so that it forms more alkL gene product in comparison with its wild type.

In the context of the present invention, the expression “first genetic modification” is understood as meaning at least one genetic modification of the microorganism in which one or more genes have been modified, i.e. increased or reduced, in their expression in comparison with the wild-type strain.

In the context of the present invention, the expression “simple carbon source” is understood as meaning carbon sources in which, in the carbon skeleton, at least one C—C bond must be broken and/or at least one carbon atom of the simple carbon source must form at least one new bond with at least one carbon atom of another molecule so as to arrive at the carbon skeleton of the “organic substance of which more is formed”.

In the context of the present invention, the expression “alkL gene product” is understood as meaning proteins which meet at least one of the following two conditions:

1.) the protein is identified as a member of the superfamily of the OmpW proteins (Protein family 3922 in the Conserved Domain Database (CDD) of the National Centre for Biotechnology Information (NCBI)), this assignment being made by an alignment of the amino acid sequence of the protein with the database entries present in the NCBI CDD that had been deposited by 22.03.2010, using the standard search parameters, an E value less than 0.01 and using the algorithm “blastp 2.2.23+”,
2.) in a search for conserved protein domains contained in the amino acid sequence of interest in the NCBI CDD (Version 2.20) by means of RPS-BLAST, the presence of the conserved domain “OmpW, Outer membrane protein W” (COG3047) with an E value less than 1×10⁻⁵ is obtained (a domain hit).

Preferred organic substances of the present invention are those which have more than one, in particular 3 to 36, preferably 6 to 24, in particular 10 to 18 carbon atoms. The organic substances may be linear, branched, saturated or unsaturated and optionally substituted by other groups.

It is preferred in accordance with the invention that the organic substance is selected from the group comprising, preferably consisting of,

carboxylic acids, in particular having 3 to 34, preferably having 6 to 22, especially preferably having 6 to 18, carbon atoms,
carboxylic acid esters, in particular having 3 to 34, preferably having 6 to 22, especially preferably having 6 to 18, carbon atoms in the carboxylic acid moiety, in which the alcohol component is derived from methanol, ethanol or other primary alcohols having 3-18 carbon atoms, in particular from methanol and ethanol,
alkanes having 3 to 34, preferably having 6 to 22, especially preferably having 6 to 18, carbon atoms,
alkenes having 3 to 34, preferably having 6 to 22, especially preferably having 6 to 18, carbon atoms,
monohydric alcohols having 3 to 34, preferably having 6 to 22, especially preferably having 6 to 18, carbon atoms,
aldehydes having 3 to 34, preferably having 6 to 22, especially preferably having 6 to 18, carbon atoms,
monovalent amines having 3 to 34, preferably having 6 to 22, especially preferably having 6 to 18, carbon atoms,
and substituted compounds of the above group members, in particular those which carry, as further substituents, one or more hydroxyl, amine, keto, carboxyl, methyl, ethyl, cyclopropyl or epoxy functions, with unsubstituted ones being preferred.

Especially preferred are the organic substances fatty acids, fatty acid esters, alkan-1-als, alkan-1-ols and alkan-1-amines, alkanes and alkenes, in particular 1-alkenes, where the esters in the abovementioned compounds are preferably those in which the alcohol component is derived from methanol, ethanol or other primary alcohols having 3-18 carbon atoms, in particular from methanol and ethanol.

The organic substances are especially preferably selected from among fatty acids and fatty acid esters in which the fatty acid component is selected from the group consisting of formic acid, acetic acid, propionic acid, butyric acid, valeric acid, caproic acid, oenanthic acid, caprylic acid, pelargonic acid, capric acid, lauric acid, myristic acid, pentadecanoic acid, palmitic acid, margaric acid, stearic acid, nonadecanoic acid, arachidic acid, behenic acid, lignoceric acid, cerotic acid, montanic acid, melissic acid, undecylenic acid, myristoleic acid, palmitoleic acid, petroselic acid, oleic acid, elaidic acid, vaccenic acid, gadoleic acid, eicosenoic acid, cetoleic acid, erucic acid, nervonic acid, pelargonic acid, linoleic acid, α-linolenic acid, γ-linolenic acid, calendulic acid, punicic acid, α-elaeostearic acid, β-elaeostearic acid, arachidonic acid, timnodonic acid, clupanodonic acid, cervonic acid, vernolic acid, ricinoleic acid

and
their derivatives in the form of corresponding alkan-1-als, alkan-1-ols, alkan-1-amines and, in the case of unsaturated fatty acids, such as, for example, palmitoleic acid, oleic acid, linoleic acid, α-linolenic acid, γ-linolenic acid, also alken-1-als, alken-1-ols, alken-1-amines, and alkanes and alkenes prepared from the abovementioned fatty acids by enzymatic reduction and decarbonylation, and alkenes, in particular 1-alkenes, prepared from the abovementioned fatty acids by enzymatic decarboxylation, if appropriate having further, nonterminal double bonds.

In this context, the expression “corresponding alkane/alkene-1 compounds” is understood as meaning that the carboxyl group of the fatty acid in question is replaced by a —COH, a —CH₂OH or a —CH₂NH₂.

If, in the context of the present invention, the following text will describe enzymatic activities which catalyse reactions in which alkanoic acids, alkan-1-als, alkan-1-ols or alkan-1-amines are involved either directly or indirectly, it shall be assumed that alkanoic acids, alkan-1-als, alkan-1-ols or alkan-1-amines which additionally have one or more nonterminal double bonds are, as a rule, included in the abovementioned enzymatic activity.

Carbohydrates such as, for example, glucose, sucrose, arabinose, xylose, lactose, fructose, maltose, molasses, starch, cellulose and hemicellulose, but also glycerol or very simple organic molecules such as CO₂, CO or synthesis gas may be employed as the carbon source.

It is preferred in accordance with the invention that, owing to the good genetic accessibility, microorganisms are employed which are selected from the group of the bacteria, especially from the group containing, preferably consisting of, Magnetococcus, Mariprofundus, Acetobacter, Acidiphilium, Afipia, Ahrensia, Asticcacaulis, Aurantimonas, Azorhizobium, Azospirillum, Bartonella, tribocorum, Beijerinckia, Bradyrhizobium, Brevundimonas, subvibrioides, Brucella, Caulobacter, Chelativorans, Citreicella, Citromicrobium, Dinoroseobacter, Erythrobacter, Fulvimarina, Gluconacetobacter, Granulibacter, Hirschia, Hoeflea, Hyphomicrobium, Hyphomonas, Ketogulonicigenium, Labrenzia, Loktanella, Magnetospirillum, Maricaulis, Maritimibacter, Mesorhizobium, Methylobacterium, Methylocystis, Methylosinus, Nitrobacter, Novosphingobium, Oceanibulbus, Oceanicaulis, Oceanicola, Ochrobactrum, Octadecabacter, Oligotropha, Paracoccus, Parvibaculum, Parvularcula, Pelagibaca, Phaeobacter, Phenylobacterium, Polymorphum, Pseudovibrio, Rhodobacter, Rhodomicrobium, Rhodopseudomonas, Rhodospirillum, Roseibium, Roseobacter, Roseomonas, Roseovarius, Ruegeria, Sagittula, Silicibacter, Sphingobium, Sphingomonas, Sphingopyxis, Starkeya, Sulfitobacter, Thalassiobium, Xanthobacter, Zymomonas, Agrobacterium, Rhizobium, Sinorhizobium, Anaplasma, Ehrlichia, Neorickettsia, Orientia, Rickettsia, Wolbachia, Bordetella, Burkholderia, Cupriavidus, taiwanensis, Lautropia, Limnobacter, Polynucleobacter, Raistonia, Chromobacterium, Eikenella, corrodens, Basfia, Kingella, Laribacter, Lutiella, Neisseria, Simonsiella, Achromobacter, Acidovorax, Alicycliphilus, Aromatoleum, Azoarcus, Comamonas, Dechloromonas, Delftia, Gallionella, Herbaspirillum, Herminiimonas, Hylemonella, Janthinobacterium, Leptothrix, Methylibium, Methylobacillus, Methylophilales, Methyloversatilis, Methylovorus, Nitrosomonas, Nitrosospira, Oxalobacter, Parasutterella, Polaromonas, Polaromonas, Pusillimonas, Rhodoferax, Rubrivivax, Sideroxydans, Sutterella, wadsworthensis, Taylorella, Thauera, Thiobacillus, Thiomonas, Variovorax, Verminephrobacter, Anaeromyxobacter, Bdellovibrio, bacteriovorus, Bilophila, Desulfarculus, Desulfatibacillum, Desulfobacca, Desulfobacterium, Desulfobulbus, Desulfococcus, Desulfohalobium, Des ulfitobacterium, Desulfomicrobium, Desulfonatronospira, Desulfotalea, Desulfovibrio, Desulfuromonas, Geobacter, Haliangium, Hippea, Lawsonia, Myxococcus, Pelobacter, Plesiocystis, Sorangium, Stigmatella, Syntrophobacter, Syntrophus, Arcobacter, Caminibacter, Campylobacter, Helicobacter, Nitratifractor, Nitratiruptor, Sulfuricurvum, Sulfurimonas, Sulfurospirillum, Sulfurovum, Wolinella, Buchnera, Blochmannia, Hamiltonella, Regiella, Riesia, Citrobacter, Cronobacter, Dickeya, Edwardsiella, Enterobacter, Erwinia, Escherichia, Klebsiella, Pantoea, Pectobacterium, Proteus, Providencia, Rahnella, Salmonella, Serratia, Shigella, Sodalis, Wigglesworthia, Glossina, Xenorhabdus, Yersinia, Acidithiobacillus, Acinetobacter, Aeromonas, Alcanivorax, Alkalilimnicola, Allochromatium, Alteromonadales, Alteromonas, Baumannia, Beggiatoa, Bermanella, Carsonella, Ruthia, Vesicomyosocius, Cardiobacterium, Chromohalobacter, Colwellia, Congregibacter, Coxiella, Dichelobacter, Endoriftia, Enhydrobacter, Ferrimonas, Francisella, Glaciecola, Hahella, Halomonas, Halorhodospira, Halothiobacillus, Idiomarina, Kangiella, Legionella, Marinobacter, Marinomonas, Methylobacter, Methylococcus, Methylomicrobium, Methylophaga, Moraxella, Moritella, Neptuniibacter, Nitrococcus, Pseudoalteromonas, Psychrobacter, Psychromonas, Reinekea, Rickettsiella, Saccharophagus, Shewanella, Succinatimonas, Teredinibacter, Thioalkalimicrobium, Thioalkalivibrio, Thiomicrospira, Tolumonas, Vibrionales, Actinobacillus, Aggregatibacter, Gallibacterium, Haemophilus, Histophilus, Mannheimia, Pasteurella, Azotobacter, Cellvibrio, Pseudomonas, Aliivibrio, Grimontia, Photobacterium, Photobacterium, Vibrio, Pseudoxanthomonas, Stenotrophomonas, Xanthomonas, Xylella, Borrelia, Brachyspira, Leptospira, Spirochaeta, Treponema, Hodgkinia, Puniceispirillum, Liberibacter, Pelagibacter, Odyssella, Accumulibacter, in particular E. coli, Pseudomonas sp., Pseudomonas fluorescens, Pseudomonas putida, Pseudomonas stutzeri, Acinetobacter sp., Burkholderia sp., Burkholderia thailandensis, cyanobacteria, Klebsiella sp., Klebsiella oxytoca, Salmonella sp., Rhizobium sp. and Rhizobium meliloti, with E. coli being especially preferred.

Preferred alkL gene products which are present in the microorganisms according to the invention are characterized in that the production of the alkL gene product in the native host is induced by dicyclopropyl ketone; in this context, it is additionally preferred that the alkL gene is expressed as part of a group of genes, for example in a regulon, such as, for example, in an operon.

alkL gene products which are present in the microorganisms according to the invention are preferably encoded by alkL genes of organisms selected from the group of the Gram-negative bacteria, in particular the group containing, preferably consisting of, Pseudomonas sp., Azotobacter sp., Desulfitobacterium sp., Burkholderia sp., preferably Burkholderia cepacia, Xanthomonas sp., Rhodobacter sp., Ralstonia sp., Delftia sp. and Rickettsia sp., Oceanicaulis sp., Caulobacter sp., Marinobacter sp. and Rhodopseudomonas sp., preferably Pseudomonas putida, Oceanicaulis alexandrii, Marinobacter aquaeolei, in particular Pseudomonas putida GPo1 and P1, Oceanicaulis alexandrii HTCC2633, Caulobacter sp. K31 and Marinobacter aquaeolei VT8.

In this context, very especially preferred alkL gene products are encoded by the alkL genes from Pseudomonas putida GPo1 and P1, which are shown by SEQ ID No. 1 and SEQ ID No. 29, and proteins with the polypeptide sequence SEQ ID No. 2, SEQ ID No. 30, SEQ ID No. 31, SEQ ID No. 32 or SEQ ID No. 33 or with a polypeptide sequence in which up to 60%, preferably up to 25%, especially preferably up to 15%, particularly up to 10, 9, 8, 7, 6, 5, 4, 3, 2, 1% of the amino acid residues are modified in comparison with SEQ ID No. 2, SEQ ID No. 30, SEQ ID No. 31, SEQ ID No. 32 or SEQ ID No. 33 by deletion, insertion, substitution or a combination thereof and which products still have at least 50%, preferably 65%, especially preferably 80%, in particular more than 90% of the activity of the protein with the respective reference sequence SEQ ID No. 2, SEQ ID No. 30, SEQ ID No. 31, SEQ ID No. 32 or SEQ ID No. 33, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, more precisely in a system as described in the exemplary embodiments, in which system glucose is reacted in an E. coli cell to give palmitoleic acid. A method of choice for determining the synthesis rate can be found in the exemplary embodiments.

The definition of the units here is the definition customary in enzyme kinetics: 1 unit of biocatalyst reacts 1 μmol of substrate in one minute to form the product.

1 U=1 μmol/min

Modifications of amino acid residues of a given polypeptide sequence that do not lead to any substantial changes of the properties and function of the given polypeptide are known to the skilled worker. For instance, some amino acids, for example, can frequently be exchanged for one another without problem; examples of such suitable amino acid substitutions are: Ala for Ser; Arg for Lys; Asn for Gln or His; Asp for Glu; Cys for Ser; Gln for Asn; Glu for Asp; Gly for Pro; His for Asn or Gln; Ile for Leu or Val; Leu for Met or Val; Lys for Arg or Gln or Glu; Met for Leu or Ile; Phe for Met or Leu or Tyr; Ser for Thr; Thr for Ser; Trp for Tyr; Tyr for Trp or Phe; Val for Ile or Leu. Likewise, it is known that modifications, especially at the N- or C-terminus of a polypeptide in the form of, for example, amino acid insertions or deletions, will frequently have no substantial effect on the function of the polypeptide.

First genetic modification for the synthesis of carboxylic acids, carboxylic acid esters and other carboxylic acid derivatives from a simple carbon source

According to the invention, the microorganisms include a first genetic modification so that they are capable of forming more organic substance, in particular carboxylic acids and carboxylic acid derivatives, from at least one simple carbon source in comparison with their wild type.

In this context, it is preferred in accordance with the invention that the first genetic modification is an activity of at least one of the enzymes selected from the group

Eᵢ acyl-ACP (Acyl Carrier Protein) thioesterase, preferably from EC 3.1.2.14 or EC 3.1.2.22, which catalyses the hydrolysis of an acyl-acyl carrier protein thioester,
Eᵢᵢ acyl-CoA (Coenzyme A) thioesterase, preferably from EC 3.1.2.2, EC 3.1.2.18, EC 3.1.2.19, EC 3.1.2.20 or EC 3.1.2.22, which catalyses the hydrolysis of an acyl-coenzyme A thioester,
E_{iib} acyl-CoA (Coenzyme A): ACP (Acyl Carrier Protein) transacylase, which preferably catalyses a reaction in which a CoA thioester is converted into an ACP thioester,
Eᵢᵢᵢ polyketide synthase, which catalyses a reaction which participates in the synthesis of carboxylic acids and carboxylic acid esters, and
Eᵢᵥ hexanoic acid synthase, a specialized fatty acid synthase of the FAS-I type, which catalyses the synthesis of hexanoic acid from two molecules malonyl-coenzyme A and one molecule acetyl-coenzyme A
which is increased in comparison with the enzymatic activity of the wild type of the microorganism.

What will now be said on increasing the enzymatic activity in cells applies not only to the increase of the activity of the enzyme Eᵢ to Eᵢᵥ, but also to all of the enzymes mentioned thereafter, whose activity may optionally be increased, and to an increased alkL gene product formation.

The expression “increased activity of an enzyme” as used hereinabove and in what will be said hereinbelow in the context of the present invention is preferably understood as meaning an increased intracellular activity; this statement also applies to an increased alkl gene product formation.

In principle, an increase of the enzymatic activity can be achieved by increasing the number of copies of the gene sequence or of the gene sequences which encode the enzyme, using a strong promoter, modifying the codon usage of the gene, increasing in various ways the half-life of the mRNA or of the enzyme, modifying the regulation of expression of the gene or using a gene or allele which encodes a corresponding enzyme with an increased activity, and optionally combining these measures. Microorganisms which are genetically modified in accordance with the invention are generated for example by transformation, transduction, conjugation or a combination of these methods using a vector which contains the desired gene, an allele of this gene or parts thereof, and a promoter which makes possible the expression of the gene. Heterologous expression is made possible, in particular, by integrating the gene or the alleles into the chromosome of the cell or a vector which replicates extrachromosomally.

An overview over the possibilities of increasing the enzymatic activity in cells with pyruvate carboxylase as example can be found in DE-A-100 31 999, which is herewith incorporated by reference and whose disclosure regarding the possibilities of increasing the enzymatic activity in cells forms part of the disclosure of the present invention.

The expression of the abovementioned, and all hereinafter mentioned, enzymes and/or genes can be detected in the gel with the aid of one- and two-dimensional protein gel separation followed by optical identification of the protein concentration using suitable evaluation software. If the increase of an enzymatic activity is based exclusively on an increase of the expression of the gene in question, the quantification of the increase of the enzymatic activity can be determined in a simple manner by a comparison of the one- or two-dimensional protein separations between the wild type and the genetically modified cell. A customary method for preparing the protein gels in the case of bacteria and for identifying the proteins is the procedure described by Hermann et al. (Electrophoresis, 22: 1712-23 (2001). The protein concentration can likewise be analysed by Western blot hybridization with an antibody which is specific for the protein to be detected (Sambrook et al., Molecular Cloning: a laboratory manual, 2nd Ed. Cold Spring Harbor Laboratory Press, Cold Spring Harbor, N.Y. USA, 1989) followed by optical evaluation with suitable software for determining the concentration (Lohaus and Meyer (1989) Biospektrum, 5: 32-39; Lottspeich (1999), Angewandte Chemie 111: 2630-2647). This is also always the method of choice when potential products of the reaction catalysed by the enzymatic activity to be determined can be metabolized rapidly in the microorganism or else when the activity in the wild type itself is too low to be able to sufficiently determine the enzymatic activity to be determined with the aid of the product formation.

Using the above-described methods, it can also be determined whether an observed microorganism forms more alkL gene product in comparison with its wild type.

The accession numbers mentioned in the context of the present invention correspond to the ProteinBank database entries of the NCBI dated 26.07.2011; as a rule, the version number of the entry is identified here by “.number”, such as, for example, “0.1”.

### Specific Enzymes Eᵢ

The reaction catalysed by Eᵢ differs from the reaction catalysed by Eᵢᵢ only in that an acyl-coenzyme A thioester is hydrolyse in place of an acyl-acyl carrier protein thioester. It is obvious that many of the enzymes Eᵢ mentioned can, due to the significant secondary activity, also be employed as Eᵢᵢ, and vice versa.

In cells which are preferred in accordance with the invention, the enzyme Eᵢ is an enzyme which comprises sequences selected from among:

AAC72881.1, ABB71579.1, CAC19934.1, AAC49180.1 (encoded by SEQ ID No.: 10), AAC49783.1, AAC49179.1, CAB60830.1, ABB71581.1, AAC49269.1, CAC 19933.1, CAA54060.1, AAC72882.1, Q39513.1, AAC49784.1, ABO38558.1, ABO38555.1, ABO38556.1, ABO38554.1, ADB79568.1, ADB79569.1, ACQ57188.1, ACQ57189.1, ABK96561.1, ACQ63293.1, ACQ57190.1, Q9SQI3.1, ABU96744.1, ABC47311.1, XP_{—}002324962.1, AAD01982.1, AAB51525.1, ACV40757.1, XP_{—}002309244.1, CBI28125.3, ABD91726.1, XP_{—}002284850.1, XP_{—}002309243.1, XP_{—}002515564.1, ACR56792.1, ACR56793.1, XP_{—}002892461.1, ABI18986.1, NP_{—}172327.1, CAA85387.1, CAA85388.1, ADA79524.1, ACR56795.1, ACR56794.1, CAN81819.1, ACF17654.1, AAB71729.1, ABH11710.1, ACQ57187.1, AAX51637.1, AAB88824.1, AAQ08202.1, AAB71731.1, AAX51636.1, CAC80370.1, CAC80371.1, AAG43858.1, ABD83939.1, AAD42220.2, AAG43860.1, AAG43861.1, AAG43857.1, AAL15645.1, AAB71730.1, NP_{—}001068400.1, EAY86877.1, NP_{—}001056776.1, XP_{—}002436457.1, NP_{—}001149963.1, ACN27901.1, EAY99617.1, ABL85052.1, XP_{—}002437226.1, NP_{—}001151366.1, ACF88154.1, NP_{—}001147887.1, XP_{—}002453522.1, BAJ99650.1, EAZ37535.1, EAZ01545.1, AAN 17328.1, EAY86884.1, EEE57469.1, Q41635.1, AAM09524.1, Q39473.1, NP_{—}001057985.1, AAC49001.1, XP_{—}001752161.1, XP_{—}001770108.1, XP_{—}001784994.1, XP_{—}002318751.1, NP_{—}001047567.1, XP_{—}002322277.1, XP_{—}002299627.1, XP_{—}002511148.1, CBI 15695.3, XP_{—}002299629.1, XP_{—}002280321.1, CAN60643.1, XP_{—}002459731.1, XP_{—}002975500.1, XP_{—}002962077.1, XP_{—}001773771.1, NP_{—}001151014.1, XP_{—}002317894.1, XP_{—}002971008.1, XP_{—}001774723.1, XP_{—}002280147.1, XP_{—}002526311.1, XP_{—}002517525.1, XP_{—}001764527.1, AB120759.1, BAD73184.1, XP_{—}002987091.1, XP_{—}002985480.1, CBI26947.3, ABI20760.1, XP_{—}002303055.1, XP_{—}002885681.1, ADH03021.1, XP_{—}002532744.1, EAY74210.1, EEC84846.1, EEE54649.1, AAG35064.1, AAC49002.1, CAD32683.1, ACF78226.1, BAJ96402.1, XP_{—}002462626.1, NP_{—}001130099.1, XP_{—}002462625.1, ABX82799.3, Q42712.1, NP_{—}193041.1, AAB51524.1, NP_{—}189147.1, ABR18461.1, XP_{—}002863277.1, AAC72883.1, AAA33019.1, CBI40881.3, XP_{—}002262721.1, AAB51523.1, NP_{—}001063601.1, ADB79567.1, AAL77443.1, AAL77445.1, AAQ08223.1, AAL79361.1, CAA52070.1, AAA33020.1, CAA52069.1, XP_{—}001785304.1, CAC39106.1, XP_{—}002992591.1, XP_{—}002968049.1, XP_{—}001770737.1, XP_{—}001752563.1, AAG43859.1, XP_{—}002978911.1, XP_{—}002977790.1, ACB29661.1, XP_{—}002314829.1, XP_{—}002991471.1, EAZ45287.1, XP_{—}002986974.1, EEC73687.1, XP_{—}002312421.1, ACJ84621.1, NP_{—}001150707.1, AAD28187.1, XP_{—}001759159.1, XP_{—}001757193.1, XP_{—}002322077.1, ABE01139.1, XP_{—}002447294.1, AAX54515.1, AAD33870.1, AEM72521.1
in particular
AAC72881.1, ABB71579.1, CAC19934.1, AAC49180.1 (encoded by SEQ ID No.: 10), AAC49783.1, AAC49179.1, CAB60830.1, ABB71581.1, AAC49269.1 (encoded by SEQ ID No.: 8), CAC19933.1, CAA54060.1, AAC72882.1, Q39513.1 (encoded by SEQ ID No.: 9), AAC49784.1, AAC72883.1, Q41635.1, AAC49001.1 (encoded by SEQ ID No.: 37), AEM72521.1 (encoded by SEQ ID No.: 35)
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢ is generally understood in particular as meaning the hydrolysis of the dodecanoyl-ACP thioester.

Microorganisms which are preferred according to the invention are those which are obtained when the microorganisms listed hereinbelow, which include a first genetic modification within the meaning of the invention, are employed as the starting point by being provided with the second genetic modification and, if appropriate, with at least one further genetic modification within the meaning of the invention.

WO2010063031 A2 describes microorganisms which include a first genetic modification so that they are capable of forming more microbial oil from at least one simple carbon source in comparison with their wild type and which are preferably employed according to the invention, in particular in sections [0007] to [0008], [0092] to [0100], [0135] to [0136], [0181] to [0186] and [0204] to [0213] and in the exemplary embodiments 4 to 8. The document also describes enzymes Eᵢ which are preferred according to the invention and their sequences, in particular in sections [0012] to [0013], [0155], [0160] to [0163], [0185] to [0190] and [0197] to [0199], FIG. 12, the exemplary embodiments 4 to 8 and Table 3.

WO2010063032 A2 describes microorganisms which include a first genetic modification so that they are capable of forming more microbial oil from at least one simple carbon source in comparison with their wild type and which are preferably employed according to the invention, in particular in sections [0007] to [0008], [0092] to [0100], [0135] to [0136], [0181] to [0186] and [0204] to [0213] and in the exemplary embodiments 4 to 8. The document also describes enzymes Eᵢ which are preferred according to the invention and their sequences, in particular in sections [0012] to [0013], [0155], [0160] to [0163], [0185] to [0190] and [0197] to [0199], FIG. 12, the exemplary embodiments 4 to 8 and Table 3.

WO2011003034 A2 describes microorganisms which include a first genetic modification so that they are capable of forming more fatty acids and fatty acid derivatives, in particular adipic acid, from at least one simple carbon source in comparison with their wild type and which are preferably employed according to the invention, in particular on page 3, second section, to page 7, first section, page 20, second section, to page 22, second section, and on page 156 to page 166, fifth section, and in Claims 1 to 100. The document also describes enzymes Eᵢ which are preferred according to the invention and their sequences, in particular on page 35, third section, and page 36, first section.

WO2011008565 A1 describes microorganisms which include a first genetic modification so that they are capable of forming more fatty acids and fatty acid derivatives, in particular fatty acids, alkan-1-als, alkan-1-ols, alkanes and fatty acid esters, from at least one simple carbon source in comparison with their wild type and which are preferably employed according to the invention, in particular in sections [0018] to [0024] and [0086] to [0102] and in the exemplary embodiments 2, 4, 7, 9 and 10. The document also describes enzymes Eᵢ which are preferred according to the invention and their sequences, in particular in sections [0009] to [0018] and [0073] to [0082], FIGS. 1 to 3 and 7, Table 4, the exemplary embodiments 1 to 10 and Claims 1 to 5 and 11 to 13.

WO2009076559 A1 describes microorganisms which include a first genetic modification so that they are capable of forming more fatty acids and fatty acid derivatives, in particular fatty acids, alkan-1-ols, alkanes or alkenes, from at least one simple carbon source in comparison with their wild type and which are preferably employed according to the invention, in particular in sections [0013] to [0051] and [0064] to [00111] and in Claims 1 to 10. The document also describes enzymes Eᵢ which are preferred according to the invention and their sequences, in particular in Table 1, sections [0021], [0024] to [0030] and [0064] to [00111] and FIG. 6.

WO2010017245 A1 describes microorganisms which include a first genetic modification so that they are capable of forming more fatty acids and fatty acid derivatives from at least one simple carbon source in comparison with their wild type and which are preferably employed according to the invention, in particular in sections [0011] to [0015] and [00114] to [00134], in the exemplary embodiment 3 and in Claims 1 to 2 and 9 to 11. The document also describes enzymes Eᵢ which are preferred according to the invention and their sequences, in particular in Tables 1, 2 and 3, sections [0080] to [00112] and Claims 3 to 8.

WO2010127318 A2 describes microorganisms which include a first genetic modification so that they are capable of forming more fatty acids and fatty acid derivatives, in particular biodiesel equivalents and other fatty acid derivatives, mainly fatty acid ethyl esters, fatty acid esters, wax esters, alkan-1-ols and alkan-1-als, from at least one simple carbon source in comparison with their wild type and which are preferably employed in accordance with the invention, in particular on pages 1 to 9 and 11 to 16, exemplary embodiments 1, 2 and 4, FIGS. 1A to 1E and Claims 23 to 43, 62 to 79 and 101 to 120. The document also describes enzymes Eᵢ which are preferred according to the invention and their sequences, in particular on pages 17, 19 to 23.

WO2008100251 A1 describes microorganisms which include a first genetic modification so that they are capable of forming more fatty acids and fatty acid derivatives, in particular fatty acid esters, wax esters and alkan-1-ols, from at least one simple carbon source in comparison with their wild type and which are preferably employed in accordance with the invention, in particular on pages 4 to 7 and 45 to 46, FIGS. 1A to 1E and Claims 9 to 13. The document also describes enzymes Eᵢ which are preferred according to the invention and their sequences, in particular on pages 4 to 5 and 45 to 46.

WO2007136762 A2 describes microorganisms which include a first genetic modification so that they are capable of forming more fatty acids and fatty acid derivatives, in particular fatty acid esters, wax esters, hydrocarbons and alkan-1-ols, from at least one simple carbon source in comparison with their wild type and which are preferably employed according to the invention, in particular on pages 2 to 4 and 17 to 18, Table 7, FIGS. 2 to 4, exemplary embodiments 2 to 8 and Claims 13 and 35. The document also describes enzymes Eᵢ which are preferred according to the invention and their sequences, in particular on pages 17 to 18, in Tables 1, 7, 8 and 10 and in FIG. 10.

WO2008113041 A2 describes microorganisms which include a first genetic modification so that they are capable of forming more fatty acids and fatty acid derivatives, in particular fatty acid esters, wax esters, hydrocarbons, aliphatic ketones and alkan-1-ols, from at least one simple carbon source in comparison with their wild type and which are preferably employed according to the invention, in particular on pages 35 to 41 and 64 to 67, FIG. 2, exemplary embodiments 6 and 10 and Claims 7 and 36. The document also describes enzymes Eᵢ which are preferred according to the invention and their sequences, in particular in FIG. 7 and exemplary embodiments 6 and 10.

WO2010126891 A1 describes microorganisms which include a first genetic modification so that they are capable of forming more fatty acids and fatty acid derivatives, in particular fatty acid esters, wax esters and alkan-1-ols, from at least one simple carbon source in comparison with their wild type and which are preferably employed according to the invention, in particular in sections [0034] to [0091], [0195] to [0222] and [0245] to [0250], FIGS. 3 to 5 and the exemplary embodiments 1 to 5. The document also describes enzymes Eᵢ which are preferred according to the invention and their sequences, in particular in sections [0245] to [0250], Table 1 and exemplary embodiments 1 to 5.

WO2010118410 A1 describes microorganisms which include a first genetic modification so that they are capable of forming more fatty acids and fatty acid derivatives, in particular fatty acid esters and wax esters, from at least one simple carbon source in comparison with their wild type and which are preferably employed according to the invention, in particular in sections [0022] to [0043], [0158] to [0197], FIGS. 1 to 4, exemplary embodiments 3 and 5 to 8 and Claims 1 to 53 and 82 to 100. The document also describes enzymes Eᵢ which are preferred according to the invention and their sequences, in particular in sections [0158] to [0197], Table 1, FIGS. 3 and 4 and exemplary embodiments 3 and 5 to 8.

WO2010118409 A1 describes microorganisms which include a first genetic modification so that they are capable of forming more fatty acids and fatty acid derivatives, in particular fatty acid esters and wax esters, from at least one simple carbon source in comparison with their wild type and which are preferably employed according to the invention, in particular in sections [0134] to [0154], FIGS. 1 to 3 and 6 and exemplary embodiment 3. The document also describes enzymes Eᵢ which are preferred according to the invention and their sequences, in particular in sections [0134] to [0154], FIGS. 3 and 6 and the exemplary embodiment 3.

WO2010075483 A2 describes microorganisms which include a first genetic modification so that they are capable of forming more fatty acids and fatty acid derivatives, in particular fatty acids, fatty acid methyl esters, fatty acid ethyl esters, alkan-1-ols, fatty alkyl acetates, alkan-1-als, fatty amines, fatty amides, fatty sulphates, fatty ethers, ketones, alkanes, internal and terminal olefins, dicarboxylic acids, α,ω-dicarboxylic acids and α,ω-diols, from at least one simple carbon source in comparison with their wild type and which are preferably employed according to the invention, in particular in sections [0061] to [0090] and [0287] to [0367], FIGS. 1, 4 and 5, exemplary embodiments 1 to 38 and Claims 18 to 26. The document also describes enzymes Eᵢ which are preferred according to the invention and their sequences, in particular in sections [0012] to [0060], Tables 7, 17, 26 and 27, FIGS. 1, 44 to 47 and 55 to 59, exemplary embodiments 1 to 38 and Claims 1 to 17.

WO2010062480 A2 describes microorganisms which include a first genetic modification so that they are capable of forming more fatty acids and fatty acid derivatives, in particular alkan-1-ols, from at least one simple carbon source in comparison with their wild type and which are preferably employed according to the invention, in particular in sections [0022] to [0174] and [0296] to [0330], exemplary embodiments 3 and 5 to 8 and Claims 17 and 24. The document also describes enzymes Eᵢ which are preferred according to the invention and their sequences, in particular in sections [0022] to [0174], Table 1 and exemplary embodiments 3 and 5 to 8.

WO2010042664 A2 describes microorganisms which include a first genetic modification so that they are capable of forming more fatty acids and fatty acid derivatives, in particular alkan-1-als, from at least one simple carbon source in comparison with their wild type and which are preferably employed according to the invention, in particular in sections [0022] to [0143] and [0241] to [0275], exemplary embodiment 2 and Claims 3 and 9. The document also describes enzymes Eᵢ which are preferred according to the invention and their sequences, in particular in Table 1, FIG. 5 and exemplary embodiment 2.

WO2011008535 A1 describes microorganisms which include a first genetic modification so that they are capable of forming more fatty acids and fatty acid derivatives, in particular carboxylic acids, hydroxycarboxylic acids and their lactones, from at least one simple carbon source in comparison with their wild type and which are preferably employed according to the invention, in particular in sections [0024] to [0032] and [0138] to [0158] and FIG. 13.

WO2010022090 A1 describes microorganisms which include a first genetic modification so that they are capable of forming more fatty acids and fatty acid derivatives, in particular fatty acid esters and wax esters, from at least one simple carbon source in comparison with their wild type and which are preferably employed according to the invention, in particular in sections [0022] to [0143] and [0238] to [0275], FIGS. 3 to 5, the exemplary embodiment 2 and Claims 5, 15, 16 and 36. The document also describes enzymes Eᵢ which are preferred according to the invention and their sequences, in particular in Table 1, FIG. 6 and exemplary embodiment 2.

WO2009140695 A1 describes microorganisms which include a first genetic modification so that they are capable of forming more fatty acids and fatty acid derivatives, in particular hydrocarbons, from at least one simple carbon source in comparison with their wild type and which are preferably employed according to the invention, in particular in sections [0214] to [0248] and exemplary embodiments 22 to 24. The document also describes enzymes Eᵢ which are preferred according to the invention and their sequences, in particular in Table 1, FIG. 40 and exemplary embodiments 22 to 24.

WO2010021711 A1 describes microorganisms which include a first genetic modification so that they are capable of forming more fatty acids and fatty acid derivatives, in particular fatty acid esters and wax esters, from at least one simple carbon source in comparison with their wild type and which are preferably employed according to the invention, in particular in sections [0009] to [0020] and [0257] to [0317], FIGS. 3 to 5 and 19, exemplary embodiments 2 to 24 and Claims 4, 5 and 30. The document also describes enzymes Eᵢ which are preferred according to the invention and their sequences, in particular in Table 3, FIG. 6 and exemplary embodiments 2 to 24.

WO2009085278 A1 describes microorganisms which include a first genetic modification so that they are capable of forming more fatty acids and fatty acid derivatives, in particular olefins, from at least one simple carbon source in comparison with their wild type and which are preferably employed according to the invention, in particular in sections [0188] to [0192] and FIG. 10. The document also describes enzymes Eᵢ which are preferred according to the invention and their sequences, in particular in Table 1 and FIG. 10.

WO2011019858 A1 describes microorganisms which include a first genetic modification so that they are capable of forming more fatty acids and fatty acid derivatives, in particular alkan-1-ols, from at least one simple carbon source in comparison with their wild type and which are preferably employed according to the invention, in particular in sections [0023], [0064] to [0074] and [0091] to [0099], exemplary embodiments 1 to 13, FIG. 1 and Claim 8. The document also describes enzymes Eᵢ which are preferred according to the invention and their sequences, in particular in sections [0085] to [0090], exemplary embodiments 1 to 13 and Table 1.

WO2009009391 A2 describes microorganisms which include a first genetic modification so that they are capable of forming more fatty acids and fatty acid derivatives, in particular fatty acid esters, wax esters and alkan-1-ols, from at least one simple carbon source in comparison with their wild type and which are preferably employed according to the invention, in particular in sections [0010] to [0019] and [0191] to [0299], FIGS. 3 to 5, exemplary embodiments 2, 4 to 6, 9 to 14, 17 and 19 and Claims 16, 39, 44 and 55 to 59. The document also describes enzymes Eᵢ which are preferred according to the invention and their sequences, in particular in sections [0010] to [0019] and [0191] to [0299], FIG. 9 and exemplary embodiments 2, 4 to 6, 9 to 14, 17 and 19.

WO2008151149 A2 describes microorganisms which include a first genetic modification so that they are capable of forming more microbial oil from at least one simple carbon source in comparison with their wild type and which are preferably employed according to the invention, in particular in sections [0009], [0015] to [0033], [0053], [0071], [0174] to [0191], [0274] and [0396], Claims 53 to 114, 188 to 206 and 344 to 355 and Tables 1 to 3. The document also describes enzymes Eᵢ which are preferred according to the invention and their sequences, in particular in Table 5.

WO2008147781 A2 describes microorganisms which include a first genetic modification so that they are capable of forming more fatty acids and fatty acid derivatives, in particular hydrocarbons, olefins and aliphatic ketones, from at least one simple carbon source in comparison with their wild type and which are preferably employed according to the invention, in particular in sections [0147] to [0156], exemplary embodiments 1 to 3, 8, 9 and 14 and Claims 65 to 71. The document also describes enzymes Eᵢ which are preferred according to the invention and their sequences, in particular in exemplary embodiments 1 to 3, 8, 9 and 14.

WO2008119082 A2 describes microorganisms which include a first genetic modification so that they are capable of forming more fatty acids and fatty acid derivatives, in particular fatty acid esters, wax esters, triglycerides, biodiesel, gasoline, jet fuel and alkan-1-ols, from at least one simple carbon source in comparison with their wild type and which are preferably employed according to the invention, in particular on pages 3 to 5, 8 to 10 and 40 to 77, in FIGS. 4 and 5, exemplary embodiments 2 to 5 and 8 to 18 and Claims 3 to 39 and 152 to 153. The document also describes enzymes Eᵢ which are preferred according to the invention and their sequences, in particular in Table 1, FIG. 1, exemplary embodiments 2 to 5 and 8 to 18 and Claims 124 to 134 and 138 to 141.

WO2010135624 A2 describes microorganisms which include a first genetic modification so that they are capable of forming more fatty acids and fatty acid derivatives, in particular alkan-1-ols, from at least one simple carbon source in comparison with their wild type and which are preferably employed according to the invention, in particular in sections [0067] to [0083] and [0095] to [0098]. The document also describes enzymes Eᵢ which are preferred according to the invention and their sequences, in particular in sections [0067] to [0083] and [0095] to [0098]. Zheng Z, Gong Q, Liu T, Deng Y, Chen J C and Chen G Q. (Thioesterase II of Escherichia coli plays an important role in 3-hydroxydecanoic acid production. Appl Environ Microbiol. 2004. 70(7):3807-13) describe microorganisms which include a first genetic modification so that they are capable of forming more fatty acids and fatty acid derivatives, in particular fatty acid esters, wax esters and alkan-1-ols, from at least one simple carbon source in comparison with their wild type and which are preferably employed according to the invention, in particular on pages 3808 to 3810 and 3012 and Table 1, 3 and 4. The document also describes enzymes Eᵢ which are preferred according to the invention and their sequences, in particular on pages 3807 and in Table 2.

Steen E J, Kang Y, Bokinsky G, Hu Z, Schirmer A, McClure A, Del Cardayre S B and Keasling J D (Microbial production of fatty-acid-derived fuels and chemicals from plant biomass. Nature. 2010. 463(7280):559-62) describe microorganisms which include a first genetic modification so that they are capable of forming more fatty acids and fatty acid derivatives, in particular fatty acids and fatty acid esters, from at least one simple carbon source in comparison with their wild type and which are preferably employed according to the invention, in particular on page 559, third section, to page 559, first section. The document also describes enzymes Eᵢ which are preferred according to the invention and their sequences, in particular in supplementary Table 1.

Lennen R M, Braden D J, West R A, Dumesic J A and Pfleger B F (A process for microbial hydrocarbon synthesis: Overproduction of fatty acids in Escherichia coli and catalytic conversion to alkanes. Biotechnol Bioeng. 2010. 106(2):193-202) describe microorganisms which include a first genetic modification so that they are capable of forming more fatty acids and fatty acid derivatives, in particular fatty acids and fatty acid esters, from at least one simple carbon source in comparison with their wild type and which are preferably employed according to the invention, in particular on page 193, first section, page 194, first and second section, page 195, second section to page 197, second section, page 198, second section to page 199, third section. The document also describes enzymes Eᵢ which are preferred according to the invention and their sequences, in particular on page 193, first section, page 194, first and second section, page 196, second section, and in supplementary material.

Liu T, Vora H and Khosla C. (Quantitative analysis and engineering of fatty acid biosynthesis in E. coli. Metab Eng. 2010 July; 12(4):378-86.) describe microorganisms which include a first genetic modification so that they are capable of forming more fatty acids and fatty acid derivatives, in particular fatty acids and fatty acid esters, from at least one simple carbon source in comparison with their wild type and which are preferably employed according to the invention, in particular in sections 2.2 and 3.1 and in Table 1 and 2. The document also describes enzymes Eᵢ which are preferred according to the invention and their sequences, in particular in Table 1.

Yuan L, Voelker T A and Hawkins D J. (Modification of the substrate specificity of an acyl-acyl carrier protein thioesterase by protein engineering. Proc Natl Acad Sci USA. 1995 Nov. 7; 92(23):10639-43) describe microorganisms which include a first genetic modification so that they are capable of forming more fatty acids and fatty acid derivatives, in particular fatty acids and fatty acid esters, from at least one simple carbon source in comparison with their wild type and which are preferably employed according to the invention, in particular on page 10641, fourth section, and in FIG. 2 and Table 1. The document also describes enzymes Eᵢ which are preferred according to the invention and their sequences, in particular on page 10639, first section, page 10640, second, third and last section, page 10641, second and third section, and in FIG. 1 and Table 1 and 2.

Lu X, Vora H and Khosla C. (Overproduction of free fatty acids in E. coli: implications for biodiesel production. Metab Eng. 2008. 10(6):333-9.) describe microorganisms which include a first genetic modification so that they are capable of forming more fatty acids and fatty acid derivatives, in particular fatty acids and fatty acid esters, from at least one simple carbon source in comparison with their wild type and which are preferably employed according to the invention in particular on page 334, second section, sections 2.2, 2.3 and 3 (first to fourth section) and in Table 1. The document also describes enzymes Eᵢ which are preferred according to the invention and their sequences, in particular in section 2.2.

Liu X, Sheng J and Curtiss IIII R. (Fatty acid production in genetically modified cyanobacteria. Proc Natl Acad Sci USA. 2011. 108(17):6899-904) describe microorganisms which include a first genetic modification so that they are capable of forming more fatty acids and fatty acid derivatives, in particular fatty acids and fatty acid esters, from at least one simple carbon source in comparison with their wild type and which are preferably employed according to the invention, in particular on page 6899, fourth and last section, page 6900, first to penultimate section, and in Table S1 of the “Supporting Information”. The document also describes enzymes Eᵢ which are preferred according to the invention and their sequences, in particular on page 6899, sixth and last section.

### Specific Enzymes Eᵢᵢ

Microorganisms which are preferred according to the invention are those which are obtained when the microorganisms listed hereinbelow, which include a first genetic modification within the meaning of the invention, are employed as the starting point by being provided with the second genetic modification and, if appropriate, with at least one further genetic modification within the meaning of the invention.

Steen E J, Kang Y, Bokinsky G, Hu Z, Schirmer A, McClure A, Del Cardayre S B and Keasling J D (Microbial production of fatty-acid-derived fuels and chemicals from plant biomass. Nature. 2010. 463(7280):559-62) describe microorganisms which include a first genetic modification so that they are capable of forming more carboxylic acids and carboxylic acid esters, in particular fatty acids and fatty acid esters, from at least one simple carbon source in comparison with their wild type and which are preferably employed according to the invention, in particular on page 559, third section, to page 559, first section. The document also describes enzymes Eᵢᵢ which are preferred according to the invention and their sequences, in particular in supplementary Table 1.

Lennen R M, Braden D J, West R A, Dumesic J A and Pfleger B F (A process for microbial hydrocarbon synthesis: Overproduction of fatty acids in Escherichia coli and catalytic conversion to alkanes. Biotechnol Bioeng. 2010. 106(2):193-202) describe microorganisms which include a first genetic modification so that they are capable of forming more carboxylic acids and carboxylic acid esters, in particular fatty acids and fatty acid esters, from at least one simple carbon source in comparison with their wild type and which are preferably employed according to the invention, in particular on page 193, first section, page 194, first and second section, page 195, second section to page 197, second section, page 198, second section to page 199, third section. The document also describes enzymes Eᵢᵢ which are preferred according to the invention and their sequences, in particular on page 193, first section, page 194, first and second section, page 196, second section, and in supplementary material. Liu T, Vora H and Khosla C. (Quantitative analysis and engineering of fatty acid biosynthesis in E. coli. Metab Eng. 2010 July; 12(4):378-86.) describe microorganisms which include a first genetic modification so that they are capable of forming more carboxylic acids and carboxylic acid esters, in particular fatty acids and fatty acid esters, from at least one simple carbon source in comparison with their wild type and which are preferably employed according to the invention, in particular in sections 2.2 and 3.1 and in Table 1 and 2. The document also describes enzymes Eᵢᵢ which are preferred according to the invention and their sequences, in particular in Table 1.

Yuan L, Voelker T A and Hawkins D J. (Modification of the substrate specificity of an acyl-acyl carrier protein thioesterase by protein engineering. Proc Natl Acad Sci USA. 1995 Nov. 7; 92(23):10639-43) describe microorganisms which include a first genetic modification so that they are capable of forming more carboxylic acids and carboxylic acid esters, in particular fatty acids and fatty acid esters, from at least one simple carbon source in comparison with their wild type and which are preferably employed according to the invention, in particular on page 10641, fourth section, and in FIG. 2 and Table 1. The document also describes enzymes Eᵢᵢ which are preferred according to the invention and their sequences, in particular on page 10639, first section, page 10640, second, third and last section, page 10641, second and third section, and in FIG. 1 and Table 1 and 2.

Lu X, Vora H and Khosla C. (Overproduction of free fatty acids in E. coli: implications for biodiesel production. Metab Eng. 2008. 10(6):333-9.) describe microorganisms which include a first genetic modification so that they are capable of forming more carboxylic acids and carboxylic acid esters, in particular fatty acids and fatty acid esters, from at least one simple carbon source in comparison with their wild type and which are preferably employed according to the invention in particular on page 334, second section, sections 2.2, 2.3 and 3 (first to fourth section) and in Table 1. The document also describes enzymes Eᵢᵢ which are preferred according to the invention and their sequences, in particular in section 2.2.

Liu X, Sheng J and Curtiss IIII R. (Fatty acid production in genetically modified cyanobacteria. Proc Natl Acad Sci USA. 2011. 108(17):6899-904) describe microorganisms which include a first genetic modification so that they are capable of forming more carboxylic acids and carboxylic acid esters, in particular fatty acids and fatty acid esters, from at least one simple carbon source in comparison with their wild type and which are preferably employed according to the invention, in particular on page 6899, fourth and last section, page 6900, first to penultimate section, and in Table S1 of the “Supporting Information”. The document also describes enzymes Eᵢᵢ which are preferred according to the invention and their sequences, in particular on page 6899, sixth and last section.

### Specific Enzymes Eᵢᵢᵢ

Microorganisms which are preferred according to the invention are those which are obtained when the microorganisms listed hereinbelow, which include a first genetic modification within the meaning of the invention, are employed as the starting point by being provided with the second genetic modification and, if appropriate, with at least one further genetic modification within the meaning of the invention.

WO2009121066 A1 describes microorganisms which include a first genetic modification so that they are capable of forming more fatty acids and fatty acid derivatives, in particular dicarboxylic acids, from at least one simple carbon source in comparison with their wild type and which are preferably employed according to the invention, in particular in Claims 8 to 14. The document also describes enzymes Eᵢᵢᵢ which are preferred according to the invention and their sequences, in particular in sections [00026] to [0054], in exemplary embodiments 1 to 6, FIGS. 4 to 10 and Claims 1 to 7.

WO2009134899 A1 describes microorganisms which include a first genetic modification so that they are capable of forming more fatty acids and fatty acid derivatives, in particular carboxylic acids, hydroxycarboxylic acids and their lactones, from at least one simple carbon source in comparison with their wild type and which are preferably employed according to the invention, in particular in sections [0079] to [0082], exemplary embodiment 1 and Claim 20. The document also describes enzymes Eᵢᵢᵢ which are preferred according to the invention and their sequences, in particular in sections [0009] to [0010] and [0044] to [0078], exemplary embodiment 1, FIGS. 1 and 5 to 8 and Claims 15 to 17 and 19.

### Specific Enzymes Eᵢᵥ

In cells which are preferred according to the invention, the enzyme Eᵢᵥ is one which comprises sequences selected from among:

AAS90071.1, XP_{—}002379948.1, AAS90024.1, XP_{—}001821514.2, BAE59512.1, AAL99898.1, AAS90001.1, AAS90049.1, XP_{—}001911518.1, ACH72901.1, XP_{—}681084.1, AAC49198.1, EFW18013.1, XP_{—}003070494.1, XP_{—}001241401.1, XP_{—}002384449.1, XP_{—}001827206.1, XP_{—}002836001.1, XP_{—}001393196.1, XP_{—}660984.1, XP_{—}001395284.1, XP_{—}002148677.1, XP_{—}001827151.2, BAE66018.1, XP_{—}001217254.1, CAK40139.1, XP_{—}001393516.2, XP_{—}002477829.1, XP_{—}002146311.1, XP_{—}002340042.1, XP_{—}002544942.1, CBF87553.1, XP_{—}002149766.1, 2UV8_A, XP_{—}682676.1, CBX98966.1, XP_{—}002560069.1, XP_{—}001273102.1, P15368.1, XP_{—}001273530.1, CBX99714.1, AAB41493.1, XP_{—}001823764.1, XP_{—}001388458.1, XP_{—}748738.1, EDP53207.1, XP_{—}001259179.1, XP_{—}001825741.2, BAE64608.1, XP_{—}001213437.1, XP_{—}002377327.1, XP_{—}002152724.1, EFZ04065.1, XP_{—}001792784.1, EGP89632.1, XP_{—}001407660.1, EFQ31023.1, XP_{—}003040066.1, 2UV9_A, XP_{—}002486436.1, XP_{—}001585982.1, EFY87204.1, XP_{—}002620504.1, XP_{—}003295647.1, EEQ86108.1, XP_{—}001938586.1, XP_{—}001547465.1, XP_{—}001906653.1, XP_{—}001402457.2, CAK40502.1, XP_{—}002568116.1, XP_{—}003230922.1, XP_{—}001647236.1, XP_{—}385497.1, EGD94294.1, EGE05134.1, XP_{—}002849847.1, XP_{—}003015737.1, EFX06093.1, XP_{—}003019052.1, EEH03423.1, XP_{—}001942351.1, EGC45478.1, XP_{—}002556020.1, XP_{—}003011025.1, CAY86729.1, EDN60916.1, EGA84463.1, EGA56454.1, EEU05652.1, NP_{—}015093.1, XP_{—}003231214.1, XP_{—}445956.1, EGA60201.1, XP_{—}003349949.1, XP_{—}003070417.1, XP_{—}001241314.1, EGR48038.1, XP_{—}002615278.1, EFW 15042.1, EGO59647.1, XP_{—}452914.1, XP_{—}962466.1, XP_{—}001537327.1, XP_{—}002796517.1, XP_{—}003305240.1, XP_{—}002543037.1, XP_{—}002499262.1, NP_{—}985412.2, XP_{—}003019770.1, EFW96269.1, XP_{—}002843350.1, EEH43965.1, XP_{—}457388.1, XP_{—}001799391.1, EEH21370.1, BAD08376.1, XP_{—}001486434.1, BAF79876.1, EFY90998.1, XP_{—}001939431.1, EER44845.1, EFZ02060.1, XP_{—}001386834.2, XP_{—}501096.1, XP_{—}003299758.1, XP_{—}002419391.1, XP_{—}002490414.1, ACZ66251.1, XP_{—}002548204.1, P43098.1, XP_{—}002176039.1, XP_{—}002479407.1, EEQ44526.1, AAA34601.1, XP_{—}001791764.1, XP_{—}003009337.1, BAA11913.1, NP_{—}593823.1, BAB62031.1, BAB62032.1, BAB62030.1, 2 PFF_A, XP_{—}380212.1, ADN94478.1, EGF83443.1, XP_{—}681149.1, EGG00662.1, ADN94479.1, ABC94883.1, XP_{—}571099.1, EFY94095.1, EFW39589.1, XP_{—}003194430.1, XP_{—}003031600.1, XP_{—}001836417.1, XP_{—}001880844.1, XP_{—}762607.1, EGN98830.1, EGO24420.1, ACD87451.1, XP_{—}003328630.1, XP_{—}002997955.1, CCA25392.1, XP_{—}002901724.1, EFY86381.1, XP_{—}002901728.1, ADN97213.1, XP_{—}759118.1, XP_{—}003325251.1, XP_{—}003169619.1, XP_{—}002555446.1, ABJ98780.1, XP_{—}723161.1, EDZ68993.1, XP_{—}001526334.1, XP_{—}001223165.1, YP_{—}889015.1, AAO43178.1, YP_{—}001702252.1, XP_{—}003026305.1, YP_{—}003659808.1, ZP_{—}08155637.1, ZP_{—}04749666.1, ZP_{—}08022190.1, YP_{—}004007770.1, YP_{—}954908.1, YP_{—}004522637.1, YP_{—}640811.1, ZP_{—}04448562.1, NP_{—}301868.1, ZP_{—}06851996.1, YP_{—}003273140.1, YP_{—}001071929.1, YP_{—}001133797.1, YP_{—}004076455.1, YP_{—}701403.1, ZP_{—}03324816.1, YP_{—}002778327.1, ZP_{—}02028077.1, YP_{—}909119.1, YP_{—}880884.1, YP_{—}002767320.1, NP_{—}961266.1, ZP_{—}07457010.1, ZP_{—}08206945.1, ZP_{—}02917151.1, ZP_{—}04387794.1, YP_{—}003359863.1, EGO39886.1, ABE96385.1, ZP_{—}05228143.1, ZP_{—}06522069.1, EGL13180.1, ZP_{—}06976698.1, YP_{—}001852225.1, ZP_{—}06596502.1, YP_{—}907384.1, ZP_{—}06518033.1, AEF27803.1, YP_{—}003374392.1, ZP_{—}07485570.1, NP_{—}217040.1, ZP_{—}03742148.1, NP_{—}856198.1, YP_{—}004724192.1, NP_{—}337093.1, AEJ51135.1, ZP_{—}05765008.1, YP_{—}004745991.1, AEJ47516.1, ZP_{—}06927266.1, ZP_{—}03646962.1, AEF31807.1, YP_{—}003939358.1, YP_{—}003971698.1, YP_{—}003986333.1, ZP_{—}05750911.1, ADD61451.1, ZP_{—}07942485.1, YP_{—}004209716.1, YP_{—}004221489.1, AEI96705.1, NP_{—}696693.1, AEG82252.1, YP_{—}004001156.1, ZP_{—}03976473.1, ZP_{—}04663991.1, ZP_{—}00121397.1, YP_{—}003662064.1, YP_{—}003646283.1, YP_{—}004630447.1, YP_{—}002323720.1, YP_{—}002835610.1, YP_{—}117466.1, ZP_{—}02963252.1, ADC85342.1, NP_{—}940183.1, NP_{—}739002.1, ZP_{—}06755645.1, ADL21513.1, YP_{—}003784047.1, ADL11108.1, ZP_{—}06608499.1, ZP_{—}07967121.1, ZP_{—}05966223.1, ZP_{—}08682531.1, ZP_{—}03918327.1, ZP_{—}07879655.1, ZP_{—}03972703.1, ZP_{—}06162645.1, ZP_{—}06837277.1, ZP_{—}07990916.1, ZP_{—}03394081.1, CAA46024.1, YP_{—}004760934.1, ZP_{—}06751771.1, ZP_{—}03934033.1, NP_{—}601696.1, BAB99888.1, YP_{—}001139316.1, ZP_{—}03926457.1, NP_{—}737523.1, ZP_{—}02044858.1, ZP_{—}07404023.1, ZP_{—}03709883.1, XP_{—}002388648.1, ZP_{—}07402466.1, ZP_{—}03710807.1, ZP_{—}08294093.1, ZP_{—}08232611.1, XP_{—}682514.1, ZP_{—}06837028.1, YP_{—}001137826.1, CAA61087.1, ZP_{—}06043461.1, YP_{—}002833817.1, YP_{—}225128.1, NP_{—}600065.1, ABU23831.1, ZP_{—}07716892.1, ZP_{—}03935133.1, ZP_{—}02549600.1, ZP_{—}05215994.1, YP_{—}004494858.1, XP_{—}001526333.1, AAS90085.1, XP_{—}002379947.1, AAS90025.1, XP_{—}001821515.1, AAL99899.1, AAS90002.1, AAS90050.1, XP_{—}001911517.1, ACH72900.1, XP_{—}681083.1, AAC49199.1, XP_{—}003070495.1, XP_{—}001241402.1, EFW18012.1, CBX98970.1, EEH03422.1, EEQ86107.1, EGC45479.1, XP_{—}002620503.1, XP_{—}001537328.1, XP_{—}002796516.1, 2UVA_G, EEH43966.1, DAA05950.1, EGR47893.1, XP_{—}003070418.1, XP_{—}001241316.1, XP_{—}001827193.1, XP_{—}002384436.1, XP_{—}682677.1, XP_{—}002486435.1, EGP88608.1, EDP53206.1, XP_{—}001259180.1, EEH21369.1, XP_{—}002543038.1, XP_{—}748739.1, XP_{—}003015735.1, EGE05135.1, XP_{—}002152723.1, XP_{—}002560068.1, XP_{—}001273529.1, XP_{—}003230923.1, EFX05327.1, XP_{—}003019051.1, XP_{—}001585981.1, XP_{—}361644.2, XP_{—}001223166.1, XP_{—}003349948.1, XP_{—}002380737.1, AAB41494.1, XP_{—}001823765.1, XP_{—}962465.1, EGO59648.1, XP_{—}001906652.1, XP_{—}003039864.1, XP_{—}001213436.1, XP_{—}385498.1, XP_{—}003295646.1, EFQ31022.1, XP_{—}002849848.1, XP_{—}002148679.1, CBX99715.1, XP_{—}002149767.1, EFY87205.1, EFZ04064.1, XP_{—}002340041.1, EGD94295.1, XP_{—}001938587.1, CAK45758.1, XP_{—}001792785.1, XP_{—}001393189.2, XP_{—}003169620.1, XP_{—}001547461.1, XP_{—}001217253.1, XP_{—}001939430.1, BAA92930.1, Q92215.1, EDK38075.2, EFW97345.1, XP_{—}002495511.1, XP_{—}451653.1, XP_{—}500912.1, CAA42211.1, XP_{—}001486502.1, XP_{—}002477835.1, XP_{—}445436.1, NP_{—}594370.1, XP_{—}001827152.2, BAE66019.1, BAA36384.1, BAB62141.1, XP_{—}003299759.1, XP_{—}002553365.1, XP_{—}002489642.1, 2UV8_G, XP_{—}457311.1, CAY80909.1, XP_{—}001395285.1, EGA61562.1, EDN60099.1, EDV12927.1, NP_{—}012739.1, XP_{—}002616181.1, XP_{—}002420328.1, XP_{—}001524822.1, XP_{—}002550943.1, XP_{—}001386364.2, NP_{—}984945.2, 227846, AAB59310.1, XP_{—}001646561.1, XP_{—}716877.1, XP_{—}001836417.1, XP_{—}002146312.1, P34731.1, EGO24420.1, XP_{—}002544941.1, EFZ02054.1, XP_{—}002175228.1, XP_{—}001393490.2, XP_{—}003031600.1, XP_{—}002479408.1, XP_{—}002568119.1, XP_{—}001825735.2, XP_{—}002377320.1, EGN98830.1, ACD87451.1, XP_{—}001880844.1, XP_{—}571100.1, ABC94882.1, XP_{—}775164.1, BAE64602.1, EFY90992.1, XP_{—}003194424.1, XP_{—}001273103.1, XP_{—}681142.1, XP_{—}003011020.1, AAA34602.1, XP_{—}003231209.1, XP_{—}003019765.1, ADN94478.1, EEQ46070.1, XP_{—}001799393.1, CAK40504.1, AAM75418.1, ADN94479.1, XP_{—}002843356.1, CAA27616.1, XP_{—}380213.1, ADN97213.1, XP_{—}759118.1, XP_{—}762607.1, CAK49094.1, EER44843.1, XP_{—}003009335.1, XP_{—}002997955.1, XP_{—}002901724.1, CCA25392.1, CAK36856.1, XP_{—}001388457.2, ABO37974.1, ABJ98780.1, XP_{—}660985.1, EDZ71063.1, XP_{—}001402459.2, XP_{—}001791765.1, XP_{—}003324647.1, EGG 10429.1, EFW 15039.1, XP_{—}002384390.1, XP_{—}003031976.1, EDZ71062.1, EFW39589.1, ACZ80683.1, XP_{—}002901728.1, XP_{—}003328630.1, XP_{—}681125.1, XP_{—}003325251.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢᵥ is generally understood in particular as meaning the conversion into hexanoic acid of two molecules malonyl-coenzyme A and one molecule acetyl-coenzyme A.

Microorganisms which are preferred according to the invention are those which are obtained when the microorganisms listed hereinbelow, which include a first genetic modification within the meaning of the invention, are employed as the starting point by being provided with the second genetic modification and, if appropriate, with at least one further genetic modification within the meaning of the invention.

WO2011003034 A2 describes microorganisms which include a first genetic modification so that they are capable of forming more fatty acids and fatty acid derivatives, in particular hexanoic acid, from at least one simple carbon source in comparison with their wild type and which are preferably employed according to the invention, in particular on page 2 to 3, page 5, third section, in exemplary embodiments 1 to 4, 7 to 9 and 12 to 14 and Claims 1 to 100. The document also describes enzymes Eᵢᵥ which are preferred according to the invention and their sequences, in particular on page 5 and in exemplary embodiment 3.

Hitchman T S, Schmidt E W, Trail F, Rarick M D, Linz J E and Townsend C A. (Hexanoate synthase, a specialized type I fatty acid synthase in aflatoxin B1 biosynthesis. Bioorg Chem.

2001. 29(5):293-307) describe microorganisms which include a first genetic modification so that they are capable of forming more fatty acids and fatty acid derivatives, in particular hexanoic acid, from at least one simple carbon source in comparison with their wild type and which are preferably employed according to the invention, in particular on page 296, penultimate section to page 298, second section. The document also describes enzymes Eᵢᵥ which are preferred according to the invention and their sequences, in particular on page 299, fourth section, to page 302, first section.

In the context of the first genetic modification, it may be beneficial to employ, in place of the enzyme Eᵢ, a combination of the activity increase in comparison with that of the wild type of an enzyme Eᵢᵢ paired with that of an enzyme E_{iib}, which catalyses a reaction in which a CoA thioester is converted into an ACP thioester.

Such enzymes E_{iib} are known as acyl-CoA (Coenzyme A):ACP (Acyl Carrier Protein) transacylases. Preferred enzymes E_{iib} are selected from among

XP_{—}003402554.1, YP_{—}002908243.1, YP_{—}001778804.1, YP_{—}001670627.1, YP_{—}004703658.1, YP_{—}001747923.1, YP_{—}004348703.1, YP_{—}004352505.1, YP_{—}004379169.1, ADR61731.1, YP_{—}001269622.1, YP_{—}001186851.1, YP_{—}004659609.1, YP_{—}003519049.1, YP_{—}001811696.1, YP_{—}004616040.1, NP_{—}252697.1, NP_{—}252169.1, NP_{—}249421.1, ZP_{—}06456665.1, ZP_{—}01167071.1, ZP_{—}08557569.1, ZP_{—}08554397.1, YP_{—}001157914.1, YP_{—}004475334.1, EGM20156.1, BAK10182.1, YP_{—}347066.1, Q9KJH8.1, YP_{—}002987902.1, ZP_{—}03794633.1, ZP_{—}03627777.1, YP_{—}004434330.1, NP_{—}743567.1, ZP_{—}03456835.1, ZP_{—}07911512.1, ZP_{—}07264431.1, ZP_{—}02265387.2, ZP_{—}03456013.1, ZP_{—}07577798.1, ZP_{—}08429367.1, YP_{—}004055319.1, YP_{—}004053883.1, YP_{—}275219.1, YP_{—}276116.1, YP_{—}003882762.1, EGH97259.1, EGH95622.1, EGH90852.1, EGH85976.1, EGH81248.1, EGH79586.1, EGH79549.1, EGH73565.1, EGH66549.1, EGH64812.1, EGH58099.1, EGH54896.1, EGH50352.1, EGH43364.1, EGH41593.1, EGH29888.1, EGH29417.1, EGH22392.1, EGH22129.1, EGH11618.1, EGH10011.1, ZP_{—}04589662.1, CCA60711.1, YP_{—}003004716.1, BAK16630.1, YP_{—}003264146.1, YP_{—}371314.1, YP_{—}439272.1, NP_{—}762892.1, ADW02533.1, YP_{—}003291774.1, EGC99875.1, ZP_{—}08139631.1, YP_{—}003333890.1, EGC08366.1, YP_{—}080427.1, YP_{—}258557.1, YP_{—}001985016.1, YP_{—}002875182.1, YP_{—}002871082.1, YP_{—}237050.1, YP_{—}236199.1, NP_{—}794008.1, NP_{—}793082.1, YP_{—}609790.1, EFW81598.1, EFW79804.1, ZP_{—}07261632.1, ZP_{—}07229875.1, ZP_{—}06458504.1, ZP_{—}05640568.1, ZP_{—}03399268.1, ZP_{—}03398232.1, ZP_{—}08004496.1, ZP_{—}06876938.1, ZP_{—}03227482.1, ZP_{—}02511781.1, ZP_{—}02503964.1, ZP_{—}02477255.1, ZP_{—}02466678.1, ZP_{—}02465791.1, ZP_{—}02461688.1, ZP_{—}02417235.1, ZP_{—}02414413.1, ZP_{—}02408727.1, ZP_{—}02376540.1, ZP_{—}02358949.1, ZP_{—}07778021.1, ZP_{—}07774051.1, ZP_{—}07795409.1, ZP_{—}07089008.1, YP_{—}776393.1, ZP_{—}07684652.1, ZP_{—}06640022.1, ZP_{—}03054335.1, ZP_{—}02907621.1, ZP_{—}02891475.1, ZP_{—}01862226.1, ZP_{—}01769192.1, ZP_{—}01367441.1, ZP_{—}01366930.1, ZP_{—}01364106.1, ZP_{—}01312991.1, ZP_{—}01173135.1, ZP_{—}07005523.1, ZP_{—}04955702.1, ZP_{—}04943305.1, ZP_{—}04936014.1, ZP_{—}04932415.1, ZP_{—}04930223.1, ZP_{—}04905334.1, ZP_{—}04893870.1, ZP_{—}04893165.1, ZP_{—}04892059.1, ZP_{—}04884056.1, YP_{—}002438575.1, YP_{—}002234939.1, YP_{—}001488024.1, YP_{—}001346487.1, YP_{—}001350135.1, YP_{—}001347031.1, YP_{—}990329.1, YP_{—}860279.1, YP_{—}789111.1, YP_{—}792557.1, YP_{—}623139.1, YP_{—}175644.1, YP_{—}111362.1, YP_{—}110557.1, YP_{—}105231.1, NP_{—}937516.1, AAU44816.1, AAA25978.1, XP_{—}002721010.1, AAK81868.1, AAK71350.1, AAK71349.1, ZP_{—}06499968.1, ZP_{—}06498781.1, YP_{—}003472045.1, ACA03779.1, ABL84756.1, AAQ16175.1, AAT51302.1, AAT51199.1, ZP_{—}05639386.1, ACH70299.1, ACA60824.1, BAB32432.1, in particular AAK81868.1, NP_{—}743567.1, AAK71349.1, YP_{—}001269622.1, ADR61731.1, AAU44816.1, AAQ16175.1, YP_{—}001670627.1, ACH70299.1, Q9KJH8.1, YP_{—}004703658.1, ZP_{—}08139631.1, YP_{—}609790.1, YP_{—}001747923.1, YP_{—}258557.1, YP_{—}347066.1, YP_{—}002871082.1, YP_{—}004352505.1, ACA60824.1, ZP_{—}07774051.1, BAB32432.1, ZP_{—}05640568.1, EGH58099.1, EGH64812.1, EGH11618.1, ZP_{—}06456665.1, YP_{—}276116.1, EFW81598.1, EGH95622.1, EGH22129.1, NP_{—}794008.1, ZP_{—}03399268.1, ZP_{—}07264431.1, EGH73565.1, YP_{—}237050.1, ZP_{—}06498781.1, EGH29888.1, EGH79586.1, EGH35052.1, YP_{—}792557.1, YP_{—}001350135.1, ZP_{—}01364106.1, ZP_{—}04932415.1, NP_{—}249421.1, YP_{—}004379169.1, ACA03779.1, YP_{—}001186851.1, YP_{—}004475334.1, ZP_{—}04589662.1, ZP_{—}03398232.1, EGH10011.1, ZP_{—}07229875.1, ZP_{—}05639386.1, EGH66549.1, YP_{—}275219.1, ZP_{—}07005523.1, EFW79804.1, ZP_{—}06458504.1, EGH85976.1, YP_{—}236199.1, EGH43364.1, ZP_{—}07261632.1, ZP_{—}06499968.1, EGH29417.1, EGH54896.1, EGH22392.1, EGH97259.1, NP_{—}793082.1, EGH90852.1, EGH41593.1, NP_{—}252169.1, ZP_{—}01366930.1, YP_{—}001347031.1, ZP_{—}07778021.1, YP_{—}002875182.1, AAA25978.1, ABL84756.1, EGH81248.1, ZP_{—}07795409.1 and especially preferably AAU44816.1, NP_{—}743567.1, YP_{—}001269622.1, ADR61731.1, AAK71349.1, YP_{—}001670627.1, AAK81868.1, AAQ16175.1, Q9KJH8.1, ACH70299.1, YP_{—}004703658.1, ZP_{—}08139631.1, YP_{—}609790.1, YP_{—}001747923.1, AAK71350.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{iib} is generally understood in particular as meaning the conversion of dodecanoyl-CoA thioester into dodecanoyl-ACP thioester.
Third Genetic Modification for the Production of Carboxylic Acid Esters from a Simple Carbon Source

It is advantageous in particular for the production of carboxylic acid esters when the microorganism additionally includes a third genetic modification which comprises an activity of at least one of the enzymes E_{iib}, Eᵥ, Eᵥᵢ, or Eᵥᵢᵢ which is increased in comparison with the enzymatic activity of the wild type of the microorganism.

In this context, it is preferred according to the invention that this genetic modification is an activity of at least one of the enzymes selected from the group

E_{iib} acyl-CoA (Coenzyme A):ACP (Acyl Carrier Protein) transacylase, which converts an ACP thioester into a CoA thioester or a CoA thioester into an ACP thioester,
Eᵥ wax ester synthase or alcohol O-acyltransferase, preferably of EC 2.3.1.75 or EC 2.3.1.84, which catalyses the synthesis of an ester from an acyl-coenzyme A thioester or an ACP thioester and an alcohol,
Eᵥₐ fatty acid O-methyl transferase, preferably of EC 2.1.1.15, which catalyses the synthesis of a fatty acid methyl ester from a fatty acid and S-adenosylmethionine,
Eᵥᵢ acyl-CoA (Coenzyme A) synthetase, preferably of EC 6.2.1.3, which catalyses the synthesis of an acyl-coenzyme A thioester, and
Eᵥᵢᵢ acyl thioesterase, preferably of EC 3.1.2.2, EC 3.1.2.4, EC 3.1.2.18, EC 3.1.2.19, EC 3.1.2.20 or EC 3.1.2.22, which catalyses the conversion of an acyl thioester with an alcohol to give a carboxylic acid ester,
which is increased in comparison with the enzymatic activity of the wild type of the microorganism.

In this context, it is especially preferred that the third genetic modification comprises combinations of the increased activities of the enzymes selected from among Eᵥ, Eᵥₐ, Eᵥᵢᵢ, EᵥₐEᵥᵢᵢ, EᵥEᵥᵢ, EᵥᵢEᵥᵢᵢ, EᵥᵢEᵥᵢᵢE_{iib}.

Preferred enzymes E_{iib} in connection with the third genetic modification correspond to the enzymes E_{iib} which have been emphasized above as being preferred in connection with the first genetic modification.

### Specific Enzymes Eᵥ

In cells which are preferred according to the invention, the enzyme Eᵥ is one which comprises sequences selected from among:

NP_{—}808414.2, NP_{—}001178653.1, XP_{—}003272721.1, XP_{—}002720111.1, NP_{—}001002254.1, XP_{—}529027.1, XP_{—}002831804.1, BAC28882.1, XP_{—}549056.2, XP_{—}002918053.1, XP_{—}001085075.1, XP_{—}002763005.1, XP_{—}002700092.1, XP_{—}599558.4, EDL95940.1, XP_{—}001496780.1, CAD89267.1, EFB28125.1,
YP_{—}004747160.1, YP_{—}004746900.1, YP_{—}004746665.1, YP_{—}004746558.1, YP_{—}004746531.1, YP_{—}004746530.1, YP_{—}004745948.1, YP_{—}004745222.1, YP_{—}004744358.1, YP_{—}004743710.1, YP_{—}002492297.1, AEK40846.1, YP_{—}001847685.1, YP_{—}001712672.1, YP_{—}001706290.1, YP_{—}004724737.1, YP_{—}004723134.1, AEJ51098.1, AEJ48174.1, AEJ47480.1, YP_{—}004392630.1, YP_{—}004099725.1, YP_{—}003912033.1, YP_{—}003652731.1, YP_{—}003301387.1, YP_{—}003298139.1, YP_{—}001509672.1, YP_{—}001505948.1, YP_{—}001432486.1, YP_{—}001432432.1, YP_{—}924893.1, YP_{—}923981.1, YP_{—}922869.1, YP_{—}922597.1, YP_{—}922419.1, ZP_{—}08629145.1, ZP_{—}08628906.1, YP_{—}001380027.1, YP_{—}001280731.1, YP_{—}001280730.1, YP_{—}888966.1, YP_{—}890540.1, YP_{—}888236.1, YP_{—}888223.1, YP_{—}888574.1, YP_{—}884705.1, YP_{—}889488.1, YP_{—}886248.1, YP_{—}882534.1, YP_{—}881069.1, YP_{—}881444.1, YP_{—}883472.1, YP_{—}879642.1, YP_{—}884073.1, YP_{—}880917.1, YP_{—}882201.1, YP_{—}879422.1, YP_{—}707862.1, YP_{—}707847.1, YP_{—}707633.1, YP_{—}707572.1, YP_{—}707571.1, YP_{—}706785.1, YP_{—}706267.1, YP_{—}705586.1, YP_{—}705294.1, YP_{—}702929.1, YP_{—}701572.1, YP_{—}700576.1, YP_{—}700081.1, YP_{—}700033.1, YP_{—}700018.1, YP_{—}700017.1, YP_{—}699999.1, CCB78299.1, CCB78283.1, CCB72233.1, YP_{—}004663601.1, YP_{—}004525283.1, YP_{—}004524901.1, YP_{—}004524237.1, YP_{—}004524223.1, YP_{—}004523752.1, YP_{—}004522677.1, YP_{—}004521797.1, YP_{—}004521441.1, YP_{—}004020500.1, YP_{—}004014348.1, EGO40684.1, EGO38684.1, EGO38655.1, EGO37244.1, EGO36970.1, EGO36701.1, YP_{—}003951335.1, YP_{—}003812176.1, YP_{—}003811992.1, YP_{—}003810691.1, YP_{—}003810418.1, YP_{—}003809501.1, ZP_{—}08574204.1, CCA19760.1, XP_{—}002900672.1, ZP_{—}06414567.1, ZP_{—}06413635.1, ZP_{—}06411773.1, ZP_{—}06411772.1, ZP_{—}06271823.1, ZP_{—}05620754.1, ZP_{—}05360001.1, ZP_{—}04752019.1, ZP_{—}04751943.1, ZP_{—}04750965.1, ZP_{—}04750465.1, ZP_{—}04750453.1, ZP_{—}04750228.1, ZP_{—}04750091.1, ZP_{—}04749363.1, ZP_{—}04749348.1, ZP_{—}04749293.1, ZP_{—}04749287.1, ZP_{—}04749022.1, ZP_{—}04748677.1, ZP_{—}04747379.1, ZP_{—}04747377.1, ZP_{—}04747348.1, ZP_{—}04747282.1, ZP_{—}04747159.1, ZP_{—}04747093.1, ZP_{—}04746958.1, ZP_{—}04717323.1, ZP_{—}04684258.1, ZP_{—}04386203.1, ZP_{—}04385082.1, ZP_{—}04384030.1, ZP_{—}04384029.1, ZP_{—}03534755.1, ZP_{—}01115502.1, ZP_{—}01102322.1, YP_{—}004583872.1, YP_{—}004583323.1, YP_{—}004573656.1, YP_{—}004571392.1, YP_{—}003513699.1, ZP_{—}08553011.1, ZP_{—}08552672.1, YP_{—}003467054.1, YP_{—}003572597.1, YP_{—}579515.1, YP_{—}001136465.1, YP_{—}001136231.1, YP_{—}001135959.1, YP_{—}001135349.1, YP_{—}001133828.1, YP_{—}001133806.1, YP_{—}001133693.1, YP_{—}001133270.1, YP_{—}001132329.1, YP_{—}001131721.1, YP_{—}001131631.1, YP_{—}001073715.1, YP_{—}001073143.1, YP_{—}001072388.1, YP_{—}001072036.1, YP_{—}001071893.1, YP_{—}001071814.1, YP_{—}001071689.1, YP_{—}001070856.1, YP_{—}001069682.1, YP_{—}001069164.1, YP_{—}001068496.1, YP_{—}939377.1, YP_{—}642242.1, YP_{—}641664.1, YP_{—}641419.1, YP_{—}640919.1, YP_{—}640783.1, YP_{—}640704.1, YP_{—}640572.1, YP_{—}640571.1, YP_{—}640494.1, YP_{—}639709.1, YP_{—}639198.1, YP_{—}638523.1, YP_{—}638030.1, YP_{—}637968.1, YP_{—}637380.1, YP_{—}446603.1, NP_{—}001185377.1, NP_{—}200151.2, NP_{—}568547.1, NP_{—}197641.1, NP_{—}200150.1, NP_{—}197139.1, NP_{—}190490.1, NP_{—}190488.1, NP_{—}177356.1, YP_{—}004495408.1, YP_{—}004495023.1, YP_{—}004494197.1, YP_{—}004494168.1, YP_{—}004493973.1, YP_{—}004493936.1, YP_{—}004493628.1, YP_{—}004493589.1, YP_{—}004493509.1, YP_{—}004493477.1, YP_{—}004493462.1, YP_{—}004492352.1, YP_{—}004492155.1, YP_{—}004492039.1, YP_{—}004491716.1, YP_{—}004491715.1, YP_{—}004491501.1, YP_{—}003375642.1, YP_{—}003411203.1, YP_{—}003410436.1, YP_{—}003395271.1, YP_{—}003395089.1, YP_{—}003393635.1, YP_{—}003384208.1, YP_{—}003379551.1, ZP_{—}04388235.1, YP_{—}002134168.1, ZP_{—}01900421.1, ZP_{—}01900085.1, ZP_{—}01899829.1, ZP_{—}01898741.1, BAK05274.1, BAJ93623.1, BAJ97841.1, BAK08349.1, BAJ93204.1, BAJ92722.1, BAK06983.1, BAJ86545.1, BAK02325.1, BAJ85619.1, BAJ84892.1, ZP_{—}05218281.1, ZP_{—}05218149.1, ZP_{—}05217310.1, ZP_{—}05216978.1, ZP_{—}05216447.1, ZP_{—}05216446.1, ZP_{—}05216025.1, ZP_{—}05214687.1, ZP_{—}08476543.1, ZP_{—}04749239.1, YP_{—}823060.1, ADP99639.1, ADP98951.1, ADP98855.1, ADP98710.1, ADP96265.1, ZP_{—}08461736.1, ZP_{—}08461735.1, ZP_{—}07608690.1, YP_{—}045555.1 (encoded by SEQ ID No.: 19), YP_{—}872243.1, YP_{—}004009106.1, YP_{—}004008736.1, YP_{—}004008003.1, YP_{—}004007600.1, YP_{—}004006799.1, YP_{—}004006436.1, YP_{—}004006072.1, YP_{—}004005008.1, YP_{—}003486913.1, NP_{—}301898.1, ZP_{—}08434757.1, YP_{—}004079491.1, YP_{—}004078785.1, YP_{—}004077880.1, YP_{—}004076486.1, YP_{—}004076464.1, YP_{—}004076350.1, YP_{—}004075391.1, YP_{—}004074864.1, ZP_{—}01103855.1, YP_{—}465274.1, ZP_{—}08403393.1, ZP_{—}08402717.1, ZP_{—}08402716.1, YP_{—}004427559.1, YP_{—}001277083.1, YP_{—}001276783.1, YP_{—}524767.1, YP_{—}522739.1, YP_{—}521788.1, YP_{—}004335162.1, YP_{—}004333708.1, YP_{—}004332973.1, YP_{—}004332349.1, YP_{—}004157731.1, YP_{—}004224204.1, YP_{—}003275673.1, YP_{—}003275371.1, YP_{—}003274979.1, YP_{—}003274924.1, YP_{—}003274705.1, YP_{—}956544.1, YP_{—}955502.1, YP_{—}955007.1, YP_{—}954887.1, YP_{—}954886.1, YP_{—}954859.1, YP_{—}954399.1, YP_{—}953715.1, YP_{—}953073.1, YP_{—}952592.1, YP_{—}951909.1, YP_{—}951298.1, YP_{—}951083.1, ZP_{—}08287899.1, ZP_{—}08272356.1, ZP_{—}08270967.1, CCA60099.1, CCA56737.1, YP_{—}983728.1, YP_{—}550833.1, YP_{—}549124.1, YP_{—}121795.1, YP_{—}120815.1, YP_{—}118589.1, YP_{—}117783.1, YP_{—}117375.1, YP_{—}003646883.1, YP_{—}003646055.1, YP_{—}003645661.1, EGE49469.1, ZP_{—}08234310.1, CBZ53121.1, YP_{—}004010866.1, EGE24961.1, EGE18726.1, EGE15701.1, EGE12950.1, EGE10026.1, EGB03968.1, ZP_{—}08206563.1, ZP_{—}08205089.1, ZP_{—}08204958.1, ZP_{—}08204416.1, ZP_{—}08203326.1, YP_{—}714381.1, YP_{—}713817.1, YP_{—}694462.1 (encoded by SEQ ID No.: 67), YP_{—}693524.1, YP_{—}003341775.1, YP_{—}003339587.1, ZP_{—}08197177.1, ADW01905.1, YP_{—}004242683.1, ZP_{—}07484742.2, ZP_{—}07441979.2, ZP_{—}07441978.2, ZP_{—}07437333.2, ZP_{—}06960424.1, ZP_{—}06801236.1, ZP_{—}06799517.1, ZP_{—}05769718.1, ZP_{—}05768326.1, ZP_{—}05767970.1, ZP_{—}05766272.1, ZP_{—}05763839.1, YP_{—}003204265.1, YP_{—}003203570.1, YP_{—}003200768.1, YP_{—}003134884.1, YP_{—}003134608.1, ZP_{—}05140320.1, NP_{—}001140997.1, EEE64643.1, EEE55448.1, EEE32548.1, ZP_{—}03534756.1, ZP_{—}03533653.1, ZP_{—}03531929.1, EEC71274.1, EAY98969.1, EAY75974.1, EAY75973.1, ADZ24988.1, ZP_{—}08157247.1, ZP_{—}08156660.1, ZP_{—}08156249.1, ZP_{—}08153292.1, ZP_{—}08152876.1, ZP_{—}08152662.1, YP_{—}002946672.1, YP_{—}960669.1, YP_{—}960629.1, YP_{—}960328.1, YP_{—}958134.1, YP_{—}957462.1, YP_{—}001022272.1, ZP_{—}08123690.1, ZP_{—}08120547.1, ZP_{—}08119498.1, EGB29195.1, EGB27143.1, YP_{—}003770089.1, YP_{—}003769971.1, YP_{—}003764703.1, YP_{—}003764513.1, YP_{—}003103950.1, YP_{—}003168536.1, YP_{—}003168331.1, YP_{—}003166844.1, CAJ88696.1, NP_{—}769520.1, YP_{—}001141853.1, YP_{—}001108534.1, YP_{—}001106516.1, YP_{—}907824.1, YP_{—}907344.1, YP_{—}906945.1, YP_{—}906856.1, YP_{—}906855.1, YP_{—}906831.1, YP_{—}906494.1, YP_{—}906243.1, YP_{—}905962.1, YP_{—}905765.1, YP_{—}905343.1, YP_{—}905239.1, YP_{—}325796.1, YP_{—}130413.1, NP_{—}625255.1, NP_{—}624462.1, NP_{—}338129.1, NP_{—}338004.1, NP_{—}337859.1, NP_{—}337740.1, NP_{—}337694.1, NP_{—}336266.1, NP_{—}335919.1, NP_{—}335351.1, NP_{—}334638.1, NP_{—}218257.1, NP_{—}218251.1, NP_{—}217997.1, NP_{—}217888.1, NP_{—}217751.1, NP_{—}217750.1, NP_{—}217646.1, NP_{—}217604.1, NP_{—}217603.1, NP_{—}217000.1, NP_{—}216801.1, NP_{—}216276.1, NP_{—}215941.1, NP_{—}215410.1, NP_{—}214735.1, ZP_{—}04661667.1, EFW44815.1, EFW44455.1, ZP_{—}08024634.1, ZP_{—}08024620.1, ZP_{—}08023777.1, ZP_{—}08023597.1, YP_{—}002784032.1, YP_{—}002783585.1, YP_{—}002782904.1, YP_{—}002782647.1, YP_{—}002780099.1, YP_{—}002779887.1, YP_{—}002778497.1, YP_{—}002777657.1, YP_{—}002777402.1, ZP_{—}07966321.1, ZP_{—}07944768.1, CBI21867.3, CBI40547.3, CBI40544.3, CBI40540.3, CBI40536.3, CBI40534.3, CBI40533.3, CBI32385.3, ZP_{—}05765756.1, ZP_{—}05765643.1, ZP_{—}05765597.1, ZP_{—}05765596.1, YP_{—}001705267.1, YP_{—}001704692.1, YP_{—}001704281.1, YP_{—}001702654.1, YP_{—}001701260.1, ZP_{—}05770434.1, ZP_{—}05766274.1, ZP_{—}05762133.1, ZP_{—}05762130.1, ZP_{—}01101223.1, YP_{—}481580.1, YP_{—}979623.1, YP_{—}979196.1, ZP_{—}07414300.2, ZP_{—}03537340.1, ZP_{—}03537339.1, ZP_{—}03536772.1, ZP_{—}03536404.1, ZP_{—}03433478.1, ZP_{—}03430367.1, ZP_{—}03430260.1, ZP_{—}03429345.1, ZP_{—}03428583.1, ZP_{—}03426905.1, ZP_{—}03426458.1, ZP_{—}03426456.1, ZP_{—}03426455.1, ZP_{—}03425014.1, ZP_{—}03424082.1, ZP_{—}03421649.1, ZP_{—}03419291.1, ZP_{—}03418394.1, ZP_{—}03417976.1, ZP_{—}03414875.1, ZP_{—}06952098.1, ZP_{—}05528769.1, ZP_{—}05527907.1, ZP_{—}05227984.1, ZP_{—}05227897.1, ZP_{—}05227653.1, ZP_{—}05227585.1, ZP_{—}05227420.1, ZP_{—}05227202.1, ZP_{—}05226387.1, ZP_{—}05226386.1, ZP_{—}05225355.1, ZP_{—}05225200.1, ZP_{—}05223431.1, ZP_{—}05223402.1, ZP_{—}04697793.1, ZP_{—}02550609.1, ZP_{—}02548969.1, EEE25493.1, ABO13188.2, ZP_{—}07205208.1, YP_{—}589436.1, BAJ33896.1, ZP_{—}07718107.1, ZP_{—}07717513.1, ZP_{—}07717390.1, ZP_{—}07716424.1, ZP_{—}04384387.1, ZP_{—}07376578.1, ZP_{—}06871097.1, ZP_{—}06852444.1, ZP_{—}06852442.1, ZP_{—}06852283.1, ZP_{—}06852150.1, ZP_{—}06852032.1, ZP_{—}06850980.1, ZP_{—}06850766.1, ZP_{—}06850644.1, ZP_{—}06849846.1, ZP_{—}06849446.1, ZP_{—}06849265.1, ZP_{—}06848894.1, ZP_{—}06848550.1, ZP_{—}06847321.1, ZP_{—}06847245.1, ZP_{—}06728640.1, ZP_{—}06155537.1, ZP_{—}03822106.1, ZP_{—}03822105.1, ZP_{—}03264909.1, ZP_{—}01915979.1, ZP_{—}01914209.1, ZP_{—}01909198.1, ZP_{—}01895985.1, ZP_{—}01893763.1, ZP_{—}01893601.1, ZP_{—}01893547.1, ZP_{—}01864269.1, ZP_{—}01736818.1, ZP_{—}01693481.1, ZP_{—}01626518.1, ZP_{—}01616172.1, ZP_{—}01461648.1, ZP_{—}01439861.1, ZP_{—}01311414.1, ZP_{—}01222733.1, ZP_{—}01038993.1, ZP_{—}00997001.1, ZP_{—}06533596.1, ZP_{—}07308012.1, ZP_{—}07282351.1, ZP_{—}07282257.1, ZP_{—}07278697.1, ZP_{—}07277986.1, ZP_{—}07277799.1, ZP_{—}07011797.1, ZP_{—}06913634.1, ZP_{—}06711075.1, ZP_{—}06575037.1, ZP_{—}06523715.1, ZP_{—}06522644.1, ZP_{—}06520408.1, ZP_{—}06518751.1, ZP_{—}06514733.1, ZP_{—}06511304.1, ZP_{—}06510466.1, ZP_{—}06509700.1, ZP_{—}06504004.1, ZP_{—}06452618.1, ZP_{—}06451687.1, ZP_{—}06450049.1, ZP_{—}06444722.1, ZP_{—}06443996.1, ZP_{—}06443677.1, ZP_{—}06438510.1, ZP_{—}06435077.1, ZP_{—}06434554.1, ZP_{—}06432969.1, ZP_{—}06431341.1, ZP_{—}06430915.1, ZP_{—}05129423.1, ZP_{—}05127637.1, ZP_{—}05126217.1, ZP_{—}05096686.1, ZP_{—}05095013.1, ZP_{—}05094400.1, ZP_{—}05093434.1, ZP_{—}05043539.1, ZP_{—}05041631.1, ZP_{—}04959394.1, ZP_{—}04956551.1, ZP_{—}01052702.1, YP_{—}437020.1, YP_{—}436128.1, YP_{—}432512.1, YP_{—}432391.1, ZP_{—}06072118.1, ZP_{—}06069021.1, ZP_{—}06065092.1, ZP_{—}06062254.1, YP_{—}003032200.1, YP_{—}003030813.1, YP_{—}002766854.1, YP_{—}002766842.1, YP_{—}002766292.1, YP_{—}002765623.1, YP_{—}002765076.1, YP_{—}002764977.1, YP_{—}002764976.1, YP_{—}002764693.1, YP_{—}002764633.1, YP_{—}002646305.1, YP_{—}002646304.1, YP_{—}001853537.1, YP_{—}001853530.1, YP_{—}001853214.1, YP_{—}001852100.1, YP_{—}001851711.1, YP_{—}001851686.1, YP_{—}001851684.1, YP_{—}001851611.1, YP_{—}001851610.1, YP_{—}001851579.1, YP_{—}001850950.1, YP_{—}001850935.1, YP_{—}001850900.1, YP_{—}001850899.1, YP_{—}001850378.1, YP_{—}001849911.1, YP_{—}001849825.1, YP_{—}001849624.1, YP_{—}001849470.1, YP_{—}001848848.1, YP_{—}001848784.1, YP_{—}001822237.1, YP_{—}001289190.1, YP_{—}001289078.1, YP_{—}001288434.1, YP_{—}001287727.1, YP_{—}001286168.1, YP_{—}001085790.1, YP_{—}856793.1, YP_{—}629387.1, YP_{—}615587.1, YP_{—}615252.1, YP_{—}457389.1, YP_{—}263530.1, NP_{—}962591.1, NP_{—}962411.1, NP_{—}962281.1, NP_{—}961234.1, NP_{—}960903.1, NP_{—}960387.1, NP_{—}960090.1, NP_{—}959281.1, NP_{—}959065.1, NP_{—}857403.1, NP_{—}857149.1, NP_{—}857148.1, NP_{—}857047.1, NP_{—}856907.1, NP_{—}856759.1, NP_{—}856156.1, NP_{—}855443.1, NP_{—}855112.1, NP_{—}853892.1, NP_{—}828432.1, NP_{—}603766.1, XP_{—}003081224.1, YP_{—}003778608.1, YP_{—}003730939.1, XP_{—}003059244.1, ADI13131.1, XP_{—}002992800.1, XP_{—}002963877.1, XP_{—}001419779.1, XP_{—}002988280.1, XP_{—}002987493.1, CBH32551.1, CBH32550.1, CBH19575.1, CBH19574.1, YP_{—}003627553.1, XP_{—}002879777.1, XP_{—}002877657.1, XP_{—}002877655.1, XP_{—}002873570.1, XP_{—}002871716.1, XP_{—}002870738.1, XP_{—}002868506.1, XP_{—}002865972.1, XP_{—}002864239.1, XP_{—}002862308.1, ZP_{—}05823139.1, NP_{—}001043877.1, ZP_{—}06693274.1, ZP_{—}06058985.1, NP_{—}001044374.1, XP_{—}002835451.1, XP_{—}002787542.1, XP_{—}002785958.1, XP_{—}002785645.1, XP_{—}002783220.1, XP_{—}002774061.1, XP_{—}002767852.1, XP_{—}002766051.1, XP_{—}002765456.1, XP_{—}002765455.1, XP_{—}002677788.1, XP_{—}002671612.1, XP_{—}002736281.1, CBA31373.1, XP_{—}002184474.1, XP_{—}002325936.1, XP_{—}002323705.1, XP_{—}002325937.1, XP_{—}002323911.1, XP_{—}002323706.1, XP_{—}002328965.1, XP_{—}002318416.1, XP_{—}002310400.1, ACY38597.1, ACY38596.1, ACY38595.1, ACY38594.1, ACY38593.1, ACY38592.1, ACY38591.1, ACY38590.1, ACX81315.1, ACX81314.1, XP_{—}001868729.1, XP_{—}001847517.1, XP_{—}001847515.1, XP_{—}002502575.1, ACU20370.1, ACU18073.1, XP_{—}002523348.1, XP_{—}002516707.1, XP_{—}002429016.1, BAH89673.1, XP_{—}002440221.1, XP_{—}002459294.1, XP_{—}002458560.1, XP_{—}320167.4, XP_{—}001780431.1, XP_{—}002364905.1, XP_{—}002263196.1, XP_{—}002263137.1, XP_{—}002263409.1, XP_{—}002263252.1, XP_{—}002268615.1, XP_{—}002278404.1, XP_{—}002274522.1, XP_{—}002282418.1, XP_{—}001633379.1, XP_{—}001632267.1, XP_{—}001632004.1, XP_{—}001622638.1, XP_{—}002155609.1, XP_{—}759225.1, XP_{—}002152406.1, XP_{—}001914129.1, XP_{—}001738032.1, XP_{—}001731626.1, XP_{—}001209859.1, CAN79451.1, CAN78449.1, CAN72806.1, CAN71951.1, CAN71950.1, CAN76656.1, CAN62907.1, AAZ08051.1, ABO21022.1, ABO21021.1, ABO21020.1, ABJ96321.1, BAF01088.1, XP_{—}758106.1, BAC42871.1, BAB09801.1, BAB09102.1, in particular YP_{—}045555.1 (encoded by SEQ ID No.: 19), YP_{—}694462.1 (encoded by SEQ ID No.: 67) and NP_{—}808414.2.
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥ is generally understood in particular as meaning the conversion of dodecanoyl-CoA thioester and/or dodecanoyl-ACP thioester with methanol into dodecanoyl methyl ester.

If the enzyme Eᵥ is an alcohol O-acyltransferase of EC 2.3.1.84, it is preferred that they are selected from among:

EGA72844.1, NP_{—}015022.1, S69991, AAP72991.1, EDN63695.1, BAA05552.1, AAP72992.1, S69992, AAP72995.1, XP_{—}002552712.1, XP_{—}001646876.1, XP_{—}002551954.1, EGA82692.1, EDN61766.1, EGA86689.1, EGA74966.1, AAU09735.1, NP_{—}011693.1, XP_{—}445666.1, BAA13067.1, AAP72993.1, EGA62172.1, XP_{—}455762.1, EGA58658.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥ is generally understood in particular as meaning the conversion of dodecanoyl-CoA thioester with methanol into dodecanoyl methyl ester.

Microorganisms which are preferred according to the invention are those which are obtained when the microorganisms listed hereinbelow featuring a third genetic modification within the meaning of the invention are employed as starting point by being equipped with a first and second genetic modification and, if appropriate, at least one further genetic modification within the meaning of the invention.

WO2007136762 A2 describes microorganisms which include a third genetic modification so that they are capable of forming more fatty acids and fatty acid derivatives, in particular fatty acid esters, wax esters, hydrocarbons and alkan-1-ols, from at least one simple carbon source in comparison with their wild type and which are preferably employed according to the invention, in particular on pages 2 to 4 and 21 to 24, FIGS. 2 to 4, exemplary embodiments 1, 2 and 5 to 7 and Claims 1, 2, 5, 6, 9 to 27 and 33. The document also describes enzymes Eᵥ which are preferred according to the invention and their sequences, in particular on pages 21 to 24, in Table 10 and FIG. 10.

### Specific Enzymes Eᵥₐ

In cells which are preferred according to the invention, the enzyme Eᵥₐ is one which comprises sequences selected from among YP_{—}001851637.1 (encoded by SEQ ID No.: 114) and proteins having a polypeptide sequence in which up to 60%, preferably up to 25%, particularly preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, particularly preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥₐ is generally understood in particular as meaning the conversion of lauric acid and S-adenosylmethionine to lauric acid methyl ester and S-adenosylhomocysteine.

### Specific Enzymes Eᵥᵢ

In cells which are preferred according to the invention, the enzyme Eᵥᵢ is one which comprises sequences selected from among YP_{—}001724804.1 (encoded by SEQ ID No.: 18)

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequence by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥᵢ is generally understood in particular as meaning the synthesis of dodecanoyl-CoA thioester.

### Specific Enzymes Eᵥᵢᵢ

Microorganisms which are preferred according to the invention are those which are obtained when the microorganisms listed hereinbelow featuring a third genetic modification within the meaning of the invention are employed as starting point by being equipped with a first and second genetic modification and, if appropriate, at least one further genetic modification within the meaning of the invention.

WO2010075483 A2 describes microorganisms which include a third genetic modification so that they are capable of forming more fatty acids and fatty acid derivatives, in particular fatty acids, fatty acid methyl esters, fatty acid ethyl esters, fatty alcohols, fatty alkyl acetates, fatty aldehydes, fatty amines, fatty amides, fatty sulphates, fatty ethers, ketones, alkanes, internal and terminal olefins, dicarboxylic acids, α,ω-dicarboxylic acids and α,ω-diols, from at least one simple carbon source in comparison with their wild type and which are preferably employed according to the invention, in particular in sections [0061] to [0090] and [0287] to [0367], FIGS. 1, 4 and 5, exemplary embodiments 1 to 38 and Claims 18 to 26. The document also describes enzymes Eᵥᵢᵢ which are preferred according to the invention and their sequences, in particular in sections [0012] to [0060], Tables 7, 17, 26 and 27, FIGS. 1, 44 to 47 and 55 to 59, exemplary embodiments 1 to 38 and Claims 1 to 17.

Fourth Genetic Modification for the Production of Alkan-1-ols, Alkan-1-als, Alkan-1-Amines, Alkanes, Olefins, Alken-1-als, Alken-1-ols and Alken-1-Amines from a Simple Carbon Source

In the event that the production of alkan-1-ols, alkan-1-als, alkan-1-amines and olefins is desired, it may be advantageous to suitably enzymatically reduce, aminate, decarboxylate or decarbonylate the corresponding carboxylic acids or carboxylic acid esters.

To this end, microorganisms which are preferred according to the invention include a fourth genetic modification which comprises an activity of at least one of the following, selected from the group

E_{iib} acyl-CoA (Coenzyme A):ACP (Acyl Carrier Protein) transacylase, which converts an ACP thioester into a CoA thioester or a CoA thioester into an ACP thioester,
Eᵥᵢ acyl-CoA (Coenzyme A) synthetase, preferably of EC 6.2.1.3, which catalyses the synthesis of an acyl-coenzyme A thioester,
Eᵥᵢᵢᵢ acyl-CoA (Coenzyme A) reductase, preferably of EC 1.2.1.42 or EC 1.2.1.50, which preferably catalyses the reduction of an acyl-coenzyme A thioester to give the corresponding alkan-1-al or alkan-1-ol,
Eᵢₓ fatty acid reductase (also fatty aldehyde dehydrogenase or arylaldehyde oxidoreductase), preferably of EC 1.2.1.3, EC 1.2.1.20 or EC 1.2.1.48, which preferably catalyses the reduction of an alkanoic acid to give the corresponding alkan-1-al,
Eₓ acyl-ACP (Acyl Carrier Protein) reductase, preferably of EC 1.2.1.80, which catalyses the reduction of an acyl-ACP thioester to give the corresponding alkan-1-al or alkan-1-ol,
Eₓᵢ cytochrome P450 fatty acid decarboxylase, which catalyses the conversion of an alkanoic acid with n carbon atoms into a corresponding terminal olefin with n−1 carbon atoms, in particular of dodecanoic acid to undec-10-enoic acid,
Eₓᵢᵢ alkan-1-al decarbonylase, which catalyses the conversion of an alkan-1-al (n carbon atoms) into a corresponding alkane (n−1 carbon atoms), and
Eₓᵢᵢᵢ alkan-1-al transaminase, which catalyses the conversion of an alkan-1-al into a corresponding alkan-1-amine,
which is increased in comparison with the enzymatic activity of the wild type of the microorganism.

In this context, it is especially preferred that the fourth genetic modification comprises combinations of increased activities of the enzymes selected from among

Eᵢₓ, Eₓ, Eₓᵢ, EᵥᵢEᵥᵢᵢᵢ, EᵥᵢEₓE_{iib}, EᵥᵢᵢᵢEₓᵢᵢ, EᵥᵢᵢᵢEₓᵢᵢᵢ, EᵢₓEₓᵢᵢ, EᵢₓEₓᵢᵢᵢ, EₓEₓᵢᵢ, EₓEₓᵢᵢᵢ, EᵥᵢEᵥᵢᵢᵢEₓᵢᵢ, EᵥᵢEᵥᵢᵢᵢEₓᵢᵢᵢ, EₓEₓᵢᵢEᵥᵢE_{iib} and EₓEₓᵢᵢᵢEᵥᵢE_{iib}.

Preferred enzymes E_{iib} in connection with the fourth genetic modification correspond to the enzymes E_{iib} emphasized above as being preferred in the context of the first and third genetic modification.

Preferred enzymes Eᵥᵢ in connection with the fourth genetic modification correspond to the enzymes Eᵥᵢ emphasized above as being preferred in the context of the third genetic modification.

### Specific Enzymes Eᵥᵢᵢᵢ

Microorganisms which are preferred according to the invention are those which are obtained when the microorganisms including a fourth genetic modification within the meaning of the invention listed hereinbelow are employed as starting point by being equipped with a first and second genetic modification and, if appropriate, at least one further genetic modification within the meaning of the invention.

WO2011008565 A1 describes microorganisms which include a fourth genetic modification so that they are capable of forming more fatty acids and fatty acid derivatives, in particular fatty acids, fatty aldehydes, fatty alcohols, alkanes and fatty acid esters, from at least one simple carbon source in comparison with their wild type and which are preferably employed according to the invention, in particular in sections [0021], [0103] to [0106], [0108] and [0129]. The document also describes enzymes Eᵥᵢᵢᵢ which are preferred according to the invention and their sequences, in particular in sections [0104] to [0106] and [0108] and [0129] and exemplary embodiment 11.

WO2008151149 A2 describes microorganisms which include a fourth genetic modification so that they are capable of forming more microbial oil in comparison with their wild type from at least one simple carbon source and which are preferably employed according to the invention, in particular in sections [0009], [0015] to [0037], [0053], [0071], [0171], [0174] to [0191], [0274] and [0396], Claims 53 to 114, 188 to 206 and 344 to 355 and Tables 1 to 3. The document also describes enzymes Eᵥᵢᵢᵢ which are preferred according to the invention and their sequences, in particular in sections [0255] to [0261] and [0269] and Tables 6 and 7.

WO2007136762 A2 describes microorganisms which include a fourth genetic modification so that they are capable of forming more fatty acids and fatty acid derivatives, in particular fatty acid esters, wax esters, hydrocarbons and fatty alcohols, from at least one simple carbon source in comparison with their wild type and which are preferably employed according to the invention, in particular on pages 2 to 4 and 19 to 20, FIGS. 2 to 4, exemplary embodiments 2 to 7 and Claims 4, 8 to 27 and 33. The document also describes enzymes Eᵥᵢᵢᵢ which are preferred according to the invention and their sequences, in particular on pages 19 to 20, in Table 10 and FIG. 10.

WO2011019858 A1 describes microorganisms which include a fourth genetic modification so that they are capable of forming more fatty acids and fatty acid derivatives, in particular fatty alcohols, from at least one simple carbon source in comparison with their wild type and which are preferably employed according to the invention, in particular in sections [0015] to [0020], [0064] to [0074], [0085] to [0086] and [0092] to [0099], exemplary embodiments 1 to 13, FIG. 1 and Claims 1 to 14. The document also describes enzymes Eᵥᵢᵢᵢ which are preferred according to the invention and their sequences, in particular in sections [0004] to [0007] and [0075] to [0080] and exemplary embodiments 1 to 13.

WO2009140695 A1 describes microorganisms which include a fourth genetic modification so that they are capable of forming more fatty acids and fatty acid derivatives, in particular hydrocarbons, from at least one simple carbon source in comparison with their wild type and which are preferably employed according to the invention in particular in sections [0031] to [0040], [0051] and [0214] to [0233], exemplary embodiments 5 to 24 and 28 to 30, Table 1, FIG. 40, and Claims 29 to 30. The document also describes enzymes Eᵥᵢᵢᵢ which are preferred according to the invention and their sequences, in particular in sections [0023] to [0030], [0056], [0066] to [0069] and [0193] to [0208], Table 1, FIG. 39, exemplary embodiments 5 to 24 and 28 to 30 and Claims 69 to 74.

WO2011008535 A1 describes microorganisms which include a fourth genetic modification so that they are capable of forming more fatty acids and fatty acid derivatives, in particular carboxylic acids, hydroxycarboxylic acids and their lactones from at least one simple carbon source in comparison with their wild type and which are preferably employed according to the invention, in particular in sections [0023] to [0024] and [0133] to [0158], FIG. 13, Claims 39 and 45 to 47 and exemplary embodiments 1 to 5. The document also describes enzymes Eᵥᵢᵢᵢ which are preferred according to the invention and their sequences, in particular in sections [0017] to [0022], [0084] to [0132], FIGS. 2 to 12, Claims 31 to 37 and 40 to 44 and exemplary embodiments 1 to 5.

WO2010063031 A2 describes microorganisms which include a fourth genetic modification so that they are capable of forming more microbial oil from at least one simple carbon source in comparison with their wild type and which are preferably employed according to the invention, in particular in sections [0007], [0092] to [0100], [0181] to [0183] and [0199] to [0213]. The document also describes enzymes Eᵥᵢᵢᵢ which are preferred according to the invention and their sequences, in particular in sections [0191] to [0194] and Tables 4 and 5.

WO2010063032 A2 describes microorganisms which include a fourth genetic modification so that they are capable of forming more microbial oil from at least one simple carbon source in comparison with their wild type and which are preferably employed according to the invention, in particular in sections [0007], [0092] to [0100], [0181] to [0183] and [0199] to [0213]. The document also describes enzymes Eᵥᵢᵢᵢ which are preferred according to the invention and their sequences, in particular in sections [0191] to [0194] and Tables 4 and 5.

### Specific Enzymes Eᵢₓ

In cells which are preferred according to the invention, the enzyme Eᵢₓ is one which comprises sequences selected from among YP_{—}887275.1 (encoded by SEQ ID No. 117), ABI83656.1 (encoded by SEQ ID No.: 122), and

proteins with a polypeptide sequence in which up to 60%, preferably up to 25%, particularly preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, particularly preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight) [U/g CDW]), compared with the activity of the biocatalyst without the presence of the reference protein, where activity in this context and in the context of the determination of the activity of the enzyme Eᵢₓ is generally understood in particular as meaning the synthesis of lauryl aldehyde, NADP, AMP and 2 Pᵢ from lauric acid, ATP, NADPH and H⁺.

Microorganisms which are preferred according to the invention are those which are obtained when the microorganisms including a fourth genetic modification within the meaning of the invention listed hereinbelow are employed as starting point by being equipped with a first and second genetic modification and, if appropriate, at least one further genetic modification within the meaning of the invention.

WO2011019858 A1 describes microorganisms which include a fourth genetic modification so that they are capable of forming more fatty acids and fatty acid derivatives, in particular fatty alcohols, from at least one simple carbon source in comparison with their wild type and which are preferably employed according to the invention, in particular in sections [0004] to [0008], [0064] to [0074], [0085] to [0086], [0095] to [0099]. The document also describes enzymes Eᵢₓ which are preferred according to the invention and their sequences, in particular in sections [0008] to [0009], [0074] and [0081] to [0082] and exemplary embodiments 1 to 13.

WO2010135624 A2 describes microorganisms which include a fourth genetic modification so that they are capable of forming more fatty acids and fatty acid derivatives, in particular carboxylic acids, hydroxycarboxylic acids and their lactones, from at least one simple carbon source in comparison with their wild type and which are preferably employed according to the invention, in particular in sections [0005], [0067] to [0085] and [0092] to [0102], Claims 13 to 17 and exemplary embodiments 1 to 4. The document also describes enzymes Eᵢₓ which are preferred according to the invention and their sequences, in particular in sections [0005] to [0006] and [0086] to [0090], FIGS. 3 to 7, Claim 28 and exemplary embodiments 1 to 4.

WO2010062480 A2 describes microorganisms which include a fourth genetic modification so that they are capable of forming more fatty acids and fatty acid derivatives, in particular fatty alcohols, from at least one simple carbon source in comparison with their wild type and which are preferably employed according to the invention, in particular in sections [0022] to [0174] and [0292] to [0316], exemplary embodiments 1 and 3 to 8, FIG. 9 and Claims 17 and 24. The document also describes enzymes Eᵢₓ which are preferred according to the invention and their sequences, in particular in sections [0019] to [0032] and [0263] to [0286], Table 1, FIGS. 6 to 8 and exemplary embodiments 1 and 3 to 8.

WO201042664 A2 describes microorganisms which include a fourth genetic modification so that they are capable of forming more fatty acids and fatty acid derivatives, in particular fatty alcohols, from at least one simple carbon source in comparison with their wild type and which are preferably employed according to the invention, in particular in sections [0236] to [0261], exemplary embodiment 2, FIGS. 1 and 5 and Claim 25. The document also describes enzymes Eᵢₓ which are preferred according to the invention and their sequences, in particular in sections [0211] to [0233], FIGS. 2 to 4 and exemplary embodiments 1 to 2.

### Specific Enzymes Eₓ

In cells which are preferred according to the invention, the enzyme Eₓ is one which comprises sequences selected from among BAB85476.1 (encoded by SEQ ID No. 77), YP_{—}047869.1 (encoded by SEQ ID No. 79 or 81), YP_{—}959486.1 (encoded by SEQ ID No. 83), YP_{—}959769.1 (encoded by SEQ ID No. 139), B9TSP7.1 (encoded by SEQ ID No. 141), and proteins with a polypeptide sequence in which up to 60%, preferably up to 25%, particularly preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, particularly preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eₓ is generally understood in particular as meaning the synthesis of lauryl alcohol and NAD(P)⁺ from lauryl-ACP, NAD(P)H and H⁺.

Microorganisms which are preferred according to the invention are those which are obtained when the microorganisms including a fourth genetic modification within the meaning of the invention listed hereinbelow are employed as starting point by being equipped with a first and second genetic modification and, if appropriate, at least one further genetic modification within the meaning of the invention.

WO2007136762 A2 describes microorganisms which include a fourth genetic modification so that they are capable of forming more fatty acids and fatty acid derivatives, in particular fatty acid esters, wax esters, hydrocarbons and fatty alcohols, from at least one simple carbon source in comparison with their wild type and which are preferably employed according to the invention, in particular on pages 2 to 4 and 19 to 20, FIGS. 2 to 4, exemplary embodiments 2 to 7 and Claims 4, 8 to 27 and 33. The document also describes enzymes Eₓ which are preferred according to the invention and their sequences, in particular on pages 19 to 20, in Table 10 and FIG. 10.

WO2011019858 A1 describes microorganisms which include a fourth genetic modification so that they are capable of forming more fatty acids and fatty acid derivatives, in particular fatty alcohols, from at least one simple carbon source in comparison with their wild type and which are preferably employed according to the invention, in particular in sections [0015] to [0020], [0064] to [0074], [0085] to [0086] and [0092] to [0099], exemplary embodiments 1 to 13, FIG. 1 and Claims 1 to 14. The document also describes enzymes Eₓ which are preferred according to the invention and their sequences, in particular in sections [0004] to [0007] and [0075] to [0080] and exemplary embodiments 1 to 13.

WO2009140695 A1 describes microorganisms which include a fourth genetic modification so that they are capable of forming more fatty acids and fatty acid derivatives, in particular hydrocarbons, from at least one simple carbon source in comparison with their wild type and which are preferably employed according to the invention, in particular in sections [0031] to [0040], [0051] and [0214] to [0233], exemplary embodiments 5 to 24 and 28 to 30, Table 1, FIG. 40, and Claims 29 to 30. The document also describes enzymes Eₓ which are preferred according to the invention and their sequences, in particular in sections [0023] to [0030], [0056], [0066] to [0069] and [0193] to [0208], Table 1, FIG. 39, exemplary embodiments 5 to 24 and 28 to 30 and Claims 69 to 74.

WO2011008535 A1 describes microorganisms which include a fourth genetic modification so that they are capable of forming more fatty acids and fatty acid derivatives, in particular carboxylic acids, hydroxycarboxylic acids and their lactones, from at least one simple carbon source in comparison with their wild type and which are preferably employed according to the invention, in particular in sections [0023] to [0024] and [0133] to [0158], FIG. 13, Claims 39 and 45 to 47 and exemplary embodiments 1 to 5. The document also describes enzymes Eₓ which are preferred according to the invention and their sequences, in particular in sections [0017] to [0022], [0084] to [0132], FIGS. 2 to 12, Claims 31 to 37 and 40 to 44 and exemplary embodiments 1 to 5.

### Specific Enzymes Eₓᵢ

In cells which are preferred according to the invention, the enzyme Eₓᵢ is one which comprises sequences selected from among ADW41779.1 (encoded by SEQ ID No. 168) and proteins with a polypeptide sequence in which up to 60%, preferably up to 25%, particularly preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, particularly preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context the determination of the activity of the enzyme Eₓᵢᵢᵢ is generally understood in particular as meaning the reaction of sodium palmitate with hydrogen peroxide to form pentadecene, CO₂ and water.

Microorganisms which are preferred according to the invention are those which are obtained when the microorganisms including a fourth genetic modification within the meaning of the invention listed hereinbelow are employed as starting point by being equipped with a first and second genetic modification and, if appropriate, at least one further genetic modification within the meaning of the invention.

WO2009085278 A1 describes microorganisms which include a fourth genetic modification so that they are capable of forming more fatty acids and fatty acid derivatives, in particular olefins, from at least one simple carbon source in comparison with their wild type and which are preferred according to the invention, in particular in sections [0033] to [0048], [0056] to [0063] and [0188] to [0202], FIG. 10, Table 8, exemplary embodiments 5 to 18 and Claims 28 to 51 and 188 to 195. The document also describes enzymes Eₓᵢ which are preferred according to the invention and their sequences, in particular in sections [0021] to [0032], [0051] to [0055], [0081] to [0084] and [0160] to [0183], Table 8, exemplary embodiments 5 to 18, Claims 1 to 25 and FIGS. 3, 7 and 9.

### Specific Enzymes Eₓᵢᵢ

Microorganisms which are preferred according to the invention are those which are obtained when the microorganisms including a fourth genetic modification within the meaning of the invention listed hereinbelow are employed as starting point by being equipped with a first and second genetic modification and, if appropriate, at least one further genetic modification within the meaning of the invention.

WO2009140695 A1 describes microorganisms which include a fourth genetic modification so that they are capable of forming more fatty acids and fatty acid derivatives, in particular hydrocarbons, from at least one simple carbon source in comparison with their wild type and which are preferred according to the invention, in particular in sections [0031] to [0040], [0051] and [0214] to [0233], exemplary embodiments 5 to 24 and 28 to 30, Table 1, FIG. 40, and Claims 29 to 30. The document also describes enzymes Eₓᵢᵢ which are preferred according to the invention and their sequences, in particular in sections [0023] to [0030], [0056], [0066] to [0069] and [0193] to [0208], Table 1, FIG. 38, exemplary embodiments 5 to 24 and 28 to 30 and Claims 69 to 74.

WO2008151149 A2 describes microorganisms which include a fourth genetic modification so that they are capable of forming more microbial oil, from at least one simple carbon source in comparison with their wild type and which are preferred according to the invention, in particular in sections [0009], [0015] to [0037], [0053], [0071], [0171], [0174] to [0191], [0274] and [0396], Claims 53 to 114, 188 to 206 and 344 to 355 and Tables 1 to 3. The document also describes enzymes Eₓᵢᵢ which are preferred according to the invention and their sequences, in particular in Table 8.

### Specific Enzymes Eₓᵢᵢᵢ

The enzyme Eₓᵢᵢᵢ is preferably according to the invention an ω-transaminase of EC 2.6.1.-. Preferred enzymes Eₓᵢᵢᵢ are selected from the group:

3HMU_A, AAD41041.1, AAK15486.1, ABE03917.1, ADR60699.1, ADR61066.1, ADR62525.1, AEL07495.1, CAZ86955.1, EFW82310.1, EFW87681.1, EGC99983.1, EGD03176.1, EGE58369.1, EGH06681.1, EGH08331.1, EGH24301.1, EGH32343.1, EGH46412.1, EGH55033.1, EGH62152.1, EGH67339.1, EGH70821.1, EGH71404.1, EGH78772.1, EGH85312.1, EGH97105.1, EGP57596.1, NP_{—}102850.1, NP_{—}106560.1, NP_{—}248912.1, NP_{—}248990.1, NP_{—}354026.2, NP 421926.1, NP_{—}637699.1, NP_{—}642792.1, NP_{—}744329.1, NP_{—}744732.1, NP_{—}747283.1, NP_{—}795039.1, NP_{—}901695.1 (encoded by SEQ ID No. 132), XP_{—}002943905.1, YP_{—}001021095.1, YP_{—}001059677.1, YP_{—}001061726.1, YP_{—}001066961.1, YP_{—}001074671.1, YP_{—}001120907.1, YP_{—}001140117.1, YP_{—}001170616.1, YP_{—}001185848.1, YP_{—}001188121.1, YP_{—}001233688.1, YP_{—}001268866.1, YP_{—}001270391.1, YP_{—}001345703.1, YP_{—}001412573.1, YP_{—}001417624.1, YP_{—}001526058.1, YP_{—}001579295.1, YP_{—}001581170.1, YP_{—}001668026.1, YP_{—}001669478.1, YP_{—}001671460.1, YP_{—}001685569.1, YP_{—}001747156.1, YP_{—}001749732.1, YP_{—}001765463.1, YP_{—}001766294.1, YP_{—}001790770.1, YP_{—}001808775.1, YP_{—}001809596.1, YP_{—}001859758.1, YP_{—}001888405.1, YP_{—}001903233.1, YP_{—}001977571.1, YP_{—}002229759.1, YP_{—}002231363.1, YP_{—}002280472.1, YP_{—}002297678.1, YP_{—}002543874.1, YP_{—}002549011.1, YP_{—}002796201.1, YP_{—}002801960.1, YP_{—}002875335.1, YP_{—}002897523.1, YP_{—}002912290.1, YP_{—}002974935.1, YP_{—}003060891.1, YP_{—}003264235.1, YP_{—}003552364.1, YP_{—}003578319.1, YP_{—}003591946.1, YP_{—}003607814.1, YP_{—}003641922.1, YP_{—}003674025.1, YP_{—}003692877.1, YP_{—}003755112.1, YP_{—}003896973.1, YP_{—}003907026.1, YP_{—}003912421.1, YP_{—}004086766.1, YP_{—}004142571.1, YP_{—}004147141.1, YP_{—}004228105.1, YP_{—}004278247.1, YP_{—}004305252.1, YP_{—}004356916.1, YP_{—}004361407.1, YP_{—}004378186.1, YP_{—}004379856.1, YP_{—}004390782.1, YP_{—}004472442.1, YP_{—}004590892.1, YP_{—}004612414.1, YP_{—}004676537.1, YP_{—}004693233.1, YP_{—}004701580.1, YP_{—}004701637.1, YP_{—}004704442.1, YP_{—}108931.1, YP_{—}110490.1, YP_{—}168667.1, YP_{—}237931.1, YP_{—}260624.1, YP_{—}262985.1, YP_{—}271307.1, YP_{—}276987.1, YP_{—}334171.1, YP_{—}337172.1, YP_{—}350660.1, YP_{—}351134.1, YP_{—}364386.1, YP_{—}366340.1, YP_{—}369710.1, YP_{—}370582.1, YP_{—}426342.1, YP_{—}440141.1, YP_{—}442361.1, YP_{—}468848.1, YP_{—}521636.1, YP_{—}554363.1, YP_{—}608454.1, YP_{—}610700.1, YP_{—}614980.1, YP_{—}622254.1, YP_{—}625753.1, YP_{—}680590.1, YP_{—}751687.1, YP_{—}767071.1, YP_{—}774090.1, YP_{—}774932.1, YP_{—}788372.1, YP_{—}858562.1, YP_{—}928515.1, YP_{—}983084.1, YP_{—}995622.1, ZP_{—}00948889.1, ZP_{—}00954344.1, ZP_{—}00959736.1, ZP_{—}00998881.1, ZP_{—}01011725.1, ZP_{—}01037109.1, ZP_{—}01058030.1, ZP_{—}01076707.1, ZP_{—}01103959.1, ZP_{—}01167926.1, ZP_{—}01224713.1, ZP_{—}01442907.1, ZP_{—}01446892.1, ZP_{—}01550953.1, ZP_{—}01625518.1, ZP_{—}01745731.1, ZP_{—}01750280.1, ZP_{—}01754305.1, ZP_{—}01763880.1, ZP_{—}01769626.1, ZP_{—}01865961.1, ZP_{—}01881393.1, ZP_{—}01901558.1, ZP_{—}02145337.1, ZP_{—}02151268.1, ZP_{—}02152332.1, ZP_{—}02167267.1, ZP_{—}02190082.1, ZP_{—}02242934.1, ZP_{—}02360937.1, ZP_{—}02367056.1, ZP_{—}02385477.1, ZP_{—}02456487.1, ZP_{—}02883670.1, ZP_{—}03263915.1, ZP_{—}03263990.1, ZP_{—}03400081.1, ZP_{—}03452573.1, ZP_{—}03456092.1, ZP_{—}03517291.1, ZP_{—}03529055.1, ZP_{—}03571515.1, ZP_{—}03572809.1, ZP_{—}03587785.1, ZP_{—}03588560.1, ZP_{—}03697266.1, ZP_{—}03697962.1, ZP_{—}04521092.1, ZP_{—}04590693.1, ZP_{—}04890914.1, ZP_{—}04891982.1, ZP_{—}04893793.1, ZP_{—}04902131.1, ZP_{—}04905327.1, ZP_{—}04941068.1, ZP_{—}04944536.1, ZP_{—}04945255.1, ZP_{—}04959332.1, ZP_{—}04964181.1, ZP_{—}05053721.1, ZP_{—}05063588.1, ZP_{—}05073059.1, ZP_{—}05077806.1, ZP_{—}05082750.1, ZP_{—}05091128.1, ZP_{—}05095488.1, ZP_{—}05101701.1, ZP_{—}05116783.1, ZP_{—}05121836.1, ZP_{—}05127756.1, ZP_{—}05637806.1, ZP_{—}05742087.1, ZP_{—}05783548.1, ZP_{—}05786246.1, ZP_{—}05843149.1, ZP_{—}05945960.1, ZP_{—}06459045.1, ZP_{—}06487195.1, ZP_{—}06492453.1, ZP_{—}06493162.1, ZP_{—}06703644.1, ZP_{—}06731146.1, ZP_{—}06839371.1, ZP_{—}07007312.1, ZP_{—}07266194.1, ZP_{—}07374050.1, ZP_{—}07662787.1, ZP_{—}07778196.1, ZP_{—}07797983.1, ZP_{—}08099459.1, ZP_{—}08138203.1, ZP_{—}08141719.1, ZP_{—}08142973.1, ZP_{—}08177102.1, ZP_{—}08185821.1, ZP_{—}08186468.1, ZP_{—}08208888.1, ZP_{—}08266590.1, ZP_{—}08402041.1, ZP_{—}08406891.1, ZP_{—}08522175.1, ZP_{—}08527488.1, ZP_{—}08631252.1, ZP_{—}08636687.1,
in particular NP_{—}901695.1 (encoded by SEQ ID No. 132), ZP_{—}03697266.1, AAD41041.1, YP_{—}002796201.1, ZP_{—}03697962.1, YP_{—}001859758.1, YP_{—}002229759.1, YP_{—}001120907.1, YP_{—}110490.1, ZP_{—}04964181.1, YP_{—}774932.1, YP_{—}001766294.1, YP_{—}001581170.1, YP_{—}622254.1, ZP_{—}03588560.1, YP_{—}001809596.1, YP_{—}370582.1, ZP_{—}03572809.1, NP_{—}248990.1, YP_{—}001888405.1, ZP_{—}04905327.1, YP_{—}001061726.1, YP_{—}001668026.1, ZP_{—}01750280.1, ZP_{—}07778196.1, EGH71404.1, NP_{—}744329.1, YP_{—}004147141.1, ADR61066.1, ZP_{—}05783548.1, YP_{—}004701637.1, YP_{—}366340.1, YP_{—}003264235.1, EGD03176.1, YP_{—}001268866.1, ZP_{—}01901558.1, ZP_{—}05121836.1, YP_{—}003692877.1, ZP_{—}03517291.1, YP_{—}002974935.1, YP_{—}001668026.1, ADR61066.1, NP_{—}744329.1, YP_{—}001268866.1, YP_{—}004701637.1, ZP_{—}08142973.1, ADR62525.1, YP_{—}610700.1, NP_{—}747283.1, ADR62525.1, YP_{—}001270391.1, YP_{—}004704442.1, YP_{—}610700.1, YP_{—}001747156.1, ZP_{—}08138203.1, ZP_{—}07266194.1, EGH70821.1, YP_{—}351134.1, EGH32343.1, EGH08331.1, EGH67339.1, YP_{—}001668026.1, YP_{—}004701637.1, YP_{—}237931.1, ZP_{—}03400081.1, ZP_{—}05116783.1, ZP_{—}01550953.1, ZP_{—}07662787.1, YP_{—}928515.1, YP_{—}788372.1, YP_{—}001021095.1, ZP_{—}07797983.1, YP_{—}003578319.1, YP_{—}004305252.1, NP_{—}248912.1, ZP_{—}08636687.1, YP_{—}003912421.1, YP_{—}751687.1, ZP_{—}08142973.1, YP_{—}271307.1, ZP_{—}05082750.1, YP_{—}001417624.1, YP_{—}353455.1,
and especially preferably NP_{—}901695.1 (encoded by SEQ ID No. 132), YP_{—}353455.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eₓᵢᵢᵢ is generally understood in particular as meaning the conversion of ω-oxo lauric acid and/or its methyl ester to give ω-amino lauric acid and/or its methyl ester.

### Eₓᵢᵥ, Auxiliary Enzyme for Eₓᵢᵢᵢ

For an increased activity of the enzyme Eₓᵢᵢᵢ, it may be beneficial to employ, in place of the enzyme Eₓᵢᵢᵢ alone, the combination of an enzyme Eₓᵢᵢᵢ paired with an enzyme Eₓᵢᵥ, which catalyses the conversion of an α-keto carboxylic acid to an amino acid, the enzyme Eₓᵢᵥ is preferably an amino acid dehydrogenase, such as, for example, serine dehydrogenases, aspartate dehydrogenases, phenylalanine dehydrogenases and glutamate dehydrogenases, especially preferably an alanine dehydrogenase of EC 1.4.1.1.

Such preferred alanine dehydrogenases are selected from among

EGR93259.1, YP_{—}004743277.1, YP_{—}004741620.1, YP_{—}004737294.1, YP_{—}002509853.1, YP_{—}002492255.1, YP_{—}002489845.1, YP_{—}002481919.1, YP_{—}001819330.1, YP_{—}004728333.1, ZP_{—}08670930.1, YP_{—}004672392.1, YP_{—}004467026.1, YP_{—}004326214.1, YP_{—}002349951.1, YP_{—}001674437.1, YP_{—}003921585.1, YP_{—}001699731.1, YP_{—}004720756.1, YP_{—}004719515.1, EGQ22316.1, EGQ21760.1, YP_{—}004689232.1, YP_{—}004698526.1, YP_{—}004694875.1, EGP67576.1, YP_{—}001832691.1, YP_{—}001760857.1, AEJ53875.1, AEJ42949.1, YP_{—}004392931.1, YP_{—}004404798.1, YP_{—}004374160.1, YP_{—}004303162.1, YP_{—}004196134.1, YP_{—}004178581.1, YP_{—}004163857.1, YP_{—}004161555.1, YP_{—}004099081.1, YP_{—}004101986.1, YP_{—}004042336.1, YP_{—}003994181.1, YP_{—}003966543.1, YP_{—}003913256.1, YP_{—}003825828.1, YP_{—}003806106.1, YP_{—}003686355.1, YP_{—}003678575.1, YP_{—}003654745.1, YP_{—}003651439.1, YP_{—}003637111.1, YP_{—}003631815.1, YP_{—}003300711.1, YP_{—}002886396.1, ZP_{—}03493991.1, YP_{—}001890813.1, YP_{—}001888849.1, YP_{—}001554753.1, YP_{—}001529018.1, YP_{—}001528954.1, YP_{—}001502090.1, YP_{—}001412833.1, YP_{—}001363812.1, YP_{—}923679.1, NP_{—}440110.1, ZP_{—}08640273.1, ZP_{—}08639751.1, ZP_{—}08637916.1, YP_{—}004171395.1, YP_{—}001366419.1, YP_{—}001327051.1, YP_{—}001262560.1, YP_{—}886996.1, YP_{—}882850.1, YP_{—}704410.1, YP_{—}703508.1, ZP_{—}08624689.1, YP_{—}001230376.1, P17557.1, P17556.1, CCB94892.1, CCB73698.1, YP_{—}001168635.1, YP_{—}004668736.1, YP_{—}911378.1, YP_{—}003686997.1, YP_{—}002263235.1, NP_{—}820115.1, YP_{—}004653761.1, YP_{—}004651159.1, YP_{—}003869397.1, YP_{—}004641708.1, YP_{—}004641134.1, YP_{—}001996597.1, YP_{—}001998297.1, YP_{—}001943676.1, YP_{—}001810799.1, YP_{—}004630087.1, YP_{—}004621893.1, YP_{—}004613083.1, ZP_{—}08621144.1, YP_{—}003954200.1, YP_{—}001372688.1, YP_{—}001233686.1, ZP_{—}08594848.1, ZP_{—}08586665.1, ZP_{—}08578896.1, ZP_{—}08575937.1, YP_{—}004604438.1, YP_{—}004600931.1, ZP_{—}08569139.1, ZP_{—}08566255.1, AEB25326.1, YP_{—}374584.1, YP_{—}004216732.1, ZP_{—}06806151.1, ZP_{—}06440291.1, ZP_{—}06369993.1, ZP_{—}06254238.1, ZP_{—}05844252.1, ZP_{—}05472927.1, ZP_{—}05365401.1, ZP_{—}04747945.1, ZP_{—}04678933.1, ZP_{—}03779761.1, ZP_{—}03728859.1, ZP_{—}03711891.1, ZP_{—}03697269.1, ZP_{—}01628294.1, ZP_{—}01546224.1, ZP_{—}01444021.1, ZP_{—}01308570.1, ZP_{—}01228194.1, ZP_{—}01164841.1, ZP_{—}01114638.1, YP_{—}004566582.1, YP_{—}004572166.1, YP_{—}004571401.1, YP_{—}004569425.1, YP_{—}003513168.1, YP_{—}004561169.1, ZP_{—}08554945.1, YP_{—}400777.1, ZP_{—}08533479.1, ZP_{—}08533412.1, ZP_{—}08525779.1, ZP_{—}08523693.1, YP_{—}004471329.1, YP_{—}004368103.1, YP_{—}001536790.1, YP_{—}001158763.1, YP_{—}662032.1, YP_{—}967824.1, YP_{—}004542206.1, YP_{—}002958019.1, YP_{—}645630.1, ZP_{—}08520595.1, AEG81976.1, YP_{—}002560779.1, YP_{—}496956.1, YP_{—}411850.1, YP_{—}300065.1, NP_{—}840123.1, ZP_{—}08514775.1, YP_{—}002250769.1, YP_{—}002155665.1, YP_{—}002137991.1, YP_{—}001135275.1, YP_{—}001070365.1, YP_{—}639268.1, NP_{—}864377.1, YP_{—}004554709.1, YP_{—}004546384.1, YP_{—}004544159.1, ZP_{—}01448725.1, ZP_{—}01255407.1, EGL88594.1, EGL87587.1, YP_{—}004536059.1, ZP_{—}08512666.1, ZP_{—}08501410.1, ZP_{—}08493566.1, ZP_{—}08486369.1, YP_{—}004497891.1, YP_{—}004494473.1, YP_{—}003945301.1, YP_{—}003835539.1, YP_{—}003634898.1, YP_{—}003503876.1, ZP_{—}06503131.1, YP_{—}003376450.1, YP_{—}003409976.1, YP_{—}003409004.1, YP_{—}003395275.1, YP_{—}003393138.1, YP_{—}003387714.1, YP_{—}003382934.1, ZP_{—}05760008.1, ZP_{—}05300490.1, ZP_{—}04387987.1, ZP_{—}03725713.1, YP_{—}002134125.1, YP_{—}001618802.1, ZP_{—}01899015.1, ZP_{—}01881250.1, ZP_{—}01731833.1, YP_{—}004529602.1, YP_{—}004512974.1, YP_{—}004479110.1, YP_{—}004434722.1, YP_{—}004430602.1, CBX28458.1, ZP_{—}05217624.1, ZP_{—}01074124.1, ZP_{—}01062209.1, ZP_{—}01011939.1, ZP_{—}00956754.1, YP_{—}388045.1, ZP_{—}07910902.1, ZP_{—}07835291.1, ZP_{—}07831081.1, ZP_{—}07704117.1, ZP_{—}07112933.1, ZP_{—}06860168.1, ZP_{—}05915689.1, YP_{—}002352943.1, YP_{—}826544.1, YP_{—}004087624.1, ADP99134.1, YP_{—}003590847.1, YP_{—}003589189.1, YP_{—}001192379.1, ZP_{—}08473868.1, ZP_{—}08469833.1, ZP_{—}08462614.1, ZP_{—}07709417.1, ZP_{—}07672507.1, ZP_{—}07608107.1, ZP_{—}07404685.1, ZP_{—}07334010.1, ZP_{—}07333254.1, ZP_{—}06888732.1, ZP_{—}06837313.1, YP_{—}873046.1, YP_{—}004060177.1, YP_{—}004007860.1, YP_{—}003492711.1, ZP_{—}08456143.1, YP_{—}003675989.1, YP_{—}003159562.1, NP_{—}302068.1, YP_{—}004461013.1, ZP_{—}08426378.1, ZP_{—}08422563.1, YP_{—}004122643.1, YP_{—}004077807.1, YP_{—}004058618.1, YP_{—}004055696.1, YP_{—}003898888.1, YP_{—}003575339.1, ZP_{—}06186049.1, YP_{—}003314861.1, YP_{—}003148148.1, YP_{—}002786543.1, YP_{—}001661762.1, YP_{—}001666058.1, YP_{—}001549204.1, YP_{—}001518627.1, YP_{—}004453289.1, YP_{—}004450492.1, YP_{—}004301609.1, YP_{—}465316.1, ZP_{—}08411512.1, YP_{—}001394062.1, YP_{—}001035553.1, YP_{—}417038.1, YP_{—}301147.1, YP_{—}014199.1, EGJ45059.1, EGJ36821.1, EGJ36552.1, EGJ19019.1, ZP_{—}08388916.1, YP_{—}004427278.1, YP_{—}003909234.1, YP_{—}002536659.1, YP_{—}001940410.1, YP_{—}001329977.1, YP_{—}001323343.1, YP_{—}001114195.1, YP_{—}001096594.1, YP_{—}949547.1, YP_{—}756289.1, YP_{—}722774.1, YP_{—}525283.1, YP_{—}461225.1, YP_{—}320697.1, YP_{—}289022.1, YP_{—}075651.1, NP_{—}988633.1, YP_{—}004399762.1, YP_{—}004335185.1, ADX76365.1, YP_{—}004203407.1, YP_{—}001917832.1, YP_{—}001642542.1, ZP_{—}08332142.1, YP_{—}041174.1, ZP_{—}08328264.1, YP_{—}004225082.1, EGG96712.1, ZP_{—}08311476.1, ZP_{—}08310170.1, ZP_{—}08267322.1, ZP_{—}08263846.1, ZP_{—}07898723.1, YP_{—}003273311.1, ZP_{—}05909597.1, YP_{—}003073095.1, YP_{—}003022905.1, YP_{—}003013384.1, YP_{—}003011072.1, ZP_{—}04777180.1, ZP_{—}04432601.1, YP_{—}001016505.1, YP_{—}953175.1, YP_{—}731492.1, ZP_{—}08302086.1, ZP_{—}08296718.1, ZP_{—}08285373.1, ZP_{—}08280138.1, ZP_{—}08270040.1, ZP_{—}08261780.1, ZP_{—}08258406.1, ZP_{—}08246570.1, YP_{—}003113209.1, YP_{—}002436565.1, ZP_{—}04409790.1, YP_{—}428767.1, EGG40837.1, CCA54694.1, YP_{—}004147180.1, YP_{—}550034.1, YP_{—}173042.1, EGF75662.1, YP_{—}004205024.1, YP_{—}003670363.1, YP_{—}003476027.1, YP_{—}003241464.1, YP_{—}863990.1, YP_{—}004149630.1, YP_{—}003646700.1, EGF24326.1, BAK15593.1, YP_{—}003991014.1, YP_{—}003988127.1, YP_{—}003722297.1, YP_{—}003254539.1, YP_{—}003251916.1, NP_{—}901692.1, EGF16043.1, EGF07290.1, YP_{—}003048854.1, YP_{—}149301.1, YP_{—}148605.1, YP_{—}004340432.1, EFT09946.1, EFS80513.1, EFS51332.1, EFS42459.1, YP_{—}003060895.1, YP_{—}003059033.1, ZP_{—}03305373.1, YP_{—}002379520.1, YP_{—}372555.1, NP_{—}085655.1, YP_{—}004321492.1, ZP_{—}08239446.1, YP_{—}003817108.1, YP_{—}002951286.1, YP_{—}002950656.1, YP_{—}002522266.1, YP_{—}001982538.1, YP_{—}001127463.1, YP_{—}001126767.1, NP_{—}764939.1, NP_{—}761756.1, NP_{—}244046.1, NP_{—}243195.1, YP_{—}003194671.1, YP_{—}003161559.1, YP_{—}002797803.1, YP_{—}002634404.1, YP_{—}439119.1, YP_{—}314402.1, YP_{—}143482.1, NP_{—}295618.1, ZP_{—}08215173.1, YP_{—}004282846.1, YP_{—}004267961.1, YP_{—}001867313.1, YP_{—}001301882.1, YP_{—}847214.1, YP_{—}004095847.1, YP_{—}003338282.1, YP_{—}003337256.1, YP_{—}355846.1, YP_{—}253131.1, ZP_{—}08197563.1, ZP_{—}08196283.1, ADW06447.1, YP_{—}003370508.1, YP_{—}003317645.1, YP_{—}003184411.1, YP_{—}003198349.1, YP_{—}003084639.1, YP_{—}004294565.1, YP_{—}004243057.1, CBZ55377.1, EGC26795.1, EGC25718.1, EGC23378.1, ZP_{—}07887872.1, YP_{—}003269716.1, YP_{—}003203632.1, YP_{—}003199972.1, YP_{—}003153148.1, YP_{—}003146304.1, YP_{—}002893498.1, ZP_{—}03230841.1, ZP_{—}03229411.1, YP_{—}001050520.1, YP_{—}963387.1, YP_{—}927645.1, YP_{—}869684.1, YP_{—}734091.1, NP_{—}372233.1, NP_{—}102173.1, ZP_{—}08170259.1, EGD36706.1, EGD32748.1, ZP_{—}08155540.1, YP_{—}004142849.1, YP_{—}002417649.1, YP_{—}001301040.1, YP_{—}001211208.1, YP_{—}266230.1, ZP_{—}08145165.1, YP_{—}001801454.1, YP_{—}001736003.1, YP_{—}833487.1, YP_{—}831236.1, YP_{—}384064.1, YP_{—}094958.1, YP_{—}009793.1, NP_{—}975075.1, NP_{—}847074.1, EGC82166.1, YP_{—}004261609.1, YP_{—}004255502.1, YP_{—}678603.1, YP_{—}004181700.1, ZP_{—}08122013.1, ADT87541.1, YP_{—}003524764.1, YP_{—}002992990.1, YP_{—}002992892.1, YP_{—}081348.1, YP_{—}080482.1, YP_{—}002476349.1, ZP_{—}08115025.1, ZP_{—}08114403.1, YP_{—}003552869.1, YP_{—}002358112.1, ZP_{—}08111138.1, YP_{—}003770046.1, YP_{—}003103898.1, ZP_{—}08101069.1, ZP_{—}08097706.1, ZP_{—}08094005.1, YP_{—}003167240.1, YP_{—}002371817.1, YP_{—}004231854.1, EGA98455.1, YP_{—}002430239.1, ZP_{—}01049900.1, NP_{—}769819.1, NP_{—}768378.1, YP_{—}001143837.1, YP_{—}001108475.1, YP_{—}906040.1, YP_{—}726477.1, YP_{—}575010.1, YP_{—}477594.1, YP_{—}474564.1, YP_{—}130399.1, YP_{—}129373.1, YP_{—}123314.1, NP_{—}810467.1, NP_{—}646469.1, NP_{—}626044.1, NP_{—}391071.1, ZP_{—}08086822.1, ZP_{—}08084776.1, ZP_{—}08083119.1, NP_{—}465104.1, NP_{—}374819.1, NP_{—}337355.1, NP_{—}217296.1, ZP_{—}08072064.1, YP_{—}004197762.1, ZP_{—}08065558.1, ZP_{—}08063535.1, ZP_{—}08061612.1, ZP_{—}08059482.1, ZP_{—}08057644.1, ZP_{—}08055701.1, ZP_{—}08049025.1, ZP_{—}08047015.1, ZP_{—}04062925.1, YP_{—}269473.1, ZP_{—}08033402.1, ZP_{—}07829339.1, ZP_{—}06603053.1, ZP_{—}08020768.1, ZP_{—}08013590.1, ZP_{—}08011832.1, YP_{—}003783744.1, YP_{—}002781576.1, YP_{—}002780533.1, ZP_{—}02195873.1, NP_{—}797482.1, ZP_{—}08006697.1, ZP_{—}08006365.1, ZP_{—}08005962.1, ZP_{—}08004522.1, EFV89241.1, ZP_{—}07980135.1, ZP_{—}07974222.1, ZP_{—}07970379.1, ZP_{—}07962751.1, ZP_{—}07953732.1, ZP_{—}07945354.1, ZP_{—}06273519.1, YP_{—}003428808.1, YP_{—}003426902.1, YP_{—}001711555.1, YP_{—}001703831.1, YP_{—}001621081.1, YP_{—}001223643.1, YP_{—}001228127.1, YP_{—}849789.1, YP_{—}759696.1, NP_{—}969291.1, NP_{—}896596.1, NP 470950.1, YP_{—}359521.1, ZP_{—}01946735.1, ZP_{—}03631968.1, ZP_{—}01101833.1, YP_{—}002826017.1, YP_{—}003796926.1, ZP_{—}07873974.1, ZP_{—}07870908.1, ZP_{—}07645051.1, ZP_{—}07643260.1, ZP_{—}06611917.1, AAT40119.1, ZP_{—}07864946.1, YP_{—}004068409.1, YP_{—}002796203.1, YP_{—}002774420.1, YP_{—}003600348.1, YP_{—}003599946.1, YP_{—}003565624.1, YP_{—}003565223.1, YP_{—}335198.1, YP_{—}423850.1, YP_{—}155059.1, ZP_{—}07843538.1, ZP_{—}07841226.1, ZP_{—}03566837.1, EFS39373.1, EFS35044.1, ZP_{—}05287373.1, ZP_{—}05280407.1, ZP_{—}05224249.1, ZP_{—}04701236.1, ZP_{—}04692180.1, ZP_{—}03561728.1, ZP_{—}03227314.1, ZP_{—}02931419.1, ZP_{—}02731551.1, ZP_{—}02465413.1, ZP_{—}02451335.1, ZP_{—}02384332.1, ZP_{—}02381808.1, ZP_{—}02330643.1, YP_{—}004047600.1, EFR99988.1, EFR93766.1, EFR90643.1, EFR84459.1, ZP_{—}04059923.1, ZP_{—}03613601.1, ZP_{—}07743242.1, ZP_{—}07740118.1, ZP_{—}07728760.1, ZP_{—}07728640.1, YP_{—}003557047.1, ZP_{—}07204792.1, ZP_{—}07033145.1, ZP_{—}06949396.1, ZP_{—}06928932.1, ZP_{—}05692073.1, ZP_{—}05687006.1, ZP_{—}04867480.1, YP_{—}775531.1, CBE70214.1, ZP_{—}07721182.1, CBW22027.1, BAJ31519.1, ZP_{—}07694389.1, ZP_{—}07653390.1, ZP_{—}07548028.1, ZP_{—}07547185.1, ZP_{—}07462497.1, ZP_{—}07458778.1, ZP_{—}07399459.1, ZP_{—}07397253.1, ZP_{—}07397250.1, ZP_{—}07390390.1, ZP_{—}07390003.1, ZP_{—}07388675.1, ZP_{—}07367724.1, ZP_{—}07206561.1, ZP_{—}07053170.1, ZP_{—}07048770.1, ZP_{—}06873224.1, ZP_{—}06852862.1, ZP_{—}06427630.1, ZP_{—}06307332.1, ZP_{—}06252577.1, ZP_{—}06175164.1, ZP_{—}06080808.1, ZP_{—}06052314.1, ZP_{—}06033748.1, ZP_{—}05945907.1, ZP_{—}05924840.1, ZP_{—}05885109.1, ZP_{—}05882095.1, ZP_{—}05877865.1, ZP_{—}05855512.1, ZP_{—}05745159.1, ZP_{—}05716384.1, ZP_{—}04866524.1, ZP_{—}04819572.1, ZP_{—}04797418.1, ZP_{—}04319784.1, ZP_{—}04302850.1, ZP_{—}04298961.1, ZP_{—}04287684.1, ZP_{—}04277177.1, ZP_{—}04248389.1, ZP_{—}04235899.1, ZP_{—}04230016.1, ZP_{—}04226233.1, ZP_{—}04219330.1, ZP_{—}04216141.1, ZP_{—}04209092.1, ZP_{—}04188247.1, ZP_{—}04184510.1, ZP_{—}04176651.1, ZP_{—}04172877.1, ZP_{—}04170954.1, ZP_{—}04166021.1, ZP_{—}04160852.1, ZP_{—}04158983.1, ZP_{—}04154769.1, ZP_{—}04153266.1, ZP_{—}04149717.1, ZP_{—}04122524.1, ZP_{—}04110635.1, ZP_{—}04109769.1, ZP_{—}04109049.1, ZP_{—}04108444.1, ZP_{—}04104350.1, ZP_{—}04100460.1, ZP_{—}04075249.1, ZP_{—}04074263.1, ZP_{—}04009917.1, ZP_{—}03916440.1, ZP_{—}03703407.1, ZP_{—}03675960.1, ZP_{—}03588177.1, ZP_{—}03569636.1, ZP_{—}03497916.1, ZP_{—}03459468.1, ZP_{—}03299979.1, ZP_{—}03127493.1, ZP_{—}03054334.1, ZP_{—}03015779.1, ZP_{—}02478038.1, ZP_{—}02434435.1, ZP_{—}01891777.1, ZP_{—}01134782.1, ZP_{—}01084087.1, ZP_{—}00959435.1, ZP_{—}06021901.1, ZP_{—}02908521.1, ZP_{—}02892318.1, ZP_{—}02883918.1, ZP_{—}02433787.1, ZP_{—}02428013.1, ZP_{—}02424229.1, ZP_{—}02420399.1, ZP_{—}02190089.1, ZP_{—}02184200.1, ZP_{—}02166566.1, ZP_{—}02159718.1, ZP_{—}02152178.1, ZP_{—}02147727.1, ZP_{—}02144676.1, ZP_{—}02078507.1, ZP_{—}02072824.1, ZP_{—}02067293.1, ZP_{—}02061844.1, ZP_{—}01996280.1, ZP_{—}01991915.1, ZP_{—}01958087.1, ZP_{—}01908911.1, ZP_{—}01901606.1, ZP_{—}01895406.1, ZP_{—}01872936.1, ZP_{—}01870578.1, ZP_{—}01863314.1, ZP_{—}01859623.1, ZP_{—}01852574.1, ZP_{—}01834861.1, ZP_{—}01816459.1, ZP_{—}01770050.1, ZP_{—}01754550.1, ZP_{—}01750331.1, ZP_{—}01746097.1, ZP_{—}01736276.1, ZP_{—}01723571.1, ZP_{—}01688551.1, ZP_{—}01666824.1, ZP_{—}01627178.1, ZP_{—}01623088.1, ZP_{—}01612926.1, ZP_{—}01470938.1, ZP_{—}01460341.1, ZP_{—}01452344.1, ZP_{—}01439206.1, ZP_{—}01386817.1, ZP_{—}01313561.1, ZP_{—}01304248.1, ZP_{—}01264036.1, ZP_{—}01261877.1, ZP_{—}01235013.1, ZP_{—}01233072.1, ZP_{—}01224625.1, ZP_{—}01223017.1, ZP_{—}01221216.1, ZP_{—}01215557.1, ZP_{—}01202668.1, ZP_{—}01159834.1, ZP_{—}01158968.1, ZP_{—}01157579.1, ZP_{—}01130649.1, ZP_{—}01126987.1, ZP_{—}01122900.1, ZP_{—}01118752.1, ZP_{—}01090470.1, ZP_{—}01067027.1, ZP_{—}01058751.1, ZP_{—}01043459.1, ZP_{—}01041526.1, ZP_{—}01036767.1, ZP_{—}01001935.1, ZP_{—}00995212.1, ZP_{—}00992904.1, ZP_{—}00962062.1, ZP_{—}00952239.1, ZP_{—}00741173.1, ZP_{—}00740055.1, ZP_{—}00738801.1, ZP_{—}00517716.1, ZP_{—}00231205.1, ZP_{—}00208007.1, YP_{—}003974610.1, YP_{—}003546595.1, YP_{—}002317127.1, ZP_{—}07313778.1, ZP_{—}07302778.1, ZP_{—}07298850.1, ZP_{—}07285992.1, ZP_{—}07282306.1, ZP_{—}07279420.1, ZP_{—}07270582.1, ZP_{—}07001670.1, YP_{—}003706150.1, ZP_{—}06916083.1, ZP_{—}06912607.1, ZP_{—}06707160.1, ZP_{—}06324727.1, ZP_{—}06199155.1, ZP_{—}06197322.1, ZP_{—}05788488.1, ZP_{—}05785587.1, ZP_{—}05779471.1, ZP_{—}05739072.1, ZP_{—}05649780.1, ZP_{—}05647025.1, ZP_{—}05546023.1, ZP_{—}05341228.1, ZP_{—}05256588.1, ZP_{—}05127284.1, ZP_{—}05121710.1, ZP_{—}05119732.1, ZP_{—}05105668.1, ZP_{—}05101668.1, ZP_{—}05095370.1, ZP_{—}05090860.1, ZP_{—}05080646.1, ZP_{—}05076859.1, ZP_{—}05069222.1, ZP_{—}05065142.1, ZP_{—}05056378.1, ZP_{—}05052029.1, ZP_{—}05046506.1, ZP_{—}05037402.1, ZP_{—}05033610.1, ZP_{—}05026858.1, ZP_{—}05001187.1, ZP_{—}04959306.1, ZP_{—}04947229.1, ZP_{—}04941878.1, ZP_{—}04896669.1, ZP_{—}04890139.1, ZP_{—}04852481.1, ZP_{—}04849996.1, ZP_{—}04608704.1, ZP_{—}04581931.1, ZP_{—}04555275.1, ZP_{—}04553607.1, ZP_{—}04545440.1, ZP_{—}04538537.1, YP_{—}002311919.1, ZP_{—}01052096.1, YP_{—}432286.1, ZP_{—}07039851.1, ZP_{—}07036831.1, ZP_{—}07035634.1, ZP_{—}06826623.1, ZP_{—}06202690.1, ZP_{—}06091438.1, ZP_{—}06060476.1, YP_{—}002955941.1, YP_{—}002764322.1, YP_{—}002761274.1, YP_{—}002754767.1, YP_{—}002605829.1, YP_{—}002544281.1, YP_{—}002453687.1, YP_{—}002444060.1, YP_{—}002369417.1, YP_{—}002365390.1, YP_{—}002297006.1, YP_{—}002233968.1, YP_{—}001861152.1, YP_{—}001850232.1, YP_{—}001827236.1, YP_{—}001815332.1, YP_{—}001661116.1, YP_{—}001647239.1, YP_{—}001643400.1, YP_{—}001625970.1, YP_{—}001584357.1, YP_{—}001488077.1, YP_{—}001473862.1, YP_{—}001450010.1, YP_{—}001444991.1, YP_{—}001424576.1, YP_{—}001422460.1, YP_{—}001376512.1, YP_{—}001373857.1, YP_{—}001217438.1, YP_{—}001155448.1, YP_{—}001117213.1, YP_{—}001094151.1, YP_{—}950353.1, YP_{—}949946.1, YP_{—}944887.1, YP_{—}854776.1, YP_{—}837848.1, YP_{—}795217.1, YP_{—}750481.1, YP_{—}746463.1, YP_{—}681383.1, YP_{—}673989.1, YP_{—}632321.1, YP_{—}624008.1, YP_{—}615612.1, YP_{—}611857.1, YP_{—}604242.1, YP_{—}562748.1, YP_{—}536656.1, YP_{—}517218.1, YP_{—}459264.1, YP_{—}382475.1, YP_{—}340233.1, YP_{—}295387.1, YP_{—}285355.1, YP_{—}204286.1, YP_{—}174267.1, YP_{—}165491.1, YP_{—}126314.1, YP_{—}111103.1, YP_{—}098760.1, YP_{—}082111.1, YP_{—}064280.1, YP_{—}064276.1, YP_{—}062161.1, YP_{—}056928.1, YP_{—}008485.1, YP_{—}005739.1, NP_{—}961822.1, NP_{—}953341.1, NP_{—}926915.1, NP_{—}875991.1, NP_{—}834329.1, NP_{—}830409.1, NP_{—}827683.1, NP_{—}694147.1, NP_{—}693109.1, NP_{—}682897.1, NP_{—}661601.1, NP_{—}621858.1, NP_{—}486395.1, NP_{—}385730.1, NP_{—}231539.1, ADL65712.1, XP_{—}003087064.1, YP_{—}003886520.1, YP_{—}003699559.1, YP_{—}003516134.1, ADI98200.1, BAI86717.1, YP_{—}003794343.1, YP_{—}003790454.1, ADI11356.1, YP_{—}003845821.1, ADK69870.1, YP_{—}003784546.1, CBW36497.1, CBW26165.1, YP_{—}003709979.1, CAQ50186.1, ZP_{—}06770463.1, CBK69442.1, YP_{—}003413835.1, YP_{—}003595089.1, ZP_{—}06807811.1, YP_{—}003582455.1, YP_{—}003464731.1, YP_{—}003496397.1, YP_{—}003421918.1, CBL07274.1, CBK64956.1, YP_{—}003508515.1, AAL87460.1, AAC23579.1, AAC23578.1, AAC23577.1, ACU78652.1, YP_{—}003471439.1, YP_{—}003452777.1, ZP_{—}06384971.1, ACY25368.1, ABC26869.1, AAP44334.1, EEZ80018.1, ZP_{—}05110458.1, 1PJB_A, ZP_{—}04717201.1, ZP_{—}04689103.1, ZP_{—}04658071.1, XP_{—}002364705.1, ACN89388.1, 2VHW_A, 2VHV_A, XP_{—}001324625.1, ABZ06259.1, ABR57171.1, CAO90307.1, CAM75354.1, CAA44791.1, BAA77513.1, EGR96638.1, EGR94699.1, ZP_{—}08693646.1, YP_{—}004740306.1, YP_{—}004738947.1, AEE73472.1, YP_{—}002478771.1, YP_{—}002018970.1, YP_{—}001953230.1, ZP_{—}08683223.1, YP_{—}004073823.1, EGQ99856.1, ZP_{—}08664912.1, EGQ79321.1, YP_{—}001681700.1, AEJ51356.1, YP_{—}004378292.1, YP_{—}004237802.1, YP_{—}004166920.1, YP_{—}004043011.1, YP_{—}003997728.1, YP_{—}002975437.1, YP_{—}002514072.1, YP_{—}001433829.1, YP_{—}001185975.1, YP_{—}004676549.1, YP_{—}004016358.1, YP_{—}911347.1, YP_{—}004658403.1, YP_{—}002015455.1, YP_{—}001996171.1, YP_{—}001998271.1, YP_{—}001960099.1, YP_{—}001942826.1, YP_{—}001130666.1, YP_{—}004608353.1, YP_{—}508400.1, YP_{—}374553.1, ZP_{—}06298411.1, ZP_{—}06044299.1, ZP_{—}04390473.1, ZP_{—}04055222.1, ZP_{—}03779980.1, ZP_{—}03729400.1, ZP_{—}03390832.1, YP_{—}004580682.1, YP_{—}001988281.1, YP_{—}644219.1, YP_{—}665459.1, NP_{—}895289.1, YP_{—}004275231.1, NP_{—}208189.1, BAJ60529.1, BAJ59008.1, BAJ57509.1, BAJ56032.1, ZP_{—}01254396.1, YP_{—}445036.1, EGL90046.1, YP_{—}004510847.1, ZP_{—}08450330.1, YP_{—}003387804.1, YP_{—}003058152.1, ZP_{—}03438664.1, ZP_{—}01884341.1, AEG33860.1, YP_{—}004429375.1, ZP_{—}08459444.1, ZP_{—}07909193.1, ZP_{—}07908670.1, EFT26139.1, EFT23947.1, EFT12708.1, EFT03750.1, EFS82814.1, EFS74272.1, EFS67128.1, ZP_{—}06844564.1, YP_{—}826658.1, YP_{—}001195249.1, YP_{—}003095978.1, YP_{—}469292.1, YP_{—}004442054.1, YP_{—}004461174.1, YP_{—}004055616.1, YP_{—}003576656.1, YP_{—}003094537.1, YP_{—}001295973.1, AEE71143.1, YP_{—}004447480.1, YP_{—}001978005.1, ZP_{—}08413507.1, ZP_{—}07820264.1, YP_{—}416780.1, EGI86036.1, YP_{—}003109321.1, YP_{—}001275268.1, YP_{—}380171.1, YP_{—}159073.1, YP_{—}004203456.1, YP_{—}003761844.1, YP_{—}040853.1, ZP_{—}08328557.1, CBL87253.1, CBL87167.1, YP_{—}004316768.1, EFS92548.1, YP_{—}001016505.1, EGG67688.1, YP_{—}003528837.1, YP_{—}002434942.1, YP_{—}117835.1, YP_{—}004150583.1, YP_{—}003755105.1, YP_{—}002526442.1, YP_{—}003120958.1, EGE94241.1, YP_{—}004345416.1, EFS79952.1, ZP_{—}06964253.1, EGE60050.1, CBZ52359.1, ADU40304.1, ADQ77229.1, YP_{—}003196038.1, YP_{—}144713.1, YP_{—}001304143.1, YP_{—}113082.1, ADO76516.1, YP_{—}003326349.1, YP_{—}003289755.1, YP_{—}003089327.1, ZP_{—}07911965.1, ZP_{—}05773583.1, ZP_{—}05765271.1, YP_{—}003154888.1, YP_{—}003142045.1, YP_{—}002280953.1, NP_{—}371963.1, NP_{—}422368.1, EGC98966.1, EGC76398.1, YP_{—}004263661.1, YP_{—}004252039.1, YP_{—}679036.1, YP_{—}499973.1, ZP_{—}08090745.1, ZP_{—}08108339.1, YP_{—}001531594.1, ZP_{—}01051588.1, NP_{—}646145.1, NP_{—}224146.1, ZP_{—}08054972.1, ZP_{—}08053009.1, YP_{—}003584878.1, ZP_{—}07939405.1, ZP_{—}03439290.1, ADU82392.1, ADU83943.1, ADU85424.1, ADU80668.1, YP_{—}001225733.1, YP_{—}003863039.1, ZP_{—}01061682.1, YP_{—}767568.1, ZP_{—}07865749.1, ZP_{—}06858058.1, YP_{—}628213.1, EFT81350.1, EFT66610.1, EFT51424.1, ZP_{—}04839161.1, ZP_{—}05633406.1, ZP_{—}05288381.1, AAR37813.1, EFS03282.1, EFS03278.1, YP_{—}004046539.1, ZP_{—}07749550.1, ZP_{—}07729731.1, ADN80650.1, ZP_{—}07088856.1, ZP_{—}07080219.1, ZP_{—}06949721.1, ZP_{—}05685436.1, YP_{—}002550450.1, YP_{—}803715.1, ZP_{—}07720023.1, ZP_{—}07469700.1, ZP_{—}07365619.1, ZP_{—}06924335.1, ZP_{—}06715776.1, ZP_{—}06303722.1, ZP_{—}06303721.1, ZP_{—}06264319.1, ZP_{—}06155528.1, ZP_{—}05745707.1, ZP_{—}04866244.1, ZP_{—}04199629.1, ZP_{—}04195783.1, ZP_{—}04067276.1, ZP_{—}03968868.1, ZP_{—}03963857.1, ZP_{—}03933079.1, ZP_{—}03497046.1, ZP_{—}03475134.1, ZP_{—}01890152.1, ZP_{—}01086712.1, ZP_{—}06021845.1, ZP_{—}02183427.1, ZP_{—}02162695.1, ZP_{—}02032824.1, ZP_{—}01993906.1, ZP_{—}01993127.1, ZP_{—}01983694.1, ZP_{—}01972527.1, ZP_{—}01819838.1, ZP_{—}01817962.1, ZP_{—}01740947.1, ZP_{—}01734991.1, ZP_{—}01694775.1, ZP_{—}01678972.1, ZP_{—}01468566.1, ZP_{—}01408749.1, ZP_{—}01386800.1, ZP_{—}01202184.1, ZP_{—}01174108.1, ZP_{—}01174047.1, ZP_{—}01118729.1, ZP_{—}01081268.1, ZP_{—}00998573.1, ZP_{—}00739793.1, YP_{—}002302140.1, ZP_{—}07358151.1, ZP_{—}06668925.1, ZP_{—}06668924.1, ZP_{—}06667106.1, ZP_{—}06324464.1, ZP_{—}06196777.1, ZP_{—}05114159.1, ZP_{—}05083968.1, ZP_{—}05070370.1, ZP_{—}05030022.1, ZP_{—}04673064.1, ZP_{—}04581752.1, ZP_{—}01052079.1, ZP_{—}07661104.1, ZP_{—}06077819.1, YP_{—}002835579.1, YP_{—}002267069.1, YP_{—}002129114.1, YP_{—}001929236.1, YP_{—}001910999.1, YP_{—}001854051.1, YP_{—}001094152.1, YP_{—}001044252.1, YP_{—}861818.1, YP_{—}915522.1, YP_{—}807371.1, YP_{—}353800.1, YP_{—}342402.1, YP_{—}065168.1, YP_{—}015797.1, YP_{—}005051.1, NP_{—}856449.1, NP_{—}661547.1, NP_{—}358448.1, YP_{—}003929442.1, YP_{—}003927769.1, ADO06185.1, ADO04689.1, ADL23243.1, YP_{—}003789202.1, ADJ79786.1, YP_{—}003516488.1, ADI97953.1, ADI35485.1, YP_{—}003716800.1, ZP_{—}00241359.1, YP_{—}003718040.1, CAQ49862.1, YP_{—}003282331.1, AAP97897.1, ACX99978.1, ACX98578.1, YP_{—}003472544.1, ZP_{—}06382734.1, EEZ79852.1, ZP_{—}05299989.1, ZP_{—}05299895.1, XP_{—}002367632.1, ZP_{—}03529835.1, ZP_{—}03517011.1, ZP_{—}03505783.1, XP_{—}001310698.1, ABK27691.1, CAB59281.2,
in particular NP_{—}391071.1, BAI86717.1, YP_{—}004205024.1, ZP_{—}06873224.1, YP_{—}003974610.1, YP_{—}001422460.1, AEB25326.1, YP_{—}003921585.1, YP_{—}080482.1, ZP_{—}03054334.1, YP_{—}001488077.1, YP_{—}081348.1, YP_{—}003426902.1, NP_{—}243195.1, ZP_{—}08004522.1, YP_{—}003565624.1, YP_{—}004095847.1, YP_{—}003600348.1, ZP_{—}08006697.1, ZP_{—}04248389.1, YP_{—}174267.1, YP_{—}001376512.1, ZP_{—}04226233.1, ZP_{—}04100460.1, YP_{—}002369417.1, ZP_{—}03229411.1, ZP_{—}04110635.1, ZP_{—}04287684.1, ZP_{—}04172877.1, ZP_{—}04158983.1, ZP_{—}04219330.1, NP_{—}830409.1, YP_{—}003790454.1, ZP_{—}04184510.1, YP_{—}001642542.1, ZP_{—}04074263.1, ZP_{—}04319784.1, NP_{—}847074.1, YP_{—}001373857.1, ZP_{—}04122524.1, ZP_{—}03230841.1, YP_{—}082111.1, NP_{—}834329.1, YP_{—}002444060.1, ZP_{—}04170954.1, YP_{—}002453687.1, ZP_{—}04153266.1, ZP_{—}04302850.1, YP_{—}002365390.1, ZP_{—}04216141.1, ZP_{—}04298961.1, ZP_{—}00740055.1, ZP_{—}04277177.1, ZP_{—}04104350.1, ZP_{—}04176651.1, YP_{—}001647239.1, ZP_{—}04188247.1, ZP_{—}04149717.1, YP_{—}003794343.1, ZP_{—}04230016.1, YP_{—}001643400.1, ZP_{—}04209092.1, ZP_{—}04235899.1, YP_{—}003428808.1, ZP_{—}08005962.1, YP_{—}003599946.1, YP_{—}003565223.1, ZP_{—}01859623.1, YP_{—}004569425.1, ZP_{—}04432601.1, ZP_{—}03227314.1, YP_{—}003699559.1, ZP_{—}07709417.1, ZP_{—}01723571.1, NP_{—}244046.1, ZP_{—}08006365.1, ZP_{—}00738801.1, ZP_{—}04160852.1, ZP_{—}04166021.1, ZP_{—}04154769.1, ZP_{—}04109769.1, ZP_{—}04109049.1, ZP_{—}04108444.1, ZP_{—}04075249.1, ZP_{—}00741173.1, ZP_{—}00739793.1, ZP_{—}01174108.1, ZP_{—}01174047.1, ZP_{—}00241359.1, ZP_{—}04195783.1, ZP_{—}04199629.1, ZP_{—}04067276.1
and especially preferably NP_{—}391071.1.
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, to be more precise in a system in which pyruvate is converted into alanine.

### Fifth Genetic Modification for Suppressing the Degradation of Carboxylic Acids and Carboxylic Acid Derivatives

Furthermore preferred according to the invention are microorganisms which include a fifth genetic modification which comprises an activity of at least one of the enzymes selected from the group

Eₐ acyl-CoA synthetase, preferably of EC 6.2.1.3, which catalyses the synthesis of an acyl-coenzyme A thioester,
E_{b} acyl-CoA dehydrogenase, preferably of EC 1.3.99.-, EC 1.3.99.3 or EC 1.3.99.13, which catalyses the oxidation of an acyl-coenzyme A thioester to give the corresponding enoyl-coenzyme A thioester,
E_{c} acyl-CoA oxidase, preferably of EC 1.3.3.6, which catalyses the oxidation of an acyl-coenzyme A thioester to give the corresponding enoyl-coenzyme A thioester,
E_{d} enoyl-CoA hydratase, preferably of EC 4.2.1.17 or EC 4.2.1.74, which catalyses the hydratization of an enoyl-coenzyme A thioester to give the corresponding 3-hydroxyacyl-coenzyme A thioester,
Eₑ 3-hydroxyacyl-CoA dehydrogenase, preferably of EC 1.1.1.35 or EC 1.1.1.211, which catalyses the oxidation of a 3-hydroxyacyl-coenzyme A thioester to give the corresponding 3-oxoacyl-coenzyme A thioester, and
E_{f} acetyl-CoA acyltransferase, preferably of EC 2.3.1.16, which catalyses the transfer of an acetyl residue from a 3-oxoacyl-coenzyme A thioester to coenzyme A and thus generates an acyl-coenzyme A thioester which is shortened by two carbon atoms,
which is reduced in comparison with the enzymatic activity of the wild type of the microorganism.

The technical effect of this is that the drain of the carboxylic acids and carboxylic acid derivatives formed in larger amounts due to the first genetic modification, but also of those formed in larger amounts due to the second, third and fourth genetic modification, is prevented.

The wording “activity which is reduced in comparison with its wild type” is preferably understood as meaning an activity which is reduced by at least 50%, especially preferably by at least 90%, more preferably by at least 99.9%, even more preferably by at least 99.99% and most preferably by at least 99.999%, based on the wild type activity. The wording “reduced activity” also comprises no detectable activity (“zero activity”). The reduction of the activity of a specific enzyme can be effected for example by the targeted mutation or by other means known to a person skilled in the art for reducing the activity of a specific enzyme. Other methods of reducing enzymatic activities in microorganisms are known to a person skilled in the art.

Methods of choice here are, in particular, molecular-biological techniques. Information on the modification and reduction of protein expressions and reduced enzymatic activity which these entail specifically for Candida, in particular for interrupting specific genes, can be found by the skilled worker in WO91/006660 and WO03/100013.

Microorganisms which are preferred according to the invention are characterized in that the reduction of the enzymatic activity is achieved by modifying a gene comprising a nucleic acid sequence encoding the abovementioned enzymes, the modification being selected from the group comprising, preferably composed of, insertion of foreign DNA into the gene, deletion of at least parts of the gene, point mutations in the gene sequence, RNA interference (siRNA), antisense RNA or modification (insertion, deletion or point mutations) of regulatory sequences which flank the gene. In this context, foreign DNA is understood as meaning any DNA sequence which is “foreign” to the gene (but not the organism). In this context it is especially preferred that the gene is interrupted by inserting a selection marker gene, the foreign DNA thus being a selection marker gene, where the insertion has preferably been effected by homologous recombination into the gene locus. In this context, it may be advantageous to extend the selection marker gene by further functionalities which, in turn, make possible a subsequent removal from the gene. This may be achieved for example by recombination systems which are foreign to the organism, such as a Cre/loxP system or FRT (Flippase Recognition Target) system or by the homologous recombination system which belongs to the organism. The reduction of the activity of the microorganism according to the invention in comparison with its wild type is determined by abovementioned methods for determining the activity using cell numbers/concentrations which are as equal as possible, the cells having been grown under identical conditions such as, for example, medium, gas supply, agitation.

### Specific Enzymes Eₐ

In cells which are preferred according to the invention, the enzyme Eₐ is one which comprises the sequence NP_{—}416319.1 (SEQ ID No.: 18)

proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eₐ is generally understood in particular as meaning the synthesis of dodecanoyl-CoA thioester.

### Specific Enzymes E_{b}

Furthermore, it is preferred according to the invention that the enzyme E_{b} in the cells according to the invention is one which comprises sequences selected from among:

YP_{—}488518.1 (encoded by SEQ ID No. 14, formerly AP_{—}000876.1), ZP_{—}08341828.1, YP_{—}002291517.1, ZP_{—}08393771.1, EFW53921.1, YP_{—}003227327.1, YP_{—}001461409.1, AEG35025.1, YP_{—}002385739.1, EGJ00024.1, ZP_{—}08352177.1, ZP_{—}03070250.1, ZP_{—}08367389.1, EGM63466.1, CBI99746.1, ZP_{—}06660773.1, ZP_{—}08372569.1, YP_{—}309282.2, YP_{—}001879017.1, YP_{—}003497883.1, ACI71032.1, YP_{—}002406464.1, EGB79412.1, EFZ76765.1, ZP_{—}07145000.1, ZP_{—}07151031.1, AAZ87047.1, EFZ56676.1, ZP_{—}06656148.1, EGB35012.1, EGB71054.1, EFW49392.1, ZP_{—}07183316.1, YP_{—}002396328.1, YP_{—}002327805.1, ZP_{—}03027602.1, AAG54546.1, YP_{—}001742341.1, ZP_{—}04538240.1, EFX12717.1, ACI71029.1, NP_{—}285938.2, ZP_{—}03064986.1, ZP_{—}07120505.1, YP_{—}539295.2, ZP_{—}03049609.1, ZP_{—}06652178.1, AAP15817.1, NP_{—}706224.3, ABI99723.1, EGB60663.1, EFW71911.1, EGH38574.1, YP_{—}668186.1, EGK29152.1, EGC05203.1, ZP_{—}02801146.2, YP_{—}687886.1, ZP_{—}08346491.1, EGJ94671.1, EGC94003.1, ZP_{—}08362542.1, YP_{—}002381459.1, AAN78852.1, NP_{—}752308.2, ZP_{—}07173894.1, ZP_{—}08357265.1, ZP_{—}08382276.1, ZP_{—}02902298.1, ZP_{—}04560694.1, ZP_{—}06354226.1, CBY94356.1, NP_{—}459307.1, EGE28353.1, ZP_{—}04657138.1, YP_{—}002225434.1, YP_{—}002635948.1, YP_{—}151638.1, ZP_{—}02663643.1, NP_{—}454921.1, YP_{—}004729161.1, YP_{—}215297.1, YP_{—}001454506.1, YP_{—}001571699.1, YP_{—}003363866.1, EGK30199.1, EGJ91900.1, EGK28208.1, ZP_{—}08497358.1, CBK85993.1, YP_{—}003611563.1, YP_{—}004592567.1, YP_{—}003441061.1, YP_{—}002240296.1, YP_{—}002917946.1, ZP_{—}06549304.1, ZP_{—}06017126.1, YP_{—}001333918.1, AAM28523.1, ZP_{—}08305363.1, YP_{—}001439185.1, EGL74026.1, YP_{—}001175495.1, ZP_{—}05968792.1, YP_{—}003209204.1, YP_{—}003943022.1, YP_{—}004499335.1, ZP_{—}06191708.1, YP_{—}001477183.1, ZP_{—}07951567.1, YP_{—}003740265.1, NP_{—}668276.1, ZP_{—}04637564.1, ZP_{—}04631714.1, CBY26031.1, YP_{—}004297237.1, YP_{—}001007400.1, ZP_{—}04625511.1, YP_{—}069424.1, ZP_{—}04616432.1, ZP_{—}04639135.1, YP_{—}001871363.1, ZP_{—}04620883.1, ZP_{—}06636999.1, ZP_{—}07377275.1, YP_{—}003929932.1, YP_{—}001722031.1, ZP_{—}04614013.1, ZP_{—}04628476.1, YP_{—}003713213.1, YP_{—}003530236.1, CBX79727.1, YP_{—}004114694.1, YP_{—}001908526.1, ADP 11689.1, YP_{—}002649711.1, YP_{—}003469212.1, YP_{—}003519171.1, YP_{—}051564.1, ZP_{—}03833764.1, ZP_{—}03827249.1, NP_{—}928504.1, YP_{—}004211704.1, ZP_{—}07681706.1, YP_{—}003018849.1, YP_{—}003260788.1, YP_{—}003042091.1, ZP_{—}05973896.1, ZP_{—}03317495.1, ZP_{—}02958330.2, EFW60358.1, EGI98786.1, ZP_{—}06127315.2, YP_{—}002150121.1, ZP_{—}03842196.1, YP_{—}003884303.1, YP_{—}003003248.1, YP_{—}003334792.1, ZP_{—}03379559.1, CBA73629.1, YP_{—}002986552.1, ZP_{—}06538530.1, ZP_{—}01258771.1, ZP_{—}04921840.1, ZP_{—}06180371.1, ZP_{—}08308836.1, ZP_{—}06174994.1, YP_{—}001446380.1, ZP_{—}01237449.1, ZP_{—}01161468.1, ZP_{—}01222040.1, ZP_{—}06038476.1, ZP_{—}05925639.1, ZP_{—}06154677.1, ZP_{—}02195704.1, ZP_{—}01989646.1, ZP_{—}01868523.1, YP_{—}131060.1, ZP_{—}05722161.1, ZP_{—}05716057.1, NP_{—}798668.1, EGF45205.1, ZP_{—}05120764.1, EGR07881.1, ZP_{—}08100412.1, ZP_{—}04919383.1, ZP_{—}06054287.1, YP_{—}002156761.1, YP_{—}205315.2, ZP_{—}04961417.1, ZP_{—}06050299.1, ZP_{—}08103013.1, ZP_{—}01949008.1, NP_{—}231862.1, AEA79156.1, ZP_{—}06081122.1, ZP_{—}04418155.1, YP_{—}001217747.1, ZP_{—}04413631.1, NP_{—}935312.1, ZP_{—}01977990.1, NP_{—}760770.1, YP_{—}004188005.1, YP_{—}002810906.1, ZP_{—}05884155.1, ZP_{—}05946273.1, ZP_{—}01065180.1, ZP_{—}01815735.1, YP_{—}002417909.1, YP_{—}002263750.1, YP_{—}856109.1, ZP_{—}07744057.1, ZP_{—}08520214.1, ZP_{—}06034047.1, YP_{—}004565576.1, ZP_{—}05881167.1, ZP_{—}00991316.1, YP_{—}734276.1, ADT86286.1, YP_{—}001142550.1, YP_{—}869958.1, ZP_{—}08566610.1, ZP_{—}05876732.1, YP_{—}001366225.1, YP_{—}001094233.1, ADV54653.1, YP_{—}963612.1, YP_{—}738268.1, YP_{—}001502248.1, YP_{—}004391846.1, YP_{—}002311644.1, YP_{—}002358241.1, YP_{—}001050670.1, ZP_{—}07390237.1, YP_{—}001674114.1, YP_{—}001554497.1, NP_{—}718122.1, YP_{—}001760976.1, YP_{—}927745.1, YP_{—}562771.1, YP_{—}003557130.1, ZP_{—}02159449.1, YP_{—}003913548.1, YP_{—}001473736.1, YP_{—}750554.1, ZP_{—}01897495.1, YP_{—}268985.1, ZP_{—}01042474.1, ZP_{—}08570996.1, YP_{—}004427315.1, ZP_{—}07010199.1, YP_{—}156047.1, ZP_{—}07097521.1, YP_{—}004467113.1, ZP_{—}01614110.1, YP_{—}340459.1, YP_{—}004434754.1, YP_{—}662062.1, YP_{—}004068195.1, ZP_{—}08409704.1, ZP_{—}08622396.1, ZP_{—}01135962.1, ZP_{—}03560927.1, ZP_{—}04716612.1, EGB41427.1, EGP48304.1, EFV84045.1, ZP_{—}08505249.1, ZP_{—}06688896.1, YP_{—}003980530.1, YP_{—}003168652.1, YP_{—}003146346.1, YP_{—}001250478.1, YP_{—}095752.1, YP_{—}124009.1, CBW99992.1, YP_{—}284763.1, YP_{—}127029.1, YP_{—}746940.1, ZP_{—}07663653.1, ZP_{—}03349444.1, YP_{—}002354470.1, YP_{—}004145615.1, YP_{—}003524477.1, ZP_{—}03698069.1, YP_{—}003376672.1, ZP_{—}06188282.1, EFW81359.1, EGH83675.1, EGH67821.1, EFW83732.1, YP_{—}273865.1, NP_{—}902393.1, ZP_{—}06457469.1, EGH99235.1, ZP_{—}03397893.1, ZP_{—}07004262.1, ZP_{—}06732661.1, ZP_{—}07263971.1, EGH75297.1, NP_{—}888341.1, EGH31566.1, EGH45251.1, NP_{—}643363.1, EGH24154.1, EGH92666.1, EGH73945.1, EGH12424.1, NP_{—}793629.1, ZP_{—}06705890.1, YP_{—}234714.1, EGH62932.1, EGH52925.1, ZP_{—}01126966.1, NP_{—}841588.1, ZP_{—}05109483.1, YP_{—}003847638.1, YP_{—}004294524.1, ZP_{—}02244088.1, NP_{—}884586.1, ZP_{—}08176463.1, ZP_{—}04588788.1, YP_{—}450732.1, ZP_{—}08185386.1, YP_{—}001914265.1, YP_{—}003527565.1, YP_{—}004696148.1, NP_{—}638218.1, ZP_{—}05046817.1, YP_{—}343737.1, ZP_{—}07652844.1, YP_{—}004227922.1, YP_{—}364921.1, YP_{—}001632020.1, NP_{—}744048.1, YP_{—}001898007.1, YP_{—}003145987.1, YP_{—}558241.1, YP_{—}410795.1, YP_{—}001895310.1, YP_{—}002980410.1, ZP_{—}06841648.1, YP_{—}258889.1, YP_{—}931967.1, YP_{—}003760619.1, YP_{—}002029446.1, YP_{—}004474743.1, YP_{—}158312.1, YP_{—}004380764.1, YP_{—}001973352.1, CBJ39115.1, YP_{—}349912.1, YP_{—}003753442.1, ZP_{—}05135288.1, YP_{—}004700980.1, YP_{—}927690.1, YP_{—}001269130.1, YP_{—}742956.1, ADR61321.1, YP_{—}001347709.1, YP_{—}004355482.1, YP_{—}003907207.1, NP_{—}251505.1, ZP_{—}04929120.1, NP_{—}518658.1, YP_{—}002871500.1, ZP_{—}01451059.1, EGM21899.1, YP_{—}001187411.1, ZP_{—}08570514.1, ZP_{—}07794119.1, YP_{—}004391835.1, YP_{—}002256385.1, ZP_{—}07774414.1, YP_{—}855885.1, YP_{—}563120.1, YP_{—}001172167.1, YP_{—}004713921.1, ZP_{—}08138366.1, AEA83572.1, YP_{—}003746704.1, ZP_{—}08521441.1, ZP_{—}05061205.1, YP_{—}001667709.1, YP_{—}750573.1, YP_{—}607261.1, ZP_{—}05118288.1, YP_{—}002311716.1, NP_{—}718079.1, YP_{—}003777020.1, ZP_{—}06052248.1, ZP_{—}00943163.1, ZP_{—}08309312.1, AEG70141.1, YP_{—}001748377.1, YP_{—}001857928.1, YP_{—}001094176.1, YP_{—}003604813.1, ZP_{—}01947893.1,
in particular
EFW81359.1, EGH83675.1, EGH67821.1, EFW83732.1, YP_{—}273865.1, ZP_{—}06457469.1, EGH99235.1, ZP_{—}03397893.1, ZP_{—}07004262.1, ZP_{—}07263971.1, EGH75297.1, EGH31566.1, EGH45251.1, EGH24154.1, EGH92666.1, EGH73945.1, EGH12424.1, NP_{—}793629.1, YP_{—}234714.1, EGH62932.1, EGH52925.1, ZP_{—}04588788.1, NP_{—}744048.1, YP_{—}258889.1, YP_{—}004474743.1, YP_{—}004380764.1, YP_{—}349912.1, YP_{—}004700980.1, YP_{—}001269130.1, ADR61321.1, YP_{—}001347709.1, YP_{—}004355482.1, NP_{—}251505.1, ZP_{—}04929120.1, YP_{—}002871500.1, EGM21899.1, YP_{—}001187411.1, ZP_{—}07794119.1, ZP_{—}07774414.1, YP_{—}001172167.1, YP_{—}004713921.1, ZP_{—}08138366.1, AEA83572.1, YP_{—}001667709.1, YP_{—}607261.1, YP_{—}001748377.1, YP_{—}260045.1, YP_{—}002873091.1, ZP_{—}07775826.1, CAC34855.1, EGH11916.1, ZP_{—}05641615.1, ZP_{—}06480669.1, ZP_{—}06480668.1, ZP_{—}05641616.1, ZP_{—}06492823.1, ZP_{—}06492821.1, EGH11920.1, EGH25319.1, ZP_{—}06492824.1, ADX52254.1, YP_{—}488518.1 (encoded by SEQ ID No. 14, formerly AP_{—}000876.1), ZP_{—}08341828.1, YP_{—}002291517.1, YP_{—}003227327.1, YP_{—}001461409.1, AEG35025.1, YP_{—}002385739.1, ZP_{—}08352177.1, ZP_{—}03070250.1, ZP_{—}08367389.1, CBI99746.1, ZP_{—}06660773.1, ZP_{—}08372569.1, YP_{—}003497883.1, ACI71032.1, YP_{—}002406464.1, EGB79412.1, EFZ76765.1, ZP_{—}07145000.1, ZP_{—}07151031.1, EFZ56676.1, ZP_{—}06656148.1, EGB35012.1, EGB71054.1, ZP_{—}07183316.1, YP_{—}002396328.1, YP_{—}002327805.1, ZP_{—}03027602.1, AAG54546.1, YP_{—}001742341.1, ABE05764.1, EFX12717.1, ACI71029.1, NP_{—}285938.2, ZP_{—}07120505.1, YP_{—}539295.2, ZP_{—}03049609.1, ZP_{—}06652178.1, ABI99723.1, EGB60663.1, EFW71911.1, EGH38574.1, YP_{—}668186.1, ZP_{—}02801146.2, ZP_{—}08346491.1, ZP_{—}08362542.1, AAN78852.1, NP_{—}752308.2, ZP_{—}07173894.1, ZP_{—}08357265.1, ZP_{—}08382276.1, AAM28523.1, ZP_{—}07097521.1, EGB41427.1, EGB41426.1, BAA07583.1, ZP_{—}07100038.1, CAX20347.1
and especially preferably
NP_{—}744048.1, YP_{—}004700980.1, YP_{—}001269130.1, ADR61321.1, YP_{—}001667709.1, YP_{—}001748377.1, YP_{—}258889.1, YP_{—}349912.1, YP_{—}002871500.1, ZP_{—}07774414.1, YP_{—}260045.1, YP_{—}002873091.1, ZP_{—}07775826.1, CAC34855.1, YP_{—}001172167.1, YP_{—}004713921.1, AEA83572.1, YP_{—}488518.1 (encoded by SEQ ID No. 14, formerly AP_{—}000876.1), BAA07583.1, ZP_{—}07594808.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to give 2-dodecenoyl-CoA thioester.

### Specific Enzymes E_{c}

Furthermore, it is preferred according to the invention that the enzyme E_{c} in the cells according to the invention is one which comprises sequences selected from among:

YP_{—}003571780.1, YP_{—}445820.1, YP_{—}634556.1, YP_{—}004665862.1, ZP_{—}01461690.1, YP_{—}921666.1, YP_{—}002778910.1, ZP_{—}08550394.1, YP_{—}003384289.1, YP_{—}001195727.1, YP_{—}702012.1, ZP_{—}04384437.1, YP_{—}002765110.1, ZP_{—}04996322.1, ZP_{—}08195144.1, ZP_{—}04700546.1, YP_{—}954595.1, YP_{—}004736804.1, ADW07059.1, YP_{—}001827916.1, ZP_{—}04691466.1, YP_{—}001109453.1, ZP_{—}08240125.1, YP_{—}003272226.1, YP_{—}004053469.1, ZP_{—}06272176.1, YP_{—}004491616.1, YP_{—}001133991.1, YP_{—}001071715.1, YP_{—}290295.1, YP_{—}003193744.1, YP_{—}001704317.1, YP_{—}004008413.1, YP_{—}004655806.1, YP_{—}640598.1, ZP_{—}08153802.1, ZP_{—}00995173.1, ZP_{—}05225674.1, YP_{—}888747.1, YP_{—}003114111.1, YP_{—}004522832.1, ZP_{—}06848773.1, ZP_{—}08203814.1, YP_{—}001851901.1, EGO40578.1, YP_{—}003134974.1, ZP_{—}07282448.1, YP_{—}003770185.1, YP_{—}881295.1, YP_{—}004336131.1, NP_{—}961035.1, YP_{—}004164861.1, YP_{—}003681133.1, ZP_{—}04749633.1, ZP_{—}07718288.1, ZP_{—}01201898.1, YP_{—}004223976.1, YP_{—}118690.1, YP_{—}905275.1, BAE47462.1, YP_{—}831622.1, YP_{—}003407476.1, ZP_{—}01129477.1, YP_{—}003645654.1, YP_{—}004454693.1, YP_{—}002487953.1, YP_{—}004084231.1, YP_{—}003836912.1, YP_{—}004241154.1, ZP_{—}07706098.1, YP_{—}001855531.1, ZP_{—}08124588.1, YP_{—}947882.1, BAE47461.1, YP_{—}003327670.1, YP_{—}001363757.1, YP_{—}004601796.1, YP_{—}001625220.1, YP_{—}003638017.1, ZP_{—}06501585.1, YP_{—}004404736.1, YP_{—}062974.1, YP_{—}002957230.1, YP_{—}003316209.1, YP_{—}003149881.1, YP_{—}001221553.1, YP_{—}003162313.1, ZP_{—}03978917.1, YP_{—}001708860.1, ZP_{—}05912043.1, ZP_{—}06806059.1, YP_{—}003155732.1, YP_{—}002835700.1, YP_{—}003916799.1, ZP_{—}03936415.1, ZP_{—}07090640.1, ZP_{—}08516453.1, AAB97825.1, YP_{—}004541029.1, YP_{—}004606508.1, YP_{—}001801238.1, ZP_{—}07989876.1, YP_{—}004761186.1, YP_{—}002883572.1, ZP_{—}08023616.1, ZP_{—}05847263.1, YP_{—}251740.1, ZP_{—}03394212.1, YP_{—}001107648.1, YP_{—}002872770.1, YP_{—}001821654.1, ZP_{—}08233739.1, AAD12170.1, ZP_{—}08215859.1, AAD40800.1, ZP_{—}05005905.1, ADW07311.1, YP_{—}348592.1, NP_{—}824883.1, NP_{—}627459.1, YP_{—}001828149.1, ZP_{—}05525554.1, ZP_{—}08240364.1, ZP_{—}07299658.1, ZP_{—}06582153.1, ZP_{—}06921827.1, ZP_{—}04703961.1, BAJ27090.1, ZP_{—}06592678.1, ZP_{—}04691265.1, YP_{—}001751500.1, BAJ31579.1, preferably YP_{—}003571780.1, YP_{—}445820.1, YP_{—}634556.1, YP_{—}004665862.1, ZP_{—}01461690.1, YP_{—}921666.1, YP_{—}002778910.1, ZP_{—}08550394.1, YP_{—}003384289.1, YP_{—}001195727.1, YP_{—}702012.1, ZP_{—}04384437.1, YP_{—}002765110.1, ZP_{—}04996322.1, ZP_{—}08195144.1, ZP_{—}04700546.1, YP_{—}954595.1, YP_{—}004736804.1, ADW07059.1, YP_{—}001827916.1, ZP_{—}04691466.1, YP_{—}001109453.1, ZP_{—}08240125.1, YP_{—}003272226.1, YP_{—}004053469.1, ZP_{—}06272176.1, YP_{—}004491616.1, YP_{—}001133991.1, YP_{—}001071715.1, YP_{—}290295.1, YP_{—}003193744.1, YP_{—}001704317.1, YP_{—}004008413.1, YP_{—}004655806.1, YP_{—}640598.1, ZP_{—}08153802.1, ZP_{—}00995173.1, ZP_{—}05225674.1, YP_{—}888747.1, YP_{—}003114111.1, YP_{—}004522832.1, ZP_{—}06848773.1, ZP_{—}08203814.1, YP_{—}001851901.1, EGO40578.1, YP_{—}003134974.1, ZP_{—}07282448.1, YP_{—}003770185.1, YP_{—}881295.1, YP_{—}004336131.1, NP_{—}961035.1, YP_{—}004164861.1, YP_{—}003681133.1, ZP_{—}04749633.1, ZP_{—}07718288.1, ZP_{—}01201898.1, YP_{—}004223976.1, YP_{—}118690.1, YP_{—}905275.1, BAE47462.1, YP_{—}831622.1, YP_{—}003407476.1, ZP_{—}01129477.1, YP_{—}003645654.1, YP_{—}004454693.1, YP_{—}002487953.1, YP_{—}004084231.1, YP_{—}003836912.1, YP_{—}004241154.1, ZP_{—}07706098.1, YP_{—}001855531.1, ZP_{—}08124588.1, YP_{—}947882.1, BAE47461.1, YP_{—}003327670.1, YP_{—}001363757.1, YP_{—}004601796.1, YP_{—}001625220.1, YP_{—}003638017.1, ZP_{—}06501585.1, YP_{—}004404736.1, YP_{—}062974.1, YP_{—}002957230.1, YP_{—}003316209.1, YP_{—}003149881.1, YP_{—}001221553.1, YP_{—}003162313.1, ZP_{—}03978917.1, YP_{—}001708860.1, ZP_{—}05912043.1, ZP_{—}06806059.1, YP_{—}003155732.1, YP_{—}002835700.1, YP_{—}003916799.1, ZP_{—}03936415.1, ZP_{—}07090640.1, ZP_{—}08516453.1, AAB97825.1, YP_{—}004541029.1, YP_{—}004606508.1, YP_{—}001801238.1, ZP_{—}07989876.1, YP_{—}004761186.1, YP_{—}002883572.1, ZP_{—}08023616.1, ZP_{—}05847263.1, YP_{—}251740.1,
and especially preferably YP_{—}002835700.1, ZP_{—}03936415.1, BAE47461.1, YP_{—}001801238.1, ZP_{—}03978917.1, ZP_{—}03394212.1, ZP_{—}05847263.1, ZP_{—}08516453.1, YP_{—}004606508.1, YP_{—}251740.1, ZP_{—}07090640.1, ZP_{—}07989876.1, YP_{—}004761186.1,
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{c} is generally understood in particular as meaning oxidation of dodecanoyl-CoA thioester to give 2-dodecenoyl-CoA thioester.

### Specific Enzymes E_{d} and Eₑ

Furthermore, it is preferred according to the invention that the enzyme E_{d} or Eₑ in the cells according to the invention is one which comprises sequences selected from among:

ZP_{—}07164313.1, NP_{—}418288.1, YP_{—}003231641.1, EGM59778.1, EFZ53307.1, AAA23750.1, ZP_{—}07192215.1, YP_{—}001460638.1, YP_{—}001727088.1, EGK16564.1, ZP_{—}08380619.1, ZP_{—}07136310.1, CAB40809.1, NP_{—}839030.1, ZP_{—}07690617.1, EGC97039.1, ZP_{—}07103516.1, ZP_{—}03027888.1, ZP_{—}07121980.1, YP_{—}002414996.1, EGP22873.1, EGJ82677.1, EGB59499.1, ZP_{—}07118761.1, YP_{—}002409078.1, YP_{—}002295407.1, EGE62412.1, EGB69560.1, ZP_{—}06655948.1, ZP_{—}06664574.1, ZP_{—}03070699.1, ZP_{—}07145404.1, ZP_{—}08376058.1, EGB85466.1, ZP_{—}07189176.1, ZP_{—}02999920.1, ZP_{—}08356523.1, ZP_{—}06659936.1, ZP_{—}07139396.1, YP_{—}001746178.1, YP_{—}002384700.1, ZP_{—}07098889.1, CBG37051.1, ZP_{—}04873109.1, CBJ03626.1, ZP_{—}08366395.1, ZP_{—}03066301.1, BAI57243.1, YP_{—}001465330.1, YP_{—}405325.1, NP_{—}312801.1, EGI89589.1, EGC09628.1, EFW73050.1, ZP_{—}07221474.1, EGB39932.1, EFW72281.1, ZP_{—}07154547.1, YP_{—}002331616.1, EGB76756.1, EFZ75005.1, ZP_{—}07449248.1, NP_{—}756652.2, ZP_{—}04006347.1, NP_{—}290476.1, EGH36687.1, YP_{—}671920.1, ZP_{—}08350773.1, EGC05062.1, ZP_{—}07174622.1, CAP78309.1, ZP_{—}08361145.1, YP_{—}002400350.1, ZP_{—}08386169.1, EFU60028.1, ZP_{—}02904283.1, YP_{—}859447.1, YP_{—}543379.2, NP_{—}462868.1, ZP_{—}02663494.1, ACY91152.1, ZP_{—}03221347.1, YP_{—}001591071.1, YP_{—}002639596.1, EFY10009.1, ZP_{—}04656823.1, ZP_{—}03213459.1, ZP_{—}02701437.1, ZP_{—}02347126.1, YP_{—}002217909.1, ZP_{—}02658976.1, YP_{—}002245833.1, YP_{—}002228260.1, YP_{—}001451793.1, EGE35935.1, YP_{—}002047992.1, ZP_{—}02834645.1, ZP_{—}02669606.1, YP_{—}001572623.1, ZP_{—}03075319.1, YP_{—}002148908.1, YP_{—}004591813.1, ZP_{—}03163685.1, YP_{—}002043211.1, NP_{—}457769.1, YP_{—}003367347.1, YP_{—}004732313.1, ZP_{—}06546517.1, ZP_{—}08495782.1, ZP_{—}04558441.1, YP_{—}002241091.1, ZP_{—}06354348.2, ZP_{—}06552493.1, YP_{—}001337975.1, YP_{—}001178655.1, CBK87780.1, ZP_{—}05970964.1, ZP_{—}06014071.1, YP_{—}002917176.1, YP_{—}152910.1, Q9F0Y7.1, ZP_{—}08302760.1, YP_{—}003615422.1, YP_{—}003943709.1, EGI93642.1, YP_{—}001439747.1, YP_{—}003208640.1, YP_{—}001476499.1, ZP_{—}06638309.1, YP_{—}004498688.1, YP_{—}004296470.1, ZP_{—}06192594.1, YP_{—}001004653.1, ZP_{—}04634366.1, CBY29055.1, ZP_{—}04641538.1, ZP_{—}04628383.1, ZP_{—}04620754.1, ZP_{—}04624649.1, YP_{—}003739635.1, ZP_{—}07953302.1, YP_{—}001399280.1, NP_{—}667802.1, YP_{—}068813.1, ADV97208.1, ZP_{—}04637125.1, YP_{—}003019595.1, YP_{—}048335.1, ZP_{—}04612255.1, ZP_{—}03831342.1, ZP_{—}03827989.1, YP_{—}003261565.1, ZP_{—}04616540.1, EFW54755.1, YP_{—}004214864.1, BAK13441.1, YP_{—}003518496.1, YP_{—}003933023.1, ZP_{—}07380063.1, YP_{—}003042702.1, YP_{—}003713991.1, YP_{—}003466462.1, YP_{—}004114076.1, YP_{—}001906200.1, NP_{—}931575.1, EGK17810.1, CBX79037.1, YP_{—}003529581.1, ZP_{—}06937250.1, YP_{—}002647270.1, ADP11112.1, ZP_{—}05974166.1, ZP_{—}03318464.1, ZP_{—}02958886.1, YP_{—}003331802.1, ZP_{—}06125606.1, YP_{—}003006180.1, YP_{—}003885045.1, YP_{—}128321.1, ZP_{—}01236908.1, ZP_{—}01161145.1, YP_{—}002989323.1, YP_{—}002154796.1, YP_{—}203408.1, ZP_{—}08310903.1, YP_{—}002264299.1, ZP_{—}01221704.1, ZP_{—}06050960.1, ZP_{—}03841335.1, ZP_{—}05883431.1, YP_{—}002153226.1, ADT85583.1, ZP_{—}05879947.1, ZP_{—}04923724.1, ZP_{—}01262258.1, ZP_{—}06179383.1, ZP_{—}05883853.1, EGF42158.1, ZP_{—}01957954.1, ZP_{—}08101926.1, ZP_{—}06177050.1, NP_{—}759944.1, NP_{—}796409.1, ZP_{—}04419618.1, ZP_{—}01987794.1, ZP_{—}05121182.1, YP_{—}001443702.1, ZP_{—}01948571.1, ZP_{—}01682057.1, ZP_{—}04405432.1, NP_{—}232384.1, ZP_{—}04409574.1, ZP_{—}01870127.1, NP_{—}932822.1, ZP_{—}06943917.1, EGR05147.1, ZP_{—}04961951.1, EGR10674.1, ZP_{—}04414292.1, ZP_{—}05718020.1, ZP_{—}08098153.1, ZP_{—}05719938.1, ZP_{—}03356468.1, ZP_{—}07742015.1, YP_{—}004564872.1, ZP_{—}01979859.1, ZP_{—}00992843.1, ZP_{—}05927571.1, ZP_{—}01065523.1, YP_{—}002415749.1, ZP_{—}01815881.1, ZP_{—}02196043.1, YP_{—}001143922.1, ZP_{—}08518445.1, ZP_{—}06156529.1, YP_{—}004394586.1, ZP_{—}01900693.1, YP_{—}854676.1, ZP_{—}05943242.1, CBA71812.1, ZP_{—}01991723.1, YP_{—}001092151.1, YP_{—}001672251.1, YP_{—}961420.1, YP_{—}003554801.1, YP_{—}003911300.1, ADV52504.1, YP_{—}001364252.1, YP_{—}001552462.1, ZP_{—}07394327.1, YP_{—}001048426.1, YP_{—}002355987.1, ZP_{—}02158912.1, ABE53312.1, YP_{—}561035.2, YP_{—}748714.1, ZP_{—}01135242.1, NP_{—}715663.1, YP_{—}732157.1, YP_{—}867675.1, YP_{—}736079.1, YP_{—}001758417.1, YP_{—}001499882.1, YP_{—}004436299.1, YP_{—}659787.1, ZP_{—}08620874.1, YP_{—}002309470.1, CBW44433.1, ZP_{—}08568624.1, YP_{—}958423.1, YP_{—}925914.1, YP_{—}001471764.1, ZP_{—}01165107.1, ZP_{—}04717156.1, ZP_{—}01042072.1, ZP_{—}08568929.1, YP_{—}004468425.1, ZP_{—}01614054.1, EGH60623.1, NP_{—}744285.1, ZP_{—}04587907.1, EGH84450.1, YP_{—}609235.1, Q93Q12.1, ZP_{—}07263341.1, YP_{—}004425808.1, EGH10831.1, ZP_{—}08142928.1, YP_{—}435877.1, YP_{—}004701152.1, ADR61111.1, EGH72107.1, ZP_{—}07255969.1, EGH76237.1, YP_{—}154404.1, EGH66371.1, ZP_{—}07005687.1, YP_{—}001268914.1, ZP_{—}03397164.1, YP_{—}267151.1, EGH45982.1, NP_{—}793297.1, YP_{—}236360.1, YP_{—}001667915.1, EGH29726.1, ZP_{—}03561781.1, YP_{—}275370.1, ABP88736.1, ZP_{—}06458302.1, YP_{—}001748526.1, YP_{—}002871195.1, ZP_{—}06478839.1, EGH95845.1, YP_{—}004067126.1, EGH21541.1, ZP_{—}05638744.1, Q9AHY3.2, YP_{—}338568.1, ZP_{—}06078672.1, YP_{—}004352961.1, ZP_{—}01892768.1, ZP_{—}06040413.1, YP_{—}349607.1, YP_{—}259059.1, ZP_{—}08409548.1, ADP97276.1, YP_{—}004713990.1, YP_{—}003626258.1, P28793.1, YP_{—}001172246.1, YP_{—}003810247.1, YP_{—}004313957.1, EGE21928.1, EGE19309.1, EGE13641.1, ZP_{—}08462037.1, EGE13529.1, ZP_{—}06034789.1, EGE12165.1, AEA83639.1, YP_{—}002798635.1, ZP_{—}01306165.1, YP_{—}004474976.1, ZP_{—}01739261.1, NP_{—}251704.1, ACP17923.1, YP_{—}004379416.1, YP_{—}001280990.1, YP_{—}003145204.1, YP_{—}001347517.1, ZP_{—}06877966.1, YP_{—}001187076.1, ZP_{—}08638729.1, YP_{—}001340441.1, ZP_{—}05128804.1, YP_{—}003896827.1, YP_{—}003073151.1, ZP_{—}05096745.1, ZP_{—}01103278.1, YP_{—}693372.1, ZP_{—}01366482.1, ZP_{—}05619303.1, ZP_{—}08328596.1, ZP_{—}05042935.1, YP_{—}574439.1, ZP_{—}01074264.1, YP_{—}004482149.1, YP_{—}045111.1, YP_{—}265216.1, ZP_{—}05362445.1, YP_{—}001715228.1, YP_{—}001844981.1, YP_{—}001708314.1, YP_{—}581488.1, ADY83798.1, ZP_{—}06692406.1, YP_{—}003733838.1, ZP_{—}05824704.1, ZP_{—}06058514.1, ZP_{—}08554004.1, ZP_{—}06068411.1, ZP_{—}06067277.1, ZP_{—}06726497.1, ADX01983.1, ZP_{—}03822268.1, ZP_{—}03347927.1, ZP_{—}01116792.1, YP_{—}527079.1, ZP_{—}06063435.1, ZP_{—}06534677.1, ZP_{—}01219812.1, ZP_{—}03347768.1, YP_{—}002798829.1, ZP_{—}07774142.1, YP_{—}003557881.1, ZP_{—}06157092.1, ZP_{—}01223872.1, ZP_{—}05946076.1, ZP_{—}06499586.1, YP_{—}003451185.1, YP_{—}002361722.1, YP_{—}003266103.1, YP_{—}285556.2, AAZ47086.1, NP_{—}968701.1, ZP_{—}06936670.1, ZP_{—}03805048.1, YP_{—}943922.1, ZP_{—}01217009.1, ADT87675.1, ZP_{—}05877956.1, ZP_{—}03355309.1, ZP_{—}05885304.1, EGK17811.1, ZP_{—}05944972.1, ZP_{—}05119053.1, ZP_{—}06039619.1, ZP_{—}05716842.1, ZP_{—}05721090.1, ZP_{—}06079171.1, ZP_{—}06033023.1, ZP_{—}08098475.1, ZP_{—}08104504.1, ZP_{—}06048048.1, ZP_{—}01677170.1, ZP_{—}01681193.1, NP_{—}230692.2, ZP_{—}05926205.1, ZP_{—}05881372.1, ZP_{—}01975051.1, ZP_{—}04412573.1, ZP_{—}01977591.1, ZP_{—}04415061.1, ZP_{—}06048243.1, YP_{—}742943.1, ZP_{—}04962518.1, ZP_{—}01955504.1, ZP_{—}07741831.1, EGK33112.1, ZP_{—}01980800.1, CBW26643.1, EGQ99075.1, ZP_{—}03561616.1, ZP_{—}06155835.1, ZP_{—}01613403.1, YP_{—}003147156.1, ZP_{—}01866421.1, ZP_{—}08569601.1, YP_{—}004068133.1, ZP_{—}01992793.1, YP_{—}003760621.1, NP_{—}760849.1, NP_{—}935233.1, YP_{—}661240.1, CBA76402.1, YP_{—}003527567.1, ZP_{—}05071916.1, YP_{—}155382.1, ZP_{—}08567109.1, ZP_{—}08410490.1, YP_{—}002357526.1, YP_{—}001473368.1, ZP_{—}05061211.1, ZP_{—}08309062.1, ZP_{—}00990722.1, ZP_{—}01813160.1, YP_{—}343735.1, YP_{—}001366977.1, ZP_{—}07393465.1, YP_{—}002312436.1, ZP_{—}03805047.1, ZP_{—}04716066.1, ZP_{—}01043968.1, YP_{—}562538.1, ZP_{—}01064421.1, YP_{—}928042.1, YP_{—}002416486.1, YP_{—}962941.1, YP_{—}001051116.1, YP_{—}004467793.1, YP_{—}004434876.1, YP_{—}001183979.1, ZP_{—}01125518.1, YP_{—}001555281.1, ZP_{—}01900341.1, YP_{—}001459147.1, ADV54930.1, ZP_{—}06054161.1, YP_{—}001674882.1, YP_{—}001381324.1, ZP_{—}02158374.1, NP_{—}718651.1, YP_{—}737529.1, YP_{—}869101.1, ZP_{—}01258852.1, ZP_{—}05978956.1, ZP_{—}06179776.1, YP_{—}733543.1, ZP_{—}01989664.1, NP_{—}798587.1, EGF45285.1, ZP_{—}05908370.1, YP_{—}001502453.1, ZP_{—}06639387.1, YP_{—}003557654.1, ZP_{—}04921889.1, YP_{—}001436988.1, YP_{—}003468880.1, YP_{—}001761392.1, YP_{—}003267851.1, YP_{—}004730996.1, EGL72460.1, YP_{—}003742516.1, YP_{—}003258850.1, ZP_{—}01132697.1, ZP_{—}01987078.1, YP_{—}004392689.1, ZP_{—}06191156.1, YP_{—}002381996.1, ZP_{—}06176023.1, EGC06853.1, ZP_{—}07196084.1, NP_{—}754768.1, ZP_{—}02901855.1, ZP_{—}08620438.1, EGE30558.1, YP_{—}003211325.1, ZP_{—}03220131.1, YP_{—}217377.1, YP_{—}003940937.1, YP_{—}004669896.1, YP_{—}633521.1, YP_{—}002041652.1, NP_{—}456929.1, YP_{—}001446296.1, ZP_{—}02699767.1, YP_{—}001586838.1, YP_{—}751355.1, ZP_{—}08384609.1, YP_{—}002216460.1, A8 GH86.2, ZP_{—}02667448.1, YP_{—}004595105.1, YP_{—}002408448.1, YP_{—}001479604.1, YP_{—}149790.1, NP_{—}461330.1, YP_{—}002227302.1, ZP_{—}07187886.1, ZP_{—}08374604.1, ZP_{—}02343362.1, ZP_{—}02683558.1, YP_{—}001141958.1, ZP_{—}02662473.1, ZP_{—}07151809.1, YP_{—}004211957.1, YP_{—}003366276.1, YP_{—}003713364.1, ZP_{—}03035287.1, ZP_{—}08364768.1, YP_{—}002413389.1, ZP_{—}07448710.1, ZP_{—}04656170.1, ZP_{—}02654823.1, ZP_{—}01222785.1, EGB63194.1, ZP_{—}08359459.1, YP_{—}002636921.1, YP_{—}002329984.1, YP_{—}001744544.1, CAP76837.1, EFZ73229.1, EFU57443.1, YP_{—}002398712.1, YP_{—}003018387.1, ZP_{—}08520753.1, YP_{—}541623.1, ZP_{—}02574174.1, ZP_{—}07144040.1, ZP_{—}08349090.1, CBG35413.1, ZP_{—}04562847.1, ZP_{—}02195785.1, ZP_{—}02773221.1, EGB40918.1, ZP_{—}03050715.1, ZP_{—}07787570.1, ZP_{—}03831301.1, YP_{—}003003682.1, ZP_{—}08354786.1, YP_{—}051168.1, YP_{—}002403607.1, AEE57458.1, YP_{—}856678.1, YP_{—}001177597.1, ZP_{—}06658276.1, NP_{—}288914.1, YP_{—}002392166.1, ZP_{—}06654274.1, ZP_{—}07102361.1, EGB72544.1, YP_{—}004501987.1, ZP_{—}03027319.1, YP_{—}670274.1, YP_{—}003913906.1, ZP_{—}07097669.1, YP_{—}001463687.1, BAI55757.1, ZP_{—}08553509.1, YP_{—}003500399.1, ZP_{—}07121648.1, ZP_{—}01235780.1, CBK87125.1, YP_{—}002293925.1, ZP_{—}05431367.1, YP_{—}129175.1, ZP_{—}03003629.1, YP_{—}002387809.1, ZP_{—}03043524.1, YP_{—}001569579.1, ZP_{—}05435840.1, ZP_{—}01464666.1, YP_{—}001724305.1, ZP_{—}03068335.1, CBJ01980.1, AEJ57562.1, NP_{—}416843.1, YP_{—}002920590.1, ZP_{—}03828462.1, EGM60943.1, ZP_{—}06351976.1, ZP_{—}05968584.1, EGK21055.1, YP_{—}003040254.1, NP_{—}708223.1, YP_{—}689824.1, ZP_{—}04625886.1, AEJ99232.1, ZP_{—}07135079.1, YP_{—}339488.1, ZP_{—}07247352.1, ZP_{—}07590743.1, ZP_{—}08303100.1, EFU96242.1, EFZ69715.1, YP_{—}001336370.1, YP_{—}001094550.1, ZP_{—}07679578.1, ZP_{—}06547779.1, EGI93593.1, YP_{—}003438264.1, YP_{—}003614165.1, YP_{—}408769.1, YP_{—}001881164.1, YP_{—}003655512.1, YP_{—}002237269.1, YP_{—}004116642.1, ZP_{—}03065203.1, ZP_{—}07951118.1, CAQ79951.1, AAZ26206.1, BAK12062.1, YP_{—}269853.2, NP_{—}930429.2, YP_{—}404102.1, ZP_{—}04620204.1, ZP_{—}08498986.1, YP_{—}001452041.1, ZP_{—}01159981.1, CAE15574.1, A1JK30.2, ZP_{—}04635573.1, ZP_{—}02904987.1, ZP_{—}02961182.1, YP_{—}001005598.1, ZP_{—}01301762.1, ZP_{—}06016509.1, CBY28037.1, ZP_{—}05060968.1, ZP_{—}04632512.1, YP_{—}002156637.1, YP_{—}002132807.1, Q5E3U1.2, YP_{—}205193.1, ZP_{—}04613435.1, ZP_{—}07380136.1, YP_{—}004299028.1, YP_{—}003334344.1, YP_{—}001610684.1, YP_{—}001720255.1, YP_{—}001400379.1, YP_{—}652007.1, NP_{—}668898.1, ZP_{—}04640314.1, ADV98116.1, ZP_{—}03840558.1, ZP_{—}07047543.1, ZP_{—}03320348.1, YP_{—}001681761.1, ZP_{—}04615169.1, ZP_{—}08182604.1, YP_{—}003520988.1, YP_{—}002151536.1, NP_{—}641653.1, ZP_{—}08188276.1, Q668V1.2, YP_{—}463621.1, ZP_{—}05032523.1, YP_{—}363100.1, YP_{—}002490860.1, YP_{—}071146.1, YP_{—}003527951.1, YP_{—}004615064.1, ZP_{—}06702935.1, YP_{—}003277339.1, ZP_{—}06729873.1, YP_{—}004552309.1, ZP_{—}08178119.1, YP_{—}558747.1, YP_{—}003059322.1, ZP_{—}04628689.1, ZP_{—}05043496.1, YP_{—}755774.1, NP_{—}106254.1, NP_{—}774461.1, YP_{—}004145058.1, NP_{—}636640.1, YP_{—}001411745.1, YP_{—}244043.1, YP_{—}003906899.1, ZP_{—}02151779.1, EFW54754.1, YP_{—}004147062.1, YP_{—}434583.1, ZP_{—}06862658.1, YP_{—}003559491.1, ZP_{—}07474361.1, ZP_{—}07478578.1, ZP_{—}03787298.1, ZP_{—}06840682.1, ZP_{—}05161835.1, ZP_{—}06794105.1, ZP_{—}05181908.1, ZP_{—}05174379.1, YP_{—}003883888.1, NP_{—}541475.1, NP_{—}949054.1, YP_{—}003931777.1, YP_{—}001993209.1, ZP_{—}06124668.1, YP_{—}001594738.1, ZP_{—}06070710.1, ZP_{—}06484372.1, YP_{—}002515449.1, YP_{—}001895558.1, YP_{—}002029364.1, ZP_{—}02891585.1, ZP_{—}04682672.1, YP_{—}003761433.1, YP_{—}004107983.1, YP_{—}223224.1, YP_{—}003812264.1, YP_{—}001622574.1, ZP_{—}05452320.1, YP_{—}002734532.1, YP_{—}001257739.1, YP_{—}001372564.1, ZP_{—}05137372.1, YP_{—}001973266.1, YP_{—}342869.1, NP_{—}699967.1, ZP_{—}05086267.1, ZP_{—}01736760.1, YP_{—}001914218.1, ZP_{—}05157647.1, YP_{—}485365.1, YP_{—}001926123.1, ZP_{—}05116437.1, ZP_{—}03544469.1, ZP_{—}08330383.1, ZP_{—}06491403.1, ZP_{—}01896167.1, ADP99705.1, ZP_{—}02883593.1, YP_{—}004228182.1, YP_{—}570677.1, ZP_{—}01225298.1, YP_{—}200487.1, YP_{—}002988196.1, ZP_{—}08269313.1, NP_{—}767800.1, YP_{—}001094989.1, ZP_{—}06065014.1, YP_{—}002981447.1, YP_{—}001260831.1, YP_{—}003817548.1, YP_{—}532099.1, ZP_{—}07676723.1, YP_{—}001242863.1, ZP_{—}02244047.1, YP_{—}982073.1, YP_{—}001899020.1, NP_{—}519880.1, ZP_{—}02379339.1, NP_{—}946171.1, ZP_{—}01615132.1, YP_{—}456953.1, ZP_{—}02168372.1, ZP_{—}08552434.1, CBJ37969.1, YP_{—}004418392.1, ZP_{—}02362492.1, YP_{—}004107339.1, YP_{—}001203133.1, ZP_{—}01546752.1, YP_{—}002974094.1, ZP_{—}02186892.1, YP_{—}001989920.1, YP_{—}002964466.1, ZP_{—}03265887.1, YP_{—}555553.1, CBA26305.1, ZP_{—}06728723.1, ZP_{—}07656835.1, ZP_{—}05620865.1, YP_{—}575713.1, YP_{—}001907090.1, YP_{—}002911224.1, YP_{—}047520.1, YP_{—}004688052.1,
in particular
EGH60623.1, NP_{—}744285.1, ZP_{—}04587907.1, EGH84450.1, YP_{—}609235.1, Q93Q12.1, ZP_{—}07263341.1, EGH10831.1, ZP_{—}08142928.1, YP_{—}004701152.1, ADR61111.1, EGH72107.1, ZP_{—}07255969.1, EGH76237.1, EGH66371.1, ZP_{—}07005687.1, YP_{—}001268914.1, ZP_{—}03397164.1, EGH45982.1, NP_{—}793297.1, YP_{—}236360.1, YP_{—}001667915.1, EGH29726.1, YP_{—}275370.1, ABP88736.1, ZP_{—}06458302.1, YP_{—}001748526.1, YP_{—}002871195.1, ZP_{—}06478839.1, EGH95845.1, EGH21541.1, ZP_{—}05638744.1, Q9AHY3.2, YP_{—}004352961.1, YP_{—}349607.1, YP_{—}259059.1, YP_{—}004713990.1, P28793.1, YP_{—}001172246.1, AEA83639.1, YP_{—}004474976.1, NP_{—}251704.1, ACP17923.1, YP_{—}004379416.1, YP_{—}001347517.1, ZP_{—}06877966.1, YP_{—}001187076.1, ZP_{—}01366482.1, ZP_{—}07774142.1, ZP_{—}06499586.1, YP_{—}791508.1, ZP_{—}07796310.1, NP_{—}250428.1, YP_{—}002441177.1, YP_{—}001348922.1, ZP_{—}06879352.1, AEA82038.1, YP_{—}001170648.1, YP_{—}004473370.1, YP_{—}004712521.1, YP_{—}004353314.1, ZP_{—}07164313.1, NP_{—}418288.1, YP_{—}003231641.1, AAA23750.1, ZP_{—}07192215.1, YP_{—}001460638.1, YP_{—}001727088.1, ZP_{—}08380619.1, ZP_{—}07136310.1, CAB40809.1, ZP_{—}07690617.1, ZP_{—}07103516.1, ZP_{—}03027888.1, ZP_{—}07121980.1, YP_{—}002414996.1, EGP22873.1, EGB59499.1, ZP_{—}07118761.1, YP_{—}002409078.1, YP_{—}002295407.1, EGE62412.1, EGB69560.1, ZP_{—}06655948.1, ZP_{—}06664574.1, ZP_{—}03070699.1, ZP_{—}07145404.1, ZP_{—}08376058.1, EGB85466.1, ZP_{—}07189176.1, ZP_{—}02999920.1, ZP_{—}08356523.1, ZP_{—}06659936.1, ZP_{—}07139396.1, YP_{—}001746178.1, ZP_{—}07098889.1, CBG37051.1, CBJ03626.1, ZP_{—}08366395.1, BAI57243.1, YP_{—}001465330.1, NP_{—}312801.1, EGC09628.1, EFW73050.1, ZP_{—}07221474.1, EGB39932.1, EFW72281.1, ZP_{—}07154547.1, YP_{—}002331616.1, EGB76756.1, EFZ75005.1, ZP_{—}07449248.1, NP_{—}756652.2, ZP_{—}04006347.1, NP_{—}290476.1, EGH36687.1, YP_{—}671920.1, ZP_{—}08350773.1, ZP_{—}07174622.1, CAP78309.1, ZP_{—}08361145.1, YP_{—}002400350.1, ZP_{—}08386169.1, EFU60028.1, YP_{—}859447.1, YP_{—}543379.2, ZP_{—}06937250.1, ZP_{—}06936670.1, YP_{—}001459147.1, ZP_{—}07196084.1, NP_{—}754768.1, ZP_{—}08384609.1, YP_{—}002408448.1, ZP_{—}07187886.1, ZP_{—}08374604.1, ZP_{—}07151809.1, ZP_{—}03035287.1, ZP_{—}08364768.1, YP_{—}002413389.1, ZP_{—}07448710.1, EGB63194.1, ZP_{—}08359459.1, YP_{—}002329984.1, YP_{—}001744544.1, CAP76837.1, EFZ73229.1, EFU57443.1, YP_{—}002398712.1, YP_{—}541623.1, ZP_{—}07144040.1, ZP_{—}08349090.1, CBG35413.1, ZP_{—}02773221.1, EGB40918.1, ZP_{—}03050715.1, ZP_{—}07787570.1, ZP_{—}08354786.1, YP_{—}002403607.1, AEE57458.1, ZP_{—}06658276.1, NP_{—}288914.1, YP_{—}002392166.1, ZP_{—}06654274.1, ZP_{—}07102361.1, EGB72544.1, ZP_{—}03027319.1, YP_{—}670274.1, ZP_{—}07097669.1, YP_{—}001463687.1, BAI55757.1, YP_{—}003500399.1, ZP_{—}07121648.1, YP_{—}002293925.1, ZP_{—}03003629.1, YP_{—}002387809.1, ZP_{—}03043524.1, YP_{—}001724305.1, ZP_{—}03068335.1, CBJ01980.1, AEJ57562.1, NP_{—}416843.1, ZP_{—}07135079.1, ZP_{—}07247352.1, ZP_{—}07590743.1, EFU96242.1, EFZ69715.1,
and especially preferably
NP_{—}744285.1, YP_{—}004701152.1, ADR61111.1, YP_{—}001268914.1, YP_{—}001667915.1, ABP88736.1, YP_{—}001748526.1, Q9AHY3.2, YP_{—}004713990.1, YP_{—}001172246.1, AEA83639.1, AEA82038.1, YP_{—}001170648.1, YP_{—}004712521.1, YP_{—}002871195.1, YP_{—}349607.1, YP_{—}259059.1, ZP_{—}07774142.1, NP_{—}418288.1, NP_{—}416843.1, ZP_{—}07593201.1, ZP_{—}07590743.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{d} and Eₑ is generally understood in particular as meaning conversion of 2-dodecenoyl-CoA thioester into 3-oxododecanoyl-CoA thioester.

### Specific Enzymes E_{f}

Furthermore, it is preferred according to the invention that the enzyme E_{f} in the cells according to the invention is one which comprises sequences selected from among:

YP_{—}026272.1, YP_{—}002389323.1, EGB30581.1, YP_{—}001460637.1, YP_{—}001727089.1, CAB40810.1, EGK16565.1, NP_{—}709649.1, YP_{—}001882545.1, ZP_{—}08356522.1, ZP_{—}06664573.1, AAA67642.1, ADA76222.1, EGK17812.1, YP_{—}405326.1, YP_{—}003236969.1, ZP_{—}06659935.1, YP_{—}410143.1, NP_{—}290475.1, ZP_{—}03027945.1, EFZ59092.1, YP_{—}002295406.1, CBG37050.1, EGP22872.1, EGE62411.1, EGC97040.1, ZP_{—}05435276.1, YP_{—}002400349.1, EGB59498.1, EFW54756.1, ZP_{—}08361144.1, YP_{—}001465329.1, YP_{—}002384701.1, YP_{—}002409079.1, ZP_{—}06655947.1, YP_{—}002414995.1, EGB69559.1, YP_{—}859446.1, EGC05061.1, ZP_{—}02904263.1, ZP_{—}08386168.1, YP_{—}543378.1, ZP_{—}08366394.1, ZP_{—}03066325.1, YP_{—}001746177.1, ZP_{—}07154548.1, ZP_{—}03070708.1, NP_{—}756651.1, YP_{—}312775.1, YP_{—}671919.1, YP_{—}002331615.1, YP_{—}003367348.1, ZP_{—}07449249.1, ZP_{—}04558440.1, ZP_{—}06354347.1, YP_{—}001451792.1, YP_{—}004732312.1, ZP_{—}06938722.1, NP_{—}457770.1, ZP_{—}02834646.1, ZP_{—}02658975.1, ZP_{—}03221210.1, EFY10008.1, YP_{—}001591070.1, YP_{—}218866.1, YP_{—}003943710.1, ZP_{—}03086141.1, ZP_{—}03163187.1, ZP_{—}08495783.1, EGE35936.1, YP_{—}003615423.1, YP_{—}002228261.1, YP_{—}002148907.1, ZP_{—}05970963.1, YP_{—}002241092.1, YP_{—}004591812.1, YP_{—}001178656.1, ZP_{—}08302761.1, YP_{—}002917175.1, YP_{—}001337974.1, Q9F0Y6.1, ZP_{—}06014072.1, YP_{—}001439748.1, YP_{—}003208639.1, 3GOA_A, YP_{—}003739634.1, ZP_{—}06192593.1, YP_{—}001906199.1, YP_{—}001476498.1, YP_{—}003019596.1, YP_{—}003261566.1, ZP_{—}03831341.1, ZP_{—}07380062.1, YP_{—}048334.1, YP_{—}003933022.1, ZP_{—}07953301.1, YP_{—}004114075.1, YP_{—}002647269.1, ADP 11111.1, ZP_{—}03827988.1, ZP_{—}06638308.1, YP_{—}004214865.1, YP_{—}003518495.1, ZP_{—}04616541.1, BAK13440.1, CBX79036.1, YP_{—}003529580.1, ZP_{—}04628384.1, ZP_{—}04634365.1, ZP_{—}04641537.1, ZP_{—}04612254.1, ZP_{—}04637126.1, YP_{—}003331801.1, YP_{—}001004652.1, YP_{—}004296469.1, YP_{—}003006181.1, ZP_{—}04620755.1, YP_{—}003885046.1, ZP_{—}04624648.1, NP_{—}667801.1, EGI89588.1, YP_{—}128320.1, ZP_{—}01221705.1, YP_{—}002989324.1, ZP_{—}01236909.1, ZP_{—}01161146.1, ZP_{—}08310904.1, YP_{—}003713992.1, YP_{—}003466461.1, NP_{—}931576.1, YP_{—}003042703.1, ZP_{—}06050959.1, EGF42157.1, ZP_{—}00992844.1, YP_{—}002415748.1, ZP_{—}01065522.1, ZP_{—}05883432.1, ZP_{—}05879948.1, ZP_{—}06156528.1, ZP_{—}05927570.1, ZP_{—}07189177.1, YP_{—}004564871.1, ZP_{—}01870126.1, ZP_{—}02196042.1, YP_{—}003911299.1, ZP_{—}01815882.1, NP_{—}796408.1, YP_{—}004394587.1, ZP_{—}08518444.1, YP_{—}854675.1, ZP_{—}07742014.1, ZP_{—}01135243.1, YP_{—}002154795.1, NP_{—}759945.1, YP_{—}001143923.1, NP_{—}932821.1, Q5E8X7.2, ZP_{—}08568928.1, ZP_{—}06078671.1, ZP_{—}05718021.1, ZP_{—}01948567.1, YP_{—}203407.3, ZP_{—}04923725.1, ZP_{—}05719937.1, YP_{—}002309469.1, ZP_{—}06179384.1, ZP_{—}06048881.1, ZP_{—}01979851.1, ZP_{—}01262259.1, ZP_{—}01957951.1, ZP_{—}04405431.1, ZP_{—}08098152.1, ZP_{—}06034494.1, YP_{—}001758416.1, ZP_{—}04414293.1, ZP_{—}06040414.1, ZP_{—}01682043.1, NP_{—}232385.1, ZP_{—}05883854.1, YP_{—}002264298.1, ZP_{—}01987792.1, YP_{—}338567.1, ZP_{—}01900694.1, YP_{—}001672250.1, YP_{—}925913.1, YP_{—}001499881.1, ZP_{—}02158913.1, YP_{—}001471763.1, NP_{—}715662.1, YP_{—}748713.1, YP_{—}736078.1, ZP_{—}08568623.1, ZP_{—}02958885.2, YP_{—}004067125.1, ZP_{—}08409549.1, YP_{—}001181547.1, ADV52503.1, YP_{—}732156.1, YP_{—}001092150.1, YP_{—}003554800.1, YP_{—}001048425.1, YP_{—}961419.1, YP_{—}561034.1, ZP_{—}06125607.2, YP_{—}867674.1, YP_{—}001443701.1, ZP_{—}05943241.1, ZP_{—}05121169.1, ZP_{—}05974167.1, ZP_{—}03318463.1, ZP_{—}08620875.1, ZP_{—}01042073.1, YP_{—}154403.1, ZP_{—}04717155.1, ZP_{—}03805050.1, YP_{—}004468426.1, ZP_{—}03841336.1, YP_{—}002153225.1, YP_{—}004425807.1, ZP_{—}03351120.1, YP_{—}659788.1, YP_{—}004436298.1, YP_{—}267150.1, ZP_{—}06034790.1, YP_{—}003145205.1, ZP_{—}03561780.1, YP_{—}003810248.1, ZP_{—}05043383.1, YP_{—}693373.1, ZP_{—}01306166.1, YP_{—}004313956.1, YP_{—}790159.1, ZP_{—}06877967.1, NP_{—}251703.1, YP_{—}004482148.1, EGH66370.1, EGH10832.1, YP_{—}349606.1, YP_{—}236359.1, ZP_{—}07263340.1, EGH95844.1, ZP_{—}03368595.1, NP_{—}793296.1, ZP_{—}01165108.1, 1WDK_C, P28790.2, YP_{—}259060.1, ZP_{—}01074263.1, ZP_{—}04587908.1, EGH45981.1, ZP_{—}07774144.1, EGH84449.1, YP_{—}958424.1, YP_{—}275369.1, ZP_{—}07005686.1, YP_{—}001172247.1, YP_{—}004352962.1, ACP17922.1, YP_{—}002871196.1, YP_{—}435876.1, ZP_{—}01739262.1, YP_{—}003557880.1, ZP_{—}01892767.1, ZP_{—}08142929.1, ZP_{—}08462036.1, YP_{—}004701153.1, YP_{—}001667916.1, YP_{—}001280989.1, YP_{—}001268913.1, Q93Q11.1, ZP_{—}05619304.1, AEA79634.1, Q9R9W0.1, NP_{—}744286.1, YP_{—}001187077.1, YP_{—}609234.1, ADP97277.1, YP_{—}045110.1, YP_{—}004379417.1, YP_{—}003626259.1, ZP_{—}06692405.1, ZP_{—}06063436.1, YP_{—}003733839.1, EGE12166.1, ZP_{—}05824703.1, EGE26385.1, EGE13530.1, YP_{—}004474975.1, YP_{—}001708315.1, EGE 16076.1, ZP_{—}06726496.1, ZP_{—}06067276.1, ZP_{—}06058513.1, ZP_{—}06068412.1, ZP_{—}06157093.1, ZP_{—}03822267.1, A3M1H9.2, YP_{—}001340442.1, ZP_{—}05362446.1, ABP88737.1, ZP_{—}01219813.1, ZP_{—}08638730.1, YP_{—}265215.1, YP_{—}581487.1, YP_{—}003896828.1, YP_{—}002798636.1, ZP_{—}01678475.1, ZP_{—}05946075.1, YP_{—}527080.1, ZP_{—}08554005.1, ZP_{—}03360083.1, YP_{—}574438.1, YP_{—}003073152.1, YP_{—}001083375.1, ZP_{—}08648989.1, YP_{—}001982171.1, ZP_{—}05096741.1, ZP_{—}03336985.1, ZP_{—}01103277.1, ZP_{—}07136312.1, ZP_{—}08328590.1, ZP_{—}05128805.1, EGH76239.1, ZP_{—}03377529.1, CBA71811.1, EFZ47010.1, ZP_{—}03377530.1, ZP_{—}07136311.1, EFZ47009.1, EGH29725.1, YP_{—}003022611.1, YP_{—}002138248.1, ZP_{—}01462439.1, ZP_{—}06499584.1, YP_{—}004669687.1, YP_{—}633289.1, ZP_{—}01907074.1, YP_{—}001611010.1, ADI22030.1, ZP_{—}03026937.1, YP_{—}580525.1, YP_{—}003265025.1, YP_{—}001525888.1, YP_{—}002298157.1, YP_{—}002945338.1, YP_{—}003271056.1, YP_{—}004198848.1, ZP_{—}01895445.1, ZP_{—}08636846.1, ADP95813.1, ZP_{—}03357270.1, YP_{—}002535575.1, YP_{—}160280.1, YP_{—}385012.1, YP_{—}004154467.1, YP_{—}742957.1, YP_{—}984918.1, ZP_{—}07949467.1, YP_{—}002552054.1, YP_{—}003439807.1, YP_{—}002919224.1, YP_{—}001475439.1, ZP_{—}07652842.1, YP_{—}001335140.1, YP_{—}972400.1, ZP_{—}08308052.1, YP_{—}001749490.1, YP_{—}004594280.1, ZP_{—}05360584.1, YP_{—}002490812.1, ZP_{—}03336986.1, YP_{—}004236457.1, ZP_{—}08387650.1, YP_{—}046370.1, ZP_{—}03823670.1, AEJ97944.1, ZP_{—}06188204.1, ABF82237.1, ZP_{—}06016043.1, YP_{—}046135.1, YP_{—}942111.1, ZP_{—}01614052.1, YP_{—}001341942.1, YP_{—}004713534.1, ZP_{—}01460231.1, YP_{—}001630800.1, YP_{—}001264278.1, CAD76924.1, ZP_{—}07200324.1, YP_{—}550745.1, YP_{—}001413963.1, YP_{—}002132758.1, ZP_{—}05972210.1, ZP_{—}06065848.1, ZP_{—}08209169.1, ZP_{—}06061642.1, ZP_{—}05109438.1, YP_{—}001419321.1, YP_{—}463572.1, YP_{—}608369.1, YP_{—}001683323.1, AEA83130.1, YP_{—}001230361.1, YP_{—}001832875.1, YP_{—}002237684.1, AAN39378.1, YP_{—}001019613.1, YP_{—}426398.1, ZP_{—}03543802.1, YP_{—}001171793.1, YP_{—}002138936.1, YP_{—}001562369.1, ZP_{—}01786296.1, YP_{—}001528043.1, NP_{—}881363.1, ZP_{—}04625099.1, ZP_{—}02187462.1, YP_{—}002355162.1, YP_{—}248479.1, ZP_{—}07043392.1, YP_{—}002028041.1, YP_{—}001900023.1, ZP_{—}01792340.1, ZP_{—}03541158.1, NP_{—}438930.1, ZP_{—}04464778.1, YP_{—}003278968.1, YP_{—}004490499.1, ZP_{—}07662038.1, AAM48101.1, YP_{—}422117.1, YP_{—}524752.1, YP_{—}918568.1, YP_{—}001264814.1, YP_{—}003807823.1, YP_{—}001260276.1, ZP_{—}07046088.1, ZP_{—}01784141.1, ZP_{—}05135853.1, YP_{—}002982015.1, EEZ80724.1, YP_{—}001292714.1, YP_{—}001971860.1, YP_{—}788379.1, ZP_{—}05783989.1, YP_{—}004415862.1, CAE45106.1, YP_{—}004618019.1, ZP_{—}01126529.1, ZP_{—}06062289.1, YP_{—}004538662.1, NP_{—}248919.1, YP_{—}001098905.1, YP_{—}003847633.1, YP_{—}002432816.1, YP_{—}003280245.1, A64092, ZP_{—}08404839.1, YP_{—}003466069.1, YP_{—}001348923.1, YP_{—}158582.1, YP_{—}004229600.1, ZP_{—}07797976.1, YP_{—}001416028.1, YP_{—}001747677.1, YP_{—}002362051.1, YP_{—}931973.1, ZP_{—}08505255.1, EGP53986.1, NP_{—}250427.1, YP_{—}366806.1, ZP_{—}04638299.1, ZP_{—}08485306.1, YP_{—}001675166.1, AAA23322.1, NP_{—}927515.1, AAR83740.1, YP_{—}433439.1, YP_{—}001668851.1, YP_{—}001713606.1, YP_{—}002354475.1, ZP_{—}06548530.1, ZP_{—}04764695.1, ZP_{—}01910282.1, YP_{—}004146469.1, YP_{—}095382.1, ZP_{—}06495825.1, YP_{—}003777379.1, ZP_{—}01914912.1, ZP_{—}06895226.1, YP_{—}004379898.1, YP_{—}003365234.1, YP_{—}001784146.1, YP_{—}003021900.1, YP_{—}004555586.1, YP_{—}001101071.1, CBW99592.1, YP_{—}003254723.1, AAG30258.1, YP_{—}004536011.1, NP_{—}884797.1, NP_{—}635761.1, YP_{—}002429235.1, YP_{—}001901798.1, ZP_{—}06485970.1, YP_{—}123631.1, YP_{—}001352245.1, ZP_{—}03697428.1, ZP_{—}05824476.1, ZP_{—}01014491.1, EGH60624.1, YP_{—}004028852.1, ZP_{—}04633718.1, YP_{—}001846659.1, ZP_{—}04933402.1, YP_{—}003731942.1, YP_{—}001345710.1, YP_{—}003979747.1, ZP_{—}00053266.1, YP_{—}126656.1, YP_{—}003442067.1, YP_{—}585810.1, ZP_{—}01614053.1, ZP_{—}06690229.1, YP_{—}001858908.1, ZP_{—}01128624.1, NP_{—}888558.1, ZP_{—}05827098.1, Q8VPF1.1, YP_{—}004473788.1, EGH77345.1, P45363.1, EGH44350.1, YP_{—}001676522.1, ZP_{—}05824514.1, ZP_{—}06487592.1, ZP_{—}02887415.1, ZP_{—}04761513.1, YP_{—}003377502.1, YP_{—}001188713.1, ZP_{—}01167911.1, ZP_{—}06690267.1, YP_{—}004680403.1, YP_{—}003731982.1, YP_{—}002800937.1, YP_{—}001758618.1, YP_{—}004380648.1, YP_{—}001188079.1, YP_{—}001707349.1, YP_{—}004687867.1, CAZ89607.1, ZP_{—}05827058.1, ZP_{—}08142248.1, YP_{—}195739.1, YP_{—}004703691.1, YP_{—}001354779.1, ZP_{—}08627639.1, ZP_{—}04936650.1, NP_{—}642338.1, ZP_{—}03451105.1, YP_{—}001713567.1, EFV87627.1, YP_{—}728366.1, YP_{—}002912837.1, YP_{—}001707333.1, YP_{—}363794.1, YP_{—}003524466.1, YP_{—}959751.1, YP_{—}606872.1, YP_{—}102034.1, YP_{—}002942733.1, YP_{—}002238110.1, ZP_{—}05032457.1, YP_{—}001846620.1, YP_{—}004153168.1, ZP_{—}02462362.1, YP_{—}003777513.1, YP_{—}199120.1, ZP_{—}08179077.1, ZP_{—}08188845.1, YP_{—}107279.1, ADP98459.1, YP_{—}004157409.1, YP_{—}610092.1, EGP55478.1, CBJ37328.1, ZP_{—}08181461.1, ZP_{—}06842278.1, ZP_{—}06703672.1, ADR61907.1, ZP_{—}06688595.1, ZP_{—}04934614.1, ZP_{—}07262554.1, YP_{—}786611.1, YP_{—}003439146.1, YP_{—}003592852.1, YP_{—}001747891.1, YP_{—}004386570.1, Q51956.1, YP_{—}004593695.1, YP_{—}560516.1, ZP_{—}06731844.1, YP_{—}001897101.1, ZP_{—}08388430.1, YP_{—}001166210.1, YP_{—}557015.1, ZP_{—}06843809.1, EGP42659.1, YP_{—}002005592.1, YP_{—}002871766.1, YP_{—}555845.1, ZP_{—}05921114.1, NP_{—}746745.1, NP_{—}637343.1, ZP_{—}02243308.1, YP_{—}001267798.1, ZP_{—}02354510.1, NP_{—}841567.1, ZP_{—}08177693.1, YP_{—}004703877.1, YP_{—}001166143.1, EGH73771.1, ZP_{—}06489206.1, ZP_{—}01892079.1, YP_{—}934562.1, ADY81955.1, EGB73439.1, NP_{—}520373.1, YP_{—}003905682.1, EGH61062.1, YP_{—}001894311.1, ZP_{—}02245330.1, YP_{—}918778.1, YP_{—}001120651.1, YP_{—}003612896.1, YP_{—}004125334.1, ZP_{—}07952596.1, YP_{—}001479268.1, ZP_{—}07043083.1, YP_{—}003644271.1, NP_{—}421210.1, ZP_{—}02882590.1, EGP25245.1, YP_{—}233920.1, EGD00226.1, YP_{—}004418315.1, ADY81914.1, ZP_{—}04944762.1, YP_{—}003603999.1, YP_{—}001060552.1, ZP_{—}03026966.1, YP_{—}441123.1, YP_{—}201177.1, ZP_{—}05117283.1, YP_{—}004232360.1, YP_{—}002802211.1, YP_{—}106085.1, YP_{—}258448.1, YP_{—}001989549.1, NP_{—}945866.1, ZP_{—}03573123.1, YP_{—}283604.1, YP_{—}004702122.1, ZP_{—}03398400.1, YP_{—}105310.1, YP_{—}001479310.1, CAC41637.1, ZP_{—}02372747.1, YP_{—}001578772.1, ZP_{—}08181762.1, ZP_{—}00439074.2, ADX92638.1, ZP_{—}03790444.1, YP_{—}110295.1, YP_{—}002439726.1, YP_{—}004361986.1, ZP_{—}04946665.1, YP_{—}003751825.1, YP_{—}001061488.1, ZP_{—}03545148.1, ZP_{—}01767462.1, ZP_{—}01769818.1, YP_{—}990241.1, YP_{—}002382777.1, YP_{—}002898389.1, YP_{—}003452421.1, EGH66881.1, CBW26817.1, YP_{—}004352451.1, EGC07165.1, YP_{—}003982691.1, ZP_{—}02906520.1, YP_{—}410799.1, YP_{—}001189077.1, YP_{—}004226900.1, ADP96997.1, YP_{—}237079.1, YP_{—}002946310.1, YP_{—}004029037.1, NP_{—}745423.1, ZP_{—}08139209.1, YP_{—}004294989.1, NP_{—}251630.1, EGH73592.1, ZP_{—}04934925.1, ZP_{—}03583227.1, ZP_{—}03584241.1, YP_{—}004684330.1, YP_{—}004501533.1, YP_{—}001186637.1, YP_{—}003980170.1, AEJ98540.1, YP_{—}004688333.1, YP_{—}003276725.1, EGH11975.1, YP_{—}276147.1, YP_{—}790233.1, ZP_{—}01736635.1, YP_{—}002440908.1, YP_{—}002230040.1, YP_{—}724980.1, YP_{—}004231717.1, ZP_{—}01226775.1, ZP_{—}03454556.1, ZP_{—}05586076.1, ZP_{—}02890239.1, YP_{—}001172996.1, ZP_{—}02377875.1, ZP_{—}07202399.1, YP_{—}774661.1, YP_{—}440314.2, YP_{—}443408.1, ZP_{—}06878044.1, ZP_{—}08274339.1, YP_{—}001618203.1, ZP_{—}08631485.1, ZP_{—}01545529.1, ZP_{—}03267843.1, ZP_{—}07797009.1, YP_{—}003376084.1, EGH21143.1, YP_{—}003753513.1, YP_{—}004282234.1, YP_{—}726356.1, ZP_{—}06014951.1, YP_{—}109637.1, ZP_{—}06461447.1, YP_{—}001795795.1, YP_{—}621981.1, ZP_{—}07794257.1, ZP_{—}05060451.1, YP_{—}002919830.1, YP_{—}001796645.1, NP_{—}794063.1, ZP_{—}01365347.1, YP_{—}003610065.1, YP_{—}001462757.1, YP_{—}001807185.1, ZP_{—}04928407.1, YP_{—}002229986.1, ZP_{—}02883901.1, YP_{—}370284.1, ZP_{—}05053491.1, AAC24332.1, ZP_{—}04929241.1, ZP_{—}00943679.1, YP_{—}001766064.1, YP_{—}001670661.1, YP_{—}003296167.1, YP_{—}003773673.1, NP_{—}250691.1, ZP_{—}05823066.1, YP_{—}004381309.1, YP_{—}004714773.1, YP_{—}746962.1, YP_{—}002513585.1, YP_{—}294674.1, YP_{—}004593822.1, YP_{—}622032.1, YP_{—}001897940.1, YP_{—}001335713.1, YP_{—}001856626.1, YP_{—}791238.1, YP_{—}004140309.1, YP_{—}001269802.1, ZP_{—}06879064.1, ZP_{—}01736318.1, ZP_{—}02886139.1, ZP_{—}04941413.1, YP_{—}001670851.1, YP_{—}371023.1, YP_{—}002980343.1, YP_{—}002795605.1, ZP_{—}06069679.1, ZP_{—}02463309.1, ZP_{—}05785212.1, YP_{—}001793049.1, YP_{—}003965283.1, YP_{—}001233153.1, YP_{—}299776.1, ZP_{—}06498740.1, AEJ99148.1, YP_{—}004685690.1, YP_{—}003746771.1, YP_{—}004381943.1, YP_{—}004378973.1, YP_{—}004314684.1, EGH79619.1, ZP_{—}04882546.1, YP_{—}347001.1, YP_{—}347471.1, YP_{—}001757758.1, YP_{—}002911324.1, NP_{—}518596.1, ZP_{—}00948908.1, YP_{—}442777.1, YP_{—}002874183.1, YP_{—}002230989.1, YP_{—}004360850.1, ABC36127.1, YP_{—}004696127.1, YP_{—}002799527.1, YP_{—}001631275.1, YP_{—}626125.1, ZP_{—}05090649.1, ZP_{—}07774002.1, ZP_{—}04940525.1, AEK60371.1, ADR60119.1, YP_{—}102981.1, YP_{—}003451423.1, NP_{—}743536.1, CAA45255.1,
in particular
YP_{—}790159.1, ZP_{—}06877967.1, NP_{—}251703.1, EGH66370.1, EGH10832.1, YP_{—}349606.1, YP_{—}236359.1, ZP_{—}07263340.1, EGH95844.1, NP_{—}793296.1, 1WDK_C, P28790.2, YP_{—}259060.1, ZP_{—}04587908.1, EGH45981.1, ZP_{—}07774144.1, EGH84449.1, YP_{—}275369.1, ZP_{—}07005686.1, YP_{—}001172247.1, YP_{—}004352962.1, ACP17922.1, YP_{—}002871196.1, ZP_{—}08142929.1, YP_{—}004701153.1, YP_{—}001667916.1, YP_{—}001268913.1, Q93Q11.1, Q9R9W0.1, NP_{—}744286.1, YP_{—}001187077.1, YP_{—}609234.1, YP_{—}004379417.1, YP_{—}004474975.1, ABP88737.1, EGH76239.1, EGH29725.1, ZP_{—}06499584.1, YP_{—}001749490.1, ABF82237.1, YP_{—}004713534.1, CAD76924.1, YP_{—}608369.1, AEA83130.1, YP_{—}001171793.1, YP_{—}788379.1, CAE45106.1, NP_{—}248919.1, YP_{—}001348923.1, ZP_{—}07797976.1, YP_{—}001747677.1, NP_{—}250427.1, AAR83740.1, YP_{—}001668851.1, ZP_{—}06495825.1, YP_{—}004379898.1, EGH60624.1, ZP_{—}04933402.1, YP_{—}001345710.1, Q8VPF1.1, YP_{—}004473788.1, EGH77345.1, EGH44350.1, YP_{—}001188713.1, YP_{—}004380648.1, YP_{—}001188079.1, ZP_{—}08142248.1, YP_{—}004703691.1, ZP_{—}04936650.1, YP_{—}606872.1, YP_{—}610092.1, ADR61907.1, ZP_{—}04934614.1, ZP_{—}07262554.1, YP_{—}001747891.1, Q51956.1, YP_{—}002871766.1, NP_{—}746745.1, YP_{—}001267798.1, YP_{—}004703877.1, EGH73771.1, EGH61062.1, YP_{—}233920.1, YP_{—}258448.1, YP_{—}004702122.1, ZP_{—}03398400.1, YP_{—}002439726.1, EGH66881.1, YP_{—}004352451.1, YP_{—}001189077.1, YP_{—}237079.1, NP_{—}745423.1, ZP_{—}08139209.1, NP_{—}251630.1, EGH73592.1, ZP_{—}04934925.1, YP_{—}001186637.1, EGH11975.1, YP_{—}276147.1, YP_{—}790233.1, YP_{—}002440908.1, YP_{—}001172996.1, ZP_{—}06878044.1, ZP_{—}07797009.1, EGH21143.1, ZP_{—}06461447.1, ZP_{—}07794257.1, NP_{—}794063.1, ZP_{—}01365347.1, ZP_{—}04928407.1, AAC24332.1, ZP_{—}04929241.1, YP_{—}001670661.1, NP_{—}250691.1, YP_{—}004381309.1, YP_{—}004714773.1, YP_{—}791238.1, YP_{—}001269802.1, ZP_{—}06879064.1, YP_{—}001670851.1, ZP_{—}06498740.1, YP_{—}004381943.1, YP_{—}004378973.1, EGH79619.1, YP_{—}347001.1, YP_{—}347471.1, YP_{—}002874183.1, ZP_{—}07774002.1, ADR60119.1, NP_{—}743536.1, YP_{—}001269653.1, ZP_{—}06482365.1, ADI95330.1, ZP_{—}07003619.1, BAB96553.1, ZP_{—}07777009.1, ABA10831.1, YP_{—}273131.1, YP_{—}259428.1, EFW86233.1, EGH85840.1, ZP_{—}07774597.1, EGH54613.1, YP_{—}004353129.1, YP_{—}002871014.1, YP_{—}001171232.1, EGH67454.1, EFW82139.1, ZP_{—}04590526.1, EGH58132.1, EGH06629.1, EGH99157.1, ZP_{—}05638078.1, NP_{—}790796.1, AEE59172.1, YP_{—}026272.1, YP_{—}002389323.1, EGB30581.1, YP_{—}001460637.1, YP_{—}001727089.1, CAB40810.1, ZP_{—}03049054.1, ZP_{—}08356522.1, ZP_{—}06664573.1, AAA67642.1, YP_{—}003236969.1, ZP_{—}06659935.1, NP_{—}290475.1, ZP_{—}03027945.1, EFZ59092.1, YP_{—}002295406.1, CBG37050.1, EGP22872.1, EGE62411.1, YP_{—}002400349.1, EGB59498.1, ZP_{—}08361144.1, YP_{—}001465329.1, YP_{—}002409079.1, ZP_{—}06655947.1, YP_{—}002414995.1, EGB69559.1, YP_{—}859446.1, ZP_{—}08386168.1, YP_{—}543378.1, ZP_{—}08366394.1, YP_{—}001746177.1, ZP_{—}07154548.1, ZP_{—}03070708.1, NP_{—}756651.1, YP_{—}671919.1, YP_{—}002331615.1, ZP_{—}07449249.1, ZP_{—}06938722.1, ZP_{—}03086141.1, ZP_{—}07189177.1, ZP_{—}07136312.1, EFZ47010.1, ZP_{—}07136311.1, EFZ47009.1, ZP_{—}03026937.1, EGB73439.1, EGP25245.1, ZP_{—}03026966.1, YP_{—}001462757.1, CAP76727.1, YP_{—}670163.1,
and especially preferably
YP_{—}026272.1, AAA67642.1, ZP_{—}07593202.1, YP_{—}004701153.1, YP_{—}001667916.1, YP_{—}001268913.1, Q9R9W0.1, NP_{—}744286.1, ABP88737.1, YP_{—}001749490.1, YP_{—}001747677.1, YP_{—}001668851.1, YP_{—}004703691.1, ADR61907.1, YP_{—}001747891.1, Q51956.1, NP_{—}746745.1, YP_{—}001267798.1, YP_{—}004703877.1, YP_{—}004702122.1, NP_{—}745423.1, AAC24332.1, YP_{—}001670661.1, YP_{—}001269802.1, YP_{—}001670851.1, ADR60119.1, NP_{—}743536.1, YP_{—}001269653.1, ADI95330.1, BAB96553.1, YP_{—}001172247.1, YP_{—}004713534.1, AEA83130.1, YP_{—}001171793.1, YP_{—}001172996.1, YP_{—}004714773.1, YP_{—}001171232.1, YP_{—}349606.1, YP_{—}259060.1, ZP_{—}07774144.1, YP_{—}002871196.1, ABF82237.1, YP_{—}002871766.1, YP_{—}258448.1, YP_{—}347001.1, YP_{—}347471.1, YP_{—}002874183.1, ZP_{—}07774002.1, ZP_{—}07777009.1, YP_{—}259428.1, ZP_{—}07774597.1, YP_{—}002871014.1,
AAC72881.1, ABB71579.1, CAC19934.1, AAC49180.1 (encoded by SEQ ID No.: 10), AAC49783.1, AAC49179.1, CAB60830.1, ABB71581.1, AAC49269.1, CAC 19933.1, CAA54060.1, AAC72882.1, 039513.1, AAC49784.1, ABO38558.1, ABO38555.1, ABO38556.1, ABO38554.1, ADB79568.1, ADB79569.1, ACQ57188.1, ACQ57189.1, ABK96561.1, ACQ63293.1, ACQ57190.1, Q9SQI3.1, ABU96744.1, ABC47311.1, XP_{—}002324962.1, AAD01982.1, AAB51525.1, ACV40757.1, XP_{—}002309244.1, CBI28125.3, ABD91726.1, XP_{—}002284850.1, XP_{—}002309243.1, XP_{—}002515564.1, ACR56792.1, ACR56793.1, XP_{—}002892461.1, ABI18986.1, NP_{—}172327.1, CAA85387.1, CAA85388.1, ADA79524.1, ACR56795.1, ACR56794.1, CAN81819.1, ACF17654.1, AAB71729.1, ABH11710.1, ACQ57187.1, AAX51637.1, AAB88824.1, AAQ08202.1, AAB71731.1, AAX51636.1, CAC80370.1, CAC80371.1, AAG43858.1, ABD83939.1, AAD42220.2, AAG43860.1, AAG43861.1, AAG43857.1, AAL15645.1, AAB71730.1, NP_{—}001068400.1, EAY86877.1, NP_{—}001056776.1, XP_{—}002436457.1, NP_{—}001149963.1, ACN27901.1, EAY99617.1, ABL85052.1, XP_{—}002437226.1, NP_{—}001151366.1, ACF88154.1, NP_{—}001147887.1, XP_{—}002453522.1, BAJ99650.1, EAZ37535.1, EAZ01545.1, AAN17328.1, EAY86884.1, EEE57469.1, Q41635.1, AAM09524.1, Q39473.1, NP_{—}001057985.1, AAC49001.1, XP_{—}001752161.1, XP_{—}001770108.1, XP_{—}001784994.1, XP_{—}002318751.1, NP_{—}001047567.1, XP_{—}002322277.1, XP_{—}002299627.1, XP_{—}002511148.1, CBI15695.3, XP_{—}002299629.1, XP_{—}002280321.1, CAN60643.1, XP_{—}002459731.1, XP_{—}002975500.1, XP_{—}002962077.1, XP_{—}001773771.1, NP_{—}001151014.1, XP_{—}002317894.1, XP_{—}002971008.1, XP_{—}001774723.1, XP_{—}002280147.1, XP_{—}002526311.1, XP_{—}002517525.1, XP_{—}001764527.1, ABI20759.1, BAD73184.1, XP_{—}002987091.1, XP_{—}002985480.1, CBI26947.3, ABI20760.1, XP_{—}002303055.1, XP_{—}002885681.1, ADH03021.1, XP_{—}002532744.1, EAY74210.1, EEC84846.1, EEE54649.1, AAG35064.1, AAC49002.1, CAD32683.1, ACF78226.1, BAJ96402.1, XP_{—}002462626.1, NP_{—}001130099.1, XP_{—}002462625.1, ABX82799.3, Q42712.1, NP_{—}193041.1, AAB51524.1, NP_{—}189147.1, ABR18461.1, XP_{—}002863277.1, AAC72883.1, AAA33019.1, CBI40881.3, XP_{—}002262721.1, AAB51523.1, NP_{—}001063601.1, ADB79567.1, AAL77443.1, AAL77445.1, AAQ08223.1, AAL79361.1, CAA52070.1, AAA33020.1, CAA52069.1, XP_{—}001785304.1, CAC39106.1, XP_{—}002992591.1, XP_{—}002968049.1, XP_{—}001770737.1, XP_{—}001752563.1, AAG43859.1, XP_{—}002978911.1, XP_{—}002977790.1, ACB29661.1, XP_{—}002314829.1, XP_{—}002991471.1, EAZ45287.1, XP_{—}002986974.1, EEC73687.1, XP_{—}002312421.1, ACJ84621.1, NP_{—}001150707.1, AAD28187.1, XP_{—}001759159.1, XP_{—}001757193.1, XP_{—}002322077.1, ABE01139.1, XP_{—}002447294.1, AAX54515.1, AAD33870.1, AAX54514.1, CBI15694.3, XP_{—}002270653.1, AAZ83073.1, CAC14164.1, XP_{—}001753224.1, CBI35766.3, ACU22895.1, BAC43222.1, XP_{—}002965875.1, AAX54516.1, XP_{—}002983123.1, XP_{—}002447046.1, ACL52706.1, CAA06001.1, XP_{—}001772711.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{f} is generally understood in particular as meaning the reaction of 3-oxododecanoyl-CoA thioester and CoA to give decanoyl-CoA thioester and acetyl-CoA.

### Sixth Genetic Modification for Enhancing the Acyl-ACP Thioester Synthesis

According to the invention, the microorganisms include a sixth genetic modification so that they are capable of forming more acyl-ACP thioester from at least one simple carbon source in comparison with their wild type. An overview over correspondingly desirable genetic modifications can be found in FIG. 1 of WO2008119082, section 1 (fatty acid production increase/product production increase).

The technical effect of this is that the formation of carboxylic acids and carboxylic acid derivatives which is increased by the first genetic modification, but also of carboxylic acids and carboxylic acid derivatives which are formed in larger amounts due to the second, third, fourth or fifth genetic modification, is increased even further.

Preferred Microorganisms for the Production of Alkan-1-ols, Alkan-1-als, Alkan-1-Amines, Alkanes, Olefins, Alken-1-als, Alken-1-ols and Alken-1-Amines

If the microorganisms according to the invention are intended to be used in a process for the production of alkan-1-ols, alkan-1-als, alkan-1-amines, alkanes, alken-1-als, alken-1-ols and alken-1-amines and terminal olefins which optionally contain further double bonds, it may be advantageous that the microorganisms according to the invention include a seventh genetic modification comprising an activity of at least one enzyme E₁ that is reduced in comparison with its wild type, selected from the group:

E₁ₐ P450 alkane hydroxylases, which preferably catalyse the following reactions:
reduced haem+alkanoic acid (ester)=oxidized haem+ω-hydroxyalkanoic acid (ester)+H₂O,
2 reduced haem+alkanoic acid (ester)=2 oxidized haem+ω-oxoalkanoic acid (ester)+2H₂O
or
3 reduced haem+alkanoic acid (ester)=alkane monoxygenase+3 oxidized haem+ω-carboxyalkanoic acid (ester)+3H₂O and preferably
component of a reaction system composed of the two enzyme components “cytochrome P450 alkane hydroxylase and NADPH-cytochrome P450 oxidoreductase of EC 1.6.2.4” or component of a reaction system composed of the three enzyme components “cytochrome P450 alkane hydroxylase of the type CYP_{—}153, ferredoxin-NAD(P)⁺ reductases of EC 1.18.1.2 or EC 1.18.1.3 and ferredoxin” and
E_{1b} AlkB alkane hydroxylases of EC 1.14.15.3, which preferably catalyse the following reactions: reduced rubredoxin+alkanoic acid (ester)=oxidized rubredoxin+ω-hydroxyalkanoic acid (ester)+H₂O,
2 reduced rubredoxins+alkanoic acid (ester)=2 oxidized rubredoxins+ω-oxoalkanoic acid (ester)+2H₂O or
3 reduced rubredoxins+alkanoic acid (ester)=alkane monoxygenase+3 oxidized rubredoxins+ω-carboxyalkanoic acid (ester)+3H₂O and preferably
component of a reaction system composed of the three enzyme components “AlkB alkane hydroxylase of EC 1.14.15.3, AlkT rubredoxin-NAD(P)⁺ reductase of EC 1.18.1.1 or EC 1.18.1.4 and rubredoxin AlkG”,
E_{1c} fatty alcohol oxidases of EC 1.1.3.20, which preferably catalyse at least one of the following irreversible reactions:
alkan-1-ol+O₂=alkan-1-al+H₂O₂ or
alkan-1-al+O₂=alkanoic acid+H₂O₂,
E_{1d} AlkJ alcohol dehydrogenases of EC 1.1.99.-, which preferably catalyse at least one of the following reversible reactions:
alkan-1-ol+oxidized acceptor=alkan-1-al+reduced acceptor or alkan-1-al+oxidized acceptor=alkanoic acid+reduced acceptor,
E₁ₑ alcohol dehydrogenases of EC 1.1.1.1 or EC 1.1.1.2, which preferably catalyse at least one of the following reversible reactions:
alkan-1-ol+NAD(P)⁺=alkan-1-al+NAD(P)H+H⁺ or
alkan-1-al+NAD(P)⁺=alkanoic acid+NAD(P)H+H⁺ and
E_{1f} aldehyde dehydrogenases of EC 1.2.1.3, EC 1.2.1.4 or EC 1.2.1.5, which preferably catalyse the following reversible reaction:
alkan-1-al+NAD(P)⁺=alkanoic acid+NAD(P)H+H⁺

It may be advantageous in particular for the production of alkan-1-ols that the microorganisms according to the invention have an activity of at least one enzyme E₁ₑ and E_{1f} in comparison with their wild type.

WO2010062480 A2 describes microorganisms which are capable of forming more fatty acids and fatty acid derivatives, in particular fatty alcohols, from at least one simple carbon source in comparison with their wild type, in particular in exemplary embodiments 3, 4, 6 and 7. The document also describes enzymes E₁ₑ which are preferred according to the invention and their sequences, in particular in FIG. 10 and in exemplary embodiments 2 to 7.

If the microorganisms according to the invention are to be used in a process for the production of alkan-1-ols, alkan-1-als, alkan-1-amines and alkanes, it is especially preferred according to the invention that the activity of such enzymes E₁ₐ to E_{1f}, which catalyse the above-described reactions of an alkan-1-al to give the corresponding alkanoic acid, is reduced.

If the microorganisms according to the invention are to be used in a process for the production of alkan-1-als, alkan-1-amines, alkanes and 1-alkenes, it is especially preferred according to the invention that the activity of such enzymes E₁ₐ to E₁ₑ, which catalyse the above-described conversion of an alkan-1-al to give the corresponding alkan-1-ol, is reduced.

If the microorganisms according to the invention are to be used in a process for the production of alkan-1-ols, it is especially preferred according to the invention that the activity of such enzymes E₁ₐ to E₁ₑ, which catalyse the above-described conversion of an alkan-1-ol to give the corresponding alkan-1-al, is reduced.

### Specific Enzymes E₁ₐ

P450 alkane hydroxylases E₁ₐ which are preferred in this context are selected from among the list

AAO73954.1, AAO73953.1, XP_{—}002546279.1, AAA34353.2, P30607.1, XP_{—}002421627.1, XP_{—}718670.1, CAA39366.1, XP_{—}001527524.1, AAO73955.1, AAO73956.1, XP_{—}002546278.1, EEQ43157.1, XP_{—}718669.1, AAA34354.1, P10615.3, XP_{—}002421628.1, 226487, P16141.3, CAA39367.1, Q9Y757.2, XP_{—}001485567.1, AAO73958.1, XP_{—}001383506.2, XP_{—}460111.2, AAO73959.1, Q12586.1, XP_{—}460112.2, AAO73960.1, Q12589.1, AAO73961.1, XP_{—}460110.2, EEQ43763.1, XP_{—}710174.1, EDK41572.2, XP_{—}001482650.1, CAA75058.1, XP_{—}002548818.1, Q12588.1, XP_{—}002422222.1, XP_{—}001383636.2, XP_{—}001525381.1, XP_{—}002548823.1, P30610.1, AAO73952.1, XP_{—}002548428.1, CAA36197.1, XP_{—}002421126.1, AAA34320.1, P16496.3, P30608.1, P24458.1, XP_{—}717999.1, XP_{—}001383817.1, Q9Y758.1, XP_{—}001482092.1, XP_{—}001383710.2, P30609.1, AAB24479.1, XP_{—}457792.1, XP_{—}001524144.1, XP_{—}457727.2, XP_{—}001525578.1, XP_{—}002616743.1, XP_{—}002614836.1, XP_{—}001525577.1, AAO73957.1, Q12585.1, XP_{—}001386440.2, XP_{—}002616857.1, XP_{—}001483276.1, XP_{—}500402.1, EDK39907.2, XP_{—}500560.1, XP_{—}001211376.1, XP_{—}002560027.1, XP_{—}504857.1, XP_{—}500855.1, XP_{—}504406.1, BAA31433.1, XP_{—}500856.1, XP_{—}501148.1, XP_{—}746567.1, XP_{—}001262425.1, XP_{—}001274843.1, XP_{—}002840588.1, XP_{—}002377641.1, XP_{—}001825995.1, XP_{—}001400739.1, XP_{—}718066.1, CAA35593.1, XP_{—}664735.1, XP_{—}002150795.1, XP_{—}500097.1, XP_{—}002483325.1, XP_{—}504311.1, XP_{—}500273.1, XP_{—}002548817.1, EDP54484.1, XP_{—}755288.1, XP_{—}001260447.1, EFY97851.1, ACD75398.1, ADK36660.1, XP_{—}001213081.1, XP_{—}002377989.1, XP_{—}001826299.1, XP_{—}001554811.1, XP_{—}501667.1, XP_{—}002148942.1, ADK36662.1, XP_{—}002565827.1, P30611.1, XP_{—}001267871.1, XP_{—}002372373.1, EFY84686.1, P43083.1, XP_{—}001263094.1, XP_{—}002148355.1, XP_{—}002568429.1, XP_{—}001817314.1, Q12587.1, XP_{—}001396435.1, XP_{—}001938589.1, XP_{—}001388497.2, XP_{—}663661.1, XP_{—}003295335.1, XP_{—}002152088.1, XP_{—}001212071.1, Q12573.1, XP_{—}002379858.1, XP_{—}001821592.1, XP_{—}002844341.1, XP_{—}001394678.1, ACD75400.1, BAK03594.1, XP_{—}003170343.1, XP_{—}001265480.1, XP_{—}002550661.1, EDP55514.1, XP_{—}001528842.1, XP_{—}749919.1, XP_{—}001593058.1, P30612.1, EGC48494.1, EEH04429.1, XP_{—}001585586.1, XP_{—}003236182.1, XP_{—}001400199.1, EEQ46951.1, XP_{—}721410.1, EGP87864.1, XP_{—}002380808.1, XP_{—}001792771.1, XP_{—}001208515.1, XP_{—}001216161.1, XP_{—}003071804.1, EFW16963.1, XP_{—}002542118.1, XP_{—}001936677.1, EGD95268.1, XP_{—}003015678.1, XP_{—}501748.1, XP_{—}003169562.1, EFY96492.1, XP_{—}682653.1, XP_{—}002421356.1, CAK43439.1, EFY93677.1, XP_{—}747767.1, XP_{—}001244958.1, XP_{—}003019635.1, XP_{—}002847463.1, EGP83273.1, EGR52487.1, XP_{—}002622526.1, XP_{—}002563618.1, CBX99718.1, XP_{—}001552081.1, XP_{—}003066638.1, XP_{—}003176049.1, ACD75402.1, BAA05145.1, XP_{—}002482834.1, XP_{—}001257501.1, XP_{—}001934574.1, XP_{—}001269972.1, XP_{—}001587438.1, XP_{—}001215856.1, XP_{—}002149824.1, XP_{—}001550556.1, XP_{—}003011982.1, XP_{—}001827121.1, XP_{—}003233566.1, XP_{—}003022481.1, EGR47044.1, EFQ34695.1, XP_{—}003170005.1, BAG09241.1, XP_{—}002796370.1, XP_{—}003019300.1, XP_{—}002563873.1, CAK40654.1, EEH19741.1, XP_{—}003012518.1, EGD95716.1, XP_{—}003239409.1, BAJ04363.1, XP_{—}001537012.1, BAE66393.1, EGP85214.1, XP_{—}002487227.1, AAV66104.1, EGE07669.1, XP_{—}362943.2, XP_{—}003016806.1, EFQ27388.1, XP_{—}002384360.1, XP_{—}002836323.1, XP_{—}001274959.1, EFZ03093.1, XP_{—}661521.1, XP_{—}002849803.1, XP_{—}001589398.1, AAR99474.1, XP_{—}003189427.1, XP_{—}001823699.1, XP_{—}364111.1, XP_{—}001262753.1, EFY86805.1, XP_{—}001390153.2, XP_{—}002384738.1, XP_{—}001941811.1, XP_{—}001220831.1, XP_{—}003296981.1, XP_{—}002480829.1, BAD83681.1, XP_{—}001827526.2, XP_{—}369556.1, CAK38224.1, EFQ26532.1, XP_{—}002562328.1, XP_{—}001904540.1, EGO52476.1, XP_{—}002382002.1, XP_{—}001225874.1, XP_{—}958030.2, XP_{—}002540883.1, XP_{—}001908957.1, XP_{—}001559255.1, XP_{—}364102.1, EDP48064.1, XP_{—}365075.1, XP_{—}381460.1, CBX95930.1, XP_{—}003054099.1, XP_{—}361347.2, XP_{—}002846867.1, XP_{—}001214985.1, EFQ35175.1, XP_{—}002479062.1, XP_{—}001908613.1, XP_{—}003345380.1, EGR50567.1, XP_{—}002479350.1, XP_{—}001394417.2, XP_{—}001394159.2, XP_{—}002146776.1, EGP86783.1, EFX02953.1, CAK45889.1, XP_{—}003006887.1, XP_{—}002541427.1, XP_{—}750735.1, XP_{—}001257962.1, EGO51720.1, XP_{—}003005336.1, EGP83197.1, XP_{—}002149832.1, XP_{—}003052680.1, XP_{—}365851.1, XP_{—}001799910.1, XP_{—}003347175.1, XP_{—}002565258.1, EGR48918.1, EGR52524.1, XP_{—}964653.2, XP_{—}002147083.1, XP_{—}002843935.1, EEH19393.1, CAC 10088.1, EEH47609.1, EEQ92528.1, XP_{—}001246560.1, XP_{—}002626168.1, XP_{—}003024880.1, XP_{—}003169255.1, XP_{—}003013780.1, XP_{—}003235691.1, XP_{—}746816.1, EGD98483.1, XP_{—}001389925.2, XP_{—}002842817.1, XP_{—}002797278.1, ADK36666.1, XP_{—}003305469.1, XP_{—}001548471.1, XP_{—}001806478.1, EFQ34989.1, XP_{—}001552987.1, CAC24473.1, XP_{—}002541530.1, EEQ89262.1, XP_{—}001247332.1, XP_{—}003066043.1, EDP47672.1, XP_{—}002628451.1, XP_{—}001910644.1, EGR44510.1, EFQ36733.1, XP_{—}003052472.1, XP_{—}001393445.2, XP_{—}001522438.1, EGO04179.1, XP_{—}001397944.2, CAK49049.1, EFQ30109.1, XP_{—}001585052.1, EGO30123.1, XP_{—}388496.1, XP_{—}003173913.1, CBF76609.1, XP_{—}003028593.1, EGO04180.1, CAK46976.1, XP_{—}370476.1, XP_{—}002145942.1, XP_{—}003004457.1, ADK36663.1, XP_{—}003040708.1, XP_{—}003351473.1, EFY84692.1, XP_{—}748328.2, XP_{—}003190325.1, XP_{—}002378813.1, EGR46513.1, XP_{—}003033448.1, XP_{—}002145326.1, XP_{—}662462.1, XP_{—}747469.1, XP_{—}001935085.1, EGR45892.1, E0001601.1, EGP89995.1, XP_{—}001222615.1, XP_{—}001224356.1, EGN93507.1, XP_{—}001934479.1, BAK09464.1, EGO30124.1, XP_{—}001267956.1, ADK36661.1, EFY97845.1, XP_{—}001834501.1, EGO03790.1, XP_{—}001884320.1, XP_{—}003028899.1, AAP79879.1, EFY84206.1, BAK09467.1, XP_{—}003030469.1, XP_{—}001412594.1, XP_{—}001834508.1, XP_{—}001839436.2, XP_{—}002583529.1, XP_{—}001886288.1, XP_{—}002843371.1, XP_{—}001587730.1, BAK09418.1, BAK09442.1, EGO28830.1, EGE03365.1, EFZ01428.1, EGO03065.1, XP_{—}001558890.1, XP_{—}002487181.1, EGO29652.1, AAX49400.1, EFY92529.1, XP_{—}002380252.1, XP_{—}001884460.1, BAK09387.1, XP_{—}001839366.2, XP_{—}003031835.1, EFY99978.1, AAL67906.1, BAG09240.1, XP_{—}002381768.1, XP_{—}001800031.1, XP_{—}001825073.2, BAE63940.1, XP_{—}003028894.1, AAL67905.1, XP_{—}002910303.1, EGO22856.1, XP_{—}003028896.1, XP_{—}681680.1, XP_{—}002486603.1, XP_{—}001838945.2, EGR50064.1, XP_{—}001884349.1, XP_{—}001883816.1, CAK37996.1, CAO91865.1, XP_{—}003031227.1, XP_{—}001258702.1, XP_{—}001586739.1, XP_{—}001560806.1, CBF69707.1, ADN43682.1, XP_{—}001593179.1, XP_{—}001886909.1, XP_{—}001934479.1, XP_{—}001587730.1, XP_{—}001886909.1, XP_{—}001831709.2, XP_{—}001392650.1, XP_{—}366716.2, CAL69594.1, XP_{—}001269140.1, XP_{—}002566307.1, XP_{—}001555473.1, XP_{—}663925.1, XP_{—}001598033.1, XP_{—}001835239.2, EGN97256.1, XP_{—}001554305.1, NP_{—}182075.1, XP_{—}001560475.1, EFQ32286.1, XP_{—}001216788.1, XP_{—}002483975.1, AAC31835.1, NP_{—}850427.1, XP_{—}002143660.1, XP_{—}003327130.1, BAJ78287.1, XP_{—}002880182.1, ACB59278.1, EFQ36688.1, BAJ78285.1, BAJ78286.1, XP_{—}001798699.1, EEH44101.1, BAJ78288.1, BAJ78284.1, EGG02425.1, EGG03011.1, AAA34334.1, NP_{—}001189747.1, EGG02601.1, XP_{—}002978645.1, EGG 11203.1, XP_{—}762610.1, XP_{—}762620.1, XP_{—}001545581.1, CAB44684.1, CAN80536.1, AAN05337.1, NP_{—}001049423.1, XP_{—}001791898.1, NP_{—}001031814.1, XP_{—}002279531.1, ABK94777.1, AAZ39646.1, XP_{—}002880183.1, ABC68403.1, XP_{—}002839066.1, EGG03014.1, XP_{—}002320074.1, NP_{—}001182854.1, CBI38795.3, XP_{—}002310605.1, NP_{—}196442.2, XP_{—}002270594.1, ABZ80830.1, XP_{—}002275905.1, CBI38796.3, XP_{—}002476978.1, CAB93726.1, EGG03624.1, EGG06527.1, NP_{—}197710.1, XP_{—}001768338.1, XP_{—}002270673.1, BAJ86572.1, XP_{—}002275806.1, CBI38797.3, XP_{—}002320072.1, CAN60189.1, XP_{—}002986290.1, XP_{—}002465888.1, CAN80040.1, XP_{—}002336104.1, XP_{—}002988354.1, XP_{—}002264277.1, EGD72898.1, XP_{—}002866853.1, EAY95236.1, XP_{—}002979701.1, XP_{—}002988762.1, XP_{—}002304502.1, XP_{—}002873349.1, XP_{—}003192947.1, CAN63571.1, NP_{—}001053615.1, NP_{—}176558.1, EGC49561.1, EGG09027.1, XP_{—}002314581.1, XP_{—}002446966.1, XP_{—}002320802.1, ABC59095.1, XP_{—}003323121.1, XP_{—}002974639.1, XP_{—}002395587.1, XP_{—}002866852.1, XP_{—}002319770.1, NP_{—}001146262.1, NP_{—}001169224.1, AAM65207.1, XP_{—}002529058.1, XP_{—}002886391.1, XP_{—}002320071.1, XP_{—}002446967.1, XP_{—}757870.1, EAY95147.1, XP_{—}002899664.1, EEH05830.1, XP_{—}002874114.1, ADO24345.1, BAJ88802.1, BAA05146.1, XP_{—}002963351.1, EAY88475.1, NP_{—}195658.3, XP_{—}002976944.1, ABC59093.1, XP_{—}002275114.1, XP_{—}003328407.1, CAN75428.1, BAJ86471.1, XP_{—}002981144.1, XP_{—}002277006.1, EAZ26110.1, ACN41008.1, XP_{—}002899542.1, XP_{—}001781614.1, EAY76187.1, BAK06758.1, XP_{—}002511745.1, XP_{—}002982626.1, XP_{—}002963763.1, NP_{—}001065111.1, ABF93892.1, XP_{—}002314117.1, BAK06287.1, XP_{—}001745327.1, NP_{—}001047674.1, XP_{—}002878665.1, XP_{—}002974847.1, NP_{—}179899.1, CAN80156.1, NP_{—}001053543.1, ABC59094.1, XP_{—}002328165.1, XP_{—}002270628.1, XP_{—}002275115.1, XP_{—}002980688.1, XP_{—}002465039.1, AAL91155.1, NP_{—}195910.1, XP_{—}002509820.1, NP_{—}200694.1, CAA62082.1, AAL75903.1, XP_{—}002468241.1, XP_{—}002883546.1, XP_{—}002862636.1, XP_{—}002312905.1, EAY79269.1, AAM12494.1, XP_{—}002875027.1, XP_{—}758010.1, XP_{—}002509524.1, AAP54707.2, XP_{—}002869292.1, NP_{—}001143079.1, ACF82946.1, XP_{—}002270497.1, XP_{—}002979685.1, XP_{—}002465041.1, XP_{—}002533544.1, AAG17470.1, XP_{—}002985393.1, NP_{—}191946.1, XP_{—}002525608.1, AAZ39642.1, XP_{—}002270428.1, XP_{—}002529227.1, CBI24485.3, XP_{—}001763206.1, EGG02922.1, XP_{—}002974848.1, NP_{—}001141467.1, CBI27149.3, NP_{—}001130907.1, XP_{—}002982474.1, NP_{—}001048917.1, XP_{—}002465889.1, ABZ80831.1, XP_{—}002464461.1, EAY88476.1, BAJ90714.1, XP_{—}002893825.1, ACN28568.1, XP_{—}002452782.1, XP_{—}002280004.1, XP_{—}001764611.1, NP_{—}001183394.1, BAJ89570.1, CBI24484.3, BAJ88840.1, ACG38359.1, CAN77648.1, BAJ91452.1, NP_{—}001141345.1, XP_{—}002282185.1, XP_{—}002980994.1, XP_{—}002299820.1, BAJ87982.1, BAJ91842.1, XP_{—}003325270.1, XP_{—}001760399.1, CBI34058.3, ADG34845.1, XP_{—}002523775.1, EEH21852.1, Q50EK3.1, BAK06748.1, XP_{—}002963764.1, ACN34158.1, XP_{—}001764503.1, XP_{—}002311750.1, XP_{—}001782495.1, XP_{—}002988642.1, XP_{—}002465625.1, XP_{—}002892051.1, XP_{—}002279649.1, NP_{—}171666.1, ABK28430.1, BAC42067.1, AED99869.1, NP_{—}174713.1, XP_{—}001781706.1, ABG66204.1, XP_{—}002964775.1, NP_{—}001064901.2, XP_{—}002961706.1, XP_{—}002519477.1, XP_{—}001559854.1, CBH32594.1, BAB92258.1, XP_{—}002264897.1, AAL59025.1, XP_{—}002862576.1, ACL53124.1, XP_{—}002521476.1, NP_{—}200045.1, BAJ89814.1, CBI38794.3, XP_{—}776769.1, NP_{—}001141372.1, EEC74485.1, EAY76557.1, XP_{—}002318861.1, NP_{—}001172660.1, XP_{—}002880978.1, AAO00706.1, BAK07606.1, XP_{—}002979336.1, BAC42841.1, BAF46296.1, XP_{—}002306380.1, XP_{—}002865907.1, ACG34921.1, XP_{—}002876375.1, NP_{—}001056685.1, XP_{—}002264292.1, XP_{—}002893443.1, NP_{—}001066096.1, EEE53477.1, CBH32607.1, EAY94753.1, NP_{—}001130939.1, NP_{—}182121.1, XP_{—}002437749.1, NP_{—}191222.1, XP_{—}002865881.1, XP_{—}569708.1, XP_{—}002279670.1, BAJ94774.1, ABF93894.1, BAD94304.1, ACG33785.1, NP_{—}194944.1, NP_{—}180337.1, AAB63277.1, BAJ85246.1, XP_{—}002456654.1, ACN27732.1, XP_{—}002445325.1, EER40289.1, XP_{—}001838184.2, BAJ85532.1, XP_{—}002866555.1, EAY88477.1, ACG47870.1, XP_{—}002310074.1, XP_{—}002457224.1, EAZ25521.1, BAJ87689.1, NP_{—}001044838.1, XP_{—}002521004.1, XP_{—}002882043.1, XP_{—}002527038.1, XP_{—}002318721.1, XP_{—}002979339.1, NP_{—}176086.1, XP_{—}001560028.1, ABC59092.1, ABF93891.1, ACR38435.1, EAY78983.1, NP_{—}179782.1, CCA21696.1, XP_{—}002334340.1, EFX88387.1, NP_{—}001044554.1, XP_{—}002321857.1, NP_{—}173862.1, NP_{—}195660.1, XP_{—}001554079.1, EAZ13864.1, EEC67630.1, EAY76183.1, AAP54710.2, NP_{—}001065112.2, ACD10924.1, XP_{—}001559275.1, EEC67338.1, XP_{—}002273811.1, ADJ68242.1, NP_{—}001065698.1, CAN66874.1, CAB41474.1, XP_{—}002868908.1, XP_{—}002904660.1, CAR47816.1, NP_{—}189243.1, EAY98229.1, XP_{—}002448320.1, 081117.2, XP_{—}002458797.1, XP_{—}002277129.1, BAJ88829.1, CAN67559.1, BAK08034.1, XP_{—}002894062.1, XP_{—}002894891.1, XP_{—}002279981.1, ABR16451.1, NP_{—}201150.1, AAM60854.1, XP_{—}002521002.1, XP_{—}002521474.1, XP_{—}002875311.1, NP_{—}195661.1, AAP79889.1, NP_{—}175193.1, P98188.1, BAK08270.1, CBI21357.3, XP_{—}002870817.1, XP_{—}002904451.1, ABA95812.1, XP_{—}002998647.1, NP_{—}001066166.2, XP_{—}002894690.1, EFY92064.1, XP_{—}002278009.1, XP_{—}002336002.1, CCA16508.1, XP_{—}002868909.1, EAZ31703.1, C96517, EAY86526.1, XP_{—}002307954.1, XP_{—}002904638.1, XP_{—}002266883.1, XP_{—}002439880.1, XP_{—}002892730.1, ADI52567.1, EGI61791.1, XP_{—}002511196.1, EGG04372.1, XP_{—}002511875.1, ACE75189.1, NP_{—}001055681.1, XP_{—}001589816.1, NP_{—}001170655.1, XP_{—}002300789.1, XP_{—}001934479.1, XP_{—}001587730.1, XP_{—}001554079.1, XP_{—}001559275.1, XP_{—}002868908.1, XP_{—}002998647.1, EFY92064.1, XP_{—}002605799.1, BAC43393.1, ABK28457.1, AAL54887.1, BAC43161.1, XP_{—}002333384.1, ZP_{—}03631129.1, AAL84318.1, BAJ99856.1, XP_{—}002593704.1, YP_{—}001965159.1, XP_{—}002454121.1, EFX88390.1, ABR16969.1, NP_{—}177109.3, XP_{—}002441724.1, NP_{—}001166017.1, BAB92256.1, ACE75340.1, AAZ39645.1, XP_{—}002312417.1, XP_{—}002887239.1, NP_{—}001172609.1, NP_{—}001065766.1, XP_{—}002515053.1, AAL54885.1, ABR16897.1, XP_{—}002878579.1, NP_{—}001140775.1, XP_{—}003275955.1, ZP_{—}08045694.1, BAJ94069.1, XP_{—}001654558.1, XP_{—}002436562.1, EAY88702.1, BAK03685.1, XP_{—}003327629.1, XP_{—}002322606.1, EEH42702.1, XP_{—}002037976.1, NP_{—}172774.1, XP_{—}002282477.1, EFX88388.1, XP_{—}002522465.1, EFZ21470.1, AAO41955.1, AAL54886.1, XP_{—}002450277.1, XP_{—}002862559.1, XP_{—}002335046.1, XP_{—}003328408.1, ACE75187.1, XP_{—}001849294.1, XP_{—}002444132.1, XP_{—}002894061.1, EFN77015.1, EGI69992.1, CBI17962.3, AAL54884.1, XP_{—}002998650.1, XP_{—}002105150.1, XP_{—}002877615.1, EFZ22412.1, XP_{—}002439815.1, XP_{—}002300790.1, CBI40391.3, AEI59774.1, XP_{—}002801151.1, XP_{—}003325267.1, XP_{—}001554577.1, EAY79865.1, XP_{—}002465796.1, XP_{—}002931035.1, ABA91371.1, ACE75338.1, XP_{—}001592850.1, XP_{—}001362981.1, XP_{—}002271246.1, EGB11905.1, NP_{—}176713.1, CBJ27248.1, NP_{—}566155.1, EFX87732.1, EEC71661.1, ACG29046.1, NP_{—}001130576.1, XP_{—}001843663.1, ABK25134.1, EGI65081.1, XP_{—}002722841.1, AAL67908.2, AAO15579.1, YP_{—}122047.1, EFA04617.1, YP_{—}001522424.1, ACB87383.1, NP_{—}001027517.1, EEE52725.1, XP_{—}002078257.1, XP_{—}002722842.1, ZP_{—}05128707.1, XP_{—}003208874.1, AAK31592.1, ABA95747.2, NP_{—}001181472.1, NP_{—}001075572.1, XP_{—}001108915.1, XP_{—}001520882.1, XP_{—}002063219.1, EFZ22408.1, AAL57721.1, EFW47740.1, AAQ20834.1, CAN74644.1, XP_{—}002722849.1, BAC30028.1, CAN75729.1, XP_{—}002115603.1, AAN72309.1, EEC68823.1, CAM18519.1, EAZ13863.1, XP_{—}002906159.1, NP_{—}001003947.1, ZP_{—}01858832.1, XP_{—}002882162.1, XP_{—}002089195.1, XP_{—}002892729.1, CAN68037.1, NP_{—}001130648.1, NP_{—}001166016.1, NP_{—}172773.4, ADJ68241.1, EGI62551.1, EFN63658.1, XP_{—}002300103.1, XP_{—}001658673.1, XP_{—}001367719.1, NP_{—}775146.1, XP_{—}001375048.1, AAH21377.1, NP_{—}727589.1, XP_{—}002271847.1, XP_{—}001809620.1, XP_{—}002897528.1, NP_{—}190421.1, XP_{—}002282468.1, XP_{—}536868.2, EEE58297.1, XP_{—}001992105.1, EAY82190.1, ADD20161.1, XP_{—}001363065.1, EAU77129.3, EAY72807.1, EGG03077.1, NP_{—}001181489.1, NP_{—}001177869.1, XP_{—}001966135.1, BAA99522.1, BAK07250.1, XP_{—}002133118.1, NP_{—}001042228.1, AAL57720.1, XP_{—}002897529.1, AAA35712.1, YP_{—}002275016.1, NP_{—}000770.2, XP_{—}002721578.1, XP_{—}321208.4, AAM09532.1, EFN61085.1, BAK06179.1, EFX88389.1, YP_{—}001602608.1, XP_{—}513140.3, NP_{—}001182438.1, AAD31068.1, NP_{—}001093242.1, XP_{—}001367758.2, EFZ18984.1, YP_{—}691921.1, CAH59968.1, AAS80270.1, CAH59967.1, ACQ99381.2, YP_{—}003810988.1, YP_{—}957888.1, CBW44755.1, ZP_{—}05042596.1, ZP_{—}01913735.1, ZP_{—}05043097.1, ADO00145.1, YP_{—}004494060.1, ZP_{—}08206912.1, BAE78452.1, NP_{—}114222.1, ACZ56357.1, YP_{—}640381.1, ZP_{—}04384919.1, ZP_{—}08025219.1, ZP_{—}07715822.1, ZP_{—}06847816.1, YP_{—}001702784.1, AEK27137.1, ZP_{—}07716433.1, ZP_{—}08199554.1, YP_{—}004495520.1, YP_{—}345718.1, ZP_{—}08022914.1, YP_{—}001851443.1, BAG50428.1, YP_{—}001135848.1, BAF95905.1, YP_{—}345695.1, ACP39691.1, ACP39664.1, ACP39635.1, ACP39633.1, ACP39710.1, ACP39698.1, ACP39711.1, BAE47475.1, BAE47474.1, ABW76858.1, ACO50699.1, ACP39643.1, ACP39639.1, ACP39708.1, ACM68663.1, ACP39642.1, ACP39684.1, ACP39636.1, ZP_{—}05095005.1, ACP39652.1, BAE47473.1, ACM68664.1, ACP39646.1, ACP39680.1, ACP39692.1, ACP39675.1, ACP39632.1, ZP_{—}05129284.1, ACP39706.1, ACP39695.1, ACM68665.1, ACP39654.1, ACP39665.1, ACP39649.1, BAE47472.1, ACM68668.1, ACP39676.1, ACP39648.1, ACP39647.1, ZP_{—}01102434.1, ACM68666.1, ACP39641.1, ACM68669.1, ZP_{—}01625037.1, ACP39690.1, ACP39696.1, ACP39697.1, ACP39707.1, ACP39682.1, ACP39650.1, ACP39638.1, ZP_{—}05126641.1, CAH04396.1, ACP39658.1, ZP_{—}01102687.1, ACJ06772.1, YP_{—}001413041.1, YP_{—}552058.1, ADE05601.1, ADI19685.1, BAE47479.1, ZP_{—}01626700.1, ZP_{—}01618279.1, CAH61448.1, YP_{—}001411305.1, YP_{—}003591161.1, ZP_{—}01615522.1, ACM68667.1, ACP39651.1, ZP_{—}05095535.1, ZP_{—}01618489.1, NP_{—}418882.1, ADI19983.1, ACP39677.1, BAE47476.1, ACP39655.1, ACP39656.1, ADI19696.1, BAE47477.1, YP_{—}001413399.1, YP_{—}459878.1, BAE47480.1, BAE47481.1, ACP39653.1, BAE47478.1, YP_{—}001681656.1, ZP_{—}01618281.1, ZP_{—}01627262.1, YP_{—}001413057.1, YP_{—}760740.1, YP_{—}001242466.1, YP_{—}001203574.1, CAH61454.1, YP_{—}002129656.1, YP_{—}001672075.1, ACP39709.1, YP_{—}001990805.1, NP_{—}946959.1, YP_{—}001203575.1, YP_{—}783213.1, YP_{—}003059227.1, YP_{—}004110202.1, ACP39645.1, YP_{—}487538.1, CAH61451.1, YP_{—}570816.1, YP_{—}534107.1, YP_{—}001413223.1, YP_{—}001242465.1, YP_{—}557448.1, ZP_{—}08631162.1, NP_{—}773883.1, ZP_{—}00997728.1, ACP39683.1, NP_{—}768493.1, NP_{—}773882.1, ZP_{—}08271781.1, CAH61449.1, YP_{—}003883668.1, YP_{—}003332953.1, YP_{—}004535688.1, YP_{—}495502.1, YP_{—}459378.1, ZP_{—}08700267.1, ZP_{—}01863452.1, ZP_{—}06860085.1, BAE47487.1, YP_{—}617903.1, ZP_{—}08207422.1, BAE47486.1, ZP_{—}01041003.1, BAE47484.1, ACR78197.1, CAH61456.1, ZP_{—}01858113.1, ACP39681.1, BAE47485.1, ACP39673.1, BAE47483.1, ACP39669.1, BAE47482.1, ACP39674.1, ACP39704.1, ACP39703.1, YP_{—}497095.1, ACP39672.1, ACP39702.1, ACP39670.1, ACP39666.1, YP_{—}458852.1, ACP39687.1, ACP39688.1, ACP39634.1, ACP39686.1, ACP39660.1, ACP39700.1, YP_{—}001411309.1, ZP_{—}01465241.1, ACP39701.1, ACP39679.1, ACP39657.1, ACP39694.1, ACP39659.1, ACP39671.1, ACP39693.1 and YP_{—}003342921.1,
in particular
AAO73954.1, AAO73953.1, XP_{—}002546279.1, AAA34353.2, P30607.1, XP_{—}002421627.1, XP_{—}718670.1, CAA39366.1, XP_{—}001527524.1, AAO73955.1, AAO73956.1, XP_{—}002546278.1, EEQ43157.1, XP_{—}718669.1, AAA34354.1, P10615.3, XP_{—}002421628.1, 226487, P16141.3, CAA39367.1, Q9Y757.2, XP_{—}001485567.1, AAO73958.1, XP_{—}001383506.2, XP_{—}460111.2, AAO73959.1, Q12586.1, XP_{—}460112.2, AAO73960.1, Q12589.1, AAO73961.1, XP_{—}460110.2, EEQ43763.1, XP_{—}710174.1, EDK41572.2, XP_{—}001482650.1, CAA75058.1, XP_{—}002548818.1, Q12588.1, XP_{—}002422222.1, XP_{—}001383636.2, XP_{—}001525381.1, XP_{—}002548823.1, P30610.1, AAO73952.1, XP_{—}002548428.1, CAA36197.1, XP_{—}002421126.1, AAA34320.1, P16496.3, P30608.1, P24458.1, XP_{—}717999.1, XP_{—}001383817.1, Q9Y758.1, XP_{—}001482092.1, XP_{—}001383710.2, P30609.1, AAB24479.1, XP_{—}457792.1, XP_{—}001524144.1, XP_{—}457727.2, XP_{—}001525578.1, XP_{—}002616743.1, XP_{—}002614836.1, XP_{—}001525577.1, AAO73957.1, Q12585.1, XP_{—}001386440.2, XP_{—}002616857.1, XP_{—}001483276.1, XP_{—}500402.1, EDK39907.2, XP_{—}500560.1, XP_{—}001211376.1, XP_{—}002560027.1, XP_{—}504857.1, XP_{—}500855.1, XP_{—}504406.1, BAA31433.1, XP_{—}500856.1, XP_{—}501148.1, XP_{—}746567.1, XP_{—}001262425.1, XP_{—}001274843.1, XP_{—}002840588.1, XP_{—}002377641.1, XP_{—}001825995.1, XP_{—}001400739.1, XP_{—}718066.1, CAA35593.1, XP_{—}664735.1, XP_{—}002150795.1, XP_{—}500097.1, XP_{—}002483325.1, XP_{—}504311.1, XP_{—}500273.1, XP_{—}002548817.1, EDP54484.1, XP_{—}755288.1, XP_{—}001260447.1, EFY97851.1, ACD75398.1, ADK36660.1, XP_{—}001213081.1, XP_{—}002377989.1, XP_{—}001826299.1, XP_{—}001554811.1, XP_{—}501667.1, XP_{—}002148942.1, ADK36662.1, XP_{—}002565827.1, P30611.1, XP_{—}001267871.1, XP_{—}002372373.1, EFY84686.1, P43083.1, XP_{—}001263094.1, XP_{—}002148355.1, XP_{—}002568429.1, XP_{—}001817314.1, Q12587.1, XP_{—}001396435.1, XP_{—}001938589.1, XP_{—}001388497.2, XP_{—}663661.1, XP_{—}003295335.1, XP_{—}002152088.1, XP_{—}001212071.1, Q12573.1, XP_{—}002379858.1, XP_{—}001821592.1, XP_{—}002844341.1, XP_{—}001394678.1, ACD75400.1, XP_{—}003170343.1, XP_{—}001265480.1, XP_{—}002550661.1, EDP55514.1, XP_{—}001528842.1, XP_{—}749919.1, XP_{—}001593058.1, P30612.1, EGC48494.1, EEH04429.1, XP_{—}001585586.1, XP_{—}003236182.1, XP_{—}001400199.1, EEQ46951.1, XP_{—}721410.1, EGP87864.1, XP_{—}002380808.1, XP_{—}001792771.1, XP_{—}001208515.1, XP_{—}001216161.1, XP_{—}003071804.1, EFW16963.1, XP_{—}002542118.1, XP_{—}001936677.1, EGD95268.1, XP_{—}003015678.1, XP_{—}501748.1, XP_{—}003169562.1, EFY96492.1, XP_{—}682653.1, XP_{—}002421356.1, CAK43439.1, EFY93677.1, XP_{—}747767.1, XP_{—}001244958.1, XP_{—}003019635.1, XP_{—}002847463.1, EGP83273.1, EGR52487.1, XP_{—}002622526.1, XP_{—}002563618.1, CBX99718.1, XP_{—}001552081.1, XP_{—}003066638.1, XP_{—}003176049.1, ACD75402.1, BAA05145.1, XP_{—}002482834.1, XP_{—}001257501.1, XP_{—}001934574.1, XP_{—}001269972.1, XP_{—}001587438.1, XP_{—}001215856.1, XP_{—}002149824.1, XP_{—}001550556.1, XP_{—}003011982.1, XP_{—}001827121.1, XP_{—}003233566.1, XP_{—}003022481.1, EGR47044.1, EFQ34695.1, XP_{—}003170005.1, BAG09241.1, XP_{—}002796370.1, XP_{—}003019300.1, XP_{—}002563873.1, CAK40654.1, EEH19741.1, XP_{—}003012518.1, EGD95716.1, XP_{—}003239409.1, BAJ04363.1, XP_{—}001537012.1, BAE66393.1, EGP85214.1, XP_{—}002487227.1, AAV66104.1, EGE07669.1, XP_{—}362943.2, XP_{—}003016806.1, EFQ27388.1, XP_{—}002384360.1, XP_{—}002836323.1, XP_{—}001274959.1, EFZ03093.1, XP_{—}661521.1, XP_{—}002849803.1, XP_{—}001589398.1, AAR99474.1, XP_{—}003189427.1, XP_{—}001823699.1, XP_{—}364111.1, XP_{—}001262753.1, EFY86805.1, XP_{—}001390153.2, XP_{—}002384738.1, XP_{—}001941811.1, XP_{—}001220831.1, XP_{—}003296981.1, XP_{—}002480829.1, BAD83681.1, XP_{—}001827526.2, XP_{—}369556.1, CAK38224.1, EFQ26532.1, XP_{—}002562328.1, XP_{—}001904540.1, EGO52476.1, XP_{—}002382002.1, XP_{—}001225874.1, XP_{—}958030.2, XP_{—}002540883.1, XP_{—}001908957.1, XP_{—}001559255.1, XP_{—}364102.1, EDP48064.1, XP_{—}365075.1, XP_{—}381460.1, CBX95930.1, XP_{—}003054099.1, XP_{—}361347.2, XP_{—}002846867.1, XP_{—}001214985.1, EFQ35175.1, XP_{—}002479062.1, XP_{—}001908613.1, XP_{—}003345380.1, EGR50567.1, XP_{—}002479350.1, XP_{—}001394417.2, XP_{—}001394159.2, XP_{—}002146776.1, EGP86783.1, EFX02953.1, CAK45889.1, XP_{—}003006887.1, XP_{—}002541427.1, XP_{—}750735.1, XP_{—}001257962.1, EGO51720.1, XP_{—}003005336.1, EGP83197.1, XP_{—}002149832.1, XP_{—}003052680.1, XP_{—}365851.1, XP_{—}001799910.1, XP_{—}003347175.1, XP_{—}002565258.1, EGR48918.1, EGR52524.1, XP_{—}964653.2, XP_{—}002147083.1, XP_{—}002843935.1, EEH19393.1, CAC10088.1, EEH47609.1, EEQ92528.1, XP_{—}001246560.1, XP_{—}002626168.1, XP_{—}003024880.1, XP_{—}003169255.1, XP_{—}003013780.1, XP_{—}003235691.1, XP_{—}746816.1, EGD98483.1, XP_{—}001389925.2, XP_{—}002842817.1, XP_{—}002797278.1, ADK36666.1, XP_{—}003305469.1, XP_{—}001548471.1, XP_{—}001806478.1, EFQ34989.1, XP_{—}001552987.1, CAC24473.1, XP_{—}002541530.1, EEQ89262.1, XP_{—}001247332.1, XP_{—}003066043.1, EDP47672.1, XP_{—}002628451.1, XP_{—}001910644.1, EGR44510.1, EFQ36733.1, XP_{—}003052472.1, XP_{—}001393445.2, XP_{—}001522438.1, XP_{—}001397944.2, CAK49049.1, EFQ30109.1, XP_{—}001585052.1, XP_{—}388496.1, XP_{—}003173913.1, CBF76609.1, CAK46976.1, XP_{—}370476.1, XP_{—}002145942.1, XP_{—}003004457.1, ADK36663.1, XP_{—}003040708.1, XP_{—}003351473.1, EFY84692.1, XP_{—}748328.2, XP_{—}003190325.1, XP_{—}002378813.1, EGR46513.1, XP_{—}002145326.1, XP_{—}662462.1, XP_{—}747469.1, XP_{—}001935085.1, EGR45892.1, EGP89995.1, XP_{—}001222615.1, XP_{—}001224356.1, XP_{—}001934479.1, XP_{—}001267956.1, ADK36661.1, EFY97845.1, EFY84206.1, XP_{—}001412594.1, XP_{—}002583529.1, XP_{—}002843371.1, XP_{—}001587730.1, EGE03365.1, EFZ01428.1, XP_{—}001558890.1, XP_{—}002487181.1, EFY92529.1, XP_{—}002380252.1, EFY99978.1, BAG09240.1, XP_{—}002381768.1, XP_{—}001800031.1, XP_{—}001825073.2, BAE63940.1, XP_{—}681680.1, XP_{—}002486603.1, EGR50064.1, CAK37996.1, CAO91865.1, XP_{—}001258702.1, XP_{—}001586739.1, XP_{—}001560806.1, CBF69707.1, ADN43682.1, XP_{—}001593179.1, XP_{—}001392650.1, XP_{—}366716.2, CAL69594.1, XP_{—}001269140.1, XP_{—}002566307.1, XP_{—}001555473.1, XP_{—}663925.1, XP_{—}001598033.1, XP_{—}001554305.1, XP_{—}001560475.1, EFQ32286.1, XP_{—}001216788.1, XP_{—}002483975.1, XP_{—}002143660.1, EFQ36688.1, XP_{—}001798699.1, EEH44101.1, AAA34334.1, XP_{—}001545581.1, XP_{—}001791898.1, XP_{—}002839066.1, EGC49561.1, EEH05830.1, BAA05146.1, EEH21852.1, XP_{—}001559854.1, EER40289.1, XP_{—}001560028.1, XP_{—}001554079.1, XP_{—}001559275.1, EFY92064.1, XP_{—}001589816.1, EEH42702.1, XP_{—}001554577.1, XP_{—}001592850.1, YP_{—}691921.1, CAH59968.1, AAS80270.1, CAH59967.1, ACQ99381.2, YP_{—}003810988.1, YP_{—}957888.1, CBW44755.1, ZP_{—}05042596.1, ZP_{—}01913735.1, ZP_{—}05043097.1, ADO00145.1, YP_{—}004494060.1, ZP_{—}08206912.1, BAE78452.1, NP_{—}114222.1, ACZ56357.1, YP_{—}640381.1, ZP_{—}04384919.1, ZP_{—}08025219.1, ZP_{—}07715822.1, ZP_{—}06847816.1, YP_{—}001702784.1, AEK27137.1, ZP_{—}07716433.1, ZP_{—}08199554.1, YP_{—}004495520.1, YP_{—}345718.1, ZP_{—}08022914.1, YP_{—}001851443.1, BAG50428.1, YP_{—}001135848.1, BAF95905.1, YP_{—}345695.1, ACP39691.1, ACP39664.1, ACP39635.1, ACP39633.1, ACP39710.1, ACP39698.1, ACP39711.1, BAE47475.1, BAE47474.1, ABW76858.1, ACO50699.1, ACP39643.1, ACP39639.1, ACP39708.1, ACM68663.1, ACP39642.1, ACP39684.1, ACP39636.1, ZP_{—}05095005.1, ACP39652.1, BAE47473.1, ACM68664.1, ACP39646.1, ACP39680.1, ACP39692.1, ACP39675.1, ACP39632.1, ZP_{—}05129284.1, ACP39706.1, ACP39695.1, ACM68665.1, ACP39654.1, ACP39665.1, ACP39649.1, BAE47472.1, ACM68668.1, ACP39676.1, ACP39648.1, ACP39647.1, ZP_{—}01102434.1, ACM68666.1, ACP39641.1, ACM68669.1, ZP_{—}01625037.1, ACP39690.1, ACP39696.1, ACP39697.1, ACP39707.1, ACP39682.1, ACP39650.1, ACP39638.1, ZP_{—}05126641.1, CAH04396.1, ACP39658.1, ZP_{—}01102687.1, ACJ06772.1, YP_{—}001413041.1, YP_{—}552058.1, ADE05601.1, ADI19685.1, BAE47479.1, ZP_{—}01626700.1, ZP_{—}01618279.1, CAH61448.1, YP_{—}001411305.1, YP_{—}003591161.1, ZP_{—}01615522.1, ACM68667.1, ACP39651.1, ZP_{—}05095535.1, ZP_{—}01618489.1, NP_{—}418882.1, ADI19983.1, ACP39677.1, BAE47476.1, ACP39655.1, ACP39656.1, ADI19696.1, BAE47477.1, YP_{—}001413399.1, YP_{—}459878.1, BAE47480.1, BAE47481.1, ACP39653.1, BAE47478.1, YP_{—}001681656.1, ZP_{—}01618281.1, ZP_{—}01627262.1, YP_{—}001413057.1, YP_{—}760740.1, YP_{—}001242466.1, YP_{—}001203574.1, CAH61454.1, YP_{—}002129656.1, YP_{—}001672075.1, ACP39709.1, YP_{—}001990805.1, NP_{—}946959.1, YP_{—}001203575.1, YP_{—}783213.1, YP_{—}003059227.1, YP_{—}004110202.1, ACP39645.1, YP_{—}487538.1, CAH61451.1, YP_{—}570816.1, YP_{—}534107.1, YP_{—}001413223.1, YP_{—}001242465.1, YP_{—}557448.1, ZP_{—}08631162.1, NP_{—}773883.1, ZP_{—}00997728.1
and especially preferably
AAO73954.1, AAO73953.1, XP_{—}002546279.1, AAA34353.2, P30607.1, XP_{—}002421627.1, XP_{—}718670.1, CAA39366.1, AAO73955.1, AAO73956.1, XP_{—}002546278.1, EEQ43157.1, XP_{—}718669.1, AAA34354.1, P10615.3, XP_{—}002421628.1, 226487, P16141.3, CAA39367.1, AAO73958.1, AAO73959.1, Q12586.1, AAO73960.1, Q12589.1, AAO73961.1, EEQ43763.1, XP_{—}710174.1, CAA75058.1, XP_{—}002548818.1, Q12588.1, XP_{—}002422222.1, XP_{—}002548823.1, P30610.1, AAO73952.1, XP_{—}002548428.1, CAA36197.1, XP_{—}002421126.1, AAA34320.1, P16496.3, P30608.1, P24458.1, XP_{—}717999.1, P30609.1, AAB24479.1, AAO73957.1, Q12585.1, XP_{—}718066.1, CAA35593.1, XP_{—}002548817.1, P30611.1, P43083.1, Q12587.1, Q12573.1, XP_{—}002550661.1, P30612.1, EEQ46951.1, XP_{—}721410.1, XP_{—}002421356.1, BAA05145.1, BAG09241.1, CAC24473.1, BAG09240.1, AAA34334.1, BAA05146.1, XP_{—}500402.1, XP_{—}500560.1, XP_{—}504857.1, XP_{—}500855.1, XP_{—}504406.1, BAA31433.1, XP_{—}500856.1, XP_{—}501148.1, XP_{—}500097.1, XP_{—}504311.1, XP_{—}500273.1, XP_{—}501667.1, XP_{—}501748.1, YP_{—}691921.1, CAH59968.1, AAS80270.1, CAH59967.1, ACQ99381.2, YP_{—}003810988.1, YP_{—}957888.1, CBW44755.1, ZP_{—}05042596.1, ZP_{—}01913735.1, ZP_{—}05043097.1, ADQ00145.1, YP_{—}004494060.1, ZP_{—}08206912.1, BAE78452.1, NP_{—}114222.1, ACZ56357.1, YP_{—}640381.1, ZP_{—}04384919.1, ZP_{—}08025219.1, ZP_{—}07715822.1, ZP_{—}06847816.1, YP_{—}001702784.1, AEK27137.1, ZP_{—}07716433.1, ZP_{—}08199554.1, YP_{—}004495520.1, YP_{—}345718.1, ZP_{—}08022914.1, YP_{—}001851443.1, BAG50428.1, YP_{—}001135848.1, BAF95905.1, YP_{—}345695.1, ACP39691.1, ACP39664.1, ACP39635.1, ACP39633.1, ACP39710.1, ACP39698.1, ACP39711.1, BAE47475.1, BAE47474.1, ABW76858.1, ACO50699.1, ACP39643.1, ACP39639.1, ACP39708.1, ACM68663.1, ACP39642.1, ACP39684.1, ACP39636.1, ZP_{—}05095005.1, ACP39652.1, BAE47473.1, ACM68664.1, ACP39646.1, ACP39680.1, ACP39692.1, ACP39675.1, ACP39632.1, ZP_{—}05129284.1, ACP39706.1, ACP39695.1, ACM68665.1, ACP39654.1, ACP39665.1, ACP39649.1, BAE47472.1, ACM68668.1, ACP39676.1, ACP39648.1, ACP39647.1, ZP_{—}01102434.1, ACM68666.1, ACP39641.1, ACM68669.1, ZP_{—}01625037.1, ACP39690.1, ACP39696.1, ACP39697.1, ACP39707.1, ACP39682.1, ACP39650.1 and ACP39638.1
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E₁ₐ is generally understood in particular as meaning the conversion of lauric acid and/or its methyl ester into ω-hydroxylauric acid and/or its methyl ester.

### Specific Enzymes E_{1b}

AlkB alkane hydroxylases E_{1b} which are preferred according to the invention are selected from among the list

YP_{—}001185946.1, Q9WWW6.1, YP_{—}957898.1, YP_{—}957728.1, YP_{—}694427.1, BAC98365.1, ZP_{—}00957064.1, CAC86944.1, YP_{—}001672212.1, CAB59525.1, ACH99213.1, ACH99215.1, ACH99216.1, AAK56792.1, ACH99229.1, ACS91348.1, AAP41820.1, ZP_{—}05128075.1, CAM58121.1, CAM58085.1, ACQ44675.1, ACZ62808.1, ZP_{—}01738706.1, ZP_{—}01916228.1, ZP_{—}01225325.1, YP_{—}001023605.1, ACJ22747.1, ACT91140.1, AAT91722.2, CBA27418.1, YP_{—}001889129.1, EGC97932.1, ACT91201.1, ZP_{—}05083049.1, YP_{—}554098.1, ZP_{—}01900149.1, ADG26619.1, ADG26657.1, ADG26640.1, ZP_{—}06838771.1, ADG26649.1, ADG26651.1, ZP_{—}02374120.1, YP_{—}368326.1, ZP_{—}02380481.1, ADG26643.1, ADG26628.1, YP_{—}442346.1, ADG26620.1, ADG26647.1, ZP_{—}07673680.1, ADG26638.1, YP_{—}002232139.1, YP_{—}001118743.1, ZP_{—}01764629.1, YP_{—}108945.1, YP_{—}334185.1, ZP_{—}04897834.1, ZP_{—}02889567.1, YP_{—}620386.1, YP_{—}002897546.1, ZP_{—}02166109.1, ZP_{—}02904755.1, ADG26639.1, YP_{—}001892637.1, ADG26642.1, ZP_{—}04939380.1, ZP_{—}02464124.1, YP_{—}102417.1, CAC36356.1, ACJ22727.1, YP_{—}001764240.1, YP_{—}002765609.1, YP_{—}001945311.1, ZP_{—}03586616.1, ACJ22665.1, ZP_{—}03574223.1, CAC37038.1, ZP_{—}02456517.1, YP_{—}001807560.1, YP_{—}002779449.1, AAK97454.1, YP_{—}002912304.1, ACR55689.1, YP_{—}003397515.1, YP_{—}004361423.1, YP_{—}772734.1, ACJ65014.1, ACT31523.1, ACJ22750.1, ZP_{—}07375042.1, YP_{—}002776786.1, ACB11552.1, ZP_{—}02363472.1, ADG26653.1, ZP_{—}04383196.1, ZP_{—}02356342.1, ACJ22751.1, YP_{—}952571.1, ACU43494.1, YP_{—}001135977.1, YP_{—}002764193.1, YP_{—}003855036.1, YP_{—}004078475.1, AAK97448.1, ZP_{—}04388098.1, ACX30747.1, ADG26632.1, ACJ22719.1, ADO21492.1, ZP_{—}05061580.1, ADR72654.1, ACZ65961.1, ACX30755.1, YP_{—}001849604.1, AAV64895.1, YP_{—}004495037.1, YP_{—}702497.1, YP_{—}001069662.1, ZP_{—}06850622.1, BAF34299.1, CAB51024.2, YP_{—}004008018.1, YP_{—}003768535.1, ACJ65013.1, ZP_{—}07282765.1, YP_{—}886209.1, ACJ22725.1, ZP_{—}08155372.1, YP_{—}004493362.1, ZP_{—}05228000.1, ZP_{—}07717360.1, BAD67020.1, YP_{—}004524245.1, ZP_{—}07715778.1, NP_{—}217769.1, ACS91349.1, YP_{—}960105.1, ZP_{—}07014137.1, YP_{—}004746682.1, ZP_{—}08022271.1, ACN62569.1, ADQ37951.1, YP_{—}003647687.1, YP_{—}003837040.1, ADG26600.1, YP_{—}002768905.1, ZP_{—}08553310.1, ADG26597.1, ACJ22749.1, ADG26598.1, YP_{—}001704327.1, ZP_{—}04385381.1, ZP_{—}04751264.1, ADG26609.1, ADG26610.1, ZP_{—}06417258.1, ADG26607.1, ADP98338.1, YP_{—}003275257.1, YP_{—}004084103.1, ADG26630.1, ADG26625.1, ADG26605.1, ADG26599.1, ZP_{—}05218167.1, ADQ37950.1, YP_{—}921354.1, ADG26645.1, ADG26612.1, YP_{—}004493370.1, YP_{—}638501.1, YP_{—}003809668.1, NP_{—}962298.1, ZP_{—}04750514.1, ADG26608.1, ADT82701.1, ACJ06773.1, YP_{—}120833.1, ADG26618.1, ADG26602.1, ADG26623.1, ZP_{—}04383566.1, ZP_{—}08122407.1, YP_{—}004077166.1, ZP_{—}05041651.1, ZP_{—}04608296.1, ABU93351.2, YP_{—}003658078.1, ADQ37949.1, ADG26652.1, YP_{—}002765850.1, AAK97447.1, CAD24434.1, CAC40954.1, ACT91203.1, YP_{—}120829.1, ZP_{—}07282558.1, YP_{—}003298195.1, YP_{—}001851790.1, ZP_{—}05827357.1, ADG26633.1, CAB51020.1, YP_{—}953908.1, ZP_{—}07990416.1, YP_{—}119532.1, ZP_{—}08442348.1, ZP_{—}08276444.1, ZP_{—}04661203.1, ABO12068.2, YP_{—}001846325.1, ADQ37952.1, ZP_{—}08198697.1, ZP_{—}00996652.1, YP_{—}001707231.1, ZP_{—}08433663.1, ZP_{—}08205256.1, YP_{—}003732372.1, YP_{—}906529.1, ACT91204.1, YP_{—}001506534.1, YP_{—}001713880.1, YP_{—}883357.1, YP_{—}004525252.1, ADG26604.1, YP_{—}001134633.1, ZP_{—}08195602.1, ZP_{—}06690500.1, ZP_{—}05826167.1, ADY81595.1, ZP_{—}06056754.1, AAK31348.1, YP_{—}251715.1, ZP_{—}08461977.1, ZP_{—}05847237.1, YP_{—}712218.1, YP_{—}001084670.1, ZP_{—}04387164.1, YP_{—}260041.1, YP_{—}002873097.1, ADG26614.1, AAK97446.1, YP_{—}001280943.1, ZP_{—}04386125.1, AAC36353.2, CCA29159.1, CAD10804.1, CCA29151.1, CAC40953.1, CCA29161.1, ABA55770.1, AAS93604.4, CCA29173.1, CCA29155.1, CCA29156.1, ABA55772.1, CCA29154.1, ABA55793.1, CCA29162.1, CCA29170.1, ZP_{—}03824539.1, CCA29166.1, CCA29136.1, ZP_{—}06065934.1, ABB54493.1, CCA29169.1, YP_{—}003112137.1, CCA29127.1, CCA29148.1, CCA29160.1, ZP_{—}06057458.1, ABA55773.1, YP_{—}004016090.1, CCA29139.1, YP_{—}480358.1, ABA55787.1, CCA29150.1, CCA29130.1, ZP_{—}07775830.1, ABA55779.1, CCA29132.1, YP_{—}003732938.1, BAB33284.1, CCA29149.1, CCA29145.1, ABA55783.1, CCA29137.1, CCA29129.1, CCA29158.1, CCA29176.1, CCA29142.1, CCA29144.1, BAB33287.1, CCA29133.1, CCA29140.1, CCA29135.1, ZP_{—}06066074.1, ZP_{—}03823182.1, CCA29171.1, CCA29152.1, CCA29131.1, ABA55780.1, CCA29163.1, CCA29143.1, CCA29153.1, YP_{—}001580600.1, CCA29134.1, CCA29138.1, YP_{—}046098.1, ZP_{—}06072466.1, ZP_{—}05361594.1, ACU43504.1, CCA29147.1, CCA29146.1, ZP_{—}06061712.1, ACT91185.1, ACT91147.1, ACT91178.1, ACT91167.1, ACT91181.1, ACT91188.1, ZP_{—}06069784.1, ACT91205.1, ZP_{—}06725872.1, ACT91171.1, CCA29128.1, ABY56787.1, ADE05602.1, ACU43474.1, ACJ22718.1, ABB90688.1, ACU43519.1, ABB96093.1, ACU43485.1, ACU43493.1, ABW76857.1, ACT91163.1, ACJ22673.1, ZP_{—}06188150.1, ACT91242.1, ACT91225.1, ACT91211.1, ACU43479.1, ACU43491.1, ACU43522.1, ACU43486.1, ACT91221.1, ACJ22662.1, ACU43506.1, ACU43487.1, ACT91259.1, AAA97866.1, ACU43502.1, YP_{—}001252544.1, ABB96084.1, ACU43520.1, ACJ22668.1, ACU43503.1, ACT91230.1, ABA55777.1, ACT91231.1, ZP_{—}01748311.1, ACJ22724.1, ACU43475.1, ACU43511.1, ACU43490.1, ZP_{—}08330953.1, ACU43484.1, CBX01596.1, ACT91168.1, YP_{—}096989.1, ACT91215.1, YP_{—}125370.1, ACT91233.1, ACU43478.1, ADE05603.1, ACJ22715.1, ACU43512.1, ACT91196.1, ACJ22692.1, ACU43510.1, ACU43521.1, ACT91174.1, ACT91213.1, ACT91142.1, ACT91206.1, ACT91216.1, ACT91182.1, ACT91255.1, ACT91246.1, ACT91217.1, ACT91155.1, ACT91240.1, ACT91207.1, ACU43495.1, YP_{—}128249.1, ACT91160.1, YP_{—}004052990.1, ACT91226.1, ACU43507.1, ABO61855.1, ACT91214.1, ACT91220.1, YP_{—}001188237.1, ACJ22689.1, ZP_{—}01689499.1, YP_{—}004379711.1, ACJ22748.1, ABB90683.1, ACT91223.1, ACT91235.1, ABO61786.1, ACU43508.1, ACU43492.1, ACT91219.1, ACT91244.1, ABO61856.1, ACT91239.1, ACU43473.1, ABO61850.1, ACT91262.1, ACT91261.1, ACT91224.1, ACU43499.1, ACU43488.1, ADO21767.1, YP_{—}004654946.1, ADO21777.1, ABB96089.1, ABO61852.1, ABO61847.1, ACT91222.1, ADO21764.1, ACU43477.1, ADO21773.1, ABO61787.1, ABB96080.1, ABO61857.1, ACT91228.1, ABB96070.1, ADO21744.1, ACT91245.1, CAG17608.1, ADO21747.1, YP_{—}001349162.1, ABK63807.1, ZP_{—}06879583.1, NP_{—}250216.1, ACT91234.1, ZP_{—}01364874.1, ABO61789.1, ADO21772.1, ACU43516.1, ACU43505.1, ACU43501.1, ACT91236.1, ZP_{—}07792758.1, ACZ64723.1, ADO21743.1, ADO21759.1, ACZ64752.1, ADO21755.1, ACD75517.1, YP_{—}790621.1, ACB11551.1, ADO21748.1, NP_{—}251264.1, ZP_{—}01365940.1, ADO21762.1, ADO21739.1, ACU43496.1, ABO61854.1, ZP_{—}06878434.1, ACU43489.1, ACU43483.1, ADO21746.1, ACT91237.1, ZP_{—}01895378.1, ACT91164.1, ADO21736.1, ACJ22711.1, ACZ64754.1, ZP_{—}05042146.1, ADO21688.1, ADO21648.1, YP_{—}001348003.1, ADP98656.1, ADO21737.1, ADO21760.1, ADO21754.1, ADO21740.1, ACZ64758.1, ACU43497.1, ZP_{—}01912185.1, ABB96111.1, ACU43482.1, ACB11549.1, ADO21775.1, CCA29157.1, ADO21681.1, ADO21668.1, ADO21656.1, ACU43517.1, ACT91165.1, ACJ22695.1, ACJ22688.1, ABB96071.1, ADO21763.1, ACT91241.1, ADO21735.1, ACB11550.1, ADO21778.1, ACT91172.1, ADO21765.1, ABB96087.1, CBJ30233.1, ACJ22752.1, ABB96105.1, ACB15251.1, ACJ22694.1, ACZ64741.1, ACZ64706.1, ABB96108.1, ACT91191.1, ABB96101.1, ABB90691.1, ACZ64745.1, YP_{—}691842.1, ABB96075.1, ABB90682.1, ABB90690.1, ADO21676.1, ADO21679.1, ABO61768.1, YP_{—}435857.1, ACJ22722.1, ACT91238.1, ACZ64725.1, CAC14062.1, ADO21682.1, ACZ64771.1, ACZ64718.1, ACZ64724.1, ADO21670.1, ADO21667.1, CAC37048.1, ACZ64708.1, ABB96092.1, ACJ22687.1, ACZ64703.1, ADO21690.1, ABB92364.1, ACB11547.1, ACZ64720.1, ADO21655.1, ACZ64717.1, ADO21680.1, ACZ64757.1, ACZ64733.1, ACT91144.1, ACU43481.1, ACT91179.1, ZP_{—}02181409.1, ACZ64704.1, ABB96073.1, ACJ22675.1, ACZ64721.1, ABB96090.1, ACJ22729.1, ACU43515.1, ZP_{—}01307000.1, ABB90685.1, YP_{—}003862088.1, ACZ64715.1, ACZ64710.1, ACJ22735.1, ABB90687.1, ADO21661.1, ADO21674.1, ACT91177.1, ABB54492.1, ABB96076.1, ABB92365.1, ACT91194.1, ADO21689.1, ACJ22691.1, ABB90681.1, ADO21649.1, ADO21671.1, ACZ64728.1, ABB96095.1, CAC40945.1, ADO21652.1, ADO21665.1, ADE08461.1, ADO21678.1, ACZ64705.1, ACJ22690.1, ADO21675.1, ADO21685.1, ABB96072.1, ACJ22736.1, ACB11540.1, ABB96091.1, AC 104540.1, ACT91251.1, ACT91146.1, ACT91166.1, ACT91156.1, ADO21752.1, ADO21673.1, ADO21725.1, ABB96104.1, ABB90694.1, ABB90696.1, ACT91173.1, ADO21647.1, ZP_{—}03700804.1, ACT91232.1, ADO21694.1, CAC40949.1, ABB92361.1, ACT91195.1, ACI04538.1, ADO21691.1, ACJ22685.1, ADO21653.1, ABS12461.1, ACZ64736.1, ACZ64772.1, ABB90680.1, ADO21659.1, ACZ64774.1, ADO21684.1, ADO21729.1, ADO21650.1, ADO21733.1, ACZ64755.1, ACZ64751.1, ABA55775.1, ADO21738.1, CCA29174.1, ADO21669.1, ACZ64744.1, ADO21654.1, ADO21768.1, ABB96106.1, CCA29168.1, ACT91176.1, ACB11555.1, ABB90695.1, ADO21660.1, ACJ22666.1, ACZ64778.1, ADO21766.1, ADO21677.1, ZP_{—}02161687.1, CCA29165.1, ADO21745.1, ACB11548.1, ABB90689.1, ABB96107.1, AAT46052.1, ADO21718.1, ADO21722.1, ABB96088.1, EFW40271.1, ADO21686.1, ABB96103.1, ACU43500.1, ACB11536.1, ABB92360.1, CCA29167.1, ACT91199.1, ACZ64770.1, ACJ22716.1, ABA55786.1, ACZ64737.1, ABB96083.1, ACJ22676.1, ACZ64735.1, ACT91212.1, ACJ22765.1, CAJ01371.1, CAC 17734.1, ABD36389.1, ACB11537.1, CAC08515.1, ACZ64714.1, ACU43513.1, ABB96082.1, ADN21387.1, ADO21711.1, ABD36392.1, ABR10770.1, CAC37049.1, ABB96098.1, ABB90692.1, ACB11535.1, ACZ64768.1, ACJ22756.1, ABB96094.1, ABA55791.1, ABB96078.1, ACT91141.1, ACZ64779.1, ACZ64750.1, CAJ01370.1, ACZ64753.1, ACU43480.1, ABA55794.1, ABB96085.1, ABB96110.1, YP_{—}004448035.1, ACZ64709.1, ABB96102.1, ACZ64773.1, CCA29175.1, ACZ64749.1, ACZ64756.1, ACZ64781.1, ABO61777.1, ACZ64759.1, ACZ64764.1, ACZ64740.1, ACT91249.1, ZP_{—}03702922.1, ACB11545.1, ACZ64775.1, ACZ64769.1, ACT91145.1, ACZ64742.1, ACT91254.1, ACZ64762.1, ACZ64716.1, ACZ64777.1, ADM26559.1, ABB96096.1, ACZ64780.1, ZP_{—}01201250.1, CAH55829.1, ZP_{—}01052921.1, ABB96077.1, ADO21658.1, ACT91161.1, ABB90684.1, ACR56750.1, ABB90697.1, ACZ64746.1, ABB92367.1, ACT91139.1, ACZ64763.1, ACT91200.1, ABO61773.1, ABB96081.1, ACZ64748.1, ACZ64782.1, ACU43498.1, ADO21651.1, ABB90679.1, BAG06233.1, ACZ64747.1, ABB96086.1, ACZ64761.1, ABB92370.1, ABO61774.1, ACT91175.1, ABB90686.1, ACB11546.1, ZP_{—}01740604.1, ABO61785.1, YP_{—}001531377.1, XP_{—}001434539.1, ABA55767.1, ABO21865.1, ABF55636.1, ABA55751.1, ABB90698.1, ADD12311.1, ACZ64765.1, ABB92366.1, ABB92368.1, ACI04539.1, XP_{—}001023288.1, ACZ64783.1, ADO21692.1, ZP_{—}01753800.1, ACZ64760.1, ACZ64700.1, ZP_{—}01055480.1, ACZ64767.1, ACZ64701.1, ABA55745.1, ABA55752.1, ACZ64766.1, YP_{—}614640.1, ABA55759.1, ADO21723.1, BAG06232.1, ZP_{—}01002389.1, ABB90693.1, ACT91264.1, ABB92358.1, BAF99026.1, ABR10769.1, ZP_{—}00959618.1, AEA08580.1, ADD22986.1, CAB51023.1, CAC40958.1, ADO21709.1, CAB51025.1, ACI15226.1, ACJ22680.1, ZP_{—}05741459.1, ACT91248.1, ABU48567.1, ABO61792.1, ACJ22754.1, EFN53276.1, AAL87644.1, ACT91209.1, ZP_{—}02147281.1, ACU43518.1, ACZ64776.1, ACB11543.1, ACT91151.1, ACJ22764.1, ACT91159.1, ABA18186.1, AEA08579.1, ADO21770.1, ABF55634.1, CAA27179.1, ABA55741.1, ADO21705.1, ZP_{—}01754375.1, ACB11541.1, ACR56751.1, ACT91250.1, ADO21769.1, ADO21753.1, ABB96097.1, ACT91208.1, ABO21867.1, ADO21757.1, ACB11554.1, ABA55749.1, CAC40951.1, ADO21719.1, ABB96074.1, ZP_{—}00954267.1, ZP_{—}05786269.1, AEH76912.1, ABA55742.1, ABA55748.1, BAG06236.1, ADO21732.1, ABA55750.1, ABA55768.1, ACT31522.1, ZP_{—}05090796.1, ACZ64739.1, YP_{—}915886.1, ADO21731.1, CAC40948.1, XP_{—}001032273.1, AEH76911.1, ABA55743.1, ABO61769.1, ABA55755.1, ZP_{—}05122263.1, ADO21756.1, ABA55744.1, ABA55746.1, ZP_{—}01901011.1, ZP_{—}02150761.1, ADO21742.1, ACR56752.1, ABA55747.1, ABF55637.1, ABA55740.1, ABA55760.1, ZP_{—}00948812.1, ABA55804.1, ADO21771.1, ZP_{—}05342453.1, ABF55638.1, YP_{—}508336.1, ABB92357.1, ZP_{—}01049702.1, ABU48546.1, ABU48555.1, ABA55764.1, ABO21866.1, ZP_{—}05079274.1, ZP_{—}01880441.1, ACZ64738.1, ZP_{—}05842058.1, ACT91218.1, ABA55769.1, ABA55739.1, ABA55803.1, ACT91247.1, ABA55782.1, ACZ17539.1, ABB92359.1, ACH69966.1, ZP_{—}01035050.1, ACZ17537.1, ABA55774.1, ACZ64729.1, ACZ17538.1, ZP_{—}01751972.1, ACZ64731.1, ACZ64702.1, AAR13803.1, AEJ28400.1, ZP_{—}05099213.1, CAB51021.1, ACZ17531.1, AEH76914.1, ZP_{—}05051648.1, ACZ64726.1, ACZ17540.1, ACZ64727.1, ZP_{—}02152773.1, ACT91253.1, ACZ17536.1, XP_{—}001423873.1, ACZ17534.1, YP_{—}168645.1, ACZ17520.1, ABY56786.1, ACB11539.1, ZP_{—}01157350.1, AEH76910.1, ABY56784.1, AAY85982.1, ACT91257.1, ACB11544.1, ACZ17532.1, ZP_{—}01746661.1, ABA55771.1, BAG06235.1, EGR32049.1, YP_{—}001166282.1, ABO61799.1, ABA55757.1, AEH76915.1, ACO59264.1, ABO26125.1, AEA08577.1, ACT91265.1, ABY56785.1, ACZ17528.1, ABO61798.1, ADO21749.1, ACT91263.1, ACT91252.1, ACZ64722.1, ABO61771.1, ACZ17526.1, ABO26123.1, ADO21714.1, ZP_{—}01000906.1, ABO61796.1, ADC29534.1, ACB15250.1, ACD47155.1, ACZ17525.1, ACB11553.1, ABD36391.1, AEH76913.1, ACZ17523.1, ABO61781.1, ACZ17524.1, ZP_{—}01914093.1, ACB11538.1, ZP_{—}01015838.1, ACJ22693.1, ACB15252.1, CAC86945.1, ACO59265.1, ABO61791.1, ACZ17521.1, ABO26124.1, ACZ64732.1, ACU43514.1, ACT91256.1, ACM63043.1, ACS75820.1, ZP_{—}08666479.1, CAH03133.1, BAG06234.1, AEH76916.1, ABO61790.1, ABE72965.1, ACZ64711.1, ACB11542.1, AAY26148.1, ABA55776.1, ACZ17522.1, ACZ64734.1, AEA08578.1, ACZ17530.1, ZP_{—}04062748.1, ACJ22755.1, NP_{—}969039.1, AAY26149.1, ACJ22761.1, ABU48543.1, ZP_{—}08414255.1, AAT91720.1, ZP_{—}01444283.1, ABA55796.1, ABU48542.1, YP_{—}001042010.1, YP_{—}001234392.1, YP_{—}351510.1, ACZ64730.1, ZP_{—}08634611.1, ACZ17529.1, ACJ22667.1, AAT91719.1, YP_{—}004283531.1, ABO61801.1, ACZ17519.1, ABO15266.1, CAB51040.1, ACZ64707.1, ACJ22766.1, ABO26121.1, ZP_{—}01878984.1, CAB51039.1, ABA55795.1, ABO15269.1, ABO15247.1, ACJ22763.1, ABO15251.1, ACZ17527.1, ABO15270.1, ACJ22769.1, ADE06670.1, ZP_{—}05780387.1, ABO61770.1, ACT91258.1, ABO15258.1, ABO15257.1, ABU48545.1, CAC86946.1, ABO15267.1, ZP_{—}01741446.1, ABU48544.1, YP_{—}002296646.1, AEH76917.1, ADC29550.1, YP_{—}002527219.1, ABK88246.1, ADN21388.1, ACT91210.1, ZP_{—}05064795.1, ABJ16487.1, XP_{—}002675644.1, ABJ16489.1, ADA71089.1, ADA71088.1, AAT46053.1, ZP_{—}01744806.1, ZP_{—}01037964.1, ZP_{—}00955262.1, ABJ 16493.1, YP_{—}001840157.1, ZP_{—}00964204.1, ABB40596.1, ACB15249.1, ADD82963.1, YP_{—}004499590.1, ZP_{—}01011524.1, ACJ22758.1, ZP_{—}01748906.1, ACV30052.1, ZP_{—}06191942.1, YP_{—}001188029.1, ACD63080.1, YP_{—}166583.1, AAV41375.1, ZP_{—}00998265.1, ACJ22757.1, ABB13506.2, ABI13999.1, ABI14004.1, ABB13509.1, YP_{—}371980.1, ZP_{—}01755711.1, ZP_{—}05065835.1, ZP_{—}00959368.1, XP_{—}001020063.1, ABJ16481.1, ABI14006.1, ZP_{—}05101918.1, ZP_{—}01913733.1, ABI14001.1, ABM92270.1, ABI14003.1, CAH03132.1, YP_{—}973211.1, ABA55797.1, YP_{—}003578527.1, ABJ16483.1, ABJ16482.1, CBY78068.1, ACT91260.1, YP_{—}509155.1, ABB13508.1, ABJ16485.1, ABO61779.1, ABI14005.1, ACM63042.1, ADC29543.1, ZP_{—}02153440.1, YP_{—}709335.1, ABI13998.1, ABI14002.1, AAB70825.1, ACX30751.1, ABI14000.1, YP_{—}003617173.1, ZP_{—}01155421.1, ACX30752.1, NP_{—}542887.1, ADC29546.1, AAC38359.1, ADC29541.1, XP_{—}001020064.1, ZP_{—}01442436.1, ZP_{—}05103090.1, ADC29544.1, ABO61809.1, AAY89939.1, ACH99235.1, CAH55830.1, ABO26095.1, YP_{—}004011670.1, ABO26084.1, ADA71083.1, ABO26087.1, ABO61806.1, ADC29531.1, ABO26109.1, ACJ22753.1, ABO26089.1, ABO26093.1, ABO26092.1, ABO61827.1, ABO26105.1, ABO26112.1, AAT91721.1, ABO26120.1, ABO26090.1, ABO26088.1, ABO61811.1, ABO61783.1, CAH55827.1, ACH99232.1, ABO61828.1, ADC29530.1, ACH99234.1, AAQ88276.1, CAH55823.1, ABO26103.1, ACH99233.1, ABO61836.1, ABO26094.1, ABO61840.1, YP_{—}004534277.1, ZP_{—}05845010.1, ABO61821.1, ACH99231.1, AAV68403.1, ABO61839.1, CAH56098.1, ABO26085.1, ABO61826.1, ABO61822.1, ABO26110.1, ABO61810.1, ABO61844.1, ABO61825.1, ABO26099.1, ACJ22767.1, ABO26102.1, YP_{—}004535707.1, ACJ22762.1, ABO26097.1, BAC65444.1, ABO61829.1, YP_{—}114083.1, CAH55828.1, ABO26106.1, YP_{—}552229.1, NP_{—}049190.1, ABO26116.1, CAH56107.1, CAM32407.1, ABO26101.1, ABO61841.1, ABM79805.1, ZP_{—}05075249.1, AAC27438.2, YP_{—}003754872.1, ADC29532.1, ADA71139.1, ADA71107.1, ADA71095.1, YP_{—}001268217.1, ADA71126.1, ADA71094.1, CAH56108.1, ADC29533.1, ADA71085.1, ZP_{—}05054453.1, ADA71097.1, ADA71086.1, ADA71114.1, ADC29548.1, ADA71101.1, ADC29547.1, ADA71138.1, ADC29542.1, ADA71098.1, ADA71128.1, ADA71105.1, ADA71093.1, ADA71135.1, ADA71100.1, YP_{—}557479.1, ADA71113.1, ADA71091.1, ADC29537.1, ADA71084.1, ADA71090.1, CAH56094.1, XP_{—}002945767.1, ADA71137.1, ADA71103.1, ADA71118.1, ADA71133.1, ADA71102.1, ADC29536.1, CAH56100.1, CAH56101.1, ACI15225.1, ACI15225.1, ABO26091.1, CAH55826.1, CAH55824.1, ZP_{—}08484419.1, ADA71111.1, ACJ22759.1, CAH55825.1, CAH56106.1, CAH56099.1, CAC40957.1, ZP_{—}05075037.1, CAH56102.1, ZP_{—}06846296.1, ABJ16491.1, ZP_{—}05067177.1, XP_{—}001698107.1, BAH10789.1, BAH10791.1, BAH10793.1, BAH10788.1, ABJ16490.1, BAH10800.1, BAH10790.1, BAH10792.1, ZP_{—}05075214.1, BAH10799.1, BAH10795.1, BAH10787.1, BAH10798.1, BAH10794.1, BAH10801.1, BAH10796.1, BAH10797.1, BAH10802.1, CAH56095.1, CAH56096.1, ADC29538.1, ABX76425.1, ZP_{—}06727686.1, ZP_{—}07774883.1, YP_{—}001615042.1,
in particular YP_{—}001185946.1, Q9WWW6.1, YP_{—}957898.1, YP_{—}957728.1, YP_{—}694427.1, BAC98365.1, ZP_{—}00957064.1, CAC86944.1, YP_{—}001672212.1, CAB59525.1, ACH99213.1, ACH99215.1, ACH99216.1, AAK56792.1, ACH99229.1, ACS91348.1, AAP41820.1
and especially preferably YP_{—}001185946.1,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{1b} is generally understood in particular as meaning the conversion of lauric acid and/or its methyl ester into w-hydroxylauric acid and/or its methyl ester.

### Specific Enzymes E_{c1}

Eukaryotic fatty alcohol oxidases E_{1c} which are preferred in this context are selected from among the list

AAS46878.1, ACX81419.1, AAS46879.1, CAB75353.1, AAS46880.1, XP_{—}712350.1, XP_{—}002422236.1, XP_{—}712386.1, EEQ43775.1, XP_{—}001525361.1, XP_{—}001386087.1, XP_{—}459506.2, CAB75351.1, CAB75352.1, XP_{—}001385255.2, EDK39369.2, XP_{—}001484086.1, XP_{—}002618046.1, XP_{—}002548766.1, XP_{—}002548765.1, XP_{—}003041566.1, XP_{—}003328562.1, XP_{—}001214264.1, XP_{—}001904377.1, XP_{—}658227.1, XP_{—}001591990.1, XP_{—}753079.1, XP_{—}002569337.1, XP_{—}001268562.1, XP_{—}003348911.1, EGP90120.1, XP_{—}001389382.1, EER37923.1, XP_{—}001264046.1, EGO58212.1, XP_{—}001554225.1, XP_{—}003298648.1, XP_{—}959005.1, XP_{—}002841296.1, XP_{—}001940486.1, EGR52262.1, EEQ89581.1, EGD99881.1, EFQ33355.1, XP_{—}001821106.1, XP_{—}002622231.1, EGG03784.1, EGC44059.1, XP_{—}003018036.1, XP_{—}003011696.1, EFY90752.1, XP_{—}001227812.1, XP_{—}758170.1, XP_{—}001243546.1, XP_{—}002479333.1, XP_{—}003344707.1, EFW 14100.1, XP_{—}003071927.1, XP_{—}003171263.1, XP_{—}003051757.1, XP_{—}002147053.1, EEH19591.1, EEH50473.1, XP_{—}001792978.1, XP_{—}387094.1, EFY98644.1, XP_{—}002788971.1, XP_{—}002842592.1, EFX04185.1, XP_{—}003231449.1, XP_{—}001729067.1, CBX94189.1, XP_{—}001413535.1, ACF22878.1, B5WWZ9.1, XP_{—}002994642.1, XP_{—}002269629.1, XP_{—}002519938.1, XP_{—}002982582.1, NP_{—}001047464.1, EEC73620.1, XP_{—}002981110.1, XP_{—}002960521.1, NP_{—}566729.1, XP_{—}001541970.1, XP_{—}002967201.1, BAK00483.1, XP_{—}002182547.1, BAK02336.1, XP_{—}002454190.1, XP_{—}002328753.1, XP_{—}002867943.1, XP_{—}002285334.1, CAC87643.1, CAN71289.1, XP_{—}002454188.1, AAL31049.1, XP_{—}002464494.1, AAL31021.1, YP_{—}117187.1, XP_{—}002543430.1, CAA18625.1, XP_{—}002883430.1, NP_{—}193673.2, XP_{—}002529832.1, XP_{—}001753124.1, NP_{—}001142399.1, ACN27562.1, XP_{—}002464495.1, ACR36691.1, BAJ86655.1, B5WWZ8.1, NP_{—}001148058.1, ABR17814.1, EAY78905.1, NP_{—}194586.1, AAM63097.1, AAK64154.1, NP_{—}001064839.2, XP_{—}002869492.1, XP_{—}002314488.1, AAL31024.1, ZP_{—}06967355.1, AAP54248.2, XP_{—}002311685.1, ACF87929.1, YP_{—}907078.1, EGE07035.1, YP_{—}001849908.1, XP_{—}002464496.1, EEC67160.1, AAL31027.1, XP_{—}001761391.1, XP_{—}002961172.1, XP_{—}002528823.1, XP_{—}002966834.1, NP_{—}001176205.1, XP_{—}001763007.1, XP_{—}002272123.1, XP_{—}002889487.1, XP_{—}003003157.1, NP_{—}285451.1, EGG23219.1, NP_{—}171895.2, YP_{—}003395677.1, Q9ZWB9.1, ACF88407.1, ZP_{—}06413771.1, EEE51131.1, YP_{—}003835264.1, YP_{—}003397164.1, YP_{—}004081922.1, XP_{—}003294587.1, EEE51130.1, YP_{—}003647529.1, YP_{—}003647985.1, CBI29206.3, XP_{—}629786.1, ZP_{—}07964664.1, EEE57396.1, EEH09589.1, YP_{—}003265796.1, YP_{—}001840752.1, ZP_{—}08620775.1, ACR36076.1, ZP_{—}05043749.1, YP_{—}980677.1, ZP_{—}05043728.1, YP_{—}692894.1, NP_{—}710223.1, EEC67159.1, AAP03110.1, EFA85697.1, YP_{—}691805.1, YP_{—}551012.1, YP_{—}001174466.1, YP_{—}002796294.1, YP_{—}004716331.1, YP_{—}001019547.1, YP_{—}585737.1, AEA86007.1, YP_{—}960830.1, YP_{—}004743970.1, ZP_{—}03431349.1, ZP_{—}06448642.1, ZP_{—}07430351.1, NP_{—}215006.2, ZP_{—}03535393.1, ZP_{—}06801690.1, YP_{—}001849132.1, NP_{—}854165.1, ZP_{—}03427234.1, CBJ27378.1, NP_{—}334920.1, ZP_{—}08571383.1, YP_{—}728161.1, ZP_{—}01896040.1, ZP_{—}03530923.1, YP_{—}551306.1, YP_{—}003167456.1, YP_{—}606070.1, ZP_{—}06850167.1, ADP99095.1, YP_{—}907986.1, ZP_{—}04924166.1, ZP_{—}08139923.1, YP_{—}001270300.1, YP_{—}521830.1, YP_{—}003147410.1, YP_{—}002007173.1, ADR62464.1, YP_{—}004382294.1, NP_{—}747223.1, YP_{—}004687462.1, NP_{—}902159.1, ZP_{—}04936784.1, YP_{—}003914667.1, ZP_{—}01306356.1, ZP_{—}04750553.1, YP_{—}002875279.1, YP_{—}004704374.1, YP_{—}001671392.1, NP_{—}249055.1, ZP_{—}06876360.1, YP_{—}001345853.1, YP_{—}002437969.1, YP_{—}004356853.1, YP_{—}351075.1, CBI23676.3, YP_{—}001189668.1, YP_{—}001528881.1, YP_{—}001613612.1, YP_{—}001747218.1, YP_{—}003393002.1, YP_{—}001365074.1, ZP_{—}07778129.1, ZP_{—}07392715.1, YP_{—}001553329.1, YP_{—}262925.1, YP_{—}751961.1, YP_{—}564183.1, YP_{—}003811876.1, YP_{—}002356821.1, YP_{—}001051828.1, YP_{—}001837525.1, NP_{—}716513.1, ZP_{—}01915079.1, ZP_{—}02156621.1, YP_{—}001184631.1, YP_{—}001475595.1, ZP_{—}05042393.1, YP_{—}962228.1, YP_{—}001612275.1, ADV55625.1, YP_{—}001675797.1, YP_{—}003555260.1, ZP_{—}01075039.1, YP_{—}003812822.1, YP_{—}001503351.1, EFN52938.1, YP_{—}001759063.1, ZP_{—}06503577.1, YP_{—}871025.1, ZP_{—}08564919.1, YP_{—}002310162.1, YP_{—}732875.1, YP_{—}001092722.1, YP_{—}739324.1, XP_{—}002333995.1, NP_{—}085596.1, YP_{—}928870.1, EGD05748.1, NP_{—}443993.1, ZP_{—}08138057.1, ZP_{—}05041587.1, ZP_{—}07011380.1, YP_{—}001612684.1, ZP_{—}07669342.1, ZP_{—}06508361.1, ZP_{—}03423639.1, YP_{—}923293.1, ZP_{—}05061865.1, ZP_{—}08181496.1, YP_{—}559605.1, ZP_{—}06841320.1, ZP_{—}01620712.1, YP_{—}001896340.1, ZP_{—}03276650.1, YP_{—}004303194.1, ZP_{—}08180715.1, ZP_{—}06382740.1, ZP_{—}01034555.1, YP_{—}004604560.1, YP_{—}001020142.1, YP_{—}935375.1, ZP_{—}01546137.1, ZP_{—}07661079.1, YP_{—}001860640.1, ZP_{—}06052841.1, ZP_{—}01881170.1, ZP_{—}05781455.1, YP_{—}932732.1, ZP_{—}08119300.1, YP_{—}004715268.1, ZP_{—}03697402.1, YP_{—}004126957.1, ZP_{—}06703136.1, NP_{—}642445.1, ZP_{—}08273900.1, YP_{—}004524313.1, ZP_{—}01902993.1, YP_{—}001900094.1, AEA84888.1, YP_{—}004690289.1, NP_{—}714358.1, YP_{—}682471.1, YP_{—}003239.1, YP_{—}997465.1, YP_{—}003452130.1, ZP_{—}01739153.1, YP_{—}004219483.1, YP_{—}001761298.1, ZP_{—}01438251.1, CBI37146.3, ZP_{—}04748383.1, YP_{—}004362245.1, ZP_{—}05912795.1, YP_{—}003390234.1, YP_{—}003122799.1, CCB77579.1, EGB06416.1, ZP_{—}08389346.1, YP_{—}191496.1, ZP_{—}05224727.1, ZP_{—}01125614.1, YP_{—}466287.1, YP_{—}001368620.1, YP_{—}001380256.1, YP_{—}002361951.1, YP_{—}002756103.1, YP_{—}001801399.1, ZP_{—}06847140.1, YP_{—}003200069.1, YP_{—}001940247.1, YP_{—}001584322.1, ZP_{—}04679227.1, YP_{—}002493674.1, YP_{—}002135530.1, YP_{—}004290424.1, YP_{—}001772011.1, ZP_{—}08189046.1, ZP_{—}03423640.1, YP_{—}001834251.1, ZP_{—}01041752.1, YP_{—}001533410.1, YP_{—}269751.1, YP_{—}002432994.1, YP_{—}003694653.1, CAD47896.1, NP_{—}769359.1, YP_{—}004239460.1, YP_{—}004605221.1, YP_{—}001961214.1, YP_{—}001837513.1, YP_{—}004335962.1, YP_{—}004358600.1, ZP_{—}05050026.1, YP_{—}003202983.1, BAD03777.1, ZP_{—}02165013.1, NP_{—}774131.1, YP_{—}432169.1, ZP_{—}05000547.1, YP_{—}001261233.1, XP_{—}002593969.1, XP_{—}002603265.1, YP_{—}003342435.1, ZP_{—}01253183.1, EGO36831.1, YP_{—}001866737.1, YP_{—}001523879.1, YP_{—}133594.1, YP_{—}003768990.1, YP_{—}001237820.1, YP_{—}003133224.1, ZP_{—}01896771.1, ZP_{—}01865125.1, NP_{—}960319.1, YP_{—}826958.1, YP_{—}003326608.1, YP_{—}002219515.1, NP_{—}217926.1, ZP_{—}07441899.2, YP_{—}001208178.1, ADM42038.1, YP_{—}002433510.1, ZP_{—}08274313.1, EGO38668.1, ZP_{—}03393221.1, NP_{—}356358.1, ZP_{—}06055780.1, YP_{—}001684562.1, ZP_{—}08528157.1, BAD03162.1, YP_{—}001800712.1, ACL37106.1, YP_{—}883489.1, ZP_{—}01075202.1, NP_{—}969446.1, ZP_{—}01129577.1, YP_{—}001530285.1, ZP_{—}04746501.1, YP_{—}001341980.1, YP_{—}905003.1, ZP_{—}05218299.1, ZP_{—}08665577.1,
preferably
AAS46878.1, ACX81419.1, AAS46879.1, CAB75353.1, AAS46880.1, XP_{—}712350.1, XP_{—}002422236.1, XP_{—}712386.1, EEQ43775.1, XP_{—}001525361.1, XP_{—}001386087.1, XP_{—}459506.2, CAB75351.1, CAB75352.1, XP_{—}001385255.2, EDK39369.2, XP_{—}001484086.1, XP_{—}002618046.1, XP_{—}002548766.1, XP_{—}002548765.1, XP_{—}003041566.1, XP_{—}001214264.1, XP_{—}001904377.1, XP_{—}658227.1, XP_{—}001591990.1, XP_{—}753079.1, XP_{—}002569337.1, XP_{—}001268562.1, XP_{—}003348911.1, EGP90120.1, XP_{—}001389382.1, EER37923.1, XP_{—}001264046.1, EGO58212.1, XP_{—}001554225.1, XP_{—}003298648.1, XP_{—}959005.1, XP_{—}002841296.1, XP_{—}001940486.1, EGR52262.1, EEQ89581.1, EGD99881.1, EFQ33355.1, XP_{—}001821106.1, XP_{—}002622231.1, EGC44059.1, XP_{—}003018036.1, XP_{—}003011696.1, EFY90752.1, XP_{—}001227812.1, XP_{—}001243546.1, XP_{—}002479333.1, XP_{—}003344707.1, EFW14100.1, XP_{—}003071927.1, XP_{—}003171263.1, XP_{—}003051757.1, XP_{—}002147053.1, EEH19591.1, EEH50473.1, XP_{—}001792978.1, XP_{—}387094.1, EFY98644.1, XP_{—}002788971.1, XP_{—}002842592.1, EFX04185.1, XP_{—}003231449.1, CBX94189.1, XP_{—}001413535.1, XP_{—}001541970.1, XP_{—}002543430.1, EGE07035.1, XP_{—}003003157.1
and especially preferably

### AAS46878.1, ACX81419.1, AAS46879.1, CAB75353.1, AAS46880.1, XP_{—}712350.1, XP_{—}002422236.1, XP_{—}712386.1, EEQ43775.1, CAB75351.1, CAB75352.1, XP_{—}002548766.1, XP_{—}002548765.1,

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{1c} is generally understood in particular as meaning the conversion of dodecan-1-ol into dodecan-1-al or dodecan-1-al into dodecanoic acid.

### Specific Enzymes E_{1d}

Such preferred AlkJ alcohol dehydrogenases are selected from among

Q00593.1, Q9WWW2.1, ZP_{—}00957061.1, YP_{—}957894.1, CAC38030.1, YP_{—}694430.1, YP_{—}957725.1, YP_{—}001672216.1, YP_{—}552061.1, YP_{—}130410.1, ZP_{—}06155535.1, ZP_{—}01222730.1, YP_{—}691907.1, YP_{—}002297804.1, YP_{—}004283522.1, YP_{—}001234383.1, YP_{—}004435031.1, ZP_{—}05110316.1, ZP_{—}05042898.1, YP_{—}004466324.1, ZP_{—}08553549.1, YP_{—}004125220.1, ADI22536.1, ADI18461.1, YP_{—}003810975.1, YP_{—}662346.1, YP_{—}004427557.1, YP_{—}692606.1, ZP_{—}05043291.1, YP_{—}440752.1, ZP_{—}02386160.1, ZP_{—}04763547.1, ZP_{—}02361232.1, YP_{—}003376674.1, ZP_{—}02354055.1, ZP_{—}05085930.1, ADQ00130.1, YP_{—}003643016.1, ZP_{—}05040520.1, YP_{—}691922.1, AAX23098.1, BAD07371.1, NP_{—}104379.1, YP_{—}002551960.1, YP_{—}003908558.1, YP_{—}987903.1, ZP_{—}05785860.1, YP_{—}004145612.1, YP_{—}004140926.1, CAZ88300.1, ZP_{—}05041901.1, YP_{—}533645.1, ZP_{—}01754259.1, CBA31223.1, YP_{—}587542.1, YP_{—}106852.1, ZP_{—}08402506.1, ZP_{—}05055020.1, ZP_{—}02400829.1, YP_{—}104747.1, ZP_{—}02409412.1, YP_{—}001057269.1, YP_{—}004229837.1, YP_{—}294429.1, YP_{—}001028112.1, ZP_{—}02479747.1, YP_{—}002874799.1, ZP_{—}03541051.1, YP_{—}003606536.1, ZP_{—}02887167.1, YP_{—}001795572.1, YP_{—}487451.1, ACZ62814.1, YP_{—}560809.1, ZP_{—}02167462.1, YP_{—}004482869.1, YP_{—}001581248.1, ZP_{—}07374066.1, YP_{—}001203981.1, ZP_{—}06840259.1, ZP_{—}01915145.1, NP_{—}774525.1, ZP_{—}03561080.1, YP_{—}001208258.1, YP_{—}001897374.1, YP_{—}001413909.1, YP_{—}366469.1, YP_{—}521854.1, YP_{—}004490642.1, YP_{—}003280349.1, ZP_{—}03588744.1, YP_{—}001562229.1, YP_{—}001120981.1, ZP_{—}03574970.1, YP_{—}004234225.1, ZP_{—}02377531.1, ZP_{—}02149954.1, YP_{—}001237360.1, ZP_{—}03266156.1, YP_{—}782821.1, YP_{—}004754039.1, BAB61732.1, ZP_{—}07046388.1, ZP_{—}02145452.1, BAF45123.1, YP_{—}002129953.1, YP_{—}003812439.1, ZP_{—}01055291.1, BAF45124.1, EGH71399.1, ZP_{—}05060389.1, ZP_{—}05090872.1, BAF45126.1, BAB07804.1, ZP_{—}06053464.1, YP_{—}001238278.1, ZP_{—}04944469.1, YP_{—}001171160.1, YP_{—}002984373.1, YP_{—}002237649.1, ZP_{—}08276443.1, BAF98451.1, ZP_{—}05124197.1, YP_{—}568640.1, ZP_{—}05785341.1, NP_{—}769037.1, YP_{—}370657.1, YP_{—}775005.1, ZP_{—}02911119.1, YP_{—}165460.1, ZP_{—}02891796.1, YP_{—}622328.1, ZP_{—}07675057.1, YP_{—}001901188.1, YP_{—}003592183.1, ZP_{—}02361040.1, NP_{—}518244.1, YP_{—}001809673.1, NP_{—}947032.1, YP_{—}001766369.1, YP_{—}002255997.1, ZP_{—}04940241.1, YP_{—}004012032.1, YP_{—}841049.1, YP_{—}002983249.1, YP_{—}003643276.1, YP_{—}003855487.1, YP_{—}003778137.1, ZP_{—}02361104.1, CBA30511.1, ZP_{—}05781295.1, YP_{—}756865.1, ZP_{—}02461782.1, YP_{—}002007988.1, YP_{—}004110133.1, YP_{—}002229680.1, ZP_{—}02386040.1, YP_{—}004684069.1, YP_{—}373268.1, YP_{—}440614.1, NP 421441.1, YP_{—}264896.1, YP_{—}004362617.1, ZP_{—}06053847.1, YP_{—}366538.1, YP_{—}003812285.1, YP_{—}004154520.1, ZP_{—}01901081.1, ZP_{—}02372179.1, ZP_{—}02453559.1, ADP98564.1, YP_{—}003747084.1, ZP_{—}02487888.1, ZP_{—}01768075.1, ZP_{—}02400664.1, YP_{—}106680.1, YP_{—}724753.1, YP_{—}002907583.1, YP_{—}004482470.1, YP_{—}167582.1, YP_{—}270109.1, YP_{—}004362333.1, ZP_{—}02504034.1, YP_{—}003189363.1, YP_{—}973212.1, ZP_{—}00952746.1, YP_{—}459665.1, YP_{—}777218.1, YP_{—}581107.1, ZP_{—}01878091.1, ZP_{—}01057973.1, YP_{—}002913124.1, ZP_{—}01035570.1, YP_{—}001777560.1, YP_{—}552627.1, ZP_{—}02890876.1, YP_{—}587146.1, YP_{—}004141814.1, YP_{—}001685369.1, ZP_{—}05343380.1, NP_{—}886000.1, ZP_{—}04942359.1, ZP_{—}01913732.1, ZP_{—}08244266.1, YP_{—}002233254.1, ZP_{—}01816670.1, YP_{—}837233.1, ZP_{—}07478008.1, ZP_{—}01985205.1, ZP_{—}07473972.1, ZP_{—}01067090.1, ZP_{—}01867788.1, ZP_{—}01754024.1, EGM19144.1, ZP_{—}07741283.1, ZP_{—}06876839.1, YP_{—}002395287.1, ZP_{—}07795498.1, NP_{—}102692.1, NP_{—}252789.1, YP_{—}004451100.1, ZP_{—}01305514.1, YP_{—}002438481.1, ZP_{—}04930310.1, YP_{—}001810189.1, YP_{—}104187.1, ZP_{—}01367534.1, YP_{—}001346382.1, ZP_{—}01878466.1, YP_{—}789017.1, YP_{—}001115422.1, ZP_{—}05067451.1, ZP_{—}05842072.1, YP_{—}001682976.1, YP_{—}761348.1, YP_{—}004611600.1, YP_{—}004188241.1, NP_{—}419761.1, EFV85163.1, YP_{—}684227.1, ZP_{—}06177455.1, NP_{—}935088.1, YP_{—}004614491.1, ZP_{—}08697916.1, YP_{—}004689366.1, ZP_{—}05052326.1, YP_{—}267420.1, YP_{—}728575.1, YP_{—}001759584.1, YP_{—}557446.1, ZP_{—}06844897.1, ZP_{—}06079799.1, YP_{—}003771143.1, ZP_{—}05094472.1, YP_{—}511622.1, ACF98205.1, YP_{—}582314.1, ZP_{—}07660450.1, YP_{—}004065269.1, YP_{—}003979606.1, YP_{—}002520401.1, YP_{—}003579281.1, ZP_{—}01749397.1, ZP_{—}03265018.1, ZP_{—}07283393.1, YP_{—}001532150.1, YP_{—}298941.1, ZP_{—}06688181.1, ZP_{—}01611660.1, ZP_{—}02367747.1, EGP42870.1, ZP_{—}00993245.1, ABY65992.1, YP_{—}354800.1, ZP_{—}01747277.1, YP_{—}561728.1, ZP_{—}02190947.1, YP_{—}605824.1, YP_{—}001991873.1, ZP_{—}00955792.1, YP_{—}003594401.1, YP_{—}004156101.1, YP_{—}001472858.1, YP_{—}001746950.1, ZP_{—}08410042.1, ZP_{—}01116604.1, ADP99912.1, ZP_{—}01692203.1, YP_{—}001328534.1, YP_{—}999236.1, YP_{—}002278452.1, ZP_{—}01306234.1, YP_{—}002871776.1, ZP_{—}02369920.1, ZP_{—}01896942.1, YP_{—}002289724.1, AEG07584.1, YP_{—}999005.1, YP_{—}003552461.1, YP_{—}270668.1, ZP_{—}06862917.1, YP_{—}001811327.1, YP_{—}001166036.1, ABW06653.1, ZP_{—}01548976.1, ZP_{—}07774606.1, ZP_{—}05888080.1, YP_{—}003301477.1, YP_{—}341748.1, ZP_{—}05100248.1, YP_{—}918038.1, YP_{—}001500869.1, YP_{—}004305296.1, YP_{—}003342584.1, NP_{—}947961.1, ZP_{—}05124765.1, ZP_{—}01904700.1, YP_{—}003696207.1, YP_{—}004156699.1, YP_{—}001241858.1, NP_{—}104253.1, YP_{—}676241.1, ZP_{—}01736903.1, ZP_{—}00960121.1, NP_{—}436019.1, YP_{—}002945716.1, YP_{—}259594.1, EFV86615.1, AAY87334.1, NP_{—}900970.1, AEG07409.1, YP_{—}349087.1, YP_{—}004141055.1, YP_{—}001169476.1, YP_{—}001566960.1, YP_{—}260472.1, ZP_{—}07028078.1, YP_{—}004610468.1, YP_{—}003066461.1, YP_{—}961096.1, ZP_{—}08666573.1, ZP_{—}02187363.1, YP_{—}001631518.1, ZP_{—}08141293.1, YP_{—}001666324.1, NP_{—}387083.1, YP_{—}001526184.1, YP_{—}165213.1, YP_{—}003694923.1, YP_{—}004433897.1, YP_{—}001265431.1, ZP_{—}05068964.1, YP_{—}002313077.1, ZP_{—}02372305.1, YP_{—}004486039.1, YP_{—}341901.1, YP_{—}001862312.1, YP_{—}004681983.1, YP_{—}617373.1, EFV86570.1, YP_{—}001673285.1, BAK39604.1, YP_{—}001669327.1, YP_{—}004353150.1, YP_{—}001888124.1, ZP_{—}08645365.1, YP_{—}003410784.1, YP_{—}841363.1, EGP44033.1, YP_{—}001633470.1, EGP42855.1, ZP_{—}01115125.1, ADR57794.1, YP_{—}784649.1, YP_{—}373898.1, Q47944.1, YP_{—}001117950.1, ZP_{—}02380339.1, ZP_{—}03697092.1, YP_{—}003187112.1, YP_{—}004065439.1, NP_{—}742226.1, YP_{—}002429878.1, YP_{—}003556403.1, AEH81535.1, YP_{—}001887935.1, YP_{—}554605.1, ZP_{—}07333059.1, YP_{—}001991668.1, YP_{—}003694210.1, YP_{—}222680.1, YP_{—}002232672.1, YP_{—}001763402.1, YP_{—}001806802.1, YP_{—}662156.1, ZP_{—}05153429.1, ZP_{—}01893457.1, ZP_{—}04595387.1, ADP99389.1, ZP_{—}02890074.1, YP_{—}001313582.1, NP_{—}387401.1, ZP_{—}01863693.1, YP_{—}750630.1, ZP_{—}04939997.1, YP_{—}268077.1, ZP_{—}05169265.1, NP_{—}888994.1, ZP_{—}08408421.1, YP_{—}001155137.1, NP_{—}699017.1, YP_{—}002008190.1, YP_{—}004493716.1, YP_{—}266277.1, YP_{—}004654190.1, YP_{—}943422.1, ZP_{—}05162503.1, ZP_{—}02905080.1, ZP_{—}02905080.1, ZP_{—}03784461.1, YP_{—}001601784.1, YP_{—}002233786.1, YP_{—}622842.1, YP_{—}002822679.1, ZP_{—}04944312.1, ZP_{—}05179897.1, YP_{—}004483124.1, YP_{—}003390414.1, YP_{—}771968.1, YP_{—}001628465.1, YP_{—}004311599.1, ZP_{—}01037150.1, ZP_{—}01611812.1, ZP_{—}03575238.1, YP_{—}002278603.1, YP_{—}001593845.1, EGD01613.1, YP_{—}297574.1, YP_{—}367509.1, YP_{—}998315.1, ZP_{—}08664883.1, ZP_{—}05114787.1, ZP_{—}05450190.1, YP_{—}298028.1, ZP_{—}01034678.1, YP_{—}002827796.1, YP_{—}372762.1, YP_{—}004466723.1, ZP_{—}01012072.1, YP_{—}320380.1, ZP_{—}01075202.1, YP_{—}001312358.1, YP_{—}681895.1, ZP_{—}07718189.1, EGP55868.1, YP_{—}003750799.1, YP_{—}002984725.1, YP_{—}002543360.1, ZP_{—}01040714.1, ZP_{—}04717111.1, YP_{—}002422932.1, YP_{—}003506115.1, ZP_{—}01444019.1, ZP_{—}03587285.1, YP_{—}771439.1, YP_{—}001947593.1, YP_{—}001049712.1, YP_{—}003979888.1, YP_{—}001553786.1, YP_{—}003980878.1, YP_{—}001578274.1, YP_{—}472442.1, YP_{—}778292.1, EGE56670.1, YP_{—}002779312.1, YP_{—}432169.1, YP_{—}560963.1, YP_{—}001265285.1, YP_{—}002822699.1, YP_{—}002278091.1, ZP_{—}08632361.1, YP_{—}002229178.1, ZP_{—}06840392.1, ZP_{—}05069105.1, ZP_{—}00998644.1, YP_{—}004487901.1, YP_{—}680905.1, YP_{—}728088.1, YP_{—}001985833.1, YP_{—}002007099.1, ZP_{—}05066777.1, ZP_{—}01551182.1, YP_{—}002973332.1, ZP_{—}04681414.1, ZP_{—}07675148.1, AEH83964.1, YP_{—}004692042.1, CBJ36337.1, EGP48473.1, ZP_{—}03585612.1, YP_{—}001369428.1, YP_{—}001897527.1, AEG08472.1, YP_{—}001166065.1, NP_{—}437018.1, NP_{—}294689.1, YP_{—}002541437.1, YP_{—}004692953.1, NP_{—}107484.1, YP_{—}995681.1, YP_{—}765267.1, YP_{—}166223.1, ZP_{—}01740635.1, YP_{—}001234127.1, ZP_{—}02186681.1, YP_{—}004140839.1, YP_{—}001584499.1, ADI17244.1, ZP_{—}08698744.1, YP_{—}001022991.1, EFV84582.1, ZP_{—}01743515.1, YP_{—}001816113.1, YP_{—}004688050.1, YP_{—}001342912.1, ZP_{—}01125614.1, EGD05029.1, ZP_{—}03569823.1, ZP_{—}05089337.1, YP_{—}001901091.1, NP_{—}886663.1, ZP_{—}07718907.1, YP_{—}004687387.1, NP_{—}521464.1, ZP_{—}06688394.1, ZP_{—}08099738.1, ZP_{—}02885452.1, YP_{—}003744085.1, YP_{—}001328823.1, ZP_{—}02488044.1, ZP_{—}01015005.1, YP_{—}002983153.1, ZP_{—}06898725.1, ZP_{—}05886707.1, ZP_{—}08101209.1, ZP_{—}03319462.1, YP_{—}003134969.1, YP_{—}001188857.1, YP_{—}004557767.1, YP_{—}004675666.1, YP_{—}004358728.1, YP_{—}002252541.1, YP_{—}684009.1, ZP_{—}05085667.1, ZP_{—}02144674.1, YP_{—}004127560.1, ZP_{—}01901604.1, YP_{—}004280074.1, AEG67402.1, YP_{—}001416516.1, ZP_{—}01054720.1, ZP_{—}08197897.1, NP_{—}107235.1, YP_{—}002909966.1, ZP_{—}01545876.1, ZP_{—}02147729.1, ZP_{—}00946537.1, ZP_{—}01903844.1, ZP_{—}05085589.1, ACV84069.1, YP_{—}367172.1, ZP_{—}02165272.1, YP_{—}701696.1, ZP_{—}04935724.1, ZP_{—}02191362.1, ZP_{—}01740154.1, ZP_{—}07662819.1, NP_{—}103908.1, YP_{—}003159313.1, YP_{—}003197010.1, ZP_{—}02152342.1, YP_{—}001907189.1, YP_{—}004387414.1, YP_{—}001413869.1, ZP_{—}01916549.1, ZP_{—}03264661.1, AAY82840.1, YP_{—}003277969.1, YP_{—}767433.1, ZP_{—}01226234.1, EGE55950.1, NP_{—}882474.1, ZP_{—}04680938.1, YP_{—}004417965.1, ZP_{—}01367142.1, EGM13684.1, YP_{—}001262083.1, ZP_{—}01881606.1, ZP_{—}01002680.1, YP_{—}003606679.1, YP_{—}001868359.1, ZP_{—}01446736.1, YP_{—}004141411.1, YP_{—}002438878.1, YP_{—}002500414.1, EGP55675.1, ZP_{—}08405873.1, YP_{—}002975318.1, YP_{—}002823637.1, ZP_{—}02188786.1, YP_{—}004617386.1, ABL61001.1, YP_{—}004190679.1, YP_{—}004418710.1, YP_{—}001264994.1, NP_{—}252399.1, ACA21517.1, YP_{—}002541208.1, YP_{—}001369943.1, YP_{—}789454.1, YP_{—}004688060.1, YP_{—}611623.1, ZP_{—}07795086.1, ZP_{—}04929943.1, YP_{—}004444316.1, ZP_{—}01866687.1, ZP_{—}05973466.1, YP_{—}004353327.1, ZP_{—}05780591.1, ZP_{—}05784784.1, NP_{—}936564.1, ZP_{—}05739211.1, ZP_{—}05113045.1, ZP_{—}06689273.1, ZP_{—}06972168.1, ZP_{—}01616404.1, ZP_{—}07659253.1, ZP_{—}05117914.1, YP_{—}585662.1, YP_{—}004230016.1, NP_{—}763554.1, NP_{—}744101.1, ZP_{—}02465308.1, ACN56476.1, YP_{—}004689565.1, YP_{—}001600608.1, ZP_{—}06792595.1, YP_{—}001258553.1, ZP_{—}05165722.1, ZP_{—}03785098.1, YP_{—}002276744.1, YP_{—}002524856.1, ADP98420.1, YP_{—}001669248.1, ZP_{—}04764988.1, ZP_{—}08528163.1, ZP_{—}08529409.1, ZP_{—}05944625.1, YP_{—}676267.1, CBA26630.1, YP_{—}001592413.1, YP_{—}003486465.1, ZP_{—}02187562.1, ZP_{—}03702891.1, YP_{—}760283.1, ZP_{—}05450850.1, YP_{—}004533595.1, ZP_{—}02153313.1, YP_{—}001859265.1, YP_{—}001524099.1, ZP_{—}06126913.1, ZP_{—}07374926.1, ZP_{—}05050787.1, ZP_{—}01035411.1, Q8YFY2.2, YP_{—}002280903.1, EGM21512.1, YP_{—}004603010.1, ZP_{—}05088581.1, YP_{—}004302488.1, YP_{—}004141219.1, NP_{—}697569.1, YP_{—}003908705.1, YP_{—}915505.1, YP_{—}001789228.1, YP_{—}001042739.1, YP_{—}133405.1, ZP_{—}05180516.1, ZP_{—}05174702.1, ZP_{—}01438051.1, ZP_{—}04590345.1, ZP_{—}08411937.1, NP_{—}356519.2, ZP_{—}00964019.1, ZP_{—}00998343.1, ZP_{—}05181994.1, YP_{—}004107969.1, ZP_{—}02168070.1, ZP_{—}01750865.1, YP_{—}574504.1, YP_{—}004579902.1, YP_{—}104440.1, ZP_{—}05452167.1, ZP_{—}05342702.1, YP_{—}001862883.1, YP_{—}004538242.1, ZP_{—}07471513.1, ZP_{—}05169558.1, ZP_{—}00956995.1, ZP_{—}05096699.1, YP_{—}004610916.1, ZP_{—}01218118.1, AAU95210.1, ZP_{—}02405087.1, ZP_{—}04890639.1, YP_{—}352237.1, ZP_{—}02413594.1, ZP_{—}07474023.1, NP_{—}541317.1, YP_{—}001993222.1, ZP_{—}08199001.1, YP_{—}471839.1, ZP_{—}02492080.1, ZP_{—}04901176.1, ZP_{—}06915396.1, ZP_{—}07474845.1, ZP_{—}07477743.1, YP_{—}004152647.1, YP_{—}004755056.1, ZP_{—}05086419.1, YP_{—}004577547.1, ACD99850.1, YP_{—}980426.1, ZP_{—}05457072.1, ZP_{—}05936041.1, NP_{—}700124.1, ADT85599.1, YP_{—}110012.1, ZP_{—}05076113.1, YP_{—}001068288.1, ZP_{—}02457871.1, ZP_{—}01014169.1, EGE60620.1, YP_{—}001346810.1, YP_{—}003408795.1, YP_{—}003769675.1, YP_{—}001257876.1, EGH93583.1, ZP_{—}01442222.1, YP_{—}331617.1, ZP_{—}05636703.1, YP_{—}001594896.1, YP_{—}002822967.1, YP_{—}118823.1, ZP_{—}01878717.1, ZP_{—}07375284.1, YP_{—}001371250.1, ZP_{—}07658682.1, YP_{—}002898825.1, ZP_{—}01547199.1, YP_{—}223070.1, ZP_{—}05161482.1, ZP_{—}04679742.1, YP_{—}002778618.1, ZP_{—}01626756.1, ZP_{—}05101564.1, YP_{—}002947374.1, NP_{—}385053.1, YP_{—}001328117.1, YP_{—}004493948.1, YP_{—}003339515.1, YP_{—}004699488.1, ZP_{—}05101969.1, YP_{—}485352.1, ZP_{—}01746033.1, ZP_{—}06712293.1, ZP_{—}01158125.1, ZP_{—}01058616.1, ZP_{—}05739755.1, NP_{—}949067.1, ZP_{—}02364657.1, YP_{—}570690.1, YP_{—}001208663.1, ZP_{—}02357557.1, ZP_{—}04751682.1, YP_{—}001326253.1, YP_{—}487666.1, ZP_{—}05167919.1, ADI18237.1, YP_{—}002825245.1, ZP_{—}02144858.1, ZP_{—}02188790.1, ZP_{—}06794586.1, YP_{—}001809828.1, YP_{—}997974.1, YP_{—}001476791.1, ZP_{—}08635286.1, YP_{—}676287.1, ZP_{—}07308228.1, ZP_{—}04596242.1, YP_{—}001622726.1, NP_{—}699590.1, ZP_{—}01446884.1, YP_{—}001168504.1, ZP_{—}01616388.1, ZP_{—}05117189.1, ZP_{—}05876432.1, ADT64694.1, ZP_{—}01754911.1, ZP_{—}05880498.1, ZP_{—}02360829.1, ZP_{—}06052433.1, ZP_{—}08663540.1, YP_{—}003768966.1, ZP_{—}02165422.1, ZP_{—}00960985.1, ZP_{—}07026655.1, YP_{—}001753039.1, YP_{—}371288.1, YP_{—}002974725.1, YP_{—}776880.1, ZP_{—}05784963.1, ZP_{—}05124380.1, YP_{—}459030.1, ZP_{—}05090690.1, ZP_{—}05064893.1, ZP_{—}02367982.1, ZP_{—}01890564.1, NP 541848.1, ZP_{—}00960263.1, ZP_{—}02961617.1, YP_{—}001242097.1, ZP_{—}05838258.1,
in particular
000593.1, Q9WWW2.1, ZP_{—}00957061.1, YP_{—}957894.1, CAC38030.1, YP_{—}694430.1, YP_{—}957725.1, YP_{—}001672216.1, YP_{—}552061.1, YP_{—}130410.1, ZP_{—}06155535.1, ZP_{—}01222730.1, YP_{—}691907.1, YP_{—}002297804.1, YP_{—}004283522.1, YP_{—}001234383.1, YP_{—}004435031.1, ZP_{—}05110316.1, ZP_{—}05042898.1, YP_{—}004466324.1, ZP_{—}08553549.1, YP_{—}004125220.1, ADI22536.1, ADI18461.1, YP_{—}003810975.1, YP_{—}662346.1, YP_{—}004427557.1, YP_{—}692606.1, ZP_{—}05043291.1, YP_{—}440752.1, ZP_{—}02386160.1, ZP_{—}04763547.1, ZP_{—}02361232.1, YP_{—}003376674.1, ZP_{—}02354055.1, ZP_{—}05085930.1, ADQ00130.1, YP_{—}003643016.1, ZP_{—}05040520.1, YP_{—}691922.1, AAX23098.1, BAD07371.1, NP_{—}104379.1, YP_{—}002551960.1, YP_{—}003908558.1, YP_{—}987903.1, ZP_{—}05785860.1, YP_{—}004145612.1, YP_{—}004140926.1, CAZ88300.1,
and especially preferably

### Q00593.1, Q9WWW2.1, ZP_{—}00957061.1, YP_{—}957894.1, CAC38030.1, YP_{—}694430.1, YP_{—}957725.1, YP_{—}001672216.1.

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{1d} is generally understood in particular as meaning the conversion of dodecan-1-ol into dodecan-1-al or dodecan-1-al into dodecanoic acid.

### Specific Enzymes E₁ₑ

Such preferred alcohol dehydrogenases of EC 1.1.1.1 or EC 1.1.1.2 are selected from among

AdhE, AdhP, YjgB, YqhD, GIdA, EutG, YiaY, AdhE, AdhP, YhhX, YahK, HdhA, HisD, SerA, Tdh, Ugd, Udg, Gmd, YefA, YbiC, YdfG, YeaU, TtuC, YeiQ, YgbJ, YgcU, YgcT, YgcV, YggP, YgjR, YliI, YqiB, YzzH, LdhA, GapA, Epd, Dld, GatD, Gcd, GlpA, GlpB, GlpC, GlpD, GpsA and YphC from bacteria, in particular E. coli,
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E₁ₑ is generally understood in particular as meaning the conversion of dodecan-1-al into dodecanoic acid.

### Specific Enzymes E_{1f}

Such preferred aldehyde dehydrogenases are selected from among Prr, Usg, MhpF, AstD, GdhA, FrmA, Feab, Asd, Sad, PuuE, GabT, YgaW, BetB, PutA, PuuC, FeaB, AldA, Prr, EutA, GabD, AIdB, TynA and YneI from bacteria, in particular E. coli,

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{1f} is generally understood in particular as meaning the conversion of dodecan-1-al into dodecanoic acid.

### Auxiliary Enzymes for E₁ₐ

It is preferred according to the invention that, when the activity of an enzyme E₁ₐ, a eukaryotic P450 alkane hydroxylase, is reduced, the microorganism according to the invention also has an activity of an NADPH-cytochrome P450 oxidoreductase of EC 1.6.2.4 which is reduced in comparison with its wild type. This has the technical effect of the activity of the eukaryotic P450 alkane hydroxylases is reduced further and the product yields of alkan-1-ols, alkan-1-als, alkan-1-amines, alkanes and terminal olefins are increased.

NADPH-cytochrome P450 oxidoreductases of EC 1.6.2.4 catalyse the following reaction: oxidized cytochrome P450+NADPH⁺=reduced cytochrome P450+NADP⁺+H⁺

It is preferred according to the invention that, when the activity of an enzyme E₁ₐ, a prokaryotic P450 alkane hydroxylase, is reduced, the microorganism according to the invention also has an activity of a ferredoxin-NAD(P)⁺ reductase of EC 1.18.1.2 or EC 1.18.1.3 and/or of a ferredoxin which is reduced in comparison with its wild type. This has the technical effect that the activity of the prokaryotic P450 alkane hydroxylase of the CYP_{—}153 type is reduced further and that the product yields of alkan-1-ols, alkan-1-als, alkan-1-amines, alkanes and terminal olefins are increased.

Ferredoxin-NAD(P)⁺ reductases of EC 1.18.1.2 or EC 1.18.1.3 catalyse the following reaction: oxidized ferredoxin+NAD(P)H+H⁺=reduced ferredoxin+NAD(P)⁺ and are preferably encoded by a gene which is located in the immediate vicinity of a gene of an abovementioned prokaryotic P450 alkane hydroxylase of the CYP_{—}153 type or of a ferredoxin described in the context of the present invention.

The expression “in the immediate vicinity” means that not more than three other structural genes are located between the genes in question.

Ferredoxins catalyse the following reactions:

alkane hydroxylase+reduced ferredoxin+alkanoic acid (ester)=alkane monoxygenase+oxidized ferredoxin+ω-hydroxyalkanoic acid (ester)+H₂O,
alkane hydroxylase+2 reduced ferredoxins+alkanoic acid (ester)=alkane hydroxylase+2 oxidized ferredoxins+ω-oxoalkanoic acid (ester)+2H₂O or
alkane hydroxylase+3 reduced ferredoxins+alkanoic acid (ester)=alkane hydroxylase+3 oxidized ferredoxins+ω-carboxyalkanoic acid (ester)+3H₂O and
are preferably encoded by a gene which is located in the immediate vicinity of a gene of an abovementioned prokaryotic P450 alkane hydroxylase of the CYP_{—}153 type or of an abovementioned ferredoxin-NAD(P)⁺ reductase of EC 1.18.1.2 or EC 1.18.1.3. The expression “in the immediate vicinity” means that not more than three other structural genes are located between the genes in question.

Preferred microorganisms have an activity of the ferredoxin-NAD(P)⁺ reductase AlkT and of a ferredoxin which is increased in comparison with their wild type.

### Auxiliary Enzymes for E_{1b}

It is preferred according to the invention that, when the activity of an enzyme E_{1b}, an AlkB alkane hydroxylase of EC 1.14.15.3, is reduced, the microorganism according to the invention likewise has an activity of an AlkT rubredoxin-NAD(P)⁺ reductase of EC 1.18.1.1 or of EC 1.18.1.4 and/or of a rubredoxin AlkG which is increased in comparison with its wild type. This has the technical effect that the activity of the AlkB alkane hydroxylase is enhanced and the product yields are increased.

AlkT rubredoxin-NAD(P)⁺ reductases of EC 1.18.1.1 or EC 1.18.1.4 catalyse the following reaction:

oxidized rubredoxin+NAD(P)H+H⁺=reduced rubredoxin+NAD(P)⁺ and are preferably encoded by a gene which is located in the immediate vicinity of a gene of an abovementioned AlkB alkane hydroxylase of EC 1.14.15.3 or of a rubredoxin AlkG described in the context of the present invention.

The expression “in the immediate vicinity” means that not more than three other structural genes are located between the genes in question.

Rubredoxins AlkG catalyse the following reactions:

alkane monoxygenase+reduced rubredoxin+alkanoic acid (ester)=alkane monoxygenase+oxidized rubredoxin+ω-hydroxyalkanoic acid (ester)+H₂O, alkane monoxygenase+2 reduced rubredoxins+alkanoic acid (ester)=alkane monoxygenase+2 oxidized rubredoxins+ω-oxoalkanoic acid (ester)+2H₂O or
alkane monoxygenase+3 reduced rubredoxins+alkanoic acid (ester)=alkane monoxygenase+3 oxidized rubredoxins+ω-carboxyalkanoic acid (ester)+3H₂O and
are preferably encoded by a gene which is located in the immediate vicinity of a gene of an abovementioned AlkB alkane hydroxylase of EC 1.14.15.3 or of an abovementioned AlkT rubredoxin-NAD(P)⁺ reductase of EC 1.18.1.1 or EC 1.18.1.4. The expression “in the immediate vicinity” means that not more than three other structural genes are located between the genes in question.

Preferred microorganisms have an activity of the AlkT rubredoxin-NAD(P)⁺ reductase and of rubredoxin AlkG which is reduced in comparison to their wild type.

### Specific Embodiments of Preferred Microorganisms and Enzymes

According to the invention, microorganisms are especially preferably selected from among those which include

a first and a second genetic modification within the meaning of the invention,
a first, a second and a fifth genetic modification within the meaning of the invention,
a first, a second and a sixth genetic modification within the meaning of the invention,
a first, a second and a seventh genetic modification within the meaning of the invention,
a first, a second, a fifth and a sixth genetic modification within the meaning of the invention,
a first, a second, a fifth and a seventh genetic modification within the meaning of the invention,
a first, a second, a sixth and a seventh genetic modification within the meaning of the invention,
a first, a second, a fifth, a sixth and a seventh genetic modification within the meaning of the invention,
a first, a second and a third genetic modification within the meaning of the invention,
a first, a second, a third and a fifth genetic modification within the meaning of the invention,
a first, a second, a third and a sixth genetic modification within the meaning of the invention,
a first, a second, a third and a seventh genetic modification within the meaning of the invention,
a first, a second, a third, a fifth and a sixth genetic modification within the meaning of the invention,
a first, a second, a third, a fifth and a seventh genetic modification within the meaning of the invention,
a first, a second, a third, a sixth and a seventh genetic modification within the meaning of the invention,
a first, a second, a third, a fifth, a sixth and a seventh genetic modification within the meaning of the invention,
a first, a second and a fourth genetic modification within the meaning of the invention,
a first, a second, a fourth and a fifth genetic modification within the meaning of the invention,
a first, a second, a fourth and a sixth genetic modification within the meaning of the invention,
a first, a second, a fourth and a seventh genetic modification within the meaning of the invention,
a first, a second, a fourth, a fifth and a sixth genetic modification within the meaning of the invention,
a first, a second, a fourth, a fifth and a seventh genetic modification within the meaning of the invention,
a first, a second, a fourth, a sixth and a seventh genetic modification within the meaning of the invention or
a first, a second, a fourth, a fifth, a sixth and a seventh genetic modification within the meaning of the invention.

Microorganisms which are especially preferred according to the invention are those which include a first genetic modification so that they are capable of forming more carboxylic acids and carboxylic acid derivatives from at least one simple carbon source in comparison with their wild type, where the first genetic modification represents an activity of at least one of the enzymes Eᵢ or one of the enzymes with a polypeptide sequence in which up to 60%, preferably up to 25%, especially preferably up to 15%, in particular up to 10, 9, 8, 7, 6, 5, 4, 3, 2, 1% amino acid residues are modified by deletion, insertion, substitution or a combination thereof over the sequences specified by references in the table hereinbelow and which still have at least 50%, preferably 65%, especially preferably 80%, in particular more than 90% of the activity of the protein with the respective reference sequence, which activity is increased in comparison with the enzymatic activity of the wild type of the microorganism and where activity in this context and in the context of the determination of the activity of the enzyme Eᵢ generally means in particular the hydrolysis of dodecanoyl-ACP thioester with the carbon chain length assigned to the individual enzymes Eᵢ in the table hereinbelow

and the carboxylic acid and carboxylic acid derivatives have a carbon chain length of the carboxylic acid moiety as represented in the table hereinbelow:

| Enzyme Eᵢ selected from among | Carbon chain length |
|---|---|
| AAC49269.1, CAB60830.1, AAC49179.1, | C8 |
| AAC49784.1, ABB71579.1, CAC19934.1 | |
| and SEQ ID No.: 26, 29, 33, 38, 40, 97 and | |
| 99 of WO2011008565 | |
| AAC49269.1, CAB60830.1, AAC49179.1, | C10 |
| AAC49784.1, ABB71579.1, CAC19934.1 | |
| and SEQ ID No.: 73, 75, 87 and 89 of | |
| WO2011008565. | |
| Q41635.1, Q39473.1, AAC49180.1, | C12 |
| CAC19934.1, AAC72881.1, AAC49783.1, | |
| AAC49784.1 and SEQ ID No.: 49 and 51 of | |
| WO2011008565. | |
| Q41635.1, Q39473.1, AAC49180.1, | C14 |
| CAC19934.1, AAC72881.1 AAC49783.1, | |
| AAC49784.1 and SEQ ID No.: 49, 51, 53, | |
| 55, 61, 63, 67, 69, 77, 79, 83 and 85 of | |
| WO2011008565. | |

The abovementioned deletions of amino acid residues over the sequences specified by references in the table hereinabove refer in particular to deletions at the N- and/or C-terminus, in particular the N-terminus. The abovementioned N-terminus is especially preferably that of a plant plastid targeting sequence. Such plant plastid targeting sequences can be predicted for example with the aid of the algorithms employed by the prediction tool TargetP 1.1 (www.cbs.dtu.dk/services/TargetP/) and described in the following publications, preferably without using cutoffs:

Predicting Subcellular Localization of Proteins Based on their N-Terminal Amino Acid Sequence.

Olof Emanuelsson, Henrik Nielsen, Søren Brunak and Gunnar von Heijne.

J. Mol. Biol., 300: 1005-1016, 2000 and Identification of prokaryotic and eukaryotic signal peptides and prediction of their cleavage sites. Henrik Nielsen, Jacob Engelbrecht, Soren Brunak and Gunnar von Heijne. Protein Engineering, 10:1-6, 1997.

Microorganisms (abbreviated to MO) which are very especially preferred according to the invention are outstandingly suitable for the production of carboxylic acids and have increased or reduced enzymatic activities (abbreviated to E) which are described in the table hereinbelow, it being possible for these enzymatic activities additionally to be advantageously combined with an enzymatic activity described for the enzyme 3-ketoacyl-ACP (acyl carrier protein) synthase III (EC 2.3.1.41), which enzymatic activity is increased in comparison with the wild type of the microorganism, in particular the enzymatic activity of plants, preferably the enzymatic activity of plants whose seeds contain fatty acids with alkyl residues shorter than 14 C atoms and especially preferably the enzymatic activity of plants of the genera Cuphea, Elaeis, Cocos, Umbellularia and Cinnamomum and gene products selected from among AccA, AccB, AccC, AccD, AceE, AceF, Lpd, AcpP, FabA, FabB, FabD, FabF, FabG, FabH, FabI, FabZ, PanD, PanK, UdhA, PntA or PntB.

Any desired combinations of at least two of these enzymatic activities may advantageously be increased.

It may be additionally advantageous that the microorganism is equipped with an enzymatic activity which is described for the gene products selected from among TdcE, PflA, PflB, PflC, PfID, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, individually or in any desired combination, and which is reduced in comparison with the wild type of the microorganism.

| | | |
|---|---|---|
| MO | Increased E | Reduced E |

| | | | |
|---|---|---|---|
| 1 | AlkL | | |
| 2 | AlkLEᵢ | | |
| 3 | AlkLEᵢᵢ | | |
| 4 | AlkLEᵢᵢE_{iib} | | |
| 5 | AlkLEᵢᵢᵢ | | |
| 6 | AlkLEᵢᵥ | | |
| 7 | AlkL | Eₐ | |
| 8 | AlkLEᵢ | Eₐ | |
| 9 | AlkLEᵢᵢ | Eₐ | |
| 10 | AlkLEᵢᵢE_{iib} | Eₐ | |
| 11 | AlkLEᵢᵢᵢ | Eₐ | |
| 12 | AlkLEᵢᵥ | Eₐ | |
| 13 | AlkL | E_{b} | |
| 14 | AlkLEᵢ | E_{b} | |
| 15 | AlkLEᵢᵢ | E_{b} | |
| 16 | AlkLEᵢᵢE_{iib} | E_{b} | |
| 17 | AlkLEᵢᵢᵢ | E_{b} | |
| 18 | AlkLEᵢᵥ | E_{b} | |
| 19 | AlkL | E_{d} | |
| 20 | AlkLEᵢ | E_{d} | |
| 21 | AlkLEᵢᵢ | E_{d} | |
| 22 | AlkLEᵢᵢE_{iib} | E_{d} | |
| 23 | AlkLEᵢᵢᵢ | E_{d} | |
| 24 | AlkLEᵢᵥ | E_{d} | |
| 25 | AlkL | Eₑ | |
| 26 | AlkLEᵢ | Eₑ | |
| 27 | AlkLEᵢᵢ | Eₑ | |
| 28 | AlkLEᵢᵢE_{iib} | Eₑ | |
| 29 | AlkLEᵢᵢᵢ | Eₑ | |
| 30 | AlkLEᵢᵥ | Eₑ | |
| 31 | AlkL | E_{f} | |
| 32 | AlkLEᵢ | E_{f} | |
| 33 | AlkLEᵢᵢ | E_{f} | |
| 34 | AlkLEᵢᵢE_{iib} | E_{f} | |
| 35 | AlkLEᵢᵢᵢ | E_{f} | |
| 36 | AlkLEᵢᵥ | E_{f} | |
| 37 | AlkL | | E₁ |
| 38 | AlkLEᵢ | | E₁ |
| 39 | AlkLEᵢᵢ | | E₁ |
| 40 | AlkLEᵢᵢE_{iib} | | E₁ |
| 41 | AlkLEᵢᵢᵢ | | E₁ |
| 42 | AlkLEᵢᵥ | | E₁ |
| 43 | AlkL | Eₐ | E₁ |
| 44 | AlkLEᵢ | Eₐ | E₁ |
| 45 | AlkLEᵢᵢ | Eₐ | E₁ |
| 46 | AlkLEᵢᵢE_{iib} | Eₐ | E₁ |
| 47 | AlkLEᵢᵢᵢ | Eₐ | E₁ |
| 48 | AlkLEᵢᵥ | Eₐ | E₁ |
| 49 | AlkL | E_{b} | E₁ |
| 50 | AlkLEᵢ | E_{b} | E₁ |
| 51 | AlkLEᵢᵢ | E_{b} | E₁ |
| 52 | AlkLEᵢᵢE_{iib} | E_{b} | E₁ |
| 53 | AlkLEᵢᵢᵢ | E_{b} | E₁ |
| 54 | AlkLEᵢᵥ | E_{b} | E₁ |
| 55 | AlkL | E_{d} | E₁ |
| 56 | AlkLEᵢ | E_{d} | E₁ |
| 57 | AlkLEᵢᵢ | E_{d} | E₁ |
| 58 | AlkLEᵢᵢE_{iib} | E_{d} | E₁ |
| 59 | AlkLEᵢᵢᵢ | E_{d} | E₁ |
| 60 | AlkLEᵢᵥ | E_{d} | E₁ |
| 61 | AlkL | Eₑ | E₁ |
| 62 | AlkLEᵢ | Eₑ | E₁ |
| 63 | AlkLEᵢᵢ | Eₑ | E₁ |
| 64 | AlkLEᵢᵢE_{iib} | Eₑ | E₁ |
| 65 | AlkLEᵢᵢᵢ | Eₑ | E₁ |
| 66 | AlkLEᵢᵥ | Eₑ | E₁ |
| 67 | AlkL | E_{f} | E₁ |
| 68 | AlkLEᵢ | E_{f} | E₁ |
| 69 | AlkLEᵢᵢ | E_{f} | E₁ |
| 70 | AlkLEᵢᵢE_{iib} | E_{f} | E₁ |
| 71 | AlkLEᵢᵢᵢ | E_{f} | E₁ |
| 72 | AlkLEᵢᵥ | E_{f} | E₁ |

Microorganisms (abbreviated to MO) which are very especially preferred according to the invention are outstandingly suitable for the production of carboxylic acid esters and have increased or reduced enzymatic activities (abbreviated to E) which are described in the table hereinbelow, it being possible for these enzymatic activities additionally to be advantageously combined with an enzymatic activity described for the enzyme 3-ketoacyl-ACP (acyl carrier protein) synthase III (EC 2.3.1.41), which enzymatic activity is increased in comparison with the wild type of the microorganism, in particular the enzymatic activity of plants, preferably the enzymatic activity of plants whose seeds contain fatty acids with alkyl residues shorter than 14 C atoms and especially preferably the enzymatic activity of plants of the genera Cuphea, Elaeis, Cocos, Umbellularia and Cinnamomum and gene products selected from among AccA, AccB, AccC, AccD, AceE, AceF, Lpd, AcpP, FabA, FabB, FabD, FabF, FabG, FabH, FabI, FabZ, PanD, PanK, UdhA, PntA or PntB.

Any desired combinations of at least two of these enzymatic activities may advantageously be increased.

It may be additionally advantageous that the microorganism is equipped with an enzymatic activity which is described for the gene products selected from among TdcE, PflA, PflB, PflC, PfID, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, individually or in any desired combination, and which is reduced in comparison with the wild type of the microorganism.

| | | |
|---|---|---|
| MO | Increased E | Reduced E |

| | | | | |
|---|---|---|---|---|
| 1 | AlkL | Eᵥ | | |
| 2 | AlkLEᵢ | Eᵥ | | |
| 3 | AlkLEᵢᵢ | Eᵥ | | |
| 4 | AlkLEᵢᵢE_{iib} | Eᵥ | | |
| 5 | AlkLEᵢᵢᵢ | Eᵥ | | |
| 6 | AlkLEᵢᵥ | Eᵥ | | |
| 7 | AlkL | Eᵥ | Eₐ | |
| 8 | AlkLEᵢ | Eᵥ | Eₐ | |
| 9 | AlkLEᵢᵢ | Eᵥ | Eₐ | |
| 10 | AlkLEᵢᵢE_{iib} | Eᵥ | Eₐ | |
| 11 | AlkLEᵢᵢᵢ | Eᵥ | Eₐ | |
| 12 | AlkLEᵢᵥ | Eᵥ | Eₐ | |
| 13 | AlkL | Eᵥ | E_{b} | |
| 14 | AlkLEᵢ | Eᵥ | E_{b} | |
| 15 | AlkLEᵢᵢ | Eᵥ | E_{b} | |
| 16 | AlkLEᵢᵢE_{iib} | Eᵥ | E_{b} | |
| 17 | AlkLEᵢᵢᵢ | Eᵥ | E_{b} | |
| 18 | AlkLEᵢᵥ | Eᵥ | E_{b} | |
| 19 | AlkL | Eᵥ | E_{d} | |
| 20 | AlkLEᵢ | Eᵥ | E_{d} | |
| 21 | AlkLEᵢᵢ | Eᵥ | E_{d} | |
| 22 | AlkLEᵢᵢE_{iib} | Eᵥ | E_{d} | |
| 23 | AlkLEᵢᵢᵢ | Eᵥ | E_{d} | |
| 24 | AlkLEᵢᵥ | Eᵥ | E_{d} | |
| 25 | AlkL | Eᵥ | Eₑ | |
| 26 | AlkLEᵢ | Eᵥ | Eₑ | |
| 27 | AlkLEᵢᵢ | Eᵥ | Eₑ | |
| 28 | AlkLEᵢᵢE_{iib} | Eᵥ | Eₑ | |
| 29 | AlkLEᵢᵢᵢ | Eᵥ | Eₑ | |
| 30 | AlkLEᵢᵥ | Eᵥ | Eₑ | |
| 31 | AlkL | Eᵥ | E_{f} | |
| 32 | AlkLEᵢ | Eᵥ | E_{f} | |
| 33 | AlkLEᵢᵢ | Eᵥ | E_{f} | |
| 34 | AlkLEᵢᵢE_{iib} | Eᵥ | E_{f} | |
| 35 | AlkLEᵢᵢᵢ | Eᵥ | E_{f} | |
| 36 | AlkLEᵢᵥ | Eᵥ | E_{f} | |
| 37 | AlkL | EᵥEᵥᵢ | | |
| 38 | AlkLEᵢ | EᵥEᵥᵢ | | |
| 39 | AlkLEᵢᵢ | EᵥEᵥᵢ | | |
| 40 | AlkLEᵢᵢE_{iib} | EᵥEᵥᵢ | | |
| 41 | AlkLEᵢᵢᵢ | EᵥEᵥᵢ | | |
| 42 | AlkLEᵢᵥ | EᵥEᵥᵢ | | |
| 43 | AlkL | EᵥEᵥᵢ | Eₐ | |
| 44 | AlkLEᵢ | EᵥEᵥᵢ | Eₐ | |
| 45 | AlkLEᵢᵢ | EᵥEᵥᵢ | Eₐ | |
| 46 | AlkLEᵢᵢE_{iib} | EᵥEᵥᵢ | Eₐ | |
| 47 | AlkLEᵢᵢᵢ | EᵥEᵥᵢ | Eₐ | |
| 48 | AlkLEᵢᵥ | EᵥEᵥᵢ | Eₐ | |
| 49 | AlkL | EᵥEᵥᵢ | E_{b} | |
| 50 | AlkLEᵢ | EᵥEᵥᵢ | E_{b} | |
| 51 | AlkLEᵢᵢ | EᵥEᵥᵢ | E_{b} | |
| 52 | AlkLEᵢᵢE_{iib} | EᵥEᵥᵢ | E_{b} | |
| 53 | AlkLEᵢᵢᵢ | EᵥEᵥᵢ | E_{b} | |
| 54 | AlkLEᵢᵥ | EᵥEᵥᵢ | E_{b} | |
| 55 | AlkL | EᵥEᵥᵢ | E_{d} | |
| 56 | AlkLEᵢ | EᵥEᵥᵢ | E_{d} | |
| 57 | AlkLEᵢᵢ | EᵥEᵥᵢ | E_{d} | |
| 58 | AlkLEᵢᵢE_{iib} | EᵥEᵥᵢ | E_{d} | |
| 59 | AlkLEᵢᵢᵢ | EᵥEᵥᵢ | E_{d} | |
| 60 | AlkLEᵢᵥ | EᵥEᵥᵢ | E_{d} | |
| 61 | AlkL | EᵥEᵥᵢ | Eₑ | |
| 62 | AlkLEᵢ | EᵥEᵥᵢ | Eₑ | |
| 63 | AlkLEᵢᵢ | EᵥEᵥᵢ | Eₑ | |
| 64 | AlkLEᵢᵢE_{iib} | EᵥEᵥᵢ | Eₑ | |
| 65 | AlkLEᵢᵢᵢ | EᵥEᵥᵢ | Eₑ | |
| 66 | AlkLEᵢᵥ | EᵥEᵥᵢ | Eₑ | |
| 67 | AlkL | EᵥEᵥᵢ | E_{f} | |
| 68 | AlkLEᵢ | EᵥEᵥᵢ | E_{f} | |
| 69 | AlkLEᵢᵢ | EᵥEᵥᵢ | E_{f} | |
| 70 | AlkLEᵢᵢE_{iib} | EᵥEᵥᵢ | E_{f} | |
| 71 | AlkLEᵢᵢᵢ | EᵥEᵥᵢ | E_{f} | |
| 72 | AlkLEᵢᵥ | EᵥEᵥᵢ | E_{f} | |
| 73 | AlkL | Eᵥᵢᵢ | | |
| 74 | AlkLEᵢ | Eᵥᵢᵢ | | |
| 75 | AlkLEᵢᵢ | Eᵥᵢᵢ | | |
| 76 | AlkLEᵢᵢE_{iib} | Eᵥᵢᵢ | | |
| 77 | AlkLEᵢᵢᵢ | Eᵥᵢᵢ | | |
| 78 | AlkLEᵢᵥ | Eᵥᵢᵢ | | |
| 79 | AlkL | Eᵥᵢᵢ | Eₐ | |
| 80 | AlkLEᵢ | Eᵥᵢᵢ | Eₐ | |
| 81 | AlkLEᵢᵢ | Eᵥᵢᵢ | Eₐ | |
| 82 | AlkLEᵢᵢE_{iib} | Eᵥᵢᵢ | Eₐ | |
| 83 | AlkLEᵢᵢᵢ | Eᵥᵢᵢ | Eₐ | |
| 84 | AlkLEᵢᵥ | Eᵥᵢᵢ | Eₐ | |
| 85 | AlkL | Eᵥᵢᵢ | E_{b} | |
| 86 | AlkLEᵢ | Eᵥᵢᵢ | E_{b} | |
| 87 | AlkLEᵢᵢ | Eᵥᵢᵢ | E_{b} | |
| 88 | AlkLEᵢᵢE_{iib} | Eᵥᵢᵢ | E_{b} | |
| 89 | AlkLEᵢᵢᵢ | Eᵥᵢᵢ | E_{b} | |
| 90 | AlkLEᵢᵥ | Eᵥᵢᵢ | E_{b} | |
| 91 | AlkL | Eᵥᵢᵢ | E_{d} | |
| 92 | AlkLEᵢ | Eᵥᵢᵢ | E_{d} | |
| 93 | AlkLEᵢᵢ | Eᵥᵢᵢ | E_{d} | |
| 94 | AlkLEᵢᵢE_{iib} | Eᵥᵢᵢ | E_{d} | |
| 95 | AlkLEᵢᵢᵢ | Eᵥᵢᵢ | E_{d} | |
| 96 | AlkLEᵢᵥ | Eᵥᵢᵢ | E_{d} | |
| 97 | AlkL | Eᵥᵢᵢ | Eₑ | |
| 98 | AlkLEᵢ | Eᵥᵢᵢ | Eₑ | |
| 99 | AlkLEᵢᵢ | Eᵥᵢᵢ | Eₑ | |
| 100 | AlkLEᵢᵢE_{iib} | Eᵥᵢᵢ | Eₑ | |
| 101 | AlkLEᵢᵢᵢ | Eᵥᵢᵢ | Eₑ | |
| 102 | AlkLEᵢᵥ | Eᵥᵢᵢ | Eₑ | |
| 103 | AlkL | Eᵥᵢᵢ | E_{f} | |
| 104 | AlkLEᵢ | Eᵥᵢᵢ | E_{f} | |
| 105 | AlkLEᵢᵢ | Eᵥᵢᵢ | E_{f} | |
| 106 | AlkLEᵢᵢE_{iib} | Eᵥᵢᵢ | E_{f} | |
| 107 | AlkLEᵢᵢᵢ | Eᵥᵢᵢ | E_{f} | |
| 108 | AlkLEᵢᵥ | Eᵥᵢᵢ | E_{f} | |
| 109 | AlkL | EᵥᵢEᵥᵢᵢ | | |
| 110 | AlkLEᵢ | EᵥᵢEᵥᵢᵢ | | |
| 111 | AlkLEᵢᵢ | EᵥᵢEᵥᵢᵢ | | |
| 112 | AlkLEᵢᵢE_{iib} | EᵥᵢEᵥᵢᵢ | | |
| 113 | AlkLEᵢᵢᵢ | EᵥᵢEᵥᵢᵢ | | |
| 114 | AlkLEᵢᵥ | EᵥᵢEᵥᵢᵢ | | |
| 115 | AlkL | EᵥᵢEᵥᵢᵢ | Eₐ | |
| 116 | AlkLEᵢ | EᵥᵢEᵥᵢᵢ | Eₐ | |
| 117 | AlkLEᵢᵢ | EᵥᵢEᵥᵢᵢ | Eₐ | |
| 118 | AlkLEᵢᵢE_{iib} | EᵥᵢEᵥᵢᵢ | Eₐ | |
| 119 | AlkLEᵢᵢᵢ | EᵥᵢEᵥᵢᵢ | Eₐ | |
| 120 | AlkLEᵢᵥ | EᵥᵢEᵥᵢᵢ | Eₐ | |
| 121 | AlkL | EᵥᵢEᵥᵢᵢ | E_{b} | |
| 122 | AlkLEᵢ | EᵥᵢEᵥᵢᵢ | E_{b} | |
| 123 | AlkLEᵢᵢ | EᵥᵢEᵥᵢᵢ | E_{b} | |
| 124 | AlkLEᵢᵢE_{iib} | EᵥᵢEᵥᵢᵢ | E_{b} | |
| 125 | AlkLEᵢᵢᵢ | EᵥᵢEᵥᵢᵢ | E_{b} | |
| 126 | AlkLEᵢᵥ | EᵥᵢEᵥᵢᵢ | E_{b} | |
| 127 | AlkL | EᵥᵢEᵥᵢᵢ | E_{d} | |
| 128 | AlkLEᵢ | EᵥᵢEᵥᵢᵢ | E_{d} | |
| 129 | AlkLEᵢᵢ | EᵥᵢEᵥᵢᵢ | E_{d} | |
| 130 | AlkLEᵢᵢE_{iib} | EᵥᵢEᵥᵢᵢ | E_{d} | |
| 131 | AlkLEᵢᵢᵢ | EᵥᵢEᵥᵢᵢ | E_{d} | |
| 132 | AlkLEᵢᵥ | EᵥᵢEᵥᵢᵢ | E_{d} | |
| 133 | AlkL | EᵥᵢEᵥᵢᵢ | Eₑ | |
| 134 | AlkLEᵢ | EᵥᵢEᵥᵢᵢ | Eₑ | |
| 135 | AlkLEᵢᵢ | EᵥᵢEᵥᵢᵢ | Eₑ | |
| 136 | AlkLEᵢᵢE_{iib} | EᵥᵢEᵥᵢᵢ | Eₑ | |
| 137 | AlkLEᵢᵢᵢ | EᵥᵢEᵥᵢᵢ | Eₑ | |
| 138 | AlkLEᵢᵥ | EᵥᵢEᵥᵢᵢ | Eₑ | |
| 139 | AlkL | EᵥᵢEᵥᵢᵢ | E_{f} | |
| 140 | AlkLEᵢ | EᵥᵢEᵥᵢᵢ | E_{f} | |
| 141 | AlkLEᵢᵢ | EᵥᵢEᵥᵢᵢ | E_{f} | |
| 142 | AlkLEᵢᵢE_{iib} | EᵥᵢEᵥᵢᵢ | E_{f} | |
| 143 | AlkLEᵢᵢᵢ | EᵥᵢEᵥᵢᵢ | E_{f} | |
| 144 | AlkLEᵢᵥ | EᵥᵢEᵥᵢᵢ | E_{f} | |
| 145 | AlkL | E_{iib}EᵥᵢEᵥᵢᵢ | | |
| 146 | AlkLEᵢ | E_{iib}EᵥᵢEᵥᵢᵢ | | |
| 147 | AlkLEᵢᵢ | E_{iib}EᵥᵢEᵥᵢᵢ | | |
| 148 | AlkLEᵢᵢE_{iib} | E_{iib}EᵥᵢEᵥᵢᵢ | | |
| 149 | AlkLEᵢᵢᵢ | E_{iib}EᵥᵢEᵥᵢᵢ | | |
| 150 | AlkLEᵢᵥ | E_{iib}EᵥᵢEᵥᵢᵢ | | |
| 151 | AlkL | E_{iib}EᵥᵢEᵥᵢᵢ | Eₐ | |
| 152 | AlkLEᵢ | E_{iib}EᵥᵢEᵥᵢᵢ | Eₐ | |
| 153 | AlkLEᵢᵢ | E_{iib}EᵥᵢEᵥᵢᵢ | Eₐ | |
| 154 | AlkLEᵢᵢE_{iib} | E_{iib}EᵥᵢEᵥᵢᵢ | Eₐ | |
| 155 | AlkLEᵢᵢᵢ | E_{iib}EᵥᵢEᵥᵢᵢ | Eₐ | |
| 156 | AlkLEᵢᵥ | E_{iib}EᵥᵢEᵥᵢᵢ | Eₐ | |
| 157 | AlkL | E_{iib}EᵥᵢEᵥᵢᵢ | E_{b} | |
| 158 | AlkLEᵢ | E_{iib}EᵥᵢEᵥᵢᵢ | E_{b} | |
| 159 | AlkLEᵢᵢ | E_{iib}EᵥᵢEᵥᵢᵢ | E_{b} | |
| 160 | AlkLEᵢᵢE_{iib} | E_{iib}EᵥᵢEᵥᵢᵢ | E_{b} | |
| 161 | AlkLEᵢᵢᵢ | E_{iib}EᵥᵢEᵥᵢᵢ | E_{b} | |
| 162 | AlkLEᵢᵥ | E_{iib}EᵥᵢEᵥᵢᵢ | E_{b} | |
| 163 | AlkL | E_{iib}EᵥᵢEᵥᵢᵢ | E_{d} | |
| 164 | AlkLEᵢ | E_{iib}EᵥᵢEᵥᵢᵢ | E_{d} | |
| 165 | AlkLEᵢᵢ | E_{iib}EᵥᵢEᵥᵢᵢ | E_{d} | |
| 166 | AlkLEᵢᵢE_{iib} | E_{iib}EᵥᵢEᵥᵢᵢ | E_{d} | |
| 167 | AlkLEᵢᵢᵢ | E_{iib}EᵥᵢEᵥᵢᵢ | E_{d} | |
| 168 | AlkLEᵢᵥ | E_{iib}EᵥᵢEᵥᵢᵢ | E_{d} | |
| 169 | AlkL | E_{iib}EᵥᵢEᵥᵢᵢ | Eₑ | |
| 170 | AlkLEᵢ | E_{iib}EᵥᵢEᵥᵢᵢ | Eₑ | |
| 171 | AlkLEᵢᵢ | E_{iib}EᵥᵢEᵥᵢᵢ | Eₑ | |
| 172 | AlkLEᵢᵢE_{iib} | E_{iib}EᵥᵢEᵥᵢᵢ | Eₑ | |
| 173 | AlkLEᵢᵢᵢ | E_{iib}EᵥᵢEᵥᵢᵢ | Eₑ | |
| 174 | AlkLEᵢᵥ | E_{iib}EᵥᵢEᵥᵢᵢ | Eₑ | |
| 175 | AlkL | E_{iib}EᵥᵢEᵥᵢᵢ | E_{f} | |
| 176 | AlkLEᵢ | E_{iib}EᵥᵢEᵥᵢᵢ | E_{f} | |
| 177 | AlkLEᵢᵢ | E_{iib}EᵥᵢEᵥᵢᵢ | E_{f} | |
| 178 | AlkLEᵢᵢE_{iib} | E_{iib}EᵥᵢEᵥᵢᵢ | E_{f} | |
| 179 | AlkLEᵢᵢᵢ | E_{iib}EᵥᵢEᵥᵢᵢ | E_{f} | |
| 180 | AlkLEᵢᵥ | E_{iib}EᵥᵢEᵥᵢᵢ | E_{f} | |
| 181 | AlkL | Eᵥ | | E₁ |
| 182 | AlkLEᵢ | Eᵥ | | E₁ |
| 183 | AlkLEᵢᵢ | Eᵥ | | E₁ |
| 184 | AlkLEᵢᵢE_{iib} | Eᵥ | | E₁ |
| 185 | AlkLEᵢᵢᵢ | Eᵥ | | E₁ |
| 186 | AlkLEᵢᵥ | Eᵥ | | E₁ |
| 187 | AlkL | Eᵥ | Eₐ | E₁ |
| 188 | AlkLEᵢ | Eᵥ | Eₐ | E₁ |
| 189 | AlkLEᵢᵢ | Eᵥ | Eₐ | E₁ |
| 190 | AlkLEᵢᵢE_{iib} | Eᵥ | Eₐ | E₁ |
| 191 | AlkLEᵢᵢᵢ | Eᵥ | Eₐ | E₁ |
| 192 | AlkLEᵢᵥ | Eᵥ | Eₐ | E₁ |
| 193 | AlkL | Eᵥ | E_{b} | E₁ |
| 194 | AlkLEᵢ | Eᵥ | E_{b} | E₁ |
| 195 | AlkLEᵢᵢ | Eᵥ | E_{b} | E₁ |
| 196 | AlkLEᵢᵢE_{iib} | Eᵥ | E_{b} | E₁ |
| 197 | AlkLEᵢᵢᵢ | Eᵥ | E_{b} | E₁ |
| 198 | AlkLEᵢᵥ | Eᵥ | E_{b} | E₁ |
| 199 | AlkL | Eᵥ | E_{d} | E₁ |
| 200 | AlkLEᵢ | Eᵥ | E_{d} | E₁ |
| 201 | AlkLEᵢᵢ | Eᵥ | E_{d} | E₁ |
| 202 | AlkLEᵢᵢE_{iib} | Eᵥ | E_{d} | E₁ |
| 203 | AlkLEᵢᵢᵢ | Eᵥ | E_{d} | E₁ |
| 204 | AlkLEᵢᵥ | Eᵥ | E_{d} | E₁ |
| 205 | AlkL | Eᵥ | Eₑ | E₁ |
| 206 | AlkLEᵢ | Eᵥ | Eₑ | E₁ |
| 207 | AlkLEᵢᵢ | Eᵥ | Eₑ | E₁ |
| 208 | AlkLEᵢᵢE_{iib} | Eᵥ | Eₑ | E₁ |
| 209 | AlkLEᵢᵢᵢ | Eᵥ | Eₑ | E₁ |
| 210 | AlkLEᵢᵥ | Eᵥ | Eₑ | E₁ |
| 211 | AlkL | Eᵥ | E_{f} | E₁ |
| 212 | AlkLEᵢ | Eᵥ | E_{f} | E₁ |
| 213 | AlkLEᵢᵢ | Eᵥ | E_{f} | E₁ |
| 214 | AlkLEᵢᵢE_{iib} | Eᵥ | E_{f} | E₁ |
| 215 | AlkLEᵢᵢᵢ | Eᵥ | E_{f} | E₁ |
| 216 | AlkLEᵢᵥ | Eᵥ | E_{f} | E₁ |
| 217 | AlkL | EᵥEᵥᵢ | | E₁ |
| 218 | AlkLEᵢ | EᵥEᵥᵢ | | E₁ |
| 219 | AlkLEᵢᵢ | EᵥEᵥᵢ | | E₁ |
| 220 | AlkLEᵢᵢE_{iib} | EᵥEᵥᵢ | | E₁ |
| 221 | AlkLEᵢᵢᵢ | EᵥEᵥᵢ | | E₁ |
| 222 | AlkLEᵢᵥ | EᵥEᵥᵢ | | E₁ |
| 223 | AlkL | EᵥEᵥᵢ | Eₐ | E₁ |
| 224 | AlkLEᵢ | EᵥEᵥᵢ | Eₐ | E₁ |
| 225 | AlkLEᵢᵢ | EᵥEᵥᵢ | Eₐ | E₁ |
| 226 | AlkLEᵢᵢE_{iib} | EᵥEᵥᵢ | Eₐ | E₁ |
| 227 | AlkLEᵢᵢᵢ | EᵥEᵥᵢ | Eₐ | E₁ |
| 228 | AlkLEᵢᵥ | EᵥEᵥᵢ | Eₐ | E₁ |
| 229 | AlkL | EᵥEᵥᵢ | E_{b} | E₁ |
| 230 | AlkLEᵢ | EᵥEᵥᵢ | E_{b} | E₁ |
| 231 | AlkLEᵢᵢ | EᵥEᵥᵢ | E_{b} | E₁ |
| 232 | AlkLEᵢᵢE_{iib} | EᵥEᵥᵢ | E_{b} | E₁ |
| 233 | AlkLEᵢᵢᵢ | EᵥEᵥᵢ | E_{b} | E₁ |
| 234 | AlkLEᵢᵥ | EᵥEᵥᵢ | E_{b} | E₁ |
| 235 | AlkL | EᵥEᵥᵢ | E_{d} | E₁ |
| 236 | AlkLEᵢ | EᵥEᵥᵢ | E_{d} | E₁ |
| 237 | AlkLEᵢᵢ | EᵥEᵥᵢ | E_{d} | E₁ |
| 238 | AlkLEᵢᵢE_{iib} | EᵥEᵥᵢ | E_{d} | E₁ |
| 239 | AlkLEᵢᵢᵢ | EᵥEᵥᵢ | E_{d} | E₁ |
| 240 | AlkLEᵢᵥ | EᵥEᵥᵢ | E_{d} | E₁ |
| 241 | AlkL | EᵥEᵥᵢ | Eₑ | E₁ |
| 242 | AlkLEᵢ | EᵥEᵥᵢ | Eₑ | E₁ |
| 243 | AlkLEᵢᵢ | EᵥEᵥᵢ | Eₑ | E₁ |
| 244 | AlkLEᵢᵢE_{iib} | EᵥEᵥᵢ | Eₑ | E₁ |
| 245 | AlkLEᵢᵢᵢ | EᵥEᵥᵢ | Eₑ | E₁ |
| 246 | AlkLEᵢᵥ | EᵥEᵥᵢ | Eₑ | E₁ |
| 247 | AlkL | EᵥEᵥᵢ | E_{f} | E₁ |
| 248 | AlkLEᵢ | EᵥEᵥᵢ | E_{f} | E₁ |
| 249 | AlkLEᵢᵢ | EᵥEᵥᵢ | E_{f} | E₁ |
| 250 | AlkLEᵢᵢE_{iib} | EᵥEᵥᵢ | E_{f} | E₁ |
| 251 | AlkLEᵢᵢᵢ | EᵥEᵥᵢ | E_{f} | E₁ |
| 252 | AlkLEᵢᵥ | EᵥEᵥᵢ | E_{f} | E₁ |
| 253 | AlkL | Eᵥᵢᵢ | | E₁ |
| 254 | AlkLEᵢ | Eᵥᵢᵢ | | E₁ |
| 255 | AlkLEᵢᵢ | Eᵥᵢᵢ | | E₁ |
| 256 | AlkLEᵢᵢE_{iib} | Eᵥᵢᵢ | | E₁ |
| 257 | AlkLEᵢᵢᵢ | Eᵥᵢᵢ | | E₁ |
| 258 | AlkLEᵢᵥ | Eᵥᵢᵢ | | E₁ |
| 259 | AlkL | Eᵥᵢᵢ | Eₐ | E₁ |
| 260 | AlkLEᵢ | Eᵥᵢᵢ | Eₐ | E₁ |
| 261 | AlkLEᵢᵢ | Eᵥᵢᵢ | Eₐ | E₁ |
| 262 | AlkLEᵢᵢE_{iib} | Eᵥᵢᵢ | Eₐ | E₁ |
| 263 | AlkLEᵢᵢᵢ | Eᵥᵢᵢ | Eₐ | E₁ |
| 264 | AlkLEᵢᵥ | Eᵥᵢᵢ | Eₐ | E₁ |
| 265 | AlkL | Eᵥᵢᵢ | E_{b} | E₁ |
| 266 | AlkLEᵢ | Eᵥᵢᵢ | E_{b} | E₁ |
| 267 | AlkLEᵢᵢ | Eᵥᵢᵢ | E_{b} | E₁ |
| 268 | AlkLEᵢᵢE_{iib} | Eᵥᵢᵢ | E_{b} | E₁ |
| 269 | AlkLEᵢᵢᵢ | Eᵥᵢᵢ | E_{b} | E₁ |
| 270 | AlkLEᵢᵥ | Eᵥᵢᵢ | E_{b} | E₁ |
| 271 | AlkL | Eᵥᵢᵢ | E_{d} | E₁ |
| 272 | AlkLEᵢ | Eᵥᵢᵢ | E_{d} | E₁ |
| 273 | AlkLEᵢᵢ | Eᵥᵢᵢ | E_{d} | E₁ |
| 274 | AlkLEᵢᵢE_{iib} | Eᵥᵢᵢ | E_{d} | E₁ |
| 275 | AlkLEᵢᵢᵢ | Eᵥᵢᵢ | E_{d} | E₁ |
| 276 | AlkLEᵢᵥ | Eᵥᵢᵢ | E_{d} | E₁ |
| 277 | AlkL | Eᵥᵢᵢ | Eₑ | E₁ |
| 278 | AlkLEᵢ | Eᵥᵢᵢ | Eₑ | E₁ |
| 279 | AlkLEᵢᵢ | Eᵥᵢᵢ | Eₑ | E₁ |
| 280 | AlkLEᵢᵢE_{iib} | Eᵥᵢᵢ | Eₑ | E₁ |
| 281 | AlkLEᵢᵢᵢ | Eᵥᵢᵢ | Eₑ | E₁ |
| 282 | AlkLEᵢᵥ | Eᵥᵢᵢ | Eₑ | E₁ |
| 283 | AlkL | Eᵥᵢᵢ | E_{f} | E₁ |
| 284 | AlkLEᵢ | Eᵥᵢᵢ | E_{f} | E₁ |
| 285 | AlkLEᵢᵢ | Eᵥᵢᵢ | E_{f} | E₁ |
| 286 | AlkLEᵢᵢE_{iib} | Eᵥᵢᵢ | E_{f} | E₁ |
| 287 | AlkLEᵢᵢᵢ | Eᵥᵢᵢ | E_{f} | E₁ |
| 288 | AlkLEᵢᵥ | Eᵥᵢᵢ | E_{f} | E₁ |
| 289 | AlkL | EᵥᵢEᵥᵢᵢ | | E₁ |
| 290 | AlkLEᵢ | EᵥᵢEᵥᵢᵢ | | E₁ |
| 291 | AlkLEᵢᵢ | EᵥᵢEᵥᵢᵢ | | E₁ |
| 292 | AlkLEᵢᵢE_{iib} | EᵥᵢEᵥᵢᵢ | | E₁ |
| 293 | AlkLEᵢᵢᵢ | EᵥᵢEᵥᵢᵢ | | E₁ |
| 294 | AlkLEᵢᵥ | EᵥᵢEᵥᵢᵢ | | E₁ |
| 295 | AlkL | EᵥᵢEᵥᵢᵢ | Eₐ | E₁ |
| 296 | AlkLEᵢ | EᵥᵢEᵥᵢᵢ | Eₐ | E₁ |
| 297 | AlkLEᵢᵢ | EᵥᵢEᵥᵢᵢ | Eₐ | E₁ |
| 298 | AlkLEᵢᵢE_{iib} | EᵥᵢEᵥᵢᵢ | Eₐ | E₁ |
| 299 | AlkLEᵢᵢᵢ | EᵥᵢEᵥᵢᵢ | Eₐ | E₁ |
| 300 | AlkLEᵢᵥ | EᵥᵢEᵥᵢᵢ | Eₐ | E₁ |
| 301 | AlkL | EᵥᵢEᵥᵢᵢ | E_{b} | E₁ |
| 302 | AlkLEᵢ | EᵥᵢEᵥᵢᵢ | E_{b} | E₁ |
| 303 | AlkLEᵢᵢ | EᵥᵢEᵥᵢᵢ | E_{b} | E₁ |
| 304 | AlkLEᵢᵢE_{iib} | EᵥᵢEᵥᵢᵢ | E_{b} | E₁ |
| 305 | AlkLEᵢᵢᵢ | EᵥᵢEᵥᵢᵢ | E_{b} | E₁ |
| 306 | AlkLEᵢᵥ | EᵥᵢEᵥᵢᵢ | E_{b} | E₁ |
| 307 | AlkL | EᵥᵢEᵥᵢᵢ | E_{d} | E₁ |
| 308 | AlkLEᵢ | EᵥᵢEᵥᵢᵢ | E_{d} | E₁ |
| 309 | AlkLEᵢᵢ | EᵥᵢEᵥᵢᵢ | E_{d} | E₁ |
| 310 | AlkLEᵢᵢE_{iib} | EᵥᵢEᵥᵢᵢ | E_{d} | E₁ |
| 311 | AlkLEᵢᵢᵢ | EᵥᵢEᵥᵢᵢ | E_{d} | E₁ |
| 312 | AlkLEᵢᵥ | EᵥᵢEᵥᵢᵢ | E_{d} | E₁ |
| 313 | AlkL | EᵥᵢEᵥᵢᵢ | Eₑ | E₁ |
| 314 | AlkLEᵢ | EᵥᵢEᵥᵢᵢ | Eₑ | E₁ |
| 315 | AlkLEᵢᵢ | EᵥᵢEᵥᵢᵢ | Eₑ | E₁ |
| 316 | AlkLEᵢᵢE_{iib} | EᵥᵢEᵥᵢᵢ | Eₑ | E₁ |
| 317 | AlkLEᵢᵢᵢ | EᵥᵢEᵥᵢᵢ | Eₑ | E₁ |
| 318 | AlkLEᵢᵥ | EᵥᵢEᵥᵢᵢ | Eₑ | E₁ |
| 319 | AlkL | EᵥᵢEᵥᵢᵢ | E_{f} | E₁ |
| 320 | AlkLEᵢ | EᵥᵢEᵥᵢᵢ | E_{f} | E₁ |
| 321 | AlkLEᵢᵢ | EᵥᵢEᵥᵢᵢ | E_{f} | E₁ |
| 322 | AlkLEᵢᵢE_{iib} | EᵥᵢEᵥᵢᵢ | E_{f} | E₁ |
| 323 | AlkLEᵢᵢᵢ | EᵥᵢEᵥᵢᵢ | E_{f} | E₁ |
| 324 | AlkLEᵢᵥ | EᵥᵢEᵥᵢᵢ | E_{f} | E₁ |
| 325 | AlkL | E_{iib}EᵥᵢEᵥᵢᵢ | | E₁ |
| 326 | AlkLEᵢ | E_{iib}EᵥᵢEᵥᵢᵢ | | E₁ |
| 327 | AlkLEᵢᵢ | E_{iib}EᵥᵢEᵥᵢᵢ | | E₁ |
| 328 | AlkLEᵢᵢE_{iib} | E_{iib}EᵥᵢEᵥᵢᵢ | | E₁ |
| 329 | AlkLEᵢᵢᵢ | E_{iib}EᵥᵢEᵥᵢᵢ | | E₁ |
| 330 | AlkLEᵢᵥ | E_{iib}EᵥᵢEᵥᵢᵢ | | E₁ |
| 331 | AlkL | E_{iib}EᵥᵢEᵥᵢᵢ | Eₐ | E₁ |
| 332 | AlkLEᵢ | E_{iib}EᵥᵢEᵥᵢᵢ | Eₐ | E₁ |
| 333 | AlkLEᵢᵢ | E_{iib}EᵥᵢEᵥᵢᵢ | Eₐ | E₁ |
| 334 | AlkLEᵢᵢE_{iib} | E_{iib}EᵥᵢEᵥᵢᵢ | Eₐ | E₁ |
| 335 | AlkLEᵢᵢᵢ | E_{iib}EᵥᵢEᵥᵢᵢ | Eₐ | E₁ |
| 336 | AlkLEᵢᵥ | E_{iib}EᵥᵢEᵥᵢᵢ | Eₐ | E₁ |
| 337 | AlkL | E_{iib}EᵥᵢEᵥᵢᵢ | E_{b} | E₁ |
| 338 | AlkLEᵢ | E_{iib}EᵥᵢEᵥᵢᵢ | E_{b} | E₁ |
| 339 | AlkLEᵢᵢ | E_{iib}EᵥᵢEᵥᵢᵢ | E_{b} | E₁ |
| 340 | AlkLEᵢᵢE_{iib} | E_{iib}EᵥᵢEᵥᵢᵢ | E_{b} | E₁ |
| 341 | AlkLEᵢᵢᵢ | E_{iib}EᵥᵢEᵥᵢᵢ | E_{b} | E₁ |
| 342 | AlkLEᵢᵥ | E_{iib}EᵥᵢEᵥᵢᵢ | E_{b} | E₁ |
| 343 | AlkL | E_{iib}EᵥᵢEᵥᵢᵢ | E_{d} | E₁ |
| 344 | AlkLEᵢ | E_{iib}EᵥᵢEᵥᵢᵢ | E_{d} | E₁ |
| 345 | AlkLEᵢᵢ | E_{iib}EᵥᵢEᵥᵢᵢ | E_{d} | E₁ |
| 346 | AlkLEᵢᵢE_{iib} | E_{iib}EᵥᵢEᵥᵢᵢ | E_{d} | E₁ |
| 347 | AlkLEᵢᵢᵢ | E_{iib}EᵥᵢEᵥᵢᵢ | E_{d} | E₁ |
| 348 | AlkLEᵢᵥ | E_{iib}EᵥᵢEᵥᵢᵢ | E_{d} | E₁ |
| 349 | AlkL | E_{iib}EᵥᵢEᵥᵢᵢ | Eₑ | E₁ |
| 350 | AlkLEᵢ | E_{iib}EᵥᵢEᵥᵢᵢ | Eₑ | E₁ |
| 351 | AlkLEᵢᵢ | E_{iib}EᵥᵢEᵥᵢᵢ | Eₑ | E₁ |
| 352 | AlkLEᵢᵢE_{iib} | E_{iib}EᵥᵢEᵥᵢᵢ | Eₑ | E₁ |
| 353 | AlkLEᵢᵢᵢ | E_{iib}EᵥᵢEᵥᵢᵢ | Eₑ | E₁ |
| 354 | AlkLEᵢᵥ | E_{iib}EᵥᵢEᵥᵢᵢ | Eₑ | E₁ |
| 355 | AlkL | E_{iib}EᵥᵢEᵥᵢᵢ | E_{f} | E₁ |
| 356 | AlkLEᵢ | E_{iib}EᵥᵢEᵥᵢᵢ | E_{f} | E₁ |
| 357 | AlkLEᵢᵢ | E_{iib}EᵥᵢEᵥᵢᵢ | E_{f} | E₁ |
| 358 | AlkLEᵢᵢE_{iib} | E_{iib}EᵥᵢEᵥᵢᵢ | E_{f} | E₁ |
| 359 | AlkLEᵢᵢᵢ | E_{iib}EᵥᵢEᵥᵢᵢ | E_{f} | E₁ |
| 360 | AlkLEᵢᵥ | E_{iib}EᵥᵢEᵥᵢᵢ | E_{f} | E₁ |

Microorganisms (abbreviated to MO) which are very especially preferred according to the invention are outstandingly suitable for the production of alkan-1-ols and alkan-1-als and have increased or reduced enzymatic activities (abbreviated to E) which are described in the table hereinbelow, it being possible for these enzymatic activities additionally to be advantageously combined with an enzymatic activity described for the 3-ketoacyl-ACP (acyl carrier protein) synthase III (EC 2.3.1.41), which enzymatic activity is increased in comparison with the wild type of the microorganism, in particular the enzymatic activity of plants, preferably the enzymatic activity of plants whose seeds contain fatty acids with alkyl residues shorter than 14 C atoms and especially preferably the enzymatic activity of plants of the genera Cuphea, Elaeis, Cocos, Umbellularia and Cinnamomum and gene products selected from among AccA, AccB, AccC, AccD, AceE, AceF, Lpd, AcpP, FabA, FabB, FabD, FabF, FabG, FabH, FabI, FabZ, PanD, PanK, UdhA, PntA or PntB.

Any desired combinations of at least two of these enzymatic activities may advantageously be increased.

It may be additionally advantageous that the microorganism is equipped with an enzymatic activity which is described for the gene products selected from among TdcE, PflA, PflB, PflC, PflD, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, individually or in any desired combination, and which is reduced in comparison with the wild type of the microorganism.

| | | |
|---|---|---|
| MO | Increased E | Reduced E |

| | | | | |
|---|---|---|---|---|
| 1 | AlkL | Eᵥᵢᵢᵢ | | |
| 2 | AlkLEᵢ | Eᵥᵢᵢᵢ | | |
| 3 | AlkLEᵢᵢ | Eᵥᵢᵢᵢ | | |
| 4 | AlkLEᵢᵢE_{iib} | Eᵥᵢᵢᵢ | | |
| 5 | AlkLEᵢᵢᵢ | Eᵥᵢᵢᵢ | | |
| 6 | AlkLEᵢᵥ | Eᵥᵢᵢᵢ | | |
| 7 | AlkL | Eᵥᵢᵢᵢ | Eₐ | |
| 8 | AlkLEᵢ | Eᵥᵢᵢᵢ | Eₐ | |
| 9 | AlkLEᵢᵢ | Eᵥᵢᵢᵢ | Eₐ | |
| 10 | AlkLEᵢᵢE_{iib} | Eᵥᵢᵢᵢ | Eₐ | |
| 11 | AlkLEᵢᵢᵢ | Eᵥᵢᵢᵢ | Eₐ | |
| 12 | AlkLEᵢᵥ | Eᵥᵢᵢᵢ | Eₐ | |
| 13 | AlkL | Eᵥᵢᵢᵢ | E_{b} | |
| 14 | AlkLEᵢ | Eᵥᵢᵢᵢ | E_{b} | |
| 15 | AlkLEᵢᵢ | Eᵥᵢᵢᵢ | E_{b} | |
| 16 | AlkLEᵢᵢE_{iib} | Eᵥᵢᵢᵢ | E_{b} | |
| 17 | AlkLEᵢᵢᵢ | Eᵥᵢᵢᵢ | E_{b} | |
| 18 | AlkLEᵢᵥ | Eᵥᵢᵢᵢ | E_{b} | |
| 19 | AlkL | Eᵥᵢᵢᵢ | E_{d} | |
| 20 | AlkLEᵢ | Eᵥᵢᵢᵢ | E_{d} | |
| 21 | AlkLEᵢᵢ | Eᵥᵢᵢᵢ | E_{d} | |
| 22 | AlkLEᵢᵢE_{iib} | Eᵥᵢᵢᵢ | E_{d} | |
| 23 | AlkLEᵢᵢᵢ | Eᵥᵢᵢᵢ | E_{d} | |
| 24 | AlkLEᵢᵥ | Eᵥᵢᵢᵢ | E_{d} | |
| 25 | AlkL | Eᵥᵢᵢᵢ | Eₑ | |
| 26 | AlkLEᵢ | Eᵥᵢᵢᵢ | Eₑ | |
| 27 | AlkLEᵢᵢ | Eᵥᵢᵢᵢ | Eₑ | |
| 28 | AlkLEᵢᵢE_{iib} | Eᵥᵢᵢᵢ | Eₑ | |
| 29 | AlkLEᵢᵢᵢ | Eᵥᵢᵢᵢ | Eₑ | |
| 30 | AlkLEᵢᵥ | Eᵥᵢᵢᵢ | Eₑ | |
| 31 | AlkL | Eᵥᵢᵢᵢ | E_{f} | |
| 32 | AlkLEᵢ | Eᵥᵢᵢᵢ | E_{f} | |
| 33 | AlkLEᵢᵢ | Eᵥᵢᵢᵢ | E_{f} | |
| 34 | AlkLEᵢᵢE_{iib} | Eᵥᵢᵢᵢ | E_{f} | |
| 35 | AlkLEᵢᵢᵢ | Eᵥᵢᵢᵢ | E_{f} | |
| 36 | AlkLEᵢᵥ | Eᵥᵢᵢᵢ | E_{f} | |
| 37 | AlkL | Eᵢₓ | | |
| 38 | AlkLEᵢ | Eᵢₓ | | |
| 39 | AlkLEᵢᵢ | Eᵢₓ | | |
| 40 | AlkLEᵢᵢE_{iib} | Eᵢₓ | | |
| 41 | AlkLEᵢᵢᵢ | Eᵢₓ | | |
| 42 | AlkLEᵢᵥ | Eᵢₓ | | |
| 43 | AlkL | Eᵢₓ | Eₐ | |
| 44 | AlkLEᵢ | Eᵢₓ | Eₐ | |
| 45 | AlkLEᵢᵢ | Eᵢₓ | Eₐ | |
| 46 | AlkLEᵢᵢE_{iib} | Eᵢₓ | Eₐ | |
| 47 | AlkLEᵢᵢᵢ | Eᵢₓ | Eₐ | |
| 48 | AlkLEᵢᵥ | Eᵢₓ | Eₐ | |
| 49 | AlkL | Eᵢₓ | E_{b} | |
| 50 | AlkLEᵢ | Eᵢₓ | E_{b} | |
| 51 | AlkLEᵢᵢ | Eᵢₓ | E_{b} | |
| 52 | AlkLEᵢᵢE_{iib} | Eᵢₓ | E_{b} | |
| 53 | AlkLEᵢᵢᵢ | Eᵢₓ | E_{b} | |
| 54 | AlkLEᵢᵥ | Eᵢₓ | E_{b} | |
| 55 | AlkL | Eᵢₓ | E_{d} | |
| 56 | AlkLEᵢ | Eᵢₓ | E_{d} | |
| 57 | AlkLEᵢᵢ | Eᵢₓ | E_{d} | |
| 58 | AlkLEᵢᵢE_{iib} | Eᵢₓ | E_{d} | |
| 59 | AlkLEᵢᵢᵢ | Eᵢₓ | E_{d} | |
| 60 | AlkLEᵢᵥ | Eᵢₓ | E_{d} | |
| 61 | AlkL | Eᵢₓ | Eₑ | |
| 62 | AlkLEᵢ | Eᵢₓ | Eₑ | |
| 63 | AlkLEᵢᵢ | Eᵢₓ | Eₑ | |
| 64 | AlkLEᵢᵢE_{iib} | Eᵢₓ | Eₑ | |
| 65 | AlkLEᵢᵢᵢ | Eᵢₓ | Eₑ | |
| 66 | AlkLEᵢᵥ | Eᵢₓ | Eₑ | |
| 67 | AlkL | Eᵢₓ | E_{f} | |
| 68 | AlkLEᵢ | Eᵢₓ | E_{f} | |
| 69 | AlkLEᵢᵢ | Eᵢₓ | E_{f} | |
| 70 | AlkLEᵢᵢE_{iib} | Eᵢₓ | E_{f} | |
| 71 | AlkLEᵢᵢᵢ | Eᵢₓ | E_{f} | |
| 72 | AlkLEᵢᵥ | Eᵢₓ | E_{f} | |
| 73 | AlkL | Eₓ | | |
| 74 | AlkLEᵢ | Eₓ | | |
| 75 | AlkLEᵢᵢ | Eₓ | | |
| 76 | AlkLEᵢᵢE_{iib} | Eₓ | | |
| 77 | AlkLEᵢᵢᵢ | Eₓ | | |
| 78 | AlkLEᵢᵥ | Eₓ | | |
| 79 | AlkL | Eₓ | Eₐ | |
| 80 | AlkLEᵢ | Eₓ | Eₐ | |
| 81 | AlkLEᵢᵢ | Eₓ | Eₐ | |
| 82 | AlkLEᵢᵢE_{iib} | Eₓ | Eₐ | |
| 83 | AlkLEᵢᵢᵢ | Eₓ | Eₐ | |
| 84 | AlkLEᵢᵥ | Eₓ | Eₐ | |
| 85 | AlkL | Eₓ | E_{b} | |
| 86 | AlkLEᵢ | Eₓ | E_{b} | |
| 87 | AlkLEᵢᵢ | Eₓ | E_{b} | |
| 88 | AlkLEᵢᵢE_{iib} | Eₓ | E_{b} | |
| 89 | AlkLEᵢᵢᵢ | Eₓ | E_{b} | |
| 90 | AlkLEᵢᵥ | Eₓ | E_{b} | |
| 91 | AlkL | Eₓ | E_{d} | |
| 92 | AlkLEᵢ | Eₓ | E_{d} | |
| 93 | AlkLEᵢᵢ | Eₓ | E_{d} | |
| 94 | AlkLEᵢᵢE_{iib} | Eₓ | E_{d} | |
| 95 | AlkLEᵢᵢᵢ | Eₓ | E_{d} | |
| 96 | AlkLEᵢᵥ | Eₓ | E_{d} | |
| 97 | AlkL | Eₓ | Eₑ | |
| 98 | AlkLEᵢ | Eₓ | Eₑ | |
| 99 | AlkLEᵢᵢ | Eₓ | Eₑ | |
| 100 | AlkLEᵢᵢE_{iib} | Eₓ | Eₑ | |
| 101 | AlkLEᵢᵢᵢ | Eₓ | Eₑ | |
| 102 | AlkLEᵢᵥ | Eₓ | Eₑ | |
| 103 | AlkL | Eₓ | E_{f} | |
| 104 | AlkLEᵢ | Eₓ | E_{f} | |
| 105 | AlkLEᵢᵢ | Eₓ | E_{f} | |
| 106 | AlkLEᵢᵢE_{iib} | Eₓ | E_{f} | |
| 107 | AlkLEᵢᵢᵢ | Eₓ | E_{f} | |
| 108 | AlkLEᵢᵥ | Eₓ | E_{f} | |
| 109 | AlkL | EᵥᵢEᵥᵢᵢᵢ | | |
| 110 | AlkLEᵢ | EᵥᵢEᵥᵢᵢᵢ | | |
| 111 | AlkLEᵢᵢ | EᵥᵢEᵥᵢᵢᵢ | | |
| 112 | AlkLEᵢᵢE_{iib} | EᵥᵢEᵥᵢᵢᵢ | | |
| 113 | AlkLEᵢᵢᵢ | EᵥᵢEᵥᵢᵢᵢ | | |
| 114 | AlkLEᵢᵥ | EᵥᵢEᵥᵢᵢᵢ | | |
| 115 | AlkL | EᵥᵢEᵥᵢᵢᵢ | Eₐ | |
| 116 | AlkLEᵢ | EᵥᵢEᵥᵢᵢᵢ | Eₐ | |
| 117 | AlkLEᵢᵢ | EᵥᵢEᵥᵢᵢᵢ | Eₐ | |
| 118 | AlkLEᵢᵢE_{iib} | EᵥᵢEᵥᵢᵢᵢ | Eₐ | |
| 119 | AlkLEᵢᵢᵢ | EᵥᵢEᵥᵢᵢᵢ | Eₐ | |
| 120 | AlkLEᵢᵥ | EᵥᵢEᵥᵢᵢᵢ | Eₐ | |
| 121 | AlkL | EᵥᵢEᵥᵢᵢᵢ | E_{b} | |
| 122 | AlkLEᵢ | EᵥᵢEᵥᵢᵢᵢ | E_{b} | |
| 123 | AlkLEᵢᵢ | EᵥᵢEᵥᵢᵢᵢ | E_{b} | |
| 124 | AlkLEᵢᵢE_{iib} | EᵥᵢEᵥᵢᵢᵢ | E_{b} | |
| 125 | AlkLEᵢᵢᵢ | EᵥᵢEᵥᵢᵢᵢ | E_{b} | |
| 126 | AlkLEᵢᵥ | EᵥᵢEᵥᵢᵢᵢ | E_{b} | |
| 127 | AlkL | EᵥᵢEᵥᵢᵢᵢ | E_{d} | |
| 128 | AlkLEᵢ | EᵥᵢEᵥᵢᵢᵢ | E_{d} | |
| 129 | AlkLEᵢᵢ | EᵥᵢEᵥᵢᵢᵢ | E_{d} | |
| 130 | AlkLEᵢᵢE_{iib} | EᵥᵢEᵥᵢᵢᵢ | E_{d} | |
| 131 | AlkLEᵢᵢᵢ | EᵥᵢEᵥᵢᵢᵢ | E_{d} | |
| 132 | AlkLEᵢᵥ | EᵥᵢEᵥᵢᵢᵢ | E_{d} | |
| 133 | AlkL | EᵥᵢEᵥᵢᵢᵢ | Eₑ | |
| 134 | AlkLEᵢ | EᵥᵢEᵥᵢᵢᵢ | Eₑ | |
| 135 | AlkLEᵢᵢ | EᵥᵢEᵥᵢᵢᵢ | Eₑ | |
| 136 | AlkLEᵢᵢE_{iib} | EᵥᵢEᵥᵢᵢᵢ | Eₑ | |
| 137 | AlkLEᵢᵢᵢ | EᵥᵢEᵥᵢᵢᵢ | Eₑ | |
| 138 | AlkLEᵢᵥ | EᵥᵢEᵥᵢᵢᵢ | Eₑ | |
| 139 | AlkL | EᵥᵢEᵥᵢᵢᵢ | E_{f} | |
| 140 | AlkLEᵢ | EᵥᵢEᵥᵢᵢᵢ | E_{f} | |
| 141 | AlkLEᵢᵢ | EᵥᵢEᵥᵢᵢᵢ | E_{f} | |
| 142 | AlkLEᵢᵢE_{iib} | EᵥᵢEᵥᵢᵢᵢ | E_{f} | |
| 143 | AlkLEᵢᵢᵢ | EᵥᵢEᵥᵢᵢᵢ | E_{f} | |
| 144 | AlkLEᵢᵥ | EᵥᵢEᵥᵢᵢᵢ | E_{f} | |
| 145 | AlkL | E_{iib}EᵥᵢEₓ | | |
| 146 | AlkLEᵢ | E_{iib}EᵥᵢEₓ | | |
| 147 | AlkLEᵢᵢ | E_{iib}EᵥᵢEₓ | | |
| 148 | AlkLEᵢᵢE_{iib} | E_{iib}EᵥᵢEₓ | | |
| 149 | AlkLEᵢᵢᵢ | E_{iib}EᵥᵢEₓ | | |
| 150 | AlkLEᵢᵥ | E_{iib}EᵥᵢEₓ | | |
| 151 | AlkL | E_{iib}EᵥᵢEₓ | Eₐ | |
| 152 | AlkLEᵢ | E_{iib}EᵥᵢEₓ | Eₐ | |
| 153 | AlkLEᵢᵢ | E_{iib}EᵥᵢEₓ | Eₐ | |
| 154 | AlkLEᵢᵢE_{iib} | E_{iib}EᵥᵢEₓ | Eₐ | |
| 155 | AlkLEᵢᵢᵢ | E_{iib}EᵥᵢEₓ | Eₐ | |
| 156 | AlkLEᵢᵥ | E_{iib}EᵥᵢEₓ | Eₐ | |
| 157 | AlkL | E_{iib}EᵥᵢEₓ | E_{b} | |
| 158 | AlkLEᵢ | E_{iib}EᵥᵢEₓ | E_{b} | |
| 159 | AlkLEᵢᵢ | E_{iib}EᵥᵢEₓ | E_{b} | |
| 160 | AlkLEᵢᵢE_{iib} | E_{iib}EᵥᵢEₓ | E_{b} | |
| 161 | AlkLEᵢᵢᵢ | E_{iib}EᵥᵢEₓ | E_{b} | |
| 162 | AlkLEᵢᵥ | E_{iib}EᵥᵢEₓ | E_{b} | |
| 163 | AlkL | E_{iib}EᵥᵢEₓ | E_{d} | |
| 164 | AlkLEᵢ | E_{iib}EᵥᵢEₓ | E_{d} | |
| 165 | AlkLEᵢᵢ | E_{iib}EᵥᵢEₓ | E_{d} | |
| 166 | AlkLEᵢᵢE_{iib} | E_{iib}EᵥᵢEₓ | E_{d} | |
| 167 | AlkLEᵢᵢᵢ | E_{iib}EᵥᵢEₓ | E_{d} | |
| 168 | AlkLEᵢᵥ | E_{iib}EᵥᵢEₓ | E_{d} | |
| 169 | AlkL | E_{iib}EᵥᵢEₓ | Eₑ | |
| 170 | AlkLEᵢ | E_{iib}EᵥᵢEₓ | Eₑ | |
| 171 | AlkLEᵢᵢ | E_{iib}EᵥᵢEₓ | Eₑ | |
| 172 | AlkLEᵢᵢE_{iib} | E_{iib}EᵥᵢEₓ | Eₑ | |
| 173 | AlkLEᵢᵢᵢ | E_{iib}EᵥᵢEₓ | Eₑ | |
| 174 | AlkLEᵢᵥ | E_{iib}EᵥᵢEₓ | Eₑ | |
| 175 | AlkL | E_{iib}EᵥᵢEₓ | E_{f} | |
| 176 | AlkLEᵢ | E_{iib}EᵥᵢEₓ | E_{f} | |
| 177 | AlkLEᵢᵢ | E_{iib}EᵥᵢEₓ | E_{f} | |
| 178 | AlkLEᵢᵢE_{iib} | E_{iib}EᵥᵢEₓ | E_{f} | |
| 179 | AlkLEᵢᵢᵢ | E_{iib}EᵥᵢEₓ | E_{f} | |
| 180 | AlkLEᵢᵥ | E_{iib}EᵥᵢEₓ | E_{f} | |
| 181 | AlkL | Eᵥᵢᵢᵢ | | E₁ |
| 182 | AlkLEᵢ | Eᵥᵢᵢᵢ | | E₁ |
| 183 | AlkLEᵢᵢ | Eᵥᵢᵢᵢ | | E₁ |
| 184 | AlkLEᵢᵢE_{iib} | Eᵥᵢᵢᵢ | | E₁ |
| 185 | AlkLEᵢᵢᵢ | Eᵥᵢᵢᵢ | | E₁ |
| 186 | AlkLEᵢᵥ | Eᵥᵢᵢᵢ | | E₁ |
| 187 | AlkL | Eᵥᵢᵢᵢ | Eₐ | E₁ |
| 188 | AlkLEᵢ | Eᵥᵢᵢᵢ | Eₐ | E₁ |
| 189 | AlkLEᵢᵢ | Eᵥᵢᵢᵢ | Eₐ | E₁ |
| 190 | AlkLEᵢᵢE_{iib} | Eᵥᵢᵢᵢ | Eₐ | E₁ |
| 191 | AlkLEᵢᵢᵢ | Eᵥᵢᵢᵢ | Eₐ | E₁ |
| 192 | AlkLEᵢᵥ | Eᵥᵢᵢᵢ | Eₐ | E₁ |
| 193 | AlkL | Eᵥᵢᵢᵢ | E_{b} | E₁ |
| 194 | AlkLEᵢ | Eᵥᵢᵢᵢ | E_{b} | E₁ |
| 195 | AlkLEᵢᵢ | Eᵥᵢᵢᵢ | E_{b} | E₁ |
| 196 | AlkLEᵢᵢE_{iib} | Eᵥᵢᵢᵢ | E_{b} | E₁ |
| 197 | AlkLEᵢᵢᵢ | Eᵥᵢᵢᵢ | E_{b} | E₁ |
| 198 | AlkLEᵢᵥ | Eᵥᵢᵢᵢ | E_{b} | E₁ |
| 199 | AlkL | Eᵥᵢᵢᵢ | E_{d} | E₁ |
| 200 | AlkLEᵢ | Eᵥᵢᵢᵢ | E_{d} | E₁ |
| 201 | AlkLEᵢᵢ | Eᵥᵢᵢᵢ | E_{d} | E₁ |
| 202 | AlkLEᵢᵢE_{iib} | Eᵥᵢᵢᵢ | E_{d} | E₁ |
| 203 | AlkLEᵢᵢᵢ | Eᵥᵢᵢᵢ | E_{d} | E₁ |
| 204 | AlkLEᵢᵥ | Eᵥᵢᵢᵢ | E_{d} | E₁ |
| 205 | AlkL | Eᵥᵢᵢᵢ | Eₑ | E₁ |
| 206 | AlkLEᵢ | Eᵥᵢᵢᵢ | Eₑ | E₁ |
| 207 | AlkLEᵢᵢ | Eᵥᵢᵢᵢ | Eₑ | E₁ |
| 208 | AlkLEᵢᵢE_{iib} | Eᵥᵢᵢᵢ | Eₑ | E₁ |
| 209 | AlkLEᵢᵢᵢ | Eᵥᵢᵢᵢ | Eₑ | E₁ |
| 210 | AlkLEᵢᵥ | Eᵥᵢᵢᵢ | Eₑ | E₁ |
| 211 | AlkL | Eᵥᵢᵢᵢ | E_{f} | E₁ |
| 212 | AlkLEᵢ | Eᵥᵢᵢᵢ | E_{f} | E₁ |
| 213 | AlkLEᵢᵢ | Eᵥᵢᵢᵢ | E_{f} | E₁ |
| 214 | AlkLEᵢᵢE_{iib} | Eᵥᵢᵢᵢ | E_{f} | E₁ |
| 215 | AlkLEᵢᵢᵢ | Eᵥᵢᵢᵢ | E_{f} | E₁ |
| 216 | AlkLEᵢᵥ | Eᵥᵢᵢᵢ | E_{f} | E₁ |
| 217 | AlkL | Eᵢₓ | | E₁ |
| 218 | AlkLEᵢ | Eᵢₓ | | E₁ |
| 219 | AlkLEᵢᵢ | Eᵢₓ | | E₁ |
| 220 | AlkLEᵢᵢE_{iib} | Eᵢₓ | | E₁ |
| 221 | AlkLEᵢᵢᵢ | Eᵢₓ | | E₁ |
| 222 | AlkLEᵢᵥ | Eᵢₓ | | E₁ |
| 223 | AlkL | Eᵢₓ | Eₐ | E₁ |
| 224 | AlkLEᵢ | Eᵢₓ | Eₐ | E₁ |
| 225 | AlkLEᵢᵢ | Eᵢₓ | Eₐ | E₁ |
| 226 | AlkLEᵢᵢE_{iib} | Eᵢₓ | Eₐ | E₁ |
| 227 | AlkLEᵢᵢᵢ | Eᵢₓ | Eₐ | E₁ |
| 228 | AlkLEᵢᵥ | Eᵢₓ | Eₐ | E₁ |
| 229 | AlkL | Eᵢₓ | E_{b} | E₁ |
| 230 | AlkLEᵢ | Eᵢₓ | E_{b} | E₁ |
| 231 | AlkLEᵢᵢ | Eᵢₓ | E_{b} | E₁ |
| 232 | AlkLEᵢᵢE_{iib} | Eᵢₓ | E_{b} | E₁ |
| 233 | AlkLEᵢᵢᵢ | Eᵢₓ | E_{b} | E₁ |
| 234 | AlkLEᵢᵥ | Eᵢₓ | E_{b} | E₁ |
| 235 | AlkL | Eᵢₓ | E_{d} | E₁ |
| 236 | AlkLEᵢ | Eᵢₓ | E_{d} | E₁ |
| 237 | AlkLEᵢᵢ | Eᵢₓ | E_{d} | E₁ |
| 238 | AlkLEᵢᵢE_{iib} | Eᵢₓ | E_{d} | E₁ |
| 239 | AlkLEᵢᵢᵢ | Eᵢₓ | E_{d} | E₁ |
| 240 | AlkLEᵢᵥ | Eᵢₓ | E_{d} | E₁ |
| 241 | AlkL | Eᵢₓ | Eₑ | E₁ |
| 242 | AlkLEᵢ | Eᵢₓ | Eₑ | E₁ |
| 243 | AlkLEᵢᵢ | Eᵢₓ | Eₑ | E₁ |
| 244 | AlkLEᵢᵢE_{iib} | Eᵢₓ | Eₑ | E₁ |
| 245 | AlkLEᵢᵢᵢ | Eᵢₓ | Eₑ | E₁ |
| 246 | AlkLEᵢᵥ | Eᵢₓ | Eₑ | E₁ |
| 247 | AlkL | Eᵢₓ | E_{f} | E₁ |
| 248 | AlkLEᵢ | Eᵢₓ | E_{f} | E₁ |
| 249 | AlkLEᵢᵢ | Eᵢₓ | E_{f} | E₁ |
| 250 | AlkLEᵢᵢE_{iib} | Eᵢₓ | E_{f} | E₁ |
| 251 | AlkLEᵢᵢᵢ | Eᵢₓ | E_{f} | E₁ |
| 252 | AlkLEᵢᵥ | Eᵢₓ | E_{f} | E₁ |
| 253 | AlkL | Eₓ | | E₁ |
| 254 | AlkLEᵢ | Eₓ | | E₁ |
| 255 | AlkLEᵢᵢ | Eₓ | | E₁ |
| 256 | AlkLEᵢᵢE_{iib} | Eₓ | | E₁ |
| 257 | AlkLEᵢᵢᵢ | Eₓ | | E₁ |
| 258 | AlkLEᵢᵥ | Eₓ | | E₁ |
| 259 | AlkL | Eₓ | Eₐ | E₁ |
| 260 | AlkLEᵢ | Eₓ | Eₐ | E₁ |
| 261 | AlkLEᵢᵢ | Eₓ | Eₐ | E₁ |
| 262 | AlkLEᵢᵢE_{iib} | Eₓ | Eₐ | E₁ |
| 263 | AlkLEᵢᵢᵢ | Eₓ | Eₐ | E₁ |
| 264 | AlkLEᵢᵥ | Eₓ | Eₐ | E₁ |
| 265 | AlkL | Eₓ | E_{b} | E₁ |
| 266 | AlkLEᵢ | Eₓ | E_{b} | E₁ |
| 267 | AlkLEᵢᵢ | Eₓ | E_{b} | E₁ |
| 268 | AlkLEᵢᵢE_{iib} | Eₓ | E_{b} | E₁ |
| 269 | AlkLEᵢᵢᵢ | Eₓ | E_{b} | E₁ |
| 270 | AlkLEᵢᵥ | Eₓ | E_{b} | E₁ |
| 271 | AlkL | Eₓ | E_{d} | E₁ |
| 272 | AlkLEᵢ | Eₓ | E_{d} | E₁ |
| 273 | AlkLEᵢᵢ | Eₓ | E_{d} | E₁ |
| 274 | AlkLEᵢᵢE_{iib} | Eₓ | E_{d} | E₁ |
| 275 | AlkLEᵢᵢᵢ | Eₓ | E_{d} | E₁ |
| 276 | AlkLEᵢᵥ | Eₓ | E_{d} | E₁ |
| 277 | AlkL | Eₓ | Eₑ | E₁ |
| 278 | AlkLEᵢ | Eₓ | Eₑ | E₁ |
| 279 | AlkLEᵢᵢ | Eₓ | Eₑ | E₁ |
| 280 | AlkLEᵢᵢE_{iib} | Eₓ | Eₑ | E₁ |
| 281 | AlkLEᵢᵢᵢ | Eₓ | Eₑ | E₁ |
| 282 | AlkLEᵢᵥ | Eₓ | Eₑ | E₁ |
| 283 | AlkL | Eₓ | E_{f} | E₁ |
| 284 | AlkLEᵢ | Eₓ | E_{f} | E₁ |
| 285 | AlkLEᵢᵢ | Eₓ | E_{f} | E₁ |
| 286 | AlkLEᵢᵢE_{iib} | Eₓ | E_{f} | E₁ |
| 287 | AlkLEᵢᵢᵢ | Eₓ | E_{f} | E₁ |
| 288 | AlkLEᵢᵥ | Eₓ | E_{f} | E₁ |
| 289 | AlkL | EᵥᵢEᵥᵢᵢᵢ | | E₁ |
| 290 | AlkLEᵢ | EᵥᵢEᵥᵢᵢᵢ | | E₁ |
| 291 | AlkLEᵢᵢ | EᵥᵢEᵥᵢᵢᵢ | | E₁ |
| 292 | AlkLEᵢᵢE_{iib} | EᵥᵢEᵥᵢᵢᵢ | | E₁ |
| 293 | AlkLEᵢᵢᵢ | EᵥᵢEᵥᵢᵢᵢ | | E₁ |
| 294 | AlkLEᵢᵥ | EᵥᵢEᵥᵢᵢᵢ | | E₁ |
| 295 | AlkL | EᵥᵢEᵥᵢᵢᵢ | Eₐ | E₁ |
| 296 | AlkLEᵢ | EᵥᵢEᵥᵢᵢᵢ | Eₐ | E₁ |
| 297 | AlkLEᵢᵢ | EᵥᵢEᵥᵢᵢᵢ | Eₐ | E₁ |
| 298 | AlkLEᵢᵢE_{iib} | EᵥᵢEᵥᵢᵢᵢ | Eₐ | E₁ |
| 299 | AlkLEᵢᵢᵢ | EᵥᵢEᵥᵢᵢᵢ | Eₐ | E₁ |
| 300 | AlkLEᵢᵥ | EᵥᵢEᵥᵢᵢᵢ | Eₐ | E₁ |
| 301 | AlkL | EᵥᵢEᵥᵢᵢᵢ | E_{b} | E₁ |
| 302 | AlkLEᵢ | EᵥᵢEᵥᵢᵢᵢ | E_{b} | E₁ |
| 303 | AlkLEᵢᵢ | EᵥᵢEᵥᵢᵢᵢ | E_{b} | E₁ |
| 304 | AlkLEᵢᵢE_{iib} | EᵥᵢEᵥᵢᵢᵢ | E_{b} | E₁ |
| 305 | AlkLEᵢᵢᵢ | EᵥᵢEᵥᵢᵢᵢ | E_{b} | E₁ |
| 306 | AlkLEᵢᵥ | EᵥᵢEᵥᵢᵢᵢ | E_{b} | E₁ |
| 307 | AlkL | EᵥᵢEᵥᵢᵢᵢ | E_{d} | E₁ |
| 308 | AlkLEᵢ | EᵥᵢEᵥᵢᵢᵢ | E_{d} | E₁ |
| 309 | AlkLEᵢᵢ | EᵥᵢEᵥᵢᵢᵢ | E_{d} | E₁ |
| 310 | AlkLEᵢᵢE_{iib} | EᵥᵢEᵥᵢᵢᵢ | E_{d} | E₁ |
| 311 | AlkLEᵢᵢᵢ | EᵥᵢEᵥᵢᵢᵢ | E_{d} | E₁ |
| 312 | AlkLEᵢᵥ | EᵥᵢEᵥᵢᵢᵢ | E_{d} | E₁ |
| 313 | AlkL | EᵥᵢEᵥᵢᵢᵢ | Eₑ | E₁ |
| 314 | AlkLEᵢ | EᵥᵢEᵥᵢᵢᵢ | Eₑ | E₁ |
| 315 | AlkLEᵢᵢ | EᵥᵢEᵥᵢᵢᵢ | Eₑ | E₁ |
| 316 | AlkLEᵢᵢE_{iib} | EᵥᵢEᵥᵢᵢᵢ | Eₑ | E₁ |
| 317 | AlkLEᵢᵢᵢ | EᵥᵢEᵥᵢᵢᵢ | Eₑ | E₁ |
| 318 | AlkLEᵢᵥ | EᵥᵢEᵥᵢᵢᵢ | Eₑ | E₁ |
| 319 | AlkL | EᵥᵢEᵥᵢᵢᵢ | E_{f} | E₁ |
| 320 | AlkLEᵢ | EᵥᵢEᵥᵢᵢᵢ | E_{f} | E₁ |
| 321 | AlkLEᵢᵢ | EᵥᵢEᵥᵢᵢᵢ | E_{f} | E₁ |
| 322 | AlkLEᵢᵢE_{iib} | EᵥᵢEᵥᵢᵢᵢ | E_{f} | E₁ |
| 323 | AlkLEᵢᵢᵢ | EᵥᵢEᵥᵢᵢᵢ | E_{f} | E₁ |
| 324 | AlkLEᵢᵥ | EᵥᵢEᵥᵢᵢᵢ | E_{f} | E₁ |
| 325 | AlkL | E_{iib}EᵥᵢEₓ | | E₁ |
| 326 | AlkLEᵢ | E_{iib}EᵥᵢEₓ | | E₁ |
| 327 | AlkLEᵢᵢ | E_{iib}EᵥᵢEₓ | | E₁ |
| 328 | AlkLEᵢᵢE_{iib} | E_{iib}EᵥᵢEₓ | | E₁ |
| 329 | AlkLEᵢᵢᵢ | E_{iib}EᵥᵢEₓ | | E₁ |
| 330 | AlkLEᵢᵥ | E_{iib}EᵥᵢEₓ | | E₁ |
| 331 | AlkL | E_{iib}EᵥᵢEₓ | Eₐ | E₁ |
| 332 | AlkLEᵢ | E_{iib}EᵥᵢEₓ | Eₐ | E₁ |
| 333 | AlkLEᵢᵢ | E_{iib}EᵥᵢEₓ | Eₐ | E₁ |
| 334 | AlkLEᵢᵢE_{iib} | E_{iib}EᵥᵢEₓ | Eₐ | E₁ |
| 335 | AlkLEᵢᵢᵢ | E_{iib}EᵥᵢEₓ | Eₐ | E₁ |
| 336 | AlkLEᵢᵥ | E_{iib}EᵥᵢEₓ | Eₐ | E₁ |
| 337 | AlkL | E_{iib}EᵥᵢEₓ | E_{b} | E₁ |
| 338 | AlkLEᵢ | E_{iib}EᵥᵢEₓ | E_{b} | E₁ |
| 339 | AlkLEᵢᵢ | E_{iib}EᵥᵢEₓ | E_{b} | E₁ |
| 340 | AlkLEᵢᵢE_{iib} | E_{iib}EᵥᵢEₓ | E_{b} | E₁ |
| 341 | AlkLEᵢᵢᵢ | E_{iib}EᵥᵢEₓ | E_{b} | E₁ |
| 342 | AlkLEᵢᵥ | E_{iib}EᵥᵢEₓ | E_{b} | E₁ |
| 343 | AlkL | E_{iib}EᵥᵢEₓ | E_{d} | E₁ |
| 344 | AlkLEᵢ | E_{iib}EᵥᵢEₓ | E_{d} | E₁ |
| 345 | AlkLEᵢᵢ | E_{iib}EᵥᵢEₓ | E_{d} | E₁ |
| 346 | AlkLEᵢᵢE_{iib} | E_{iib}EᵥᵢEₓ | E_{d} | E₁ |
| 347 | AlkLEᵢᵢᵢ | E_{iib}EᵥᵢEₓ | E_{d} | E₁ |
| 348 | AlkLEᵢᵥ | E_{iib}EᵥᵢEₓ | E_{d} | E₁ |
| 349 | AlkL | E_{iib}EᵥᵢEₓ | Eₑ | E₁ |
| 350 | AlkLEᵢ | E_{iib}EᵥᵢEₓ | Eₑ | E₁ |
| 351 | AlkLEᵢᵢ | E_{iib}EᵥᵢEₓ | Eₑ | E₁ |
| 352 | AlkLEᵢᵢE_{iib} | E_{iib}EᵥᵢEₓ | Eₑ | E₁ |
| 353 | AlkLEᵢᵢᵢ | E_{iib}EᵥᵢEₓ | Eₑ | E₁ |
| 354 | AlkLEᵢᵥ | E_{iib}EᵥᵢEₓ | Eₑ | E₁ |
| 355 | AlkL | E_{iib}EᵥᵢEₓ | E_{f} | E₁ |
| 356 | AlkLEᵢ | E_{iib}EᵥᵢEₓ | E_{f} | E₁ |
| 357 | AlkLEᵢᵢ | E_{iib}EᵥᵢEₓ | E_{f} | E₁ |
| 358 | AlkLEᵢᵢE_{iib} | E_{iib}EᵥᵢEₓ | E_{f} | E₁ |
| 359 | AlkLEᵢᵢᵢ | E_{iib}EᵥᵢEₓ | E_{f} | E₁ |
| 360 | AlkLEᵢᵥ | E_{iib}EᵥᵢEₓ | E_{f} | E₁ |

Microorganisms (abbreviated to MO) which are very especially preferred according to the invention are outstandingly suitable for the production of alkanes and have increased or reduced enzymatic activities (abbreviated to E) which are described in the table hereinbelow, it being possible for these enzymatic activities additionally to be advantageously combined with an enzymatic activity described for the enzyme 3-ketoacyl-ACP (acyl carrier protein) synthase III (EC 2.3.1.41), which enzymatic activity is increased in comparison with the wild type of the microorganism, in particular the enzymatic activity of plants, preferably the enzymatic activity of plants whose seeds contain fatty acids with alkyl residues shorter than 14 C atoms and especially preferably the enzymatic activity of plants of the genera Cuphea, Elaeis, Cocos, Umbellularia and Cinnamomum and gene products selected from among AccA, AccB, AccC, AccD, AceE, AceF, Lpd, AcpP, FabA, FabB, FabD, FabF, FabG, FabH, FabI, FabZ, PanD, PanK, UdhA, PntA or PntB.

Any desired combinations of at least two of these enzymatic activities may advantageously be increased.

It may be additionally advantageous that the microorganism is equipped with an enzymatic activity which is described for the gene products selected from among TdcE, PflA, PflB, PflC, PfID, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlvI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, individually or in any desired combination, and which is reduced in comparison with the wild type of the microorganism.

| | | |
|---|---|---|
| MO | Increased E | Reduced E |

| | | | | |
|---|---|---|---|---|
| 1 | AlkLEₓᵢᵢ | Eᵥᵢᵢᵢ | | |
| 2 | AlkLEₓᵢᵢEᵢ | Eᵥᵢᵢᵢ | | |
| 3 | AlkLEₓᵢᵢEᵢᵢ | Eᵥᵢᵢᵢ | | |
| 4 | AlkLEₓᵢᵢEᵢᵢE_{iib} | Eᵥᵢᵢᵢ | | |
| 5 | AlkLEₓᵢᵢEᵢᵢᵢ | Eᵥᵢᵢᵢ | | |
| 6 | AlkLEₓᵢᵢEᵢᵥ | Eᵥᵢᵢᵢ | | |
| 7 | AlkLEₓᵢᵢ | Eᵥᵢᵢᵢ | Eₐ | |
| 8 | AlkLEₓᵢᵢEᵢ | Eᵥᵢᵢᵢ | Eₐ | |
| 9 | AlkLEₓᵢᵢEᵢᵢ | Eᵥᵢᵢᵢ | Eₐ | |
| 10 | AlkLEₓᵢᵢEᵢᵢE_{iib} | Eᵥᵢᵢᵢ | Eₐ | |
| 11 | AlkLEₓᵢᵢEᵢᵢᵢ | Eᵥᵢᵢᵢ | Eₐ | |
| 12 | AlkLEₓᵢᵢEᵢᵥ | Eᵥᵢᵢᵢ | Eₐ | |
| 13 | AlkLEₓᵢᵢ | Eᵥᵢᵢᵢ | E_{b} | |
| 14 | AlkLEₓᵢᵢEᵢ | Eᵥᵢᵢᵢ | E_{b} | |
| 15 | AlkLEₓᵢᵢEᵢᵢ | Eᵥᵢᵢᵢ | E_{b} | |
| 16 | AlkLEₓᵢᵢEᵢᵢE_{iib} | Eᵥᵢᵢᵢ | E_{b} | |
| 17 | AlkLEₓᵢᵢEᵢᵢᵢ | Eᵥᵢᵢᵢ | E_{b} | |
| 18 | AlkLEₓᵢᵢEᵢᵥ | Eᵥᵢᵢᵢ | E_{b} | |
| 19 | AlkLEₓᵢᵢ | Eᵥᵢᵢᵢ | E_{d} | |
| 20 | AlkLEₓᵢᵢEᵢ | Eᵥᵢᵢᵢ | E_{d} | |
| 21 | AlkLEₓᵢᵢEᵢᵢ | Eᵥᵢᵢᵢ | E_{d} | |
| 22 | AlkLEₓᵢᵢEᵢᵢE_{iib} | Eᵥᵢᵢᵢ | E_{d} | |
| 23 | AlkLEₓᵢᵢEᵢᵢᵢ | Eᵥᵢᵢᵢ | E_{d} | |
| 24 | AlkLEₓᵢᵢEᵢᵥ | Eᵥᵢᵢᵢ | E_{d} | |
| 25 | AlkLEₓᵢᵢ | Eᵥᵢᵢᵢ | Eₑ | |
| 26 | AlkLEₓᵢᵢEᵢ | Eᵥᵢᵢᵢ | Eₑ | |
| 27 | AlkLEₓᵢᵢEᵢᵢ | Eᵥᵢᵢᵢ | Eₑ | |
| 28 | AlkLEₓᵢᵢEᵢᵢE_{iib} | Eᵥᵢᵢᵢ | Eₑ | |
| 29 | AlkLEₓᵢᵢEᵢᵢᵢ | Eᵥᵢᵢᵢ | Eₑ | |
| 30 | AlkLEₓᵢᵢEᵢᵥ | Eᵥᵢᵢᵢ | Eₑ | |
| 31 | AlkLEₓᵢᵢ | Eᵥᵢᵢᵢ | E_{f} | |
| 32 | AlkLEₓᵢᵢEᵢ | Eᵥᵢᵢᵢ | E_{f} | |
| 33 | AlkLEₓᵢᵢEᵢᵢ | Eᵥᵢᵢᵢ | E_{f} | |
| 34 | AlkLEₓᵢᵢEᵢᵢE_{iib} | Eᵥᵢᵢᵢ | E_{f} | |
| 35 | AlkLEₓᵢᵢEᵢᵢᵢ | Eᵥᵢᵢᵢ | E_{f} | |
| 36 | AlkLEₓᵢᵢEᵢᵥ | Eᵥᵢᵢᵢ | E_{f} | |
| 37 | AlkLEₓᵢᵢ | Eᵢₓ | | |
| 38 | AlkLEₓᵢᵢEᵢ | Eᵢₓ | | |
| 39 | AlkLEₓᵢᵢEᵢᵢ | Eᵢₓ | | |
| 40 | AlkLEₓᵢᵢEᵢᵢE_{iib} | Eᵢₓ | | |
| 41 | AlkLEₓᵢᵢEᵢᵢᵢ | Eᵢₓ | | |
| 42 | AlkLEₓᵢᵢEᵢᵥ | Eᵢₓ | | |
| 43 | AlkLEₓᵢᵢ | Eᵢₓ | Eₐ | |
| 44 | AlkLEₓᵢᵢEᵢ | Eᵢₓ | Eₐ | |
| 45 | AlkLEₓᵢᵢEᵢᵢ | Eᵢₓ | Eₐ | |
| 46 | AlkLEₓᵢᵢEᵢᵢE_{iib} | Eᵢₓ | Eₐ | |
| 47 | AlkLEₓᵢᵢEᵢᵢᵢ | Eᵢₓ | Eₐ | |
| 48 | AlkLEₓᵢᵢEᵢᵥ | Eᵢₓ | Eₐ | |
| 49 | AlkLEₓᵢᵢ | Eᵢₓ | E_{b} | |
| 50 | AlkLEₓᵢᵢEᵢ | Eᵢₓ | E_{b} | |
| 51 | AlkLEₓᵢᵢEᵢᵢ | Eᵢₓ | E_{b} | |
| 52 | AlkLEₓᵢᵢEᵢᵢE_{iib} | Eᵢₓ | E_{b} | |
| 53 | AlkLEₓᵢᵢEᵢᵢᵢ | Eᵢₓ | E_{b} | |
| 54 | AlkLEₓᵢᵢEᵢᵥ | Eᵢₓ | E_{b} | |
| 55 | AlkLEₓᵢᵢ | Eᵢₓ | E_{d} | |
| 56 | AlkLEₓᵢᵢEᵢ | Eᵢₓ | E_{d} | |
| 57 | AlkLEₓᵢᵢEᵢᵢ | Eᵢₓ | E_{d} | |
| 58 | AlkLEₓᵢᵢEᵢᵢE_{iib} | Eᵢₓ | E_{d} | |
| 59 | AlkLEₓᵢᵢEᵢᵢᵢ | Eᵢₓ | E_{d} | |
| 60 | AlkLEₓᵢᵢEᵢᵥ | Eᵢₓ | E_{d} | |
| 61 | AlkLEₓᵢᵢ | Eᵢₓ | Eₑ | |
| 62 | AlkLEₓᵢᵢEᵢ | Eᵢₓ | Eₑ | |
| 63 | AlkLEₓᵢᵢEᵢᵢ | Eᵢₓ | Eₑ | |
| 64 | AlkLEₓᵢᵢEᵢᵢE_{iib} | Eᵢₓ | Eₑ | |
| 65 | AlkLEₓᵢᵢEᵢᵢᵢ | Eᵢₓ | Eₑ | |
| 66 | AlkLEₓᵢᵢEᵢᵥ | Eᵢₓ | Eₑ | |
| 67 | AlkLEₓᵢᵢ | Eᵢₓ | E_{f} | |
| 68 | AlkLEₓᵢᵢEᵢ | Eᵢₓ | E_{f} | |
| 69 | AlkLEₓᵢᵢEᵢᵢ | Eᵢₓ | E_{f} | |
| 70 | AlkLEₓᵢᵢEᵢᵢE_{iib} | Eᵢₓ | E_{f} | |
| 71 | AlkLEₓᵢᵢEᵢᵢᵢ | Eᵢₓ | E_{f} | |
| 72 | AlkLEₓᵢᵢEᵢᵥ | Eᵢₓ | E_{f} | |
| 73 | AlkLEₓᵢᵢ | Eₓ | | |
| 74 | AlkLEₓᵢᵢEᵢ | Eₓ | | |
| 75 | AlkLEₓᵢᵢEᵢᵢ | Eₓ | | |
| 76 | AlkLEₓᵢᵢEᵢᵢE_{iib} | Eₓ | | |
| 77 | AlkLEₓᵢᵢEᵢᵢᵢ | Eₓ | | |
| 78 | AlkLEₓᵢᵢEᵢᵥ | Eₓ | | |
| 79 | AlkLEₓᵢᵢ | Eₓ | Eₐ | |
| 80 | AlkLEₓᵢᵢEᵢ | Eₓ | Eₐ | |
| 81 | AlkLEₓᵢᵢEᵢᵢ | Eₓ | Eₐ | |
| 82 | AlkLEₓᵢᵢEᵢᵢE_{iib} | Eₓ | Eₐ | |
| 83 | AlkLEₓᵢᵢEᵢᵢᵢ | Eₓ | Eₐ | |
| 84 | AlkLEₓᵢᵢEᵢᵥ | Eₓ | Eₐ | |
| 85 | AlkLEₓᵢᵢ | Eₓ | E_{b} | |
| 86 | AlkLEₓᵢᵢEᵢ | Eₓ | E_{b} | |
| 87 | AlkLEₓᵢᵢEᵢᵢ | Eₓ | E_{b} | |
| 88 | AlkLEₓᵢᵢEᵢᵢE_{iib} | Eₓ | E_{b} | |
| 89 | AlkLEₓᵢᵢEᵢᵢᵢ | Eₓ | E_{b} | |
| 90 | AlkLEₓᵢᵢEᵢᵥ | Eₓ | E_{b} | |
| 91 | AlkLEₓᵢᵢ | Eₓ | E_{d} | |
| 92 | AlkLEₓᵢᵢEᵢ | Eₓ | E_{d} | |
| 93 | AlkLEₓᵢᵢEᵢᵢ | Eₓ | E_{d} | |
| 94 | AlkLEₓᵢᵢEᵢᵢE_{iib} | Eₓ | E_{d} | |
| 95 | AlkLEₓᵢᵢEᵢᵢᵢ | Eₓ | E_{d} | |
| 96 | AlkLEₓᵢᵢEᵢᵥ | Eₓ | E_{d} | |
| 97 | AlkLEₓᵢᵢ | Eₓ | Eₑ | |
| 98 | AlkLEₓᵢᵢEᵢ | Eₓ | Eₑ | |
| 99 | AlkLEₓᵢᵢEᵢᵢ | Eₓ | Eₑ | |
| 100 | AlkLEₓᵢᵢEᵢᵢE_{iib} | Eₓ | Eₑ | |
| 101 | AlkLEₓᵢᵢEᵢᵢᵢ | Eₓ | Eₑ | |
| 102 | AlkLEₓᵢᵢEᵢᵥ | Eₓ | Eₑ | |
| 103 | AlkLEₓᵢᵢ | Eₓ | E_{f} | |
| 104 | AlkLEₓᵢᵢEᵢ | Eₓ | E_{f} | |
| 105 | AlkLEₓᵢᵢEᵢᵢ | Eₓ | E_{f} | |
| 106 | AlkLEₓᵢᵢEᵢᵢE_{iib} | Eₓ | E_{f} | |
| 107 | AlkLEₓᵢᵢEᵢᵢᵢ | Eₓ | E_{f} | |
| 108 | AlkLEₓᵢᵢEᵢᵥ | Eₓ | E_{f} | |
| 109 | AlkLEₓᵢᵢ | EᵥᵢEᵥᵢᵢᵢ | | |
| 110 | AlkLEₓᵢᵢEᵢ | EᵥᵢEᵥᵢᵢᵢ | | |
| 111 | AlkLEₓᵢᵢEᵢᵢ | EᵥᵢEᵥᵢᵢᵢ | | |
| 112 | AlkLEₓᵢᵢEᵢᵢE_{iib} | EᵥᵢEᵥᵢᵢᵢ | | |
| 113 | AlkLEₓᵢᵢEᵢᵢᵢ | EᵥᵢEᵥᵢᵢᵢ | | |
| 114 | AlkLEₓᵢᵢEᵢᵥ | EᵥᵢEᵥᵢᵢᵢ | | |
| 115 | AlkLEₓᵢᵢ | EᵥᵢEᵥᵢᵢᵢ | Eₐ | |
| 116 | AlkLEₓᵢᵢEᵢ | EᵥᵢEᵥᵢᵢᵢ | Eₐ | |
| 117 | AlkLEₓᵢᵢEᵢᵢ | EᵥᵢEᵥᵢᵢᵢ | Eₐ | |
| 118 | AlkLEₓᵢᵢEᵢᵢE_{iib} | EᵥᵢEᵥᵢᵢᵢ | Eₐ | |
| 119 | AlkLEₓᵢᵢEᵢᵢᵢ | EᵥᵢEᵥᵢᵢᵢ | Eₐ | |
| 120 | AlkLEₓᵢᵢEᵢᵥ | EᵥᵢEᵥᵢᵢᵢ | Eₐ | |
| 121 | AlkLEₓᵢᵢ | EᵥᵢEᵥᵢᵢᵢ | E_{b} | |
| 122 | AlkLEₓᵢᵢEᵢ | EᵥᵢEᵥᵢᵢᵢ | E_{b} | |
| 123 | AlkLEₓᵢᵢEᵢᵢ | EᵥᵢEᵥᵢᵢᵢ | E_{b} | |
| 124 | AlkLEₓᵢᵢEᵢᵢE_{iib} | EᵥᵢEᵥᵢᵢᵢ | E_{b} | |
| 125 | AlkLEₓᵢᵢEᵢᵢᵢ | EᵥᵢEᵥᵢᵢᵢ | E_{b} | |
| 126 | AlkLEₓᵢᵢEᵢᵥ | EᵥᵢEᵥᵢᵢᵢ | E_{b} | |
| 127 | AlkLEₓᵢᵢ | EᵥᵢEᵥᵢᵢᵢ | E_{d} | |
| 128 | AlkLEₓᵢᵢEᵢ | EᵥᵢEᵥᵢᵢᵢ | E_{d} | |
| 129 | AlkLEₓᵢᵢEᵢᵢ | EᵥᵢEᵥᵢᵢᵢ | E_{d} | |
| 130 | AlkLEₓᵢᵢEᵢᵢE_{iib} | EᵥᵢEᵥᵢᵢᵢ | E_{d} | |
| 131 | AlkLEₓᵢᵢEᵢᵢᵢ | EᵥᵢEᵥᵢᵢᵢ | E_{d} | |
| 132 | AlkLEₓᵢᵢEᵢᵥ | EᵥᵢEᵥᵢᵢᵢ | E_{d} | |
| 133 | AlkLEₓᵢᵢ | EᵥᵢEᵥᵢᵢᵢ | Eₑ | |
| 134 | AlkLEₓᵢᵢEᵢ | EᵥᵢEᵥᵢᵢᵢ | Eₑ | |
| 135 | AlkLEₓᵢᵢEᵢᵢ | EᵥᵢEᵥᵢᵢᵢ | Eₑ | |
| 136 | AlkLEₓᵢᵢEᵢᵢE_{iib} | EᵥᵢEᵥᵢᵢᵢ | Eₑ | |
| 137 | AlkLEₓᵢᵢEᵢᵢᵢ | EᵥᵢEᵥᵢᵢᵢ | Eₑ | |
| 138 | AlkLEₓᵢᵢEᵢᵥ | EᵥᵢEᵥᵢᵢᵢ | Eₑ | |
| 139 | AlkLEₓᵢᵢ | EᵥᵢEᵥᵢᵢᵢ | E_{f} | |
| 140 | AlkLEₓᵢᵢEᵢ | EᵥᵢEᵥᵢᵢᵢ | E_{f} | |
| 141 | AlkLEₓᵢᵢEᵢᵢ | EᵥᵢEᵥᵢᵢᵢ | E_{f} | |
| 142 | AlkLEₓᵢᵢEᵢᵢE_{iib} | EᵥᵢEᵥᵢᵢᵢ | E_{f} | |
| 143 | AlkLEₓᵢᵢEᵢᵢᵢ | EᵥᵢEᵥᵢᵢᵢ | E_{f} | |
| 144 | AlkLEₓᵢᵢEᵢᵥ | EᵥᵢEᵥᵢᵢᵢ | E_{f} | |
| 145 | AlkLEₓᵢᵢ | E_{iib}EᵥᵢEₓ | | |
| 146 | AlkLEₓᵢᵢEᵢ | E_{iib}EᵥᵢEₓ | | |
| 147 | AlkLEₓᵢᵢEᵢᵢ | E_{iib}EᵥᵢEₓ | | |
| 148 | AlkLEₓᵢᵢEᵢᵢE_{iib} | E_{iib}EᵥᵢEₓ | | |
| 149 | AlkLEₓᵢᵢEᵢᵢᵢ | E_{iib}EᵥᵢEₓ | | |
| 150 | AlkLEₓᵢᵢEᵢᵥ | E_{iib}EᵥᵢEₓ | | |
| 151 | AlkLEₓᵢᵢ | E_{iib}EᵥᵢEₓ | Eₐ | |
| 152 | AlkLEₓᵢᵢEᵢ | E_{iib}EᵥᵢEₓ | Eₐ | |
| 153 | AlkLEₓᵢᵢEᵢᵢ | E_{iib}EᵥᵢEₓ | Eₐ | |
| 154 | AlkLEₓᵢᵢEᵢᵢE_{iib} | E_{iib}EᵥᵢEₓ | Eₐ | |
| 155 | AlkLEₓᵢᵢEᵢᵢᵢ | E_{iib}EᵥᵢEₓ | Eₐ | |
| 156 | AlkLEₓᵢᵢEᵢᵥ | E_{iib}EᵥᵢEₓ | Eₐ | |
| 157 | AlkLEₓᵢᵢ | E_{iib}EᵥᵢEₓ | E_{b} | |
| 158 | AlkLEₓᵢᵢEᵢ | E_{iib}EᵥᵢEₓ | E_{b} | |
| 159 | AlkLEₓᵢᵢEᵢᵢ | E_{iib}EᵥᵢEₓ | E_{b} | |
| 160 | AlkLEₓᵢᵢEᵢᵢE_{iib} | E_{iib}EᵥᵢEₓ | E_{b} | |
| 161 | AlkLEₓᵢᵢEᵢᵢᵢ | E_{iib}EᵥᵢEₓ | E_{b} | |
| 162 | AlkLEₓᵢᵢEᵢᵥ | E_{iib}EᵥᵢEₓ | E_{b} | |
| 163 | AlkLEₓᵢᵢ | E_{iib}EᵥᵢEₓ | E_{d} | |
| 164 | AlkLEₓᵢᵢEᵢ | E_{iib}EᵥᵢEₓ | E_{d} | |
| 165 | AlkLEₓᵢᵢEᵢᵢ | E_{iib}EᵥᵢEₓ | E_{d} | |
| 166 | AlkLEₓᵢᵢEᵢᵢE_{iib} | E_{iib}EᵥᵢEₓ | E_{d} | |
| 167 | AlkLEₓᵢᵢEᵢᵢᵢ | E_{iib}EᵥᵢEₓ | E_{d} | |
| 168 | AlkLEₓᵢᵢEᵢᵥ | E_{iib}EᵥᵢEₓ | E_{d} | |
| 169 | AlkLEₓᵢᵢ | E_{iib}EᵥᵢEₓ | Eₑ | |
| 170 | AlkLEₓᵢᵢEᵢ | E_{iib}EᵥᵢEₓ | Eₑ | |
| 171 | AlkLEₓᵢᵢEᵢᵢ | E_{iib}EᵥᵢEₓ | Eₑ | |
| 172 | AlkLEₓᵢᵢEᵢᵢE_{iib} | E_{iib}EᵥᵢEₓ | Eₑ | |
| 173 | AlkLEₓᵢᵢEᵢᵢᵢ | E_{iib}EᵥᵢEₓ | Eₑ | |
| 174 | AlkLEₓᵢᵢEᵢᵥ | E_{iib}EᵥᵢEₓ | Eₑ | |
| 175 | AlkLEₓᵢᵢ | E_{iib}EᵥᵢEₓ | E_{f} | |
| 176 | AlkLEₓᵢᵢEᵢ | E_{iib}EᵥᵢEₓ | E_{f} | |
| 177 | AlkLEₓᵢᵢEᵢᵢ | E_{iib}EᵥᵢEₓ | E_{f} | |
| 178 | AlkLEₓᵢᵢEᵢᵢE_{iib} | E_{iib}EᵥᵢEₓ | E_{f} | |
| 179 | AlkLEₓᵢᵢEᵢᵢᵢ | E_{iib}EᵥᵢEₓ | E_{f} | |
| 180 | AlkLEₓᵢᵢEᵢᵥ | E_{iib}EᵥᵢEₓ | E_{f} | |
| 181 | AlkLEₓᵢᵢ | Eᵥᵢᵢᵢ | | E₁ |
| 182 | AlkLEₓᵢᵢEᵢ | Eᵥᵢᵢᵢ | | E₁ |
| 183 | AlkLEₓᵢᵢEᵢᵢ | Eᵥᵢᵢᵢ | | E₁ |
| 184 | AlkLEₓᵢᵢEᵢᵢE_{iib} | Eᵥᵢᵢᵢ | | E₁ |
| 185 | AlkLEₓᵢᵢEᵢᵢᵢ | Eᵥᵢᵢᵢ | | E₁ |
| 186 | AlkLEₓᵢᵢEᵢᵥ | Eᵥᵢᵢᵢ | | E₁ |
| 187 | AlkLEₓᵢᵢ | Eᵥᵢᵢᵢ | Eₐ | E₁ |
| 188 | AlkLEₓᵢᵢEᵢ | Eᵥᵢᵢᵢ | Eₐ | E₁ |
| 189 | AlkLEₓᵢᵢEᵢᵢ | Eᵥᵢᵢᵢ | Eₐ | E₁ |
| 190 | AlkLEₓᵢᵢEᵢᵢE_{iib} | Eᵥᵢᵢᵢ | Eₐ | E₁ |
| 191 | AlkLEₓᵢᵢEᵢᵢᵢ | Eᵥᵢᵢᵢ | Eₐ | E₁ |
| 192 | AlkLEₓᵢᵢEᵢᵥ | Eᵥᵢᵢᵢ | Eₐ | E₁ |
| 193 | AlkLEₓᵢᵢ | Eᵥᵢᵢᵢ | E_{b} | E₁ |
| 194 | AlkLEₓᵢᵢEᵢ | Eᵥᵢᵢᵢ | E_{b} | E₁ |
| 195 | AlkLEₓᵢᵢEᵢᵢ | Eᵥᵢᵢᵢ | E_{b} | E₁ |
| 196 | AlkLEₓᵢᵢEᵢᵢE_{iib} | Eᵥᵢᵢᵢ | E_{b} | E₁ |
| 197 | AlkLEₓᵢᵢEᵢᵢᵢ | Eᵥᵢᵢᵢ | E_{b} | E₁ |
| 198 | AlkLEₓᵢᵢEᵢᵥ | Eᵥᵢᵢᵢ | E_{b} | E₁ |
| 199 | AlkLEₓᵢᵢ | Eᵥᵢᵢᵢ | E_{d} | E₁ |
| 200 | AlkLEₓᵢᵢEᵢ | Eᵥᵢᵢᵢ | E_{d} | E₁ |
| 201 | AlkLEₓᵢᵢEᵢᵢ | Eᵥᵢᵢᵢ | E_{d} | E₁ |
| 202 | AlkLEₓᵢᵢEᵢᵢE_{iib} | Eᵥᵢᵢᵢ | E_{d} | E₁ |
| 203 | AlkLEₓᵢᵢEᵢᵢᵢ | Eᵥᵢᵢᵢ | E_{d} | E₁ |
| 204 | AlkLEₓᵢᵢEᵢᵥ | Eᵥᵢᵢᵢ | E_{d} | E₁ |
| 205 | AlkLEₓᵢᵢ | Eᵥᵢᵢᵢ | Eₑ | E₁ |
| 206 | AlkLEₓᵢᵢEᵢ | Eᵥᵢᵢᵢ | Eₑ | E₁ |
| 207 | AlkLEₓᵢᵢEᵢᵢ | Eᵥᵢᵢᵢ | Eₑ | E₁ |
| 208 | AlkLEₓᵢᵢEᵢᵢE_{iib} | Eᵥᵢᵢᵢ | Eₑ | E₁ |
| 209 | AlkLEₓᵢᵢEᵢᵢᵢ | Eᵥᵢᵢᵢ | Eₑ | E₁ |
| 210 | AlkLEₓᵢᵢEᵢᵥ | Eᵥᵢᵢᵢ | Eₑ | E₁ |
| 211 | AlkLEₓᵢᵢ | Eᵥᵢᵢᵢ | E_{f} | E₁ |
| 212 | AlkLEₓᵢᵢEᵢ | Eᵥᵢᵢᵢ | E_{f} | E₁ |
| 213 | AlkLEₓᵢᵢEᵢᵢ | Eᵥᵢᵢᵢ | E_{f} | E₁ |
| 214 | AlkLEₓᵢᵢEᵢᵢE_{iib} | Eᵥᵢᵢᵢ | E_{f} | E₁ |
| 215 | AlkLEₓᵢᵢEᵢᵢᵢ | Eᵥᵢᵢᵢ | E_{f} | E₁ |
| 216 | AlkLEₓᵢᵢEᵢᵥ | Eᵥᵢᵢᵢ | E_{f} | E₁ |
| 217 | AlkLEₓᵢᵢ | Eᵢₓ | | E₁ |
| 218 | AlkLEₓᵢᵢEᵢ | Eᵢₓ | | E₁ |
| 219 | AlkLEₓᵢᵢEᵢᵢ | Eᵢₓ | | E₁ |
| 220 | AlkLEₓᵢᵢEᵢᵢE_{iib} | Eᵢₓ | | E₁ |
| 221 | AlkLEₓᵢᵢEᵢᵢᵢ | Eᵢₓ | | E₁ |
| 222 | AlkLEₓᵢᵢEᵢᵥ | Eᵢₓ | | E₁ |
| 223 | AlkLEₓᵢᵢ | Eᵢₓ | Eₐ | E₁ |
| 224 | AlkLEₓᵢᵢEᵢ | Eᵢₓ | Eₐ | E₁ |
| 225 | AlkLEₓᵢᵢEᵢᵢ | Eᵢₓ | Eₐ | E₁ |
| 226 | AlkLEₓᵢᵢEᵢᵢE_{iib} | Eᵢₓ | Eₐ | E₁ |
| 227 | AlkLEₓᵢᵢEᵢᵢᵢ | Eᵢₓ | Eₐ | E₁ |
| 228 | AlkLEₓᵢᵢEᵢᵥ | Eᵢₓ | Eₐ | E₁ |
| 229 | AlkLEₓᵢᵢ | Eᵢₓ | E_{b} | E₁ |
| 230 | AlkLEₓᵢᵢEᵢ | Eᵢₓ | E_{b} | E₁ |
| 231 | AlkLEₓᵢᵢEᵢᵢ | Eᵢₓ | E_{b} | E₁ |
| 232 | AlkLEₓᵢᵢEᵢᵢE_{iib} | Eᵢₓ | E_{b} | E₁ |
| 233 | AlkLEₓᵢᵢEᵢᵢᵢ | Eᵢₓ | E_{b} | E₁ |
| 234 | AlkLEₓᵢᵢEᵢᵥ | Eᵢₓ | E_{b} | E₁ |
| 235 | AlkLEₓᵢᵢ | Eᵢₓ | E_{d} | E₁ |
| 236 | AlkLEₓᵢᵢEᵢ | Eᵢₓ | E_{d} | E₁ |
| 237 | AlkLEₓᵢᵢEᵢᵢ | Eᵢₓ | E_{d} | E₁ |
| 238 | AlkLEₓᵢᵢEᵢᵢE_{iib} | Eᵢₓ | E_{d} | E₁ |
| 239 | AlkLEₓᵢᵢEᵢᵢᵢ | Eᵢₓ | E_{d} | E₁ |
| 240 | AlkLEₓᵢᵢEᵢᵥ | Eᵢₓ | E_{d} | E₁ |
| 241 | AlkLEₓᵢᵢ | Eᵢₓ | Eₑ | E₁ |
| 242 | AlkLEₓᵢᵢEᵢ | Eᵢₓ | Eₑ | E₁ |
| 243 | AlkLEₓᵢᵢEᵢᵢ | Eᵢₓ | Eₑ | E₁ |
| 244 | AlkLEₓᵢᵢEᵢᵢE_{iib} | Eᵢₓ | Eₑ | E₁ |
| 245 | AlkLEₓᵢᵢEᵢᵢᵢ | Eᵢₓ | Eₑ | E₁ |
| 246 | AlkLEₓᵢᵢEᵢᵥ | Eᵢₓ | Eₑ | E₁ |
| 247 | AlkLEₓᵢᵢ | Eᵢₓ | E_{f} | E₁ |
| 248 | AlkLEₓᵢᵢEᵢ | Eᵢₓ | E_{f} | E₁ |
| 249 | AlkLEₓᵢᵢEᵢᵢ | Eᵢₓ | E_{f} | E₁ |
| 250 | AlkLEₓᵢᵢEᵢᵢE_{iib} | Eᵢₓ | E_{f} | E₁ |
| 251 | AlkLEₓᵢᵢEᵢᵢᵢ | Eᵢₓ | E_{f} | E₁ |
| 252 | AlkLEₓᵢᵢEᵢᵥ | Eᵢₓ | E_{f} | E₁ |
| 253 | AlkLEₓᵢᵢ | Eₓ | | E₁ |
| 254 | AlkLEₓᵢᵢEᵢ | Eₓ | | E₁ |
| 255 | AlkLEₓᵢᵢEᵢᵢ | Eₓ | | E₁ |
| 256 | AlkLEₓᵢᵢEᵢᵢE_{iib} | Eₓ | | E₁ |
| 257 | AlkLEₓᵢᵢEᵢᵢᵢ | Eₓ | | E₁ |
| 258 | AlkLEₓᵢᵢEᵢᵥ | Eₓ | | E₁ |
| 259 | AlkLEₓᵢᵢ | Eₓ | Eₐ | E₁ |
| 260 | AlkLEₓᵢᵢEᵢ | Eₓ | Eₐ | E₁ |
| 261 | AlkLEₓᵢᵢEᵢᵢ | Eₓ | Eₐ | E₁ |
| 262 | AlkLEₓᵢᵢEᵢᵢE_{iib} | Eₓ | Eₐ | E₁ |
| 263 | AlkLEₓᵢᵢEᵢᵢᵢ | Eₓ | Eₐ | E₁ |
| 264 | AlkLEₓᵢᵢEᵢᵥ | Eₓ | Eₐ | E₁ |
| 265 | AlkLEₓᵢᵢ | Eₓ | E_{b} | E₁ |
| 266 | AlkLEₓᵢᵢEᵢ | Eₓ | E_{b} | E₁ |
| 267 | AlkLEₓᵢᵢEᵢᵢ | Eₓ | E_{b} | E₁ |
| 268 | AlkLEₓᵢᵢEᵢᵢE_{iib} | Eₓ | E_{b} | E₁ |
| 269 | AlkLEₓᵢᵢEᵢᵢᵢ | Eₓ | E_{b} | E₁ |
| 270 | AlkLEₓᵢᵢEᵢᵥ | Eₓ | E_{b} | E₁ |
| 271 | AlkLEₓᵢᵢ | Eₓ | E_{d} | E₁ |
| 272 | AlkLEₓᵢᵢEᵢ | Eₓ | E_{d} | E₁ |
| 273 | AlkLEₓᵢᵢEᵢᵢ | Eₓ | E_{d} | E₁ |
| 274 | AlkLEₓᵢᵢEᵢᵢE_{iib} | Eₓ | E_{d} | E₁ |
| 275 | AlkLEₓᵢᵢEᵢᵢᵢ | Eₓ | E_{d} | E₁ |
| 276 | AlkLEₓᵢᵢEᵢᵥ | Eₓ | E_{d} | E₁ |
| 277 | AlkLEₓᵢᵢ | Eₓ | Eₑ | E₁ |
| 278 | AlkLEₓᵢᵢEᵢ | Eₓ | Eₑ | E₁ |
| 279 | AlkLEₓᵢᵢEᵢᵢ | Eₓ | Eₑ | E₁ |
| 280 | AlkLEₓᵢᵢEᵢᵢE_{iib} | Eₓ | Eₑ | E₁ |
| 281 | AlkLEₓᵢᵢEᵢᵢᵢ | Eₓ | Eₑ | E₁ |
| 282 | AlkLEₓᵢᵢEᵢᵥ | Eₓ | Eₑ | E₁ |
| 283 | AlkLEₓᵢᵢ | Eₓ | E_{f} | E₁ |
| 284 | AlkLEₓᵢᵢEᵢ | Eₓ | E_{f} | E₁ |
| 285 | AlkLEₓᵢᵢEᵢᵢ | Eₓ | E_{f} | E₁ |
| 286 | AlkLEₓᵢᵢEᵢᵢE_{iib} | Eₓ | E_{f} | E₁ |
| 287 | AlkLEₓᵢᵢEᵢᵢᵢ | Eₓ | E_{f} | E₁ |
| 288 | AlkLEₓᵢᵢEᵢᵥ | Eₓ | E_{f} | E₁ |
| 289 | AlkLEₓᵢᵢ | EᵥᵢEᵥᵢᵢᵢ | | E₁ |
| 290 | AlkLEₓᵢᵢEᵢ | EᵥᵢEᵥᵢᵢᵢ | | E₁ |
| 291 | AlkLEₓᵢᵢEᵢᵢ | EᵥᵢEᵥᵢᵢᵢ | | E₁ |
| 292 | AlkLEₓᵢᵢEᵢᵢE_{iib} | EᵥᵢEᵥᵢᵢᵢ | | E₁ |
| 293 | AlkLEₓᵢᵢEᵢᵢᵢ | EᵥᵢEᵥᵢᵢᵢ | | E₁ |
| 294 | AlkLEₓᵢᵢEᵢᵥ | EᵥᵢEᵥᵢᵢᵢ | | E₁ |
| 295 | AlkLEₓᵢᵢ | EᵥᵢEᵥᵢᵢᵢ | Eₐ | E₁ |
| 296 | AlkLEₓᵢᵢEᵢ | EᵥᵢEᵥᵢᵢᵢ | Eₐ | E₁ |
| 297 | AlkLEₓᵢᵢEᵢᵢ | EᵥᵢEᵥᵢᵢᵢ | Eₐ | E₁ |
| 298 | AlkLEₓᵢᵢEᵢᵢE_{iib} | EᵥᵢEᵥᵢᵢᵢ | Eₐ | E₁ |
| 299 | AlkLEₓᵢᵢEᵢᵢᵢ | EᵥᵢEᵥᵢᵢᵢ | Eₐ | E₁ |
| 300 | AlkLEₓᵢᵢEᵢᵥ | EᵥᵢEᵥᵢᵢᵢ | Eₐ | E₁ |
| 301 | AlkLEₓᵢᵢ | EᵥᵢEᵥᵢᵢᵢ | E_{b} | E₁ |
| 302 | AlkLEₓᵢᵢEᵢ | EᵥᵢEᵥᵢᵢᵢ | E_{b} | E₁ |
| 303 | AlkLEₓᵢᵢEᵢᵢ | EᵥᵢEᵥᵢᵢᵢ | E_{b} | E₁ |
| 304 | AlkLEₓᵢᵢEᵢᵢE_{iib} | EᵥᵢEᵥᵢᵢᵢ | E_{b} | E₁ |
| 305 | AlkLEₓᵢᵢEᵢᵢᵢ | EᵥᵢEᵥᵢᵢᵢ | E_{b} | E₁ |
| 306 | AlkLEₓᵢᵢEᵢᵥ | EᵥᵢEᵥᵢᵢᵢ | E_{b} | E₁ |
| 307 | AlkLEₓᵢᵢ | EᵥᵢEᵥᵢᵢᵢ | E_{d} | E₁ |
| 308 | AlkLEₓᵢᵢEᵢ | EᵥᵢEᵥᵢᵢᵢ | E_{d} | E₁ |
| 309 | AlkLEₓᵢᵢEᵢᵢ | EᵥᵢEᵥᵢᵢᵢ | E_{d} | E₁ |
| 310 | AlkLEₓᵢᵢEᵢᵢE_{iib} | EᵥᵢEᵥᵢᵢᵢ | E_{d} | E₁ |
| 311 | AlkLEₓᵢᵢEᵢᵢᵢ | EᵥᵢEᵥᵢᵢᵢ | E_{d} | E₁ |
| 312 | AlkLEₓᵢᵢEᵢᵥ | EᵥᵢEᵥᵢᵢᵢ | E_{d} | E₁ |
| 313 | AlkLEₓᵢᵢ | EᵥᵢEᵥᵢᵢᵢ | Eₑ | E₁ |
| 314 | AlkLEₓᵢᵢEᵢ | EᵥᵢEᵥᵢᵢᵢ | Eₑ | E₁ |
| 315 | AlkLEₓᵢᵢEᵢᵢ | EᵥᵢEᵥᵢᵢᵢ | Eₑ | E₁ |
| 316 | AlkLEₓᵢᵢEᵢᵢE_{iib} | EᵥᵢEᵥᵢᵢᵢ | Eₑ | E₁ |
| 317 | AlkLEₓᵢᵢEᵢᵢᵢ | EᵥᵢEᵥᵢᵢᵢ | Eₑ | E₁ |
| 318 | AlkLEₓᵢᵢEᵢᵥ | EᵥᵢEᵥᵢᵢᵢ | Eₑ | E₁ |
| 319 | AlkLEₓᵢᵢ | EᵥᵢEᵥᵢᵢᵢ | E_{f} | E₁ |
| 320 | AlkLEₓᵢᵢEᵢ | EᵥᵢEᵥᵢᵢᵢ | E_{f} | E₁ |
| 321 | AlkLEₓᵢᵢEᵢᵢ | EᵥᵢEᵥᵢᵢᵢ | E_{f} | E₁ |
| 322 | AlkLEₓᵢᵢEᵢᵢE_{iib} | EᵥᵢEᵥᵢᵢᵢ | E_{f} | E₁ |
| 323 | AlkLEₓᵢᵢEᵢᵢᵢ | EᵥᵢEᵥᵢᵢᵢ | E_{f} | E₁ |
| 324 | AlkLEₓᵢᵢEᵢᵥ | EᵥᵢEᵥᵢᵢᵢ | E_{f} | E₁ |
| 325 | AlkLEₓᵢᵢ | E_{iib}EᵥᵢEₓ | | E₁ |
| 326 | AlkLEₓᵢᵢEᵢ | E_{iib}EᵥᵢEₓ | | E₁ |
| 327 | AlkLEₓᵢᵢEᵢᵢ | E_{iib}EᵥᵢEₓ | | E₁ |
| 328 | AlkLEₓᵢᵢEᵢᵢE_{iib} | E_{iib}EᵥᵢEₓ | | E₁ |
| 329 | AlkLEₓᵢᵢEᵢᵢᵢ | E_{iib}EᵥᵢEₓ | | E₁ |
| 330 | AlkLEₓᵢᵢEᵢᵥ | E_{iib}EᵥᵢEₓ | | E₁ |
| 331 | AlkLEₓᵢᵢ | E_{iib}EᵥᵢEₓ | Eₐ | E₁ |
| 332 | AlkLEₓᵢᵢEᵢ | E_{iib}EᵥᵢEₓ | Eₐ | E₁ |
| 333 | AlkLEₓᵢᵢEᵢᵢ | E_{iib}EᵥᵢEₓ | Eₐ | E₁ |
| 334 | AlkLEₓᵢᵢEᵢᵢE_{iib} | E_{iib}EᵥᵢEₓ | Eₐ | E₁ |
| 335 | AlkLEₓᵢᵢEᵢᵢᵢ | E_{iib}EᵥᵢEₓ | Eₐ | E₁ |
| 336 | AlkLEₓᵢᵢEᵢᵥ | E_{iib}EᵥᵢEₓ | Eₐ | E₁ |
| 337 | AlkLEₓᵢᵢ | E_{iib}EᵥᵢEₓ | E_{b} | E₁ |
| 338 | AlkLEₓᵢᵢEᵢ | E_{iib}EᵥᵢEₓ | E_{b} | E₁ |
| 339 | AlkLEₓᵢᵢEᵢᵢ | E_{iib}EᵥᵢEₓ | E_{b} | E₁ |
| 340 | AlkLEₓᵢᵢEᵢᵢE_{iib} | E_{iib}EᵥᵢEₓ | E_{b} | E₁ |
| 341 | AlkLEₓᵢᵢEᵢᵢᵢ | E_{iib}EᵥᵢEₓ | E_{b} | E₁ |
| 342 | AlkLEₓᵢᵢEᵢᵥ | E_{iib}EᵥᵢEₓ | E_{b} | E₁ |
| 343 | AlkLEₓᵢᵢ | E_{iib}EᵥᵢEₓ | E_{d} | E₁ |
| 344 | AlkLEₓᵢᵢEᵢ | E_{iib}EᵥᵢEₓ | E_{d} | E₁ |
| 345 | AlkLEₓᵢᵢEᵢᵢ | E_{iib}EᵥᵢEₓ | E_{d} | E₁ |
| 346 | AlkLEₓᵢᵢEᵢᵢE_{iib} | E_{iib}EᵥᵢEₓ | E_{d} | E₁ |
| 347 | AlkLEₓᵢᵢEᵢᵢᵢ | E_{iib}EᵥᵢEₓ | E_{d} | E₁ |
| 348 | AlkLEₓᵢᵢEᵢᵥ | E_{iib}EᵥᵢEₓ | E_{d} | E₁ |
| 349 | AlkLEₓᵢᵢ | E_{iib}EᵥᵢEₓ | Eₑ | E₁ |
| 350 | AlkLEₓᵢᵢEᵢ | E_{iib}EᵥᵢEₓ | Eₑ | E₁ |
| 351 | AlkLEₓᵢᵢEᵢᵢ | E_{iib}EᵥᵢEₓ | Eₑ | E₁ |
| 352 | AlkLEₓᵢᵢEᵢᵢE_{iib} | E_{iib}EᵥᵢEₓ | Eₑ | E₁ |
| 353 | AlkLEₓᵢᵢEᵢᵢᵢ | E_{iib}EᵥᵢEₓ | Eₑ | E₁ |
| 354 | AlkLEₓᵢᵢEᵢᵥ | E_{iib}EᵥᵢEₓ | Eₑ | E₁ |
| 355 | AlkLEₓᵢᵢ | E_{iib}EᵥᵢEₓ | E_{f} | E₁ |
| 356 | AlkLEₓᵢᵢEᵢ | E_{iib}EᵥᵢEₓ | E_{f} | E₁ |
| 357 | AlkLEₓᵢᵢEᵢᵢ | E_{iib}EᵥᵢEₓ | E_{f} | E₁ |
| 358 | AlkLEₓᵢᵢEᵢᵢE_{iib} | E_{iib}EᵥᵢEₓ | E_{f} | E₁ |
| 359 | AlkLEₓᵢᵢEᵢᵢᵢ | E_{iib}EᵥᵢEₓ | E_{f} | E₁ |
| 360 | AlkLEₓᵢᵢEᵢᵥ | E_{iib}EᵥᵢEₓ | E_{f} | E₁ |

Microorganisms (abbreviated to MO) which are very especially preferred according to the invention are outstandingly suitable for the production of terminal olefins and have increased or reduced enzymatic activities (abbreviated to E) which are described in the table hereinbelow, it being possible for these enzymatic activities additionally to be advantageously combined with an enzymatic activity described for the enzyme 3-ketoacyl-ACP (acyl carrier protein) synthase III (EC 2.3.1.41), which enzymatic activity is increased in comparison with the wild type of the microorganism, in particular the enzymatic activity of plants, preferably the enzymatic activity of plants whose seeds contain fatty acids with alkyl residues shorter than 14 C atoms and especially preferably the enzymatic activity of plants of the genera Cuphea, Elaeis, Cocos, Umbellularia and Cinnamomum and gene products selected from among AccA, AccB, AccC, AccD, AceE, AceF, Lpd, AcpP, FabA, FabB, FabD, FabF, FabG, FabH, FabI, FabZ, PanD, PanK, UdhA, PntA or PntB.

Any desired combinations of at least two of these enzymatic activities may advantageously be increased.

It may be additionally advantageous that the microorganism is equipped with an enzymatic activity which is described for the gene products selected from among TdcE, PflA, PflB, PflC, PfID, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, individually or in any desired combination, and which is reduced in comparison with the wild type of the microorganism.

| | | |
|---|---|---|
| MO | Increased E | Reduced E |

| | | | | |
|---|---|---|---|---|
| 1 | AlkL | Eₓᵢ | | |
| 2 | AlkLEᵢ | Eₓᵢ | | |
| 3 | AlkLEᵢᵢ | Eₓᵢ | | |
| 4 | AlkLEᵢᵢE_{iib} | Eₓᵢ | | |
| 5 | AlkLEᵢᵢᵢ | Eₓᵢ | | |
| 6 | AlkLEᵢᵥ | Eₓᵢ | | |
| 7 | AlkL | Eₓᵢ | Eₐ | |
| 8 | AlkLEᵢ | Eₓᵢ | Eₐ | |
| 9 | AlkLEᵢᵢ | Eₓᵢ | Eₐ | |
| 10 | AlkLEᵢᵢE_{iib} | Eₓᵢ | Eₐ | |
| 11 | AlkLEᵢᵢᵢ | Eₓᵢ | Eₐ | |
| 12 | AlkLEᵢᵥ | Eₓᵢ | Eₐ | |
| 13 | AlkL | Eₓᵢ | E_{b} | |
| 14 | AlkLEᵢ | Eₓᵢ | E_{b} | |
| 15 | AlkLEᵢᵢ | Eₓᵢ | E_{b} | |
| 16 | AlkLEᵢᵢE_{iib} | Eₓᵢ | E_{b} | |
| 17 | AlkLEᵢᵢᵢ | Eₓᵢ | E_{b} | |
| 18 | AlkLEᵢᵥ | Eₓᵢ | E_{b} | |
| 19 | AlkL | Eₓᵢ | E_{d} | |
| 20 | AlkLEᵢ | Eₓᵢ | E_{d} | |
| 21 | AlkLEᵢᵢ | Eₓᵢ | E_{d} | |
| 22 | AlkLEᵢᵢE_{iib} | Eₓᵢ | E_{d} | |
| 23 | AlkLEᵢᵢᵢ | Eₓᵢ | E_{d} | |
| 24 | AlkLEᵢᵥ | Eₓᵢ | E_{d} | |
| 25 | AlkL | Eₓᵢ | Eₑ | |
| 26 | AlkLEᵢ | Eₓᵢ | Eₑ | |
| 27 | AlkLEᵢᵢ | Eₓᵢ | Eₑ | |
| 28 | AlkLEᵢᵢE_{iib} | Eₓᵢ | Eₑ | |
| 29 | AlkLEᵢᵢᵢ | Eₓᵢ | Eₑ | |
| 30 | AlkLEᵢᵥ | Eₓᵢ | Eₑ | |
| 31 | AlkL | Eₓᵢ | E_{f} | |
| 32 | AlkLEᵢ | Eₓᵢ | E_{f} | |
| 33 | AlkLEᵢᵢ | Eₓᵢ | E_{f} | |
| 34 | AlkLEᵢᵢE_{iib} | Eₓᵢ | E_{f} | |
| 35 | AlkLEᵢᵢᵢ | Eₓᵢ | E_{f} | |
| 36 | AlkLEᵢᵥ | Eₓᵢ | E_{f} | |
| 37 | AlkL | Eₓᵢ | | E₁ |
| 38 | AlkLEᵢ | Eₓᵢ | | E₁ |
| 39 | AlkLEᵢᵢ | Eₓᵢ | | E₁ |
| 40 | AlkLEᵢᵢE_{iib} | Eₓᵢ | | E₁ |
| 41 | AlkLEᵢᵢᵢ | Eₓᵢ | | E₁ |
| 42 | AlkLEᵢᵥ | Eₓᵢ | | E₁ |
| 43 | AlkL | Eₓᵢ | Eₐ | E₁ |
| 44 | AlkLEᵢ | Eₓᵢ | Eₐ | E₁ |
| 45 | AlkLEᵢᵢ | Eₓᵢ | Eₐ | E₁ |
| 46 | AlkLEᵢᵢE_{iib} | Eₓᵢ | Eₐ | E₁ |
| 47 | AlkLEᵢᵢᵢ | Eₓᵢ | Eₐ | E₁ |
| 48 | AlkLEᵢᵥ | Eₓᵢ | Eₐ | E₁ |
| 49 | AlkL | Eₓᵢ | E_{b} | E₁ |
| 50 | AlkLEᵢ | Eₓᵢ | E_{b} | E₁ |
| 51 | AlkLEᵢᵢ | Eₓᵢ | E_{b} | E₁ |
| 52 | AlkLEᵢᵢE_{iib} | Eₓᵢ | E_{b} | E₁ |
| 53 | AlkLEᵢᵢᵢ | Eₓᵢ | E_{b} | E₁ |
| 54 | AlkLEᵢᵥ | Eₓᵢ | E_{b} | E₁ |
| 55 | AlkL | Eₓᵢ | E_{d} | E₁ |
| 56 | AlkLEᵢ | Eₓᵢ | E_{d} | E₁ |
| 57 | AlkLEᵢᵢ | Eₓᵢ | E_{d} | E₁ |
| 58 | AlkLEᵢᵢE_{iib} | Eₓᵢ | E_{d} | E₁ |
| 59 | AlkLEᵢᵢᵢ | Eₓᵢ | E_{d} | E₁ |
| 60 | AlkLEᵢᵥ | Eₓᵢ | E_{d} | E₁ |
| 61 | AlkL | Eₓᵢ | Eₑ | E₁ |
| 62 | AlkLEᵢ | Eₓᵢ | Eₑ | E₁ |
| 63 | AlkLEᵢᵢ | Eₓᵢ | Eₑ | E₁ |
| 64 | AlkLEᵢᵢE_{iib} | Eₓᵢ | Eₑ | E₁ |
| 65 | AlkLEᵢᵢᵢ | Eₓᵢ | Eₑ | E₁ |
| 66 | AlkLEᵢᵥ | Eₓᵢ | Eₑ | E₁ |
| 67 | AlkL | Eₓᵢ | E_{f} | E₁ |
| 68 | AlkLEᵢ | Eₓᵢ | E_{f} | E₁ |
| 69 | AlkLEᵢᵢ | Eₓᵢ | E_{f} | E₁ |
| 70 | AlkLEᵢᵢE_{iib} | Eₓᵢ | E_{f} | E₁ |
| 71 | AlkLEᵢᵢᵢ | Eₓᵢ | E_{f} | E₁ |
| 72 | AlkLEᵢᵥ | Eₓᵢ | E_{f} | E₁ |

Microorganisms (abbreviated to MO) which are very especially preferred according to the invention are outstandingly suitable for the production of alkan-1-amines and have increased or reduced enzymatic activities (abbreviated to E) which are described in the table hereinbelow, it being possible for these enzymatic activities additionally to be advantageously combined with an enzymatic activity described for the enzyme 3-ketoacyl-ACP (acyl carrier protein) synthase III (EC 2.3.1.41), which enzymatic activity is increased in comparison with the wild type of the microorganism, in particular the enzymatic activity of plants, preferably the enzymatic activity of plants whose seeds contain fatty acids with alkyl residues shorter than 14 C atoms and especially preferably the enzymatic activity of plants of the genera Cuphea, Elaeis, Cocos, Umbellularia and Cinnamomum and gene products selected from among AccA, AccB, AccC, AccD, AceE, AceF, Lpd, AcpP, FabA, FabB, FabD, FabF, FabG, FabH, FabI, FabZ, PanD, PanK, UdhA, PntA or PntB.

Any desired combinations of at least two of these enzymatic activities may advantageously be increased.

It may be additionally advantageous that the microorganism is equipped with an enzymatic activity which is described for the gene products selected from among TdcE, PflA, PflB, PflC, PfID, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GRA, FabR, FhuA, Dld, LldA or LldP, individually or in any desired combination, and which is reduced in comparison with the wild type of the microorganism.

| | | |
|---|---|---|
| MO | Increased E | Reduced E |

| | | | | |
|---|---|---|---|---|
| 1 | AlkLEᵥᵢᵢ | Eᵥᵢᵢᵢ | | |
| 2 | AlkLEᵥᵢᵢEᵢ | Eᵥᵢᵢᵢ | | |
| 3 | AlkLEᵥᵢᵢEᵢᵢ | Eᵥᵢᵢᵢ | | |
| 4 | AlkLEᵥᵢᵢEᵢᵢE_{iib} | Eᵥᵢᵢᵢ | | |
| 5 | AlkLEᵥᵢᵢEᵢᵢᵢ | Eᵥᵢᵢᵢ | | |
| 6 | AlkLEᵥᵢᵢEᵢᵥ | Eᵥᵢᵢᵢ | | |
| 7 | AlkLEᵥᵢᵢ | Eᵥᵢᵢᵢ | Eₐ | |
| 8 | AlkLEᵥᵢᵢEᵢ | Eᵥᵢᵢᵢ | Eₐ | |
| 9 | AlkLEᵥᵢᵢEᵢᵢ | Eᵥᵢᵢᵢ | Eₐ | |
| 10 | AlkLEᵥᵢᵢEᵢᵢE_{iib} | Eᵥᵢᵢᵢ | Eₐ | |
| 11 | AlkLEᵥᵢᵢEᵢᵢᵢ | Eᵥᵢᵢᵢ | Eₐ | |
| 12 | AlkLEᵥᵢᵢEᵢᵥ | Eᵥᵢᵢᵢ | Eₐ | |
| 13 | AlkLEᵥᵢᵢ | Eᵥᵢᵢᵢ | E_{b} | |
| 14 | AlkLEᵥᵢᵢEᵢ | Eᵥᵢᵢᵢ | E_{b} | |
| 15 | AlkLEᵥᵢᵢEᵢᵢ | Eᵥᵢᵢᵢ | E_{b} | |
| 16 | AlkLEᵥᵢᵢEᵢᵢE_{iib} | Eᵥᵢᵢᵢ | E_{b} | |
| 17 | AlkLEᵥᵢᵢEᵢᵢᵢ | Eᵥᵢᵢᵢ | E_{b} | |
| 18 | AlkLEᵥᵢᵢEᵢᵥ | Eᵥᵢᵢᵢ | E_{b} | |
| 19 | AlkLEᵥᵢᵢ | Eᵥᵢᵢᵢ | E_{d} | |
| 20 | AlkLEᵥᵢᵢEᵢ | Eᵥᵢᵢᵢ | E_{d} | |
| 21 | AlkLEᵥᵢᵢEᵢᵢ | Eᵥᵢᵢᵢ | E_{d} | |
| 22 | AlkLEᵥᵢᵢEᵢᵢE_{iib} | Eᵥᵢᵢᵢ | E_{d} | |
| 23 | AlkLEᵥᵢᵢEᵢᵢᵢ | Eᵥᵢᵢᵢ | E_{d} | |
| 24 | AlkLEᵥᵢᵢEᵢᵥ | Eᵥᵢᵢᵢ | E_{d} | |
| 25 | AlkLEᵥᵢᵢ | Eᵥᵢᵢᵢ | Eₑ | |
| 26 | AlkLEᵥᵢᵢEᵢ | Eᵥᵢᵢᵢ | Eₑ | |
| 27 | AlkLEᵥᵢᵢEᵢᵢ | Eᵥᵢᵢᵢ | Eₑ | |
| 28 | AlkLEᵥᵢᵢEᵢᵢE_{iib} | Eᵥᵢᵢᵢ | Eₑ | |
| 29 | AlkLEᵥᵢᵢEᵢᵢᵢ | Eᵥᵢᵢᵢ | Eₑ | |
| 30 | AlkLEᵥᵢᵢEᵢᵥ | Eᵥᵢᵢᵢ | Eₑ | |
| 31 | AlkLEᵥᵢᵢ | Eᵥᵢᵢᵢ | E_{f} | |
| 32 | AlkLEᵥᵢᵢEᵢ | Eᵥᵢᵢᵢ | E_{f} | |
| 33 | AlkLEᵥᵢᵢEᵢᵢ | Eᵥᵢᵢᵢ | E_{f} | |
| 34 | AlkLEᵥᵢᵢEᵢᵢE_{iib} | Eᵥᵢᵢᵢ | E_{f} | |
| 35 | AlkLEᵥᵢᵢEᵢᵢᵢ | Eᵥᵢᵢᵢ | E_{f} | |
| 36 | AlkLEᵥᵢᵢEᵢᵥ | Eᵥᵢᵢᵢ | E_{f} | |
| 37 | AlkLEᵥᵢᵢ | Eᵢₓ | | |
| 38 | AlkLEᵥᵢᵢEᵢ | Eᵢₓ | | |
| 39 | AlkLEᵥᵢᵢEᵢᵢ | Eᵢₓ | | |
| 40 | AlkLEᵥᵢᵢEᵢᵢE_{iib} | Eᵢₓ | | |
| 41 | AlkLEᵥᵢᵢEᵢᵢᵢ | Eᵢₓ | | |
| 42 | AlkLEᵥᵢᵢEᵢᵥ | Eᵢₓ | | |
| 43 | AlkLEᵥᵢᵢ | Eᵢₓ | Eₐ | |
| 44 | AlkLEᵥᵢᵢEᵢ | Eᵢₓ | Eₐ | |
| 45 | AlkLEᵥᵢᵢEᵢᵢ | Eᵢₓ | Eₐ | |
| 46 | AlkLEᵥᵢᵢEᵢᵢE_{iib} | Eᵢₓ | Eₐ | |
| 47 | AlkLEᵥᵢᵢEᵢᵢᵢ | Eᵢₓ | Eₐ | |
| 48 | AlkLEᵥᵢᵢEᵢᵥ | Eᵢₓ | Eₐ | |
| 49 | AlkLEᵥᵢᵢ | Eᵢₓ | E_{b} | |
| 50 | AlkLEᵥᵢᵢEᵢ | Eᵢₓ | E_{b} | |
| 51 | AlkLEᵥᵢᵢEᵢᵢ | Eᵢₓ | E_{b} | |
| 52 | AlkLEᵥᵢᵢEᵢᵢE_{iib} | Eᵢₓ | E_{b} | |
| 53 | AlkLEᵥᵢᵢEᵢᵢᵢ | Eᵢₓ | E_{b} | |
| 54 | AlkLEᵥᵢᵢEᵢᵥ | Eᵢₓ | E_{b} | |
| 55 | AlkLEᵥᵢᵢ | Eᵢₓ | E_{d} | |
| 56 | AlkLEᵥᵢᵢEᵢ | Eᵢₓ | E_{d} | |
| 57 | AlkLEᵥᵢᵢEᵢᵢ | Eᵢₓ | E_{d} | |
| 58 | AlkLEᵥᵢᵢEᵢᵢE_{iib} | Eᵢₓ | E_{d} | |
| 59 | AlkLEᵥᵢᵢEᵢᵢᵢ | Eᵢₓ | E_{d} | |
| 60 | AlkLEᵥᵢᵢEᵢᵥ | Eᵢₓ | E_{d} | |
| 61 | AlkLEᵥᵢᵢ | Eᵢₓ | Eₑ | |
| 62 | AlkLEᵥᵢᵢEᵢ | Eᵢₓ | Eₑ | |
| 63 | AlkLEᵥᵢᵢEᵢᵢ | Eᵢₓ | Eₑ | |
| 64 | AlkLEᵥᵢᵢEᵢᵢE_{iib} | Eᵢₓ | Eₑ | |
| 65 | AlkLEᵥᵢᵢEᵢᵢᵢ | Eᵢₓ | Eₑ | |
| 66 | AlkLEᵥᵢᵢEᵢᵥ | Eᵢₓ | Eₑ | |
| 67 | AlkLEᵥᵢᵢ | Eᵢₓ | E_{f} | |
| 68 | AlkLEᵥᵢᵢEᵢ | Eᵢₓ | E_{f} | |
| 69 | AlkLEᵥᵢᵢEᵢᵢ | Eᵢₓ | E_{f} | |
| 70 | AlkLEᵥᵢᵢEᵢᵢE_{iib} | Eᵢₓ | E_{f} | |
| 71 | AlkLEᵥᵢᵢEᵢᵢᵢ | Eᵢₓ | E_{f} | |
| 72 | AlkLEᵥᵢᵢEᵢᵥ | Eᵢₓ | E_{f} | |
| 73 | AlkLEᵥᵢᵢ | Eₓ | | |
| 74 | AlkLEᵥᵢᵢEᵢ | Eₓ | | |
| 75 | AlkLEᵥᵢᵢEᵢᵢ | Eₓ | | |
| 76 | AlkLEᵥᵢᵢEᵢᵢE_{iib} | Eₓ | | |
| 77 | AlkLEᵥᵢᵢEᵢᵢᵢ | Eₓ | | |
| 78 | AlkLEᵥᵢᵢEᵢᵥ | Eₓ | | |
| 79 | AlkLEᵥᵢᵢ | Eₓ | Eₐ | |
| 80 | AlkLEᵥᵢᵢEᵢ | Eₓ | Eₐ | |
| 81 | AlkLEᵥᵢᵢEᵢᵢ | Eₓ | Eₐ | |
| 82 | AlkLEᵥᵢᵢEᵢᵢE_{iib} | Eₓ | Eₐ | |
| 83 | AlkLEᵥᵢᵢEᵢᵢᵢ | Eₓ | Eₐ | |
| 84 | AlkLEᵥᵢᵢEᵢᵥ | Eₓ | Eₐ | |
| 85 | AlkLEᵥᵢᵢ | Eₓ | E_{b} | |
| 86 | AlkLEᵥᵢᵢEᵢ | Eₓ | E_{b} | |
| 87 | AlkLEᵥᵢᵢEᵢᵢ | Eₓ | E_{b} | |
| 88 | AlkLEᵥᵢᵢEᵢᵢE_{iib} | Eₓ | E_{b} | |
| 89 | AlkLEᵥᵢᵢEᵢᵢᵢ | Eₓ | E_{b} | |
| 90 | AlkLEᵥᵢᵢEᵢᵥ | Eₓ | E_{b} | |
| 91 | AlkLEᵥᵢᵢ | Eₓ | E_{d} | |
| 92 | AlkLEᵥᵢᵢEᵢ | Eₓ | E_{d} | |
| 93 | AlkLEᵥᵢᵢEᵢᵢ | Eₓ | E_{d} | |
| 94 | AlkLEᵥᵢᵢEᵢᵢE_{iib} | Eₓ | E_{d} | |
| 95 | AlkLEᵥᵢᵢEᵢᵢᵢ | Eₓ | E_{d} | |
| 96 | AlkLEᵥᵢᵢEᵢᵥ | Eₓ | E_{d} | |
| 97 | AlkLEᵥᵢᵢ | Eₓ | Eₑ | |
| 98 | AlkLEᵥᵢᵢEᵢ | Eₓ | Eₑ | |
| 99 | AlkLEᵥᵢᵢEᵢᵢ | Eₓ | Eₑ | |
| 100 | AlkLEᵥᵢᵢEᵢᵢE_{iib} | Eₓ | Eₑ | |
| 101 | AlkLEᵥᵢᵢEᵢᵢᵢ | Eₓ | Eₑ | |
| 102 | AlkLEᵥᵢᵢEᵢᵥ | Eₓ | Eₑ | |
| 103 | AlkLEᵥᵢᵢ | Eₓ | E_{f} | |
| 104 | AlkLEᵥᵢᵢEᵢ | Eₓ | E_{f} | |
| 105 | AlkLEᵥᵢᵢEᵢᵢ | Eₓ | E_{f} | |
| 106 | AlkLEᵥᵢᵢEᵢᵢE_{iib} | Eₓ | E_{f} | |
| 107 | AlkLEᵥᵢᵢEᵢᵢᵢ | Eₓ | E_{f} | |
| 108 | AlkLEᵥᵢᵢEᵢᵥ | Eₓ | E_{f} | |
| 109 | AlkLEᵥᵢᵢ | EᵥᵢEᵥᵢᵢᵢ | | |
| 110 | AlkLEᵥᵢᵢEᵢ | EᵥᵢEᵥᵢᵢᵢ | | |
| 111 | AlkLEᵥᵢᵢEᵢᵢ | EᵥᵢEᵥᵢᵢᵢ | | |
| 112 | AlkLEᵥᵢᵢEᵢᵢE_{iib} | EᵥᵢEᵥᵢᵢᵢ | | |
| 113 | AlkLEᵥᵢᵢEᵢᵢᵢ | EᵥᵢEᵥᵢᵢᵢ | | |
| 114 | AlkLEᵥᵢᵢEᵢᵥ | EᵥᵢEᵥᵢᵢᵢ | | |
| 115 | AlkLEᵥᵢᵢ | EᵥᵢEᵥᵢᵢᵢ | Eₐ | |
| 116 | AlkLEᵥᵢᵢEᵢ | EᵥᵢEᵥᵢᵢᵢ | Eₐ | |
| 117 | AlkLEᵥᵢᵢEᵢᵢ | EᵥᵢEᵥᵢᵢᵢ | Eₐ | |
| 118 | AlkLEᵥᵢᵢEᵢᵢE_{iib} | EᵥᵢEᵥᵢᵢᵢ | Eₐ | |
| 119 | AlkLEᵥᵢᵢEᵢᵢᵢ | EᵥᵢEᵥᵢᵢᵢ | Eₐ | |
| 120 | AlkLEᵥᵢᵢEᵢᵥ | EᵥᵢEᵥᵢᵢᵢ | Eₐ | |
| 121 | AlkLEᵥᵢᵢ | EᵥᵢEᵥᵢᵢᵢ | E_{b} | |
| 122 | AlkLEᵥᵢᵢEᵢ | EᵥᵢEᵥᵢᵢᵢ | E_{b} | |
| 123 | AlkLEᵥᵢᵢEᵢᵢ | EᵥᵢEᵥᵢᵢᵢ | E_{b} | |
| 124 | AlkLEᵥᵢᵢEᵢᵢE_{iib} | EᵥᵢEᵥᵢᵢᵢ | E_{b} | |
| 125 | AlkLEᵥᵢᵢEᵢᵢᵢ | EᵥᵢEᵥᵢᵢᵢ | E_{b} | |
| 126 | AlkLEᵥᵢᵢEᵢᵥ | EᵥᵢEᵥᵢᵢᵢ | E_{b} | |
| 127 | AlkLEᵥᵢᵢ | EᵥᵢEᵥᵢᵢᵢ | E_{d} | |
| 128 | AlkLEᵥᵢᵢEᵢ | EᵥᵢEᵥᵢᵢᵢ | E_{d} | |
| 129 | AlkLEᵥᵢᵢEᵢᵢ | EᵥᵢEᵥᵢᵢᵢ | E_{d} | |
| 130 | AlkLEᵥᵢᵢEᵢᵢE_{iib} | EᵥᵢEᵥᵢᵢᵢ | E_{d} | |
| 131 | AlkLEᵥᵢᵢEᵢᵢᵢ | EᵥᵢEᵥᵢᵢᵢ | E_{d} | |
| 132 | AlkLEᵥᵢᵢEᵢᵥ | EᵥᵢEᵥᵢᵢᵢ | E_{d} | |
| 133 | AlkLEᵥᵢᵢ | EᵥᵢEᵥᵢᵢᵢ | Eₑ | |
| 134 | AlkLEᵥᵢᵢEᵢ | EᵥᵢEᵥᵢᵢᵢ | Eₑ | |
| 135 | AlkLEᵥᵢᵢEᵢᵢ | EᵥᵢEᵥᵢᵢᵢ | Eₑ | |
| 136 | AlkLEᵥᵢᵢEᵢᵢE_{iib} | EᵥᵢEᵥᵢᵢᵢ | Eₑ | |
| 137 | AlkLEᵥᵢᵢEᵢᵢᵢ | EᵥᵢEᵥᵢᵢᵢ | Eₑ | |
| 138 | AlkLEᵥᵢᵢEᵢᵥ | EᵥᵢEᵥᵢᵢᵢ | Eₑ | |
| 139 | AlkLEᵥᵢᵢ | EᵥᵢEᵥᵢᵢᵢ | E_{f} | |
| 140 | AlkLEᵥᵢᵢEᵢ | EᵥᵢEᵥᵢᵢᵢ | E_{f} | |
| 141 | AlkLEᵥᵢᵢEᵢᵢ | EᵥᵢEᵥᵢᵢᵢ | E_{f} | |
| 142 | AlkLEᵥᵢᵢEᵢᵢE_{iib} | EᵥᵢEᵥᵢᵢᵢ | E_{f} | |
| 143 | AlkLEᵥᵢᵢEᵢᵢᵢ | EᵥᵢEᵥᵢᵢᵢ | E_{f} | |
| 144 | AlkLEᵥᵢᵢEᵢᵥ | EᵥᵢEᵥᵢᵢᵢ | E_{f} | |
| 145 | AlkLEᵥᵢᵢ | E_{iib}EᵥᵢEₓ | | |
| 146 | AlkLEᵥᵢᵢEᵢ | E_{iib}EᵥᵢEₓ | | |
| 147 | AlkLEᵥᵢᵢEᵢᵢ | E_{iib}EᵥᵢEₓ | | |
| 148 | AlkLEᵥᵢᵢEᵢᵢE_{iib} | E_{iib}EᵥᵢEₓ | | |
| 149 | AlkLEᵥᵢᵢEᵢᵢᵢ | E_{iib}EᵥᵢEₓ | | |
| 150 | AlkLEᵥᵢᵢEᵢᵥ | E_{iib}EᵥᵢEₓ | | |
| 151 | AlkLEᵥᵢᵢ | E_{iib}EᵥᵢEₓ | | |
| 152 | AlkLEᵥᵢᵢEᵢ | E_{iib}EᵥᵢEₓ | | |
| 153 | AlkLEᵥᵢᵢEᵢᵢ | E_{iib}EᵥᵢEₓ | | |
| 154 | AlkLEᵥᵢᵢEᵢᵢE_{iib} | E_{iib}EᵥᵢEₓ | | |
| 155 | AlkLEᵥᵢᵢEᵢᵢᵢ | E_{iib}EᵥᵢEₓ | | |
| 156 | AlkLEᵥᵢᵢEᵢᵥ | E_{iib}EᵥᵢEₓ | | |
| 157 | AlkLEᵥᵢᵢ | E_{iib}EᵥᵢEₓ | Eₐ | |
| 158 | AlkLEᵥᵢᵢEᵢ | E_{iib}EᵥᵢEₓ | Eₐ | |
| 159 | AlkLEᵥᵢᵢEᵢᵢ | E_{iib}EᵥᵢEₓ | Eₐ | |
| 160 | AlkLEᵥᵢᵢEᵢᵢE_{iib} | E_{iib}EᵥᵢEₓ | Eₐ | |
| 161 | AlkLEᵥᵢᵢEᵢᵢᵢ | E_{iib}EᵥᵢEₓ | Eₐ | |
| 162 | AlkLEᵥᵢᵢEᵢᵥ | E_{iib}EᵥᵢEₓ | Eₐ | |
| 163 | AlkLEᵥᵢᵢ | E_{iib}EᵥᵢEₓ | E_{b} | |
| 164 | AlkLEᵥᵢᵢEᵢ | E_{iib}EᵥᵢEₓ | E_{b} | |
| 165 | AlkLEᵥᵢᵢEᵢᵢ | E_{iib}EᵥᵢEₓ | E_{b} | |
| 166 | AlkLEᵥᵢᵢEᵢᵢE_{iib} | E_{iib}EᵥᵢEₓ | E_{b} | |
| 167 | AlkLEᵥᵢᵢEᵢᵢᵢ | E_{iib}EᵥᵢEₓ | E_{b} | |
| 168 | AlkLEᵥᵢᵢEᵢᵥ | E_{iib}EᵥᵢEₓ | E_{b} | |
| 169 | AlkLEᵥᵢᵢ | E_{iib}EᵥᵢEₓ | E_{d} | |
| 170 | AlkLEᵥᵢᵢEᵢ | E_{iib}EᵥᵢEₓ | E_{d} | |
| 171 | AlkLEᵥᵢᵢEᵢᵢ | E_{iib}EᵥᵢEₓ | E_{d} | |
| 172 | AlkLEᵥᵢᵢEᵢᵢE_{iib} | E_{iib}EᵥᵢEₓ | E_{d} | |
| 173 | AlkLEᵥᵢᵢEᵢᵢᵢ | E_{iib}EᵥᵢEₓ | E_{d} | |
| 174 | AlkLEᵥᵢᵢEᵢᵥ | E_{iib}EᵥᵢEₓ | E_{d} | |
| 175 | AlkLEᵥᵢᵢ | E_{iib}EᵥᵢEₓ | Eₑ | |
| 176 | AlkLEᵥᵢᵢEᵢ | E_{iib}EᵥᵢEₓ | Eₑ | |
| 177 | AlkLEᵥᵢᵢEᵢᵢ | E_{iib}EᵥᵢEₓ | Eₑ | |
| 178 | AlkLEᵥᵢᵢEᵢᵢE_{iib} | E_{iib}EᵥᵢEₓ | Eₑ | |
| 179 | AlkLEᵥᵢᵢEᵢᵢᵢ | E_{iib}EᵥᵢEₓ | Eₑ | |
| 180 | AlkLEᵥᵢᵢEᵢᵥ | E_{iib}EᵥᵢEₓ | Eₑ | |
| 181 | AlkLEᵥᵢᵢ | E_{iib}EᵥᵢEₓ | E_{f} | |
| 182 | AlkLEᵥᵢᵢEᵢ | E_{iib}EᵥᵢEₓ | E_{f} | |
| 183 | AlkLEᵥᵢᵢEᵢᵢ | E_{iib}EᵥᵢEₓ | E_{f} | |
| 184 | AlkLEᵥᵢᵢEᵢᵢE_{iib} | E_{iib}EᵥᵢEₓ | E_{f} | |
| 185 | AlkLEᵥᵢᵢEᵢᵢᵢ | E_{iib}EᵥᵢEₓ | E_{f} | |
| 186 | AlkLEᵥᵢᵢEᵢᵥ | E_{iib}EᵥᵢEₓ | E_{f} | |
| 187 | AlkLEᵥᵢᵢ | E_{iib}EᵥᵢEₓ | | |
| 188 | AlkLEᵥᵢᵢEᵢ | E_{iib}EᵥᵢEₓ | | |
| 189 | AlkLEᵥᵢᵢEᵢᵢ | E_{iib}EᵥᵢEₓ | | |
| 190 | AlkLEᵥᵢᵢEᵢᵢE_{iib} | E_{iib}EᵥᵢEₓ | | |
| 191 | AlkLEᵥᵢᵢEᵢᵢᵢ | E_{iib}EᵥᵢEₓ | | |
| 192 | AlkLEᵥᵢᵢEᵢᵥ | E_{iib}EᵥᵢEₓ | | |
| 193 | AlkLEₓᵢᵢᵢEᵥᵢᵢ | Eᵥᵢᵢᵢ | | |
| 194 | AlkLEₓᵢᵢᵢEᵥᵢᵢEᵢ | Eᵥᵢᵢᵢ | | |
| 195 | AlkLEₓᵢᵢᵢEᵥᵢᵢEᵢᵢ | Eᵥᵢᵢᵢ | | |
| 196 | AlkLEₓᵢᵢᵢEᵥᵢᵢEᵢᵢE_{iib} | Eᵥᵢᵢᵢ | | |
| 197 | AlkLEₓᵢᵢᵢEᵥᵢᵢEᵢᵢᵢ | Eᵥᵢᵢᵢ | | |
| 198 | AlkLEₓᵢᵢᵢEᵥᵢᵢEᵢᵥ | Eᵥᵢᵢᵢ | | |
| 199 | AlkLEₓᵢᵢᵢEᵥᵢᵢ | Eᵥᵢᵢᵢ | Eₐ | |
| 200 | AlkLEₓᵢᵢᵢEᵥᵢᵢEᵢ | Eᵥᵢᵢᵢ | Eₐ | |
| 201 | AlkLEₓᵢᵢᵢEᵥᵢᵢEᵢᵢ | Eᵥᵢᵢᵢ | Eₐ | |
| 202 | AlkLEₓᵢᵢᵢEᵥᵢᵢEᵢᵢE_{iib} | Eᵥᵢᵢᵢ | Eₐ | |
| 203 | AlkLEₓᵢᵢᵢEᵥᵢᵢEᵢᵢᵢ | Eᵥᵢᵢᵢ | Eₐ | |
| 204 | AlkLEₓᵢᵢᵢEᵥᵢᵢEᵢᵥ | Eᵥᵢᵢᵢ | Eₐ | |
| 205 | AlkLEₓᵢᵢᵢEᵥᵢᵢ | Eᵥᵢᵢᵢ | E_{b} | |
| 206 | AlkLEₓᵢᵢᵢEᵥᵢᵢEᵢ | Eᵥᵢᵢᵢ | E_{b} | |
| 207 | AlkLEₓᵢᵢᵢEᵥᵢᵢEᵢᵢ | Eᵥᵢᵢᵢ | E_{b} | |
| 208 | AlkLEₓᵢᵢᵢEᵥᵢᵢEᵢᵢE_{iib} | Eᵥᵢᵢᵢ | E_{b} | |
| 209 | AlkLEₓᵢᵢᵢEᵥᵢᵢEᵢᵢᵢ | Eᵥᵢᵢᵢ | E_{b} | |
| 210 | AlkLEₓᵢᵢᵢEᵥᵢᵢEᵢᵥ | Eᵥᵢᵢᵢ | E_{b} | |
| 211 | AlkLEₓᵢᵢᵢEᵥᵢᵢ | Eᵥᵢᵢᵢ | E_{d} | |
| 212 | AlkLEₓᵢᵢᵢEᵥᵢᵢEᵢ | Eᵥᵢᵢᵢ | E_{d} | |
| 213 | AlkLEₓᵢᵢᵢEᵥᵢᵢEᵢᵢ | Eᵥᵢᵢᵢ | E_{d} | |
| 214 | AlkLEₓᵢᵢᵢEᵥᵢᵢEᵢᵢE_{iib} | Eᵥᵢᵢᵢ | E_{d} | |
| 215 | AlkLEₓᵢᵢᵢEᵥᵢᵢEᵢᵢᵢ | Eᵥᵢᵢᵢ | E_{d} | |
| 216 | AlkLEₓᵢᵢᵢEᵥᵢᵢEᵢᵥ | Eᵥᵢᵢᵢ | E_{d} | |
| 217 | AlkLEₓᵢᵢᵢEᵥᵢᵢ | Eᵥᵢᵢᵢ | Eₑ | |
| 218 | AlkLEₓᵢᵢᵢEᵥᵢᵢEᵢ | Eᵥᵢᵢᵢ | Eₑ | |
| 219 | AlkLEₓᵢᵢᵢEᵥᵢᵢEᵢᵢ | Eᵥᵢᵢᵢ | Eₑ | |
| 220 | AlkLEₓᵢᵢᵢEᵥᵢᵢEᵢᵢE_{iib} | Eᵥᵢᵢᵢ | Eₑ | |
| 221 | AlkLEₓᵢᵢᵢEᵥᵢᵢEᵢᵢᵢ | Eᵥᵢᵢᵢ | Eₑ | |
| 222 | AlkLEₓᵢᵢᵢEᵥᵢᵢEᵢᵥ | Eᵥᵢᵢᵢ | Eₑ | |
| 223 | AlkLEₓᵢᵢᵢEᵥᵢᵢ | Eᵥᵢᵢᵢ | E_{f} | |
| 224 | AlkLEₓᵢᵢᵢEᵥᵢᵢEᵢ | Eᵥᵢᵢᵢ | E_{f} | |
| 225 | AlkLEₓᵢᵢᵢEᵥᵢᵢEᵢᵢ | Eᵥᵢᵢᵢ | E_{f} | |
| 226 | AlkLEₓᵢᵢᵢEᵥᵢᵢEᵢᵢE_{iib} | Eᵥᵢᵢᵢ | E_{f} | |
| 227 | AlkLEₓᵢᵢᵢEᵥᵢᵢEᵢᵢᵢ | Eᵥᵢᵢᵢ | E_{f} | |
| 228 | AlkLEₓᵢᵢᵢEᵥᵢᵢEᵢᵥ | Eᵥᵢᵢᵢ | E_{f} | |
| 229 | AlkLEₓᵢᵢᵢEᵥᵢᵢ | Eᵢₓ | | |
| 230 | AlkLEₓᵢᵢᵢEᵥᵢᵢEᵢ | Eᵢₓ | | |
| 231 | AlkLEₓᵢᵢᵢEᵥᵢᵢEᵢᵢ | Eᵢₓ | | |
| 232 | AlkLEₓᵢᵢᵢEᵥᵢᵢEᵢᵢE_{iib} | Eᵢₓ | | |
| 233 | AlkLEₓᵢᵢᵢEᵥᵢᵢEᵢᵢᵢ | Eᵢₓ | | |
| 234 | AlkLEₓᵢᵢᵢEᵥᵢᵢEᵢᵥ | Eᵢₓ | | |
| 235 | AlkLEₓᵢᵢᵢEᵥᵢᵢ | Eᵢₓ | Eₐ | |
| 236 | AlkLEₓᵢᵢᵢEᵥᵢᵢEᵢ | Eᵢₓ | Eₐ | |
| 237 | AlkLEₓᵢᵢᵢEᵥᵢᵢEᵢᵢ | Eᵢₓ | Eₐ | |
| 238 | AlkLEₓᵢᵢᵢEᵥᵢᵢEᵢᵢE_{iib} | Eᵢₓ | Eₐ | |
| 239 | AlkLEₓᵢᵢᵢEᵥᵢᵢEᵢᵢᵢ | Eᵢₓ | Eₐ | |
| 240 | AlkLEₓᵢᵢᵢEᵥᵢᵢEᵢᵥ | Eᵢₓ | Eₐ | |
| 241 | AlkLEₓᵢᵢᵢEᵥᵢᵢ | Eᵢₓ | E_{b} | |
| 242 | AlkLEₓᵢᵢᵢEᵥᵢᵢEᵢ | Eᵢₓ | E_{b} | |
| 243 | AlkLEₓᵢᵢᵢEᵥᵢᵢEᵢᵢ | Eᵢₓ | E_{b} | |
| 244 | AlkLEₓᵢᵢᵢEᵥᵢᵢEᵢᵢE_{iib} | Eᵢₓ | E_{b} | |
| 245 | AlkLEₓᵢᵢᵢEᵥᵢᵢEᵢᵢᵢ | Eᵢₓ | E_{b} | |
| 246 | AlkLEₓᵢᵢᵢEᵥᵢᵢEᵢᵥ | Eᵢₓ | E_{b} | |
| 247 | AlkLEₓᵢᵢᵢEᵥᵢᵢ | Eᵢₓ | E_{d} | |
| 248 | AlkLEₓᵢᵢᵢEᵥᵢᵢEᵢ | Eᵢₓ | E_{d} | |
| 249 | AlkLEₓᵢᵢᵢEᵥᵢᵢEᵢᵢ | Eᵢₓ | E_{d} | |
| 250 | AlkLEₓᵢᵢᵢEᵥᵢᵢEᵢᵢE_{iib} | Eᵢₓ | E_{d} | |
| 251 | AlkLEₓᵢᵢᵢEᵥᵢᵢEᵢᵢᵢ | Eᵢₓ | E_{d} | |
| 252 | AlkLEₓᵢᵢᵢEᵥᵢᵢEᵢᵥ | Eᵢₓ | E_{d} | |
| 253 | AlkLEₓᵢᵢᵢEᵥᵢᵢ | Eᵢₓ | Eₑ | |
| 254 | AlkLEₓᵢᵢᵢEᵥᵢᵢEᵢ | Eᵢₓ | Eₑ | |
| 255 | AlkLEₓᵢᵢᵢEᵥᵢᵢEᵢᵢ | Eᵢₓ | Eₑ | |
| 256 | AlkLEₓᵢᵢᵢEᵥᵢᵢEᵢᵢE_{iib} | Eᵢₓ | Eₑ | |
| 257 | AlkLEₓᵢᵢᵢEᵥᵢᵢEᵢᵢᵢ | Eᵢₓ | Eₑ | |
| 258 | AlkLEₓᵢᵢᵢEᵥᵢᵢEᵢᵥ | Eᵢₓ | Eₑ | |
| 259 | AlkLEₓᵢᵢᵢEᵥᵢᵢ | Eᵢₓ | E_{f} | |
| 260 | AlkLEₓᵢᵢᵢEᵥᵢᵢEᵢ | Eᵢₓ | E_{f} | |
| 261 | AlkLEₓᵢᵢᵢEᵥᵢᵢEᵢᵢ | Eᵢₓ | E_{f} | |
| 262 | AlkLEₓᵢᵢᵢEᵥᵢᵢEᵢᵢE_{iib} | Eᵢₓ | E_{f} | |
| 263 | AlkLEₓᵢᵢᵢEᵥᵢᵢEᵢᵢᵢ | Eᵢₓ | E_{f} | |
| 264 | AlkLEₓᵢᵢᵢEᵥᵢᵢEᵢᵥ | Eᵢₓ | E_{f} | |
| 265 | AlkLEₓᵢᵢᵢEᵥᵢᵢ | Eₓ | | |
| 266 | AlkLEₓᵢᵢᵢEᵥᵢᵢEᵢ | Eₓ | | |
| 267 | AlkLEₓᵢᵢᵢEᵥᵢᵢEᵢᵢ | Eₓ | | |
| 268 | AlkLEₓᵢᵢᵢEᵥᵢᵢEᵢᵢE_{iib} | Eₓ | | |
| 269 | AlkLEₓᵢᵢᵢEᵥᵢᵢEᵢᵢᵢ | Eₓ | | |
| 270 | AlkLEₓᵢᵢᵢEᵥᵢᵢEᵢᵥ | Eₓ | | |
| 271 | AlkLEₓᵢᵢᵢEᵥᵢᵢ | Eₓ | Eₐ | |
| 272 | AlkLEₓᵢᵢᵢEᵥᵢᵢEᵢ | Eₓ | Eₐ | |
| 273 | AlkLEₓᵢᵢᵢEᵥᵢᵢEᵢᵢ | Eₓ | Eₐ | |
| 274 | AlkLEₓᵢᵢᵢEᵥᵢᵢEᵢᵢE_{iib} | Eₓ | Eₐ | |
| 275 | AlkLEₓᵢᵢᵢEᵥᵢᵢEᵢᵢᵢ | Eₓ | Eₐ | |
| 276 | AlkLEₓᵢᵢᵢEᵥᵢᵢEᵢᵥ | Eₓ | Eₐ | |
| 277 | AlkLEₓᵢᵢᵢEᵥᵢᵢ | Eₓ | E_{b} | |
| 278 | AlkLEₓᵢᵢᵢEᵥᵢᵢEᵢ | Eₓ | E_{b} | |
| 279 | AlkLEₓᵢᵢᵢEᵥᵢᵢEᵢᵢ | Eₓ | E_{b} | |
| 280 | AlkLEₓᵢᵢᵢEᵥᵢᵢEᵢᵢE_{iib} | Eₓ | E_{b} | |
| 281 | AlkLEₓᵢᵢᵢEᵥᵢᵢEᵢᵢᵢ | Eₓ | E_{b} | |
| 282 | AlkLEₓᵢᵢᵢEᵥᵢᵢEᵢᵥ | Eₓ | E_{b} | |
| 283 | AlkLEₓᵢᵢᵢEᵥᵢᵢ | Eₓ | E_{d} | |
| 284 | AlkLEₓᵢᵢᵢEᵥᵢᵢEᵢ | Eₓ | E_{d} | |
| 285 | AlkLEₓᵢᵢᵢEᵥᵢᵢEᵢᵢ | Eₓ | E_{d} | |
| 286 | AlkLEₓᵢᵢᵢEᵥᵢᵢEᵢᵢE_{iib} | Eₓ | E_{d} | |
| 287 | AlkLEₓᵢᵢᵢEᵥᵢᵢEᵢᵢᵢ | Eₓ | E_{d} | |
| 288 | AlkLEₓᵢᵢᵢEᵥᵢᵢEᵢᵥ | Eₓ | E_{d} | |
| 289 | AlkLEₓᵢᵢᵢEᵥᵢᵢ | Eₓ | Eₑ | |
| 290 | AlkLEₓᵢᵢᵢEᵥᵢᵢEᵢ | Eₓ | Eₑ | |
| 291 | AlkLEₓᵢᵢᵢEᵥᵢᵢEᵢᵢ | Eₓ | Eₑ | |
| 292 | AlkLEₓᵢᵢᵢEᵥᵢᵢEᵢᵢE_{iib} | Eₓ | Eₑ | |
| 293 | AlkLEₓᵢᵢᵢEᵥᵢᵢEᵢᵢᵢ | Eₓ | Eₑ | |
| 294 | AlkLEₓᵢᵢᵢEᵥᵢᵢEᵢᵥ | Eₓ | Eₑ | |
| 295 | AlkLEₓᵢᵢᵢEᵥᵢᵢ | Eₓ | E_{f} | |
| 296 | AlkLEₓᵢᵢᵢEᵥᵢᵢEᵢ | Eₓ | E_{f} | |
| 297 | AlkLEₓᵢᵢᵢEᵥᵢᵢEᵢᵢ | Eₓ | E_{f} | |
| 298 | AlkLEₓᵢᵢᵢEᵥᵢᵢEᵢᵢE_{iib} | Eₓ | E_{f} | |
| 299 | AlkLEₓᵢᵢᵢEᵥᵢᵢEᵢᵢᵢ | Eₓ | E_{f} | |
| 300 | AlkLEₓᵢᵢᵢEᵥᵢᵢEᵢᵥ | Eₓ | E_{f} | |
| 301 | AlkLEₓᵢᵢᵢEᵥᵢᵢ | EᵥᵢEᵥᵢᵢᵢ | | |
| 302 | AlkLEₓᵢᵢᵢEᵥᵢᵢEᵢ | EᵥᵢEᵥᵢᵢᵢ | | |
| 303 | AlkLEₓᵢᵢᵢEᵥᵢᵢEᵢᵢ | EᵥᵢEᵥᵢᵢᵢ | | |
| 304 | AlkLEₓᵢᵢᵢEᵥᵢᵢEᵢᵢE_{iib} | EᵥᵢEᵥᵢᵢᵢ | | |
| 305 | AlkLEₓᵢᵢᵢEᵥᵢᵢEᵢᵢᵢ | EᵥᵢEᵥᵢᵢᵢ | | |
| 306 | AlkLEₓᵢᵢᵢEᵥᵢᵢEᵢᵥ | EᵥᵢEᵥᵢᵢᵢ | | |
| 307 | AlkLEₓᵢᵢᵢEᵥᵢᵢ | EᵥᵢEᵥᵢᵢᵢ | Eₐ | |
| 308 | AlkLEₓᵢᵢᵢEᵥᵢᵢEᵢ | EᵥᵢEᵥᵢᵢᵢ | Eₐ | |
| 309 | AlkLEₓᵢᵢᵢEᵥᵢᵢEᵢᵢ | EᵥᵢEᵥᵢᵢᵢ | Eₐ | |
| 310 | AlkLEₓᵢᵢᵢEᵥᵢᵢEᵢᵢE_{iib} | EᵥᵢEᵥᵢᵢᵢ | Eₐ | |
| 311 | AlkLEₓᵢᵢᵢEᵥᵢᵢEᵢᵢᵢ | EᵥᵢEᵥᵢᵢᵢ | Eₐ | |
| 312 | AlkLEₓᵢᵢᵢEᵥᵢᵢEᵢᵥ | EᵥᵢEᵥᵢᵢᵢ | Eₐ | |
| 313 | AlkLEₓᵢᵢᵢEᵥᵢᵢ | EᵥᵢEᵥᵢᵢᵢ | E_{b} | |
| 314 | AlkLEₓᵢᵢᵢEᵥᵢᵢEᵢ | EᵥᵢEᵥᵢᵢᵢ | E_{b} | |
| 315 | AlkLEₓᵢᵢᵢEᵥᵢᵢEᵢᵢ | EᵥᵢEᵥᵢᵢᵢ | E_{b} | |
| 316 | AlkLEₓᵢᵢᵢEᵥᵢᵢEᵢᵢE_{iib} | EᵥᵢEᵥᵢᵢᵢ | E_{b} | |
| 317 | AlkLEₓᵢᵢᵢEᵥᵢᵢEᵢᵢᵢ | EᵥᵢEᵥᵢᵢᵢ | E_{b} | |
| 318 | AlkLEₓᵢᵢᵢEᵥᵢᵢEᵢᵥ | EᵥᵢEᵥᵢᵢᵢ | E_{b} | |
| 319 | AlkLEₓᵢᵢᵢEᵥᵢᵢ | EᵥᵢEᵥᵢᵢᵢ | E_{d} | |
| 320 | AlkLEₓᵢᵢᵢEᵥᵢᵢEᵢ | EᵥᵢEᵥᵢᵢᵢ | E_{d} | |
| 321 | AlkLEₓᵢᵢᵢEᵥᵢᵢEᵢᵢ | EᵥᵢEᵥᵢᵢᵢ | E_{d} | |
| 322 | AlkLEₓᵢᵢᵢEᵥᵢᵢEᵢᵢE_{iib} | EᵥᵢEᵥᵢᵢᵢ | E_{d} | |
| 323 | AlkLEₓᵢᵢᵢEᵥᵢᵢEᵢᵢᵢ | EᵥᵢEᵥᵢᵢᵢ | E_{d} | |
| 324 | AlkLEₓᵢᵢᵢEᵥᵢᵢEᵢᵥ | EᵥᵢEᵥᵢᵢᵢ | E_{d} | |
| 325 | AlkLEₓᵢᵢᵢEᵥᵢᵢ | EᵥᵢEᵥᵢᵢᵢ | Eₑ | |
| 326 | AlkLEₓᵢᵢᵢEᵥᵢᵢEᵢ | EᵥᵢEᵥᵢᵢᵢ | Eₑ | |
| 327 | AlkLEₓᵢᵢᵢEᵥᵢᵢEᵢᵢ | EᵥᵢEᵥᵢᵢᵢ | Eₑ | |
| 328 | AlkLEₓᵢᵢᵢEᵥᵢᵢEᵢᵢE_{iib} | EᵥᵢEᵥᵢᵢᵢ | Eₑ | |
| 329 | AlkLEₓᵢᵢᵢEᵥᵢᵢEᵢᵢᵢ | EᵥᵢEᵥᵢᵢᵢ | Eₑ | |
| 330 | AlkLEₓᵢᵢᵢEᵥᵢᵢEᵢᵥ | EᵥᵢEᵥᵢᵢᵢ | Eₑ | |
| 331 | AlkLEₓᵢᵢᵢEᵥᵢᵢ | EᵥᵢEᵥᵢᵢᵢ | E_{f} | |
| 332 | AlkLEₓᵢᵢᵢEᵥᵢᵢEᵢ | EᵥᵢEᵥᵢᵢᵢ | E_{f} | |
| 333 | AlkLEₓᵢᵢᵢEᵥᵢᵢEᵢᵢ | EᵥᵢEᵥᵢᵢᵢ | E_{f} | |
| 334 | AlkLEₓᵢᵢᵢEᵥᵢᵢEᵢᵢE_{iib} | EᵥᵢEᵥᵢᵢᵢ | E_{f} | |
| 335 | AlkLEₓᵢᵢᵢEᵥᵢᵢEᵢᵢᵢ | EᵥᵢEᵥᵢᵢᵢ | E_{f} | |
| 336 | AlkLEₓᵢᵢᵢEᵥᵢᵢEᵢᵥ | EᵥᵢEᵥᵢᵢᵢ | E_{f} | |
| 337 | AlkLEₓᵢᵢᵢEᵥᵢᵢ | E_{iib}EᵥᵢEₓ | | |
| 338 | AlkLEₓᵢᵢᵢEᵥᵢᵢEᵢ | E_{iib}EᵥᵢEₓ | | |
| 339 | AlkLEₓᵢᵢᵢEᵥᵢᵢEᵢᵢ | E_{iib}EᵥᵢEₓ | | |
| 340 | AlkLEₓᵢᵢᵢEᵥᵢᵢEᵢᵢE_{iib} | E_{iib}EᵥᵢEₓ | | |
| 341 | AlkLEₓᵢᵢᵢEᵥᵢᵢEᵢᵢᵢ | E_{iib}EᵥᵢEₓ | | |
| 342 | AlkLEₓᵢᵢᵢEᵥᵢᵢEᵢᵥ | E_{iib}EᵥᵢEₓ | | |
| 343 | AlkLEₓᵢᵢᵢEᵥᵢᵢ | E_{iib}EᵥᵢEₓ | Eₐ | |
| 344 | AlkLEₓᵢᵢᵢEᵥᵢᵢEᵢ | E_{iib}EᵥᵢEₓ | Eₐ | |
| 345 | AlkLEₓᵢᵢᵢEᵥᵢᵢEᵢᵢ | E_{iib}EᵥᵢEₓ | Eₐ | |
| 346 | AlkLEₓᵢᵢᵢEᵥᵢᵢEᵢᵢE_{iib} | E_{iib}EᵥᵢEₓ | Eₐ | |
| 347 | AlkLEₓᵢᵢᵢEᵥᵢᵢEᵢᵢᵢ | E_{iib}EᵥᵢEₓ | Eₐ | |
| 348 | AlkLEₓᵢᵢᵢEᵥᵢᵢEᵢᵥ | E_{iib}EᵥᵢEₓ | Eₐ | |
| 349 | AlkLEₓᵢᵢᵢEᵥᵢᵢ | E_{iib}EᵥᵢEₓ | E_{b} | |
| 350 | AlkLEₓᵢᵢᵢEᵥᵢᵢEᵢ | E_{iib}EᵥᵢEₓ | E_{b} | |
| 351 | AlkLEₓᵢᵢᵢEᵥᵢᵢEᵢᵢ | E_{iib}EᵥᵢEₓ | E_{b} | |
| 352 | AlkLEₓᵢᵢᵢEᵥᵢᵢEᵢᵢE_{iib} | E_{iib}EᵥᵢEₓ | E_{b} | |
| 353 | AlkLEₓᵢᵢᵢEᵥᵢᵢEᵢᵢᵢ | E_{iib}EᵥᵢEₓ | E_{b} | |
| 354 | AlkLEₓᵢᵢᵢEᵥᵢᵢEᵢᵥ | E_{iib}EᵥᵢEₓ | E_{b} | |
| 355 | AlkLEₓᵢᵢᵢEᵥᵢᵢ | E_{iib}EᵥᵢEₓ | E_{d} | |
| 356 | AlkLEₓᵢᵢᵢEᵥᵢᵢEᵢ | E_{iib}EᵥᵢEₓ | E_{d} | |
| 357 | AlkLEₓᵢᵢᵢEᵥᵢᵢEᵢᵢ | E_{iib}EᵥᵢEₓ | E_{d} | |
| 358 | AlkLEₓᵢᵢᵢEᵥᵢᵢEᵢᵢE_{iib} | E_{iib}EᵥᵢEₓ | E_{d} | |
| 359 | AlkLEₓᵢᵢᵢEᵥᵢᵢEᵢᵢᵢ | E_{iib}EᵥᵢEₓ | E_{d} | |
| 360 | AlkLEₓᵢᵢᵢEᵥᵢᵢEᵢᵥ | E_{iib}EᵥᵢEₓ | E_{d} | |
| 361 | AlkLEₓᵢᵢᵢEᵥᵢᵢ | E_{iib}EᵥᵢEₓ | Eₑ | |
| 362 | AlkLEₓᵢᵢᵢEᵥᵢᵢEᵢ | E_{iib}EᵥᵢEₓ | Eₑ | |
| 363 | AlkLEₓᵢᵢᵢEᵥᵢᵢEᵢᵢ | E_{iib}EᵥᵢEₓ | Eₑ | |
| 364 | AlkLEₓᵢᵢᵢEᵥᵢᵢEᵢᵢE_{iib} | E_{iib}EᵥᵢEₓ | Eₑ | |
| 365 | AlkLEₓᵢᵢᵢEᵥᵢᵢEᵢᵢᵢ | E_{iib}EᵥᵢEₓ | Eₑ | |
| 366 | AlkLEₓᵢᵢᵢEᵥᵢᵢEᵢᵥ | E_{iib}EᵥᵢEₓ | Eₑ | |
| 367 | AlkLEₓᵢᵢᵢEᵥᵢᵢ | E_{iib}EᵥᵢEₓ | E_{f} | |
| 368 | AlkLEₓᵢᵢᵢEᵥᵢᵢEᵢ | E_{iib}EᵥᵢEₓ | E_{f} | |
| 369 | AlkLEₓᵢᵢᵢEᵥᵢᵢEᵢᵢ | E_{iib}EᵥᵢEₓ | E_{f} | |
| 370 | AlkLEₓᵢᵢᵢEᵥᵢᵢEᵢᵢE_{iib} | E_{iib}EᵥᵢEₓ | E_{f} | |
| 371 | AlkLEₓᵢᵢᵢEᵥᵢᵢEᵢᵢᵢ | E_{iib}EᵥᵢEₓ | E_{f} | |
| 372 | AlkLEₓᵢᵢᵢEᵥᵢᵢEᵢᵥ | E_{iib}EᵥᵢEₓ | E_{f} | |
| 373 | AlkLEᵥᵢᵢ | Eᵥᵢᵢᵢ | | E₁ |
| 374 | AlkLEᵥᵢᵢEᵢ | Eᵥᵢᵢᵢ | | E₁ |
| 375 | AlkLEᵥᵢᵢEᵢᵢ | Eᵥᵢᵢᵢ | | E₁ |
| 376 | AlkLEᵥᵢᵢEᵢᵢE_{iib} | Eᵥᵢᵢᵢ | | E₁ |
| 377 | AlkLEᵥᵢᵢEᵢᵢᵢ | Eᵥᵢᵢᵢ | | E₁ |
| 378 | AlkLEᵥᵢᵢEᵢᵥ | Eᵥᵢᵢᵢ | | E₁ |
| 379 | AlkLEᵥᵢᵢ | Eᵥᵢᵢᵢ | Eₐ | E₁ |
| 380 | AlkLEᵥᵢᵢEᵢ | Eᵥᵢᵢᵢ | Eₐ | E₁ |
| 381 | AlkLEᵥᵢᵢEᵢᵢ | Eᵥᵢᵢᵢ | Eₐ | E₁ |
| 382 | AlkLEᵥᵢᵢEᵢᵢE_{iib} | Eᵥᵢᵢᵢ | Eₐ | E₁ |
| 383 | AlkLEᵥᵢᵢEᵢᵢᵢ | Eᵥᵢᵢᵢ | Eₐ | E₁ |
| 384 | AlkLEᵥᵢᵢEᵢᵥ | Eᵥᵢᵢᵢ | Eₐ | E₁ |
| 385 | AlkLEᵥᵢᵢ | Eᵥᵢᵢᵢ | E_{b} | E₁ |
| 386 | AlkLEᵥᵢᵢEᵢ | Eᵥᵢᵢᵢ | E_{b} | E₁ |
| 387 | AlkLEᵥᵢᵢEᵢᵢ | Eᵥᵢᵢᵢ | E_{b} | E₁ |
| 388 | AlkLEᵥᵢᵢEᵢᵢE_{iib} | Eᵥᵢᵢᵢ | E_{b} | E₁ |
| 389 | AlkLEᵥᵢᵢEᵢᵢᵢ | Eᵥᵢᵢᵢ | E_{b} | E₁ |
| 390 | AlkLEᵥᵢᵢEᵢᵥ | Eᵥᵢᵢᵢ | E_{b} | E₁ |
| 391 | AlkLEᵥᵢᵢ | Eᵥᵢᵢᵢ | E_{d} | E₁ |
| 392 | AlkLEᵥᵢᵢEᵢ | Eᵥᵢᵢᵢ | E_{d} | E₁ |
| 393 | AlkLEᵥᵢᵢEᵢᵢ | Eᵥᵢᵢᵢ | E_{d} | E₁ |
| 394 | AlkLEᵥᵢᵢEᵢᵢE_{iib} | Eᵥᵢᵢᵢ | E_{d} | E₁ |
| 395 | AlkLEᵥᵢᵢEᵢᵢᵢ | Eᵥᵢᵢᵢ | E_{d} | E₁ |
| 396 | AlkLEᵥᵢᵢEᵢᵥ | Eᵥᵢᵢᵢ | E_{d} | E₁ |
| 397 | AlkLEᵥᵢᵢ | Eᵥᵢᵢᵢ | Eₑ | E₁ |
| 398 | AlkLEᵥᵢᵢEᵢ | Eᵥᵢᵢᵢ | Eₑ | E₁ |
| 399 | AlkLEᵥᵢᵢEᵢᵢ | Eᵥᵢᵢᵢ | Eₑ | E₁ |
| 400 | AlkLEᵥᵢᵢEᵢᵢE_{iib} | Eᵥᵢᵢᵢ | Eₑ | E₁ |
| 401 | AlkLEᵥᵢᵢEᵢᵢᵢ | Eᵥᵢᵢᵢ | Eₑ | E₁ |
| 402 | AlkLEᵥᵢᵢEᵢᵥ | Eᵥᵢᵢᵢ | Eₑ | E₁ |
| 403 | AlkLEᵥᵢᵢ | Eᵥᵢᵢᵢ | E_{f} | E₁ |
| 404 | AlkLEᵥᵢᵢEᵢ | Eᵥᵢᵢᵢ | E_{f} | E₁ |
| 405 | AlkLEᵥᵢᵢEᵢᵢ | Eᵥᵢᵢᵢ | E_{f} | E₁ |
| 406 | AlkLEᵥᵢᵢEᵢᵢE_{iib} | Eᵥᵢᵢᵢ | E_{f} | E₁ |
| 407 | AlkLEᵥᵢᵢEᵢᵢᵢ | Eᵥᵢᵢᵢ | E_{f} | E₁ |
| 408 | AlkLEᵥᵢᵢEᵢᵥ | Eᵥᵢᵢᵢ | E_{f} | E₁ |
| 409 | AlkLEᵥᵢᵢ | Eᵢₓ | | E₁ |
| 410 | AlkLEᵥᵢᵢEᵢ | Eᵢₓ | | E₁ |
| 411 | AlkLEᵥᵢᵢEᵢᵢ | Eᵢₓ | | E₁ |
| 412 | AlkLEᵥᵢᵢEᵢᵢE_{iib} | Eᵢₓ | | E₁ |
| 413 | AlkLEᵥᵢᵢEᵢᵢᵢ | Eᵢₓ | | E₁ |
| 414 | AlkLEᵥᵢᵢEᵢᵥ | Eᵢₓ | | E₁ |
| 415 | AlkLEᵥᵢᵢ | Eᵢₓ | Eₐ | E₁ |
| 416 | AlkLEᵥᵢᵢEᵢ | Eᵢₓ | Eₐ | E₁ |
| 417 | AlkLEᵥᵢᵢEᵢᵢ | Eᵢₓ | Eₐ | E₁ |
| 418 | AlkLEᵥᵢᵢEᵢᵢE_{iib} | Eᵢₓ | Eₐ | E₁ |
| 419 | AlkLEᵥᵢᵢEᵢᵢᵢ | Eᵢₓ | Eₐ | E₁ |
| 420 | AlkLEᵥᵢᵢEᵢᵥ | Eᵢₓ | Eₐ | E₁ |
| 421 | AlkLEᵥᵢᵢ | Eᵢₓ | E_{b} | E₁ |
| 422 | AlkLEᵥᵢᵢEᵢ | Eᵢₓ | E_{b} | E₁ |
| 423 | AlkLEᵥᵢᵢEᵢᵢ | Eᵢₓ | E_{b} | E₁ |
| 424 | AlkLEᵥᵢᵢEᵢᵢE_{iib} | Eᵢₓ | E_{b} | E₁ |
| 425 | AlkLEᵥᵢᵢEᵢᵢᵢ | Eᵢₓ | E_{b} | E₁ |
| 426 | AlkLEᵥᵢᵢEᵢᵥ | Eᵢₓ | E_{b} | E₁ |
| 427 | AlkLEᵥᵢᵢ | Eᵢₓ | E_{d} | E₁ |
| 428 | AlkLEᵥᵢᵢEᵢ | Eᵢₓ | E_{d} | E₁ |
| 429 | AlkLEᵥᵢᵢEᵢᵢ | Eᵢₓ | E_{d} | E₁ |
| 430 | AlkLEᵥᵢᵢEᵢᵢE_{iib} | Eᵢₓ | E_{d} | E₁ |
| 431 | AlkLEᵥᵢᵢEᵢᵢᵢ | Eᵢₓ | E_{d} | E₁ |
| 432 | AlkLEᵥᵢᵢEᵢᵥ | Eᵢₓ | E_{d} | E₁ |
| 433 | AlkLEᵥᵢᵢ | Eᵢₓ | Eₑ | E₁ |
| 434 | AlkLEᵥᵢᵢEᵢ | Eᵢₓ | Eₑ | E₁ |
| 435 | AlkLEᵥᵢᵢEᵢᵢ | Eᵢₓ | Eₑ | E₁ |
| 436 | AlkLEᵥᵢᵢEᵢᵢE_{iib} | Eᵢₓ | Eₑ | E₁ |
| 437 | AlkLEᵥᵢᵢEᵢᵢᵢ | Eᵢₓ | Eₑ | E₁ |
| 438 | AlkLEᵥᵢᵢEᵢᵥ | Eᵢₓ | Eₑ | E₁ |
| 439 | AlkLEᵥᵢᵢ | Eᵢₓ | E_{f} | E₁ |
| 440 | AlkLEᵥᵢᵢEᵢ | Eᵢₓ | E_{f} | E₁ |
| 441 | AlkLEᵥᵢᵢEᵢᵢ | Eᵢₓ | E_{f} | E₁ |
| 442 | AlkLEᵥᵢᵢEᵢᵢE_{iib} | Eᵢₓ | E_{f} | E₁ |
| 443 | AlkLEᵥᵢᵢEᵢᵢᵢ | Eᵢₓ | E_{f} | E₁ |
| 444 | AlkLEᵥᵢᵢEᵢᵥ | Eᵢₓ | E_{f} | E₁ |
| 445 | AlkLEᵥᵢᵢ | Eₓ | | E₁ |
| 446 | AlkLEᵥᵢᵢEᵢ | Eₓ | | E₁ |
| 447 | AlkLEᵥᵢᵢEᵢᵢ | Eₓ | | E₁ |
| 448 | AlkLEᵥᵢᵢEᵢᵢE_{iib} | Eₓ | | E₁ |
| 449 | AlkLEᵥᵢᵢEᵢᵢᵢ | Eₓ | | E₁ |
| 450 | AlkLEᵥᵢᵢEᵢᵥ | Eₓ | | E₁ |
| 451 | AlkLEᵥᵢᵢ | Eₓ | Eₐ | E₁ |
| 452 | AlkLEᵥᵢᵢEᵢ | Eₓ | Eₐ | E₁ |
| 453 | AlkLEᵥᵢᵢEᵢᵢ | Eₓ | Eₐ | E₁ |
| 454 | AlkLEᵥᵢᵢEᵢᵢE_{iib} | Eₓ | Eₐ | E₁ |
| 455 | AlkLEᵥᵢᵢEᵢᵢᵢ | Eₓ | Eₐ | E₁ |
| 456 | AlkLEᵥᵢᵢEᵢᵥ | Eₓ | Eₐ | E₁ |
| 457 | AlkLEᵥᵢᵢ | Eₓ | E_{b} | E₁ |
| 458 | AlkLEᵥᵢᵢEᵢ | Eₓ | E_{b} | E₁ |
| 459 | AlkLEᵥᵢᵢEᵢᵢ | Eₓ | E_{b} | E₁ |
| 460 | AlkLEᵥᵢᵢEᵢᵢE_{iib} | Eₓ | E_{b} | E₁ |
| 461 | AlkLEᵥᵢᵢEᵢᵢᵢ | Eₓ | E_{b} | E₁ |
| 462 | AlkLEᵥᵢᵢEᵢᵥ | Eₓ | E_{b} | E₁ |
| 463 | AlkLEᵥᵢᵢ | Eₓ | E_{d} | E₁ |
| 464 | AlkLEᵥᵢᵢEᵢ | Eₓ | E_{d} | E₁ |
| 465 | AlkLEᵥᵢᵢEᵢᵢ | Eₓ | E_{d} | E₁ |
| 466 | AlkLEᵥᵢᵢEᵢᵢE_{iib} | Eₓ | E_{d} | E₁ |
| 467 | AlkLEᵥᵢᵢEᵢᵢᵢ | Eₓ | E_{d} | E₁ |
| 468 | AlkLEᵥᵢᵢEᵢᵥ | Eₓ | E_{d} | E₁ |
| 469 | AlkLEᵥᵢᵢ | Eₓ | Eₑ | E₁ |
| 470 | AlkLEᵥᵢᵢEᵢ | Eₓ | Eₑ | E₁ |
| 471 | AlkLEᵥᵢᵢEᵢᵢ | Eₓ | Eₑ | E₁ |
| 472 | AlkLEᵥᵢᵢEᵢᵢE_{iib} | Eₓ | Eₑ | E₁ |
| 473 | AlkLEᵥᵢᵢEᵢᵢᵢ | Eₓ | Eₑ | E₁ |
| 474 | AlkLEᵥᵢᵢEᵢᵥ | Eₓ | Eₑ | E₁ |
| 475 | AlkLEᵥᵢᵢ | Eₓ | E_{f} | E₁ |
| 476 | AlkLEᵥᵢᵢEᵢ | Eₓ | E_{f} | E₁ |
| 477 | AlkLEᵥᵢᵢEᵢᵢ | Eₓ | E_{f} | E₁ |
| 478 | AlkLEᵥᵢᵢEᵢᵢE_{iib} | Eₓ | E_{f} | E₁ |
| 479 | AlkLEᵥᵢᵢEᵢᵢᵢ | Eₓ | E_{f} | E₁ |
| 480 | AlkLEᵥᵢᵢEᵢᵥ | Eₓ | E_{f} | E₁ |
| 481 | AlkLEᵥᵢᵢ | EᵥᵢEᵥᵢᵢᵢ | | E₁ |
| 482 | AlkLEᵥᵢᵢEᵢ | EᵥᵢEᵥᵢᵢᵢ | | E₁ |
| 483 | AlkLEᵥᵢᵢEᵢᵢ | EᵥᵢEᵥᵢᵢᵢ | | E₁ |
| 484 | AlkLEᵥᵢᵢEᵢᵢE_{iib} | EᵥᵢEᵥᵢᵢᵢ | | E₁ |
| 485 | AlkLEᵥᵢᵢEᵢᵢᵢ | EᵥᵢEᵥᵢᵢᵢ | | E₁ |
| 486 | AlkLEᵥᵢᵢEᵢᵥ | EᵥᵢEᵥᵢᵢᵢ | | E₁ |
| 487 | AlkLEᵥᵢᵢ | EᵥᵢEᵥᵢᵢᵢ | Eₐ | E₁ |
| 488 | AlkLEᵥᵢᵢEᵢ | EᵥᵢEᵥᵢᵢᵢ | Eₐ | E₁ |
| 489 | AlkLEᵥᵢᵢEᵢᵢ | EᵥᵢEᵥᵢᵢᵢ | Eₐ | E₁ |
| 490 | AlkLEᵥᵢᵢEᵢᵢE_{iib} | EᵥᵢEᵥᵢᵢᵢ | Eₐ | E₁ |
| 491 | AlkLEᵥᵢᵢEᵢᵢᵢ | EᵥᵢEᵥᵢᵢᵢ | Eₐ | E₁ |
| 492 | AlkLEᵥᵢᵢEᵢᵥ | EᵥᵢEᵥᵢᵢᵢ | Eₐ | E₁ |
| 493 | AlkLEᵥᵢᵢ | EᵥᵢEᵥᵢᵢᵢ | E_{b} | E₁ |
| 494 | AlkLEᵥᵢᵢEᵢ | EᵥᵢEᵥᵢᵢᵢ | E_{b} | E₁ |
| 495 | AlkLEᵥᵢᵢEᵢᵢ | EᵥᵢEᵥᵢᵢᵢ | E_{b} | E₁ |
| 496 | AlkLEᵥᵢᵢEᵢᵢE_{iib} | EᵥᵢEᵥᵢᵢᵢ | E_{b} | E₁ |
| 497 | AlkLEᵥᵢᵢEᵢᵢᵢ | EᵥᵢEᵥᵢᵢᵢ | E_{b} | E₁ |
| 498 | AlkLEᵥᵢᵢEᵢᵥ | EᵥᵢEᵥᵢᵢᵢ | E_{b} | E₁ |
| 499 | AlkLEᵥᵢᵢ | EᵥᵢEᵥᵢᵢᵢ | E_{d} | E₁ |
| 500 | AlkLEᵥᵢᵢEᵢ | EᵥᵢEᵥᵢᵢᵢ | E_{d} | E₁ |
| 501 | AlkLEᵥᵢᵢEᵢᵢ | EᵥᵢEᵥᵢᵢᵢ | E_{d} | E₁ |
| 502 | AlkLEᵥᵢᵢEᵢᵢE_{iib} | EᵥᵢEᵥᵢᵢᵢ | E_{d} | E₁ |
| 503 | AlkLEᵥᵢᵢEᵢᵢᵢ | EᵥᵢEᵥᵢᵢᵢ | E_{d} | E₁ |
| 504 | AlkLEᵥᵢᵢEᵢᵥ | EᵥᵢEᵥᵢᵢᵢ | E_{d} | E₁ |
| 505 | AlkLEᵥᵢᵢ | EᵥᵢEᵥᵢᵢᵢ | Eₑ | E₁ |
| 506 | AlkLEᵥᵢᵢEᵢ | EᵥᵢEᵥᵢᵢᵢ | Eₑ | E₁ |
| 507 | AlkLEᵥᵢᵢEᵢᵢ | EᵥᵢEᵥᵢᵢᵢ | Eₑ | E₁ |
| 508 | AlkLEᵥᵢᵢEᵢᵢE_{iib} | EᵥᵢEᵥᵢᵢᵢ | Eₑ | E₁ |
| 509 | AlkLEᵥᵢᵢEᵢᵢᵢ | EᵥᵢEᵥᵢᵢᵢ | Eₑ | E₁ |
| 510 | AlkLEᵥᵢᵢEᵢᵥ | EᵥᵢEᵥᵢᵢᵢ | Eₑ | E₁ |
| 511 | AlkLEᵥᵢᵢ | EᵥᵢEᵥᵢᵢᵢ | E_{f} | E₁ |
| 512 | AlkLEᵥᵢᵢEᵢ | EᵥᵢEᵥᵢᵢᵢ | E_{f} | E₁ |
| 513 | AlkLEᵥᵢᵢEᵢᵢ | EᵥᵢEᵥᵢᵢᵢ | E_{f} | E₁ |
| 514 | AlkLEᵥᵢᵢEᵢᵢE_{iib} | EᵥᵢEᵥᵢᵢᵢ | E_{f} | E₁ |
| 515 | AlkLEᵥᵢᵢEᵢᵢᵢ | EᵥᵢEᵥᵢᵢᵢ | E_{f} | E₁ |
| 516 | AlkLEᵥᵢᵢEᵢᵥ | EᵥᵢEᵥᵢᵢᵢ | E_{f} | E₁ |
| 517 | AlkLEᵥᵢᵢ | E_{iib}EᵥᵢEₓ | | E₁ |
| 518 | AlkLEᵥᵢᵢEᵢ | E_{iib}EᵥᵢEₓ | | E₁ |
| 519 | AlkLEᵥᵢᵢEᵢᵢ | E_{iib}EᵥᵢEₓ | | E₁ |
| 520 | AlkLEᵥᵢᵢEᵢᵢE_{iib} | E_{iib}EᵥᵢEₓ | | E₁ |
| 521 | AlkLEᵥᵢᵢEᵢᵢᵢ | E_{iib}EᵥᵢEₓ | | E₁ |
| 522 | AlkLEᵥᵢᵢEᵢᵥ | E_{iib}EᵥᵢEₓ | | E₁ |
| 523 | AlkLEᵥᵢᵢ | E_{iib}EᵥᵢEₓ | | E₁ |
| 524 | AlkLEᵥᵢᵢEᵢ | E_{iib}EᵥᵢEₓ | | E₁ |
| 525 | AlkLEᵥᵢᵢEᵢᵢ | E_{iib}EᵥᵢEₓ | | E₁ |
| 526 | AlkLEᵥᵢᵢEᵢᵢE_{iib} | E_{iib}EᵥᵢEₓ | | E₁ |
| 527 | AlkLEᵥᵢᵢEᵢᵢᵢ | E_{iib}EᵥᵢEₓ | | E₁ |
| 528 | AlkLEᵥᵢᵢEᵢᵥ | E_{iib}EᵥᵢEₓ | | E₁ |
| 529 | AlkLEᵥᵢᵢ | E_{iib}EᵥᵢEₓ | Eₐ | E₁ |
| 530 | AlkLEᵥᵢᵢEᵢ | E_{iib}EᵥᵢEₓ | Eₐ | E₁ |
| 531 | AlkLEᵥᵢᵢEᵢᵢ | E_{iib}EᵥᵢEₓ | Eₐ | E₁ |
| 532 | AlkLEᵥᵢᵢEᵢᵢE_{iib} | E_{iib}EᵥᵢEₓ | Eₐ | E₁ |
| 533 | AlkLEᵥᵢᵢEᵢᵢᵢ | E_{iib}EᵥᵢEₓ | Eₐ | E₁ |
| 534 | AlkLEᵥᵢᵢEᵢᵥ | E_{iib}EᵥᵢEₓ | Eₐ | E₁ |
| 535 | AlkLEᵥᵢᵢ | E_{iib}EᵥᵢEₓ | E_{b} | E₁ |
| 536 | AlkLEᵥᵢᵢEᵢ | E_{iib}EᵥᵢEₓ | E_{b} | E₁ |
| 537 | AlkLEᵥᵢᵢEᵢᵢ | E_{iib}EᵥᵢEₓ | E_{b} | E₁ |
| 538 | AlkLEᵥᵢᵢEᵢᵢE_{iib} | E_{iib}EᵥᵢEₓ | E_{b} | E₁ |
| 539 | AlkLEᵥᵢᵢEᵢᵢᵢ | E_{iib}EᵥᵢEₓ | E_{b} | E₁ |
| 540 | AlkLEᵥᵢᵢEᵢᵥ | E_{iib}EᵥᵢEₓ | E_{b} | E₁ |
| 541 | AlkLEᵥᵢᵢ | E_{iib}EᵥᵢEₓ | E_{d} | E₁ |
| 542 | AlkLEᵥᵢᵢEᵢ | E_{iib}EᵥᵢEₓ | E_{d} | E₁ |
| 543 | AlkLEᵥᵢᵢEᵢᵢ | E_{iib}EᵥᵢEₓ | E_{d} | E₁ |
| 544 | AlkLEᵥᵢᵢEᵢᵢE_{iib} | E_{iib}EᵥᵢEₓ | E_{d} | E₁ |
| 545 | AlkLEᵥᵢᵢEᵢᵢᵢ | E_{iib}EᵥᵢEₓ | E_{d} | E₁ |
| 546 | AlkLEᵥᵢᵢEᵢᵥ | E_{iib}EᵥᵢEₓ | E_{d} | E₁ |
| 547 | AlkLEᵥᵢᵢ | E_{iib}EᵥᵢEₓ | Eₑ | E₁ |
| 548 | AlkLEᵥᵢᵢEᵢ | E_{iib}EᵥᵢEₓ | Eₑ | E₁ |
| 549 | AlkLEᵥᵢᵢEᵢᵢ | E_{iib}EᵥᵢEₓ | Eₑ | E₁ |
| 550 | AlkLEᵥᵢᵢEᵢᵢE_{iib} | E_{iib}EᵥᵢEₓ | Eₑ | E₁ |
| 551 | AlkLEᵥᵢᵢEᵢᵢᵢ | E_{iib}EᵥᵢEₓ | Eₑ | E₁ |
| 552 | AlkLEᵥᵢᵢEᵢᵥ | E_{iib}EᵥᵢEₓ | Eₑ | E₁ |
| 553 | AlkLEᵥᵢᵢ | E_{iib}EᵥᵢEₓ | E_{f} | E₁ |
| 554 | AlkLEᵥᵢᵢEᵢ | E_{iib}EᵥᵢEₓ | E_{f} | E₁ |
| 555 | AlkLEᵥᵢᵢEᵢᵢ | E_{iib}EᵥᵢEₓ | E_{f} | E₁ |
| 556 | AlkLEᵥᵢᵢEᵢᵢE_{iib} | E_{iib}EᵥᵢEₓ | E_{f} | E₁ |
| 557 | AlkLEᵥᵢᵢEᵢᵢᵢ | E_{iib}EᵥᵢEₓ | E_{f} | E₁ |
| 558 | AlkLEᵥᵢᵢEᵢᵥ | E_{iib}EᵥᵢEₓ | E_{f} | E₁ |
| 559 | AlkLEᵥᵢᵢ | E_{iib}EᵥᵢEₓ | | E₁ |
| 560 | AlkLEᵥᵢᵢEᵢ | E_{iib}EᵥᵢEₓ | | E₁ |
| 561 | AlkLEᵥᵢᵢEᵢᵢ | E_{iib}EᵥᵢEₓ | | E₁ |
| 562 | AlkLEᵥᵢᵢEᵢᵢE_{iib} | E_{iib}EᵥᵢEₓ | | E₁ |
| 563 | AlkLEᵥᵢᵢEᵢᵢᵢ | E_{iib}EᵥᵢEₓ | | E₁ |
| 564 | AlkLEᵥᵢᵢEᵢᵥ | E_{iib}EᵥᵢEₓ | | E₁ |
| 565 | AlkLEₓᵢᵢᵢEᵥᵢᵢ | Eᵥᵢᵢᵢ | | E₁ |
| 566 | AlkLEₓᵢᵢᵢEᵥᵢᵢEᵢ | Eᵥᵢᵢᵢ | | E₁ |
| 567 | AlkLEₓᵢᵢᵢEᵥᵢᵢEᵢᵢ | Eᵥᵢᵢᵢ | | E₁ |
| 568 | AlkLEₓᵢᵢᵢEᵥᵢᵢEᵢᵢE_{iib} | Eᵥᵢᵢᵢ | | E₁ |
| 569 | AlkLEₓᵢᵢᵢEᵥᵢᵢEᵢᵢᵢ | Eᵥᵢᵢᵢ | | E₁ |
| 570 | AlkLEₓᵢᵢᵢEᵥᵢᵢEᵢᵥ | Eᵥᵢᵢᵢ | | E₁ |
| 571 | AlkLEₓᵢᵢᵢEᵥᵢᵢ | Eᵥᵢᵢᵢ | Eₐ | E₁ |
| 572 | AlkLEₓᵢᵢᵢEᵥᵢᵢEᵢ | Eᵥᵢᵢᵢ | Eₐ | E₁ |
| 573 | AlkLEₓᵢᵢᵢEᵥᵢᵢEᵢᵢ | Eᵥᵢᵢᵢ | Eₐ | E₁ |
| 574 | AlkLEₓᵢᵢᵢEᵥᵢᵢEᵢᵢE_{iib} | Eᵥᵢᵢᵢ | Eₐ | E₁ |
| 575 | AlkLEₓᵢᵢᵢEᵥᵢᵢEᵢᵢᵢ | Eᵥᵢᵢᵢ | Eₐ | E₁ |
| 576 | AlkLEₓᵢᵢᵢEᵥᵢᵢEᵢᵥ | Eᵥᵢᵢᵢ | Eₐ | E₁ |
| 577 | AlkLEₓᵢᵢᵢEᵥᵢᵢ | Eᵥᵢᵢᵢ | E_{b} | E₁ |
| 578 | AlkLEₓᵢᵢᵢEᵥᵢᵢEᵢ | Eᵥᵢᵢᵢ | E_{b} | E₁ |
| 579 | AlkLEₓᵢᵢᵢEᵥᵢᵢEᵢᵢ | Eᵥᵢᵢᵢ | E_{b} | E₁ |
| 580 | AlkLEₓᵢᵢᵢEᵥᵢᵢEᵢᵢE_{iib} | Eᵥᵢᵢᵢ | E_{b} | E₁ |
| 581 | AlkLEₓᵢᵢᵢEᵥᵢᵢEᵢᵢᵢ | Eᵥᵢᵢᵢ | E_{b} | E₁ |
| 582 | AlkLEₓᵢᵢᵢEᵥᵢᵢEᵢᵥ | Eᵥᵢᵢᵢ | E_{b} | E₁ |
| 583 | AlkLEₓᵢᵢᵢEᵥᵢᵢ | Eᵥᵢᵢᵢ | E_{d} | E₁ |
| 584 | AlkLEₓᵢᵢᵢEᵥᵢᵢEᵢ | Eᵥᵢᵢᵢ | E_{d} | E₁ |
| 585 | AlkLEₓᵢᵢᵢEᵥᵢᵢEᵢᵢ | Eᵥᵢᵢᵢ | E_{d} | E₁ |
| 586 | AlkLEₓᵢᵢᵢEᵥᵢᵢEᵢᵢE_{iib} | Eᵥᵢᵢᵢ | E_{d} | E₁ |
| 587 | AlkLEₓᵢᵢᵢEᵥᵢᵢEᵢᵢᵢ | Eᵥᵢᵢᵢ | E_{d} | E₁ |
| 588 | AlkLEₓᵢᵢᵢEᵥᵢᵢEᵢᵥ | Eᵥᵢᵢᵢ | E_{d} | E₁ |
| 589 | AlkLEₓᵢᵢᵢEᵥᵢᵢ | Eᵥᵢᵢᵢ | Eₑ | E₁ |
| 590 | AlkLEₓᵢᵢᵢEᵥᵢᵢEᵢ | Eᵥᵢᵢᵢ | Eₑ | E₁ |
| 591 | AlkLEₓᵢᵢᵢEᵥᵢᵢEᵢᵢ | Eᵥᵢᵢᵢ | Eₑ | E₁ |
| 592 | AlkLEₓᵢᵢᵢEᵥᵢᵢEᵢᵢE_{iib} | Eᵥᵢᵢᵢ | Eₑ | E₁ |
| 593 | AlkLEₓᵢᵢᵢEᵥᵢᵢEᵢᵢᵢ | Eᵥᵢᵢᵢ | Eₑ | E₁ |
| 594 | AlkLEₓᵢᵢᵢEᵥᵢᵢEᵢᵥ | Eᵥᵢᵢᵢ | Eₑ | E₁ |
| 595 | AlkLEₓᵢᵢᵢEᵥᵢᵢ | Eᵥᵢᵢᵢ | E_{f} | E₁ |
| 596 | AlkLEₓᵢᵢᵢEᵥᵢᵢEᵢ | Eᵥᵢᵢᵢ | E_{f} | E₁ |
| 597 | AlkLEₓᵢᵢᵢEᵥᵢᵢEᵢᵢ | Eᵥᵢᵢᵢ | E_{f} | E₁ |
| 598 | AlkLEₓᵢᵢᵢEᵥᵢᵢEᵢᵢE_{iib} | Eᵥᵢᵢᵢ | E_{f} | E₁ |
| 599 | AlkLEₓᵢᵢᵢEᵥᵢᵢEᵢᵢᵢ | Eᵥᵢᵢᵢ | E_{f} | E₁ |
| 600 | AlkLEₓᵢᵢᵢEᵥᵢᵢEᵢᵥ | Eᵥᵢᵢᵢ | E_{f} | E₁ |
| 601 | AlkLEₓᵢᵢᵢEᵥᵢᵢ | Eᵢₓ | | E₁ |
| 602 | AlkLEₓᵢᵢᵢEᵥᵢᵢEᵢ | Eᵢₓ | | E₁ |
| 603 | AlkLEₓᵢᵢᵢEᵥᵢᵢEᵢᵢ | Eᵢₓ | | E₁ |
| 604 | AlkLEₓᵢᵢᵢEᵥᵢᵢEᵢᵢE_{iib} | Eᵢₓ | | E₁ |
| 605 | AlkLEₓᵢᵢᵢEᵥᵢᵢEᵢᵢᵢ | Eᵢₓ | | E₁ |
| 606 | AlkLEₓᵢᵢᵢEᵥᵢᵢEᵢᵥ | Eᵢₓ | | E₁ |
| 607 | AlkLEₓᵢᵢᵢEᵥᵢᵢ | Eᵢₓ | Eₐ | E₁ |
| 608 | AlkLEₓᵢᵢᵢEᵥᵢᵢEᵢ | Eᵢₓ | Eₐ | E₁ |
| 609 | AlkLEₓᵢᵢᵢEᵥᵢᵢEᵢᵢ | Eᵢₓ | Eₐ | E₁ |
| 610 | AlkLEₓᵢᵢᵢEᵥᵢᵢEᵢᵢE_{iib} | Eᵢₓ | Eₐ | E₁ |
| 611 | AlkLEₓᵢᵢᵢEᵥᵢᵢEᵢᵢᵢ | Eᵢₓ | Eₐ | E₁ |
| 612 | AlkLEₓᵢᵢᵢEᵥᵢᵢEᵢᵥ | Eᵢₓ | Eₐ | E₁ |
| 613 | AlkLEₓᵢᵢᵢEᵥᵢᵢ | Eᵢₓ | E_{b} | E₁ |
| 614 | AlkLEₓᵢᵢᵢEᵥᵢᵢEᵢ | Eᵢₓ | E_{b} | E₁ |
| 615 | AlkLEₓᵢᵢᵢEᵥᵢᵢEᵢᵢ | Eᵢₓ | E_{b} | E₁ |
| 616 | AlkLEₓᵢᵢᵢEᵥᵢᵢEᵢᵢE_{iib} | Eᵢₓ | E_{b} | E₁ |
| 617 | AlkLEₓᵢᵢᵢEᵥᵢᵢEᵢᵢᵢ | Eᵢₓ | E_{b} | E₁ |
| 618 | AlkLEₓᵢᵢᵢEᵥᵢᵢEᵢᵥ | Eᵢₓ | E_{b} | E₁ |
| 619 | AlkLEₓᵢᵢᵢEᵥᵢᵢ | Eᵢₓ | E_{d} | E₁ |
| 620 | AlkLEₓᵢᵢᵢEᵥᵢᵢEᵢ | Eᵢₓ | E_{d} | E₁ |
| 621 | AlkLEₓᵢᵢᵢEᵥᵢᵢEᵢᵢ | Eᵢₓ | E_{d} | E₁ |
| 622 | AlkLEₓᵢᵢᵢEᵥᵢᵢEᵢᵢE_{iib} | Eᵢₓ | E_{d} | E₁ |
| 623 | AlkLEₓᵢᵢᵢEᵥᵢᵢEᵢᵢᵢ | Eᵢₓ | E_{d} | E₁ |
| 624 | AlkLEₓᵢᵢᵢEᵥᵢᵢEᵢᵥ | Eᵢₓ | E_{d} | E₁ |
| 625 | AlkLEₓᵢᵢᵢEᵥᵢᵢ | Eᵢₓ | Eₑ | E₁ |
| 626 | AlkLEₓᵢᵢᵢEᵥᵢᵢEᵢ | Eᵢₓ | Eₑ | E₁ |
| 627 | AlkLEₓᵢᵢᵢEᵥᵢᵢEᵢᵢ | Eᵢₓ | Eₑ | E₁ |
| 628 | AlkLEₓᵢᵢᵢEᵥᵢᵢEᵢᵢE_{iib} | Eᵢₓ | Eₑ | E₁ |
| 629 | AlkLEₓᵢᵢᵢEᵥᵢᵢEᵢᵢᵢ | Eᵢₓ | Eₑ | E₁ |
| 630 | AlkLEₓᵢᵢᵢEᵥᵢᵢEᵢᵥ | Eᵢₓ | Eₑ | E₁ |
| 631 | AlkLEₓᵢᵢᵢEᵥᵢᵢ | Eᵢₓ | E_{f} | E₁ |
| 632 | AlkLEₓᵢᵢᵢEᵥᵢᵢEᵢ | Eᵢₓ | E_{f} | E₁ |
| 633 | AlkLEₓᵢᵢᵢEᵥᵢᵢEᵢᵢ | Eᵢₓ | E_{f} | E₁ |
| 634 | AlkLEₓᵢᵢᵢEᵥᵢᵢEᵢᵢE_{iib} | Eᵢₓ | E_{f} | E₁ |
| 635 | AlkLEₓᵢᵢᵢEᵥᵢᵢEᵢᵢᵢ | Eᵢₓ | E_{f} | E₁ |
| 636 | AlkLEₓᵢᵢᵢEᵥᵢᵢEᵢᵥ | Eᵢₓ | E_{f} | E₁ |
| 637 | AlkLEₓᵢᵢᵢEᵥᵢᵢ | Eₓ | | E₁ |
| 638 | AlkLEₓᵢᵢᵢEᵥᵢᵢEᵢ | Eₓ | | E₁ |
| 639 | AlkLEₓᵢᵢᵢEᵥᵢᵢEᵢᵢ | Eₓ | | E₁ |
| 640 | AlkLEₓᵢᵢᵢEᵥᵢᵢEᵢᵢE_{iib} | Eₓ | | E₁ |
| 641 | AlkLEₓᵢᵢᵢEᵥᵢᵢEᵢᵢᵢ | Eₓ | | E₁ |
| 642 | AlkLEₓᵢᵢᵢEᵥᵢᵢEᵢᵥ | Eₓ | | E₁ |
| 643 | AlkLEₓᵢᵢᵢEᵥᵢᵢ | Eₓ | Eₐ | E₁ |
| 644 | AlkLEₓᵢᵢᵢEᵥᵢᵢEᵢ | Eₓ | Eₐ | E₁ |
| 645 | AlkLEₓᵢᵢᵢEᵥᵢᵢEᵢᵢ | Eₓ | Eₐ | E₁ |
| 646 | AlkLEₓᵢᵢᵢEᵥᵢᵢEᵢᵢE_{iib} | Eₓ | Eₐ | E₁ |
| 647 | AlkLEₓᵢᵢᵢEᵥᵢᵢEᵢᵢᵢ | Eₓ | Eₐ | E₁ |
| 648 | AlkLEₓᵢᵢᵢEᵥᵢᵢEᵢᵥ | Eₓ | Eₐ | E₁ |
| 649 | AlkLEₓᵢᵢᵢEᵥᵢᵢ | Eₓ | E_{b} | E₁ |
| 650 | AlkLEₓᵢᵢᵢEᵥᵢᵢEᵢ | Eₓ | E_{b} | E₁ |
| 651 | AlkLEₓᵢᵢᵢEᵥᵢᵢEᵢᵢ | Eₓ | E_{b} | E₁ |
| 652 | AlkLEₓᵢᵢᵢEᵥᵢᵢEᵢᵢE_{iib} | Eₓ | E_{b} | E₁ |
| 653 | AlkLEₓᵢᵢᵢEᵥᵢᵢEᵢᵢᵢ | Eₓ | E_{b} | E₁ |
| 654 | AlkLEₓᵢᵢᵢEᵥᵢᵢEᵢᵥ | Eₓ | E_{b} | E₁ |
| 655 | AlkLEₓᵢᵢᵢEᵥᵢᵢ | Eₓ | E_{d} | E₁ |
| 656 | AlkLEₓᵢᵢᵢEᵥᵢᵢEᵢ | Eₓ | E_{d} | E₁ |
| 657 | AlkLEₓᵢᵢᵢEᵥᵢᵢEᵢᵢ | Eₓ | E_{d} | E₁ |
| 658 | AlkLEₓᵢᵢᵢEᵥᵢᵢEᵢᵢE_{iib} | Eₓ | E_{d} | E₁ |
| 659 | AlkLEₓᵢᵢᵢEᵥᵢᵢEᵢᵢᵢ | Eₓ | E_{d} | E₁ |
| 660 | AlkLEₓᵢᵢᵢEᵥᵢᵢEᵢᵥ | Eₓ | E_{d} | E₁ |
| 661 | AlkLEₓᵢᵢᵢEᵥᵢᵢ | Eₓ | Eₑ | E₁ |
| 662 | AlkLEₓᵢᵢᵢEᵥᵢᵢEᵢ | Eₓ | Eₑ | E₁ |
| 663 | AlkLEₓᵢᵢᵢEᵥᵢᵢEᵢᵢ | Eₓ | Eₑ | E₁ |
| 664 | AlkLEₓᵢᵢᵢEᵥᵢᵢEᵢᵢE_{iib} | Eₓ | Eₑ | E₁ |
| 665 | AlkLEₓᵢᵢᵢEᵥᵢᵢEᵢᵢᵢ | Eₓ | Eₑ | E₁ |
| 666 | AlkLEₓᵢᵢᵢEᵥᵢᵢEᵢᵥ | Eₓ | Eₑ | E₁ |
| 667 | AlkLEₓᵢᵢᵢEᵥᵢᵢ | Eₓ | E_{f} | E₁ |
| 668 | AlkLEₓᵢᵢᵢEᵥᵢᵢEᵢ | Eₓ | E_{f} | E₁ |
| 669 | AlkLEₓᵢᵢᵢEᵥᵢᵢEᵢᵢ | Eₓ | E_{f} | E₁ |
| 670 | AlkLEₓᵢᵢᵢEᵥᵢᵢEᵢᵢE_{iib} | Eₓ | E_{f} | E₁ |
| 671 | AlkLEₓᵢᵢᵢEᵥᵢᵢEᵢᵢᵢ | Eₓ | E_{f} | E₁ |
| 672 | AlkLEₓᵢᵢᵢEᵥᵢᵢEᵢᵥ | Eₓ | E_{f} | E₁ |
| 673 | AlkLEₓᵢᵢᵢEᵥᵢᵢ | EᵥᵢEᵥᵢᵢᵢ | | E₁ |
| 674 | AlkLEₓᵢᵢᵢEᵥᵢᵢEᵢ | EᵥᵢEᵥᵢᵢᵢ | | E₁ |
| 675 | AlkLEₓᵢᵢᵢEᵥᵢᵢEᵢᵢ | EᵥᵢEᵥᵢᵢᵢ | | E₁ |
| 676 | AlkLEₓᵢᵢᵢEᵥᵢᵢEᵢᵢE_{iib} | EᵥᵢEᵥᵢᵢᵢ | | E₁ |
| 677 | AlkLEₓᵢᵢᵢEᵥᵢᵢEᵢᵢᵢ | EᵥᵢEᵥᵢᵢᵢ | | E₁ |
| 678 | AlkLEₓᵢᵢᵢEᵥᵢᵢEᵢᵥ | EᵥᵢEᵥᵢᵢᵢ | | E₁ |
| 679 | AlkLEₓᵢᵢᵢEᵥᵢᵢ | EᵥᵢEᵥᵢᵢᵢ | Eₐ | E₁ |
| 680 | AlkLEₓᵢᵢᵢEᵥᵢᵢEᵢ | EᵥᵢEᵥᵢᵢᵢ | Eₐ | E₁ |
| 681 | AlkLEₓᵢᵢᵢEᵥᵢᵢEᵢᵢ | EᵥᵢEᵥᵢᵢᵢ | Eₐ | E₁ |
| 682 | AlkLEₓᵢᵢᵢEᵥᵢᵢEᵢᵢE_{iib} | EᵥᵢEᵥᵢᵢᵢ | Eₐ | E₁ |
| 683 | AlkLEₓᵢᵢᵢEᵥᵢᵢEᵢᵢᵢ | EᵥᵢEᵥᵢᵢᵢ | Eₐ | E₁ |
| 684 | AlkLEₓᵢᵢᵢEᵥᵢᵢEᵢᵥ | EᵥᵢEᵥᵢᵢᵢ | Eₐ | E₁ |
| 685 | AlkLEₓᵢᵢᵢEᵥᵢᵢ | EᵥᵢEᵥᵢᵢᵢ | E_{b} | E₁ |
| 686 | AlkLEₓᵢᵢᵢEᵥᵢᵢEᵢ | EᵥᵢEᵥᵢᵢᵢ | E_{b} | E₁ |
| 687 | AlkLEₓᵢᵢᵢEᵥᵢᵢEᵢᵢ | EᵥᵢEᵥᵢᵢᵢ | E_{b} | E₁ |
| 688 | AlkLEₓᵢᵢᵢEᵥᵢᵢEᵢᵢE_{iib} | EᵥᵢEᵥᵢᵢᵢ | E_{b} | E₁ |
| 689 | AlkLEₓᵢᵢᵢEᵥᵢᵢEᵢᵢᵢ | EᵥᵢEᵥᵢᵢᵢ | E_{b} | E₁ |
| 690 | AlkLEₓᵢᵢᵢEᵥᵢᵢEᵢᵥ | EᵥᵢEᵥᵢᵢᵢ | E_{b} | E₁ |
| 691 | AlkLEₓᵢᵢᵢEᵥᵢᵢ | EᵥᵢEᵥᵢᵢᵢ | E_{d} | E₁ |
| 692 | AlkLEₓᵢᵢᵢEᵥᵢᵢEᵢ | EᵥᵢEᵥᵢᵢᵢ | E_{d} | E₁ |
| 693 | AlkLEₓᵢᵢᵢEᵥᵢᵢEᵢᵢ | EᵥᵢEᵥᵢᵢᵢ | E_{d} | E₁ |
| 694 | AlkLEₓᵢᵢᵢEᵥᵢᵢEᵢᵢE_{iib} | EᵥᵢEᵥᵢᵢᵢ | E_{d} | E₁ |
| 695 | AlkLEₓᵢᵢᵢEᵥᵢᵢEᵢᵢᵢ | EᵥᵢEᵥᵢᵢᵢ | E_{d} | E₁ |
| 696 | AlkLEₓᵢᵢᵢEᵥᵢᵢEᵢᵥ | EᵥᵢEᵥᵢᵢᵢ | E_{d} | E₁ |
| 697 | AlkLEₓᵢᵢᵢEᵥᵢᵢ | EᵥᵢEᵥᵢᵢᵢ | Eₑ | E₁ |
| 698 | AlkLEₓᵢᵢᵢEᵥᵢᵢEᵢ | EᵥᵢEᵥᵢᵢᵢ | Eₑ | E₁ |
| 699 | AlkLEₓᵢᵢᵢEᵥᵢᵢEᵢᵢ | EᵥᵢEᵥᵢᵢᵢ | Eₑ | E₁ |
| 700 | AlkLEₓᵢᵢᵢEᵥᵢᵢEᵢᵢE_{iib} | EᵥᵢEᵥᵢᵢᵢ | Eₑ | E₁ |
| 701 | AlkLEₓᵢᵢᵢEᵥᵢᵢEᵢᵢᵢ | EᵥᵢEᵥᵢᵢᵢ | Eₑ | E₁ |
| 702 | AlkLEₓᵢᵢᵢEᵥᵢᵢEᵢᵥ | EᵥᵢEᵥᵢᵢᵢ | Eₑ | E₁ |
| 703 | AlkLEₓᵢᵢᵢEᵥᵢᵢ | EᵥᵢEᵥᵢᵢᵢ | E_{f} | E₁ |
| 704 | AlkLEₓᵢᵢᵢEᵥᵢᵢEᵢ | EᵥᵢEᵥᵢᵢᵢ | E_{f} | E₁ |
| 705 | AlkLEₓᵢᵢᵢEᵥᵢᵢEᵢᵢ | EᵥᵢEᵥᵢᵢᵢ | E_{f} | E₁ |
| 706 | AlkLEₓᵢᵢᵢEᵥᵢᵢEᵢᵢE_{iib} | EᵥᵢEᵥᵢᵢᵢ | E_{f} | E₁ |
| 707 | AlkLEₓᵢᵢᵢEᵥᵢᵢEᵢᵢᵢ | EᵥᵢEᵥᵢᵢᵢ | E_{f} | E₁ |
| 708 | AlkLEₓᵢᵢᵢEᵥᵢᵢEᵢᵥ | EᵥᵢEᵥᵢᵢᵢ | E_{f} | E₁ |
| 709 | AlkLEₓᵢᵢᵢEᵥᵢᵢ | E_{iib}EᵥᵢEₓ | | E₁ |
| 710 | AlkLEₓᵢᵢᵢEᵥᵢᵢEᵢ | E_{iib}EᵥᵢEₓ | | E₁ |
| 711 | AlkLEₓᵢᵢᵢEᵥᵢᵢEᵢᵢ | E_{iib}EᵥᵢEₓ | | E₁ |
| 712 | AlkLEₓᵢᵢᵢEᵥᵢᵢEᵢᵢE_{iib} | E_{iib}EᵥᵢEₓ | | E₁ |
| 713 | AlkLEₓᵢᵢᵢEᵥᵢᵢEᵢᵢᵢ | E_{iib}EᵥᵢEₓ | | E₁ |
| 714 | AlkLEₓᵢᵢᵢEᵥᵢᵢEᵢᵥ | E_{iib}EᵥᵢEₓ | | E₁ |
| 715 | AlkLEₓᵢᵢᵢEᵥᵢᵢ | E_{iib}EᵥᵢEₓ | Eₐ | E₁ |
| 716 | AlkLEₓᵢᵢᵢEᵥᵢᵢEᵢ | E_{iib}EᵥᵢEₓ | Eₐ | E₁ |
| 717 | AlkLEₓᵢᵢᵢEᵥᵢᵢEᵢᵢ | E_{iib}EᵥᵢEₓ | Eₐ | E₁ |
| 718 | AlkLEₓᵢᵢᵢEᵥᵢᵢEᵢᵢE_{iib} | E_{iib}EᵥᵢEₓ | Eₐ | E₁ |
| 719 | AlkLEₓᵢᵢᵢEᵥᵢᵢEᵢᵢᵢ | E_{iib}EᵥᵢEₓ | Eₐ | E₁ |
| 720 | AlkLEₓᵢᵢᵢEᵥᵢᵢEᵢᵥ | E_{iib}EᵥᵢEₓ | Eₐ | E₁ |
| 721 | AlkLEₓᵢᵢᵢEᵥᵢᵢ | E_{iib}EᵥᵢEₓ | E_{b} | E₁ |
| 722 | AlkLEₓᵢᵢᵢEᵥᵢᵢEᵢ | E_{iib}EᵥᵢEₓ | E_{b} | E₁ |
| 723 | AlkLEₓᵢᵢᵢEᵥᵢᵢEᵢᵢ | E_{iib}EᵥᵢEₓ | E_{b} | E₁ |
| 724 | AlkLEₓᵢᵢᵢEᵥᵢᵢEᵢᵢE_{iib} | E_{iib}EᵥᵢEₓ | E_{b} | E₁ |
| 725 | AlkLEₓᵢᵢᵢEᵥᵢᵢEᵢᵢᵢ | E_{iib}EᵥᵢEₓ | E_{b} | E₁ |
| 726 | AlkLEₓᵢᵢᵢEᵥᵢᵢEᵢᵥ | E_{iib}EᵥᵢEₓ | E_{b} | E₁ |
| 727 | AlkLEₓᵢᵢᵢEᵥᵢᵢ | E_{iib}EᵥᵢEₓ | E_{d} | E₁ |
| 728 | AlkLEₓᵢᵢᵢEᵥᵢᵢEᵢ | E_{iib}EᵥᵢEₓ | E_{d} | E₁ |
| 729 | AlkLEₓᵢᵢᵢEᵥᵢᵢEᵢᵢ | E_{iib}EᵥᵢEₓ | E_{d} | E₁ |
| 730 | AlkLEₓᵢᵢᵢEᵥᵢᵢEᵢᵢE_{iib} | E_{iib}EᵥᵢEₓ | E_{d} | E₁ |
| 731 | AlkLEₓᵢᵢᵢEᵥᵢᵢEᵢᵢᵢ | E_{iib}EᵥᵢEₓ | E_{d} | E₁ |
| 732 | AlkLEₓᵢᵢᵢEᵥᵢᵢEᵢᵥ | E_{iib}EᵥᵢEₓ | E_{d} | E₁ |
| 733 | AlkLEₓᵢᵢᵢEᵥᵢᵢ | E_{iib}EᵥᵢEₓ | Eₑ | E₁ |
| 734 | AlkLEₓᵢᵢᵢEᵥᵢᵢEᵢ | E_{iib}EᵥᵢEₓ | Eₑ | E₁ |
| 735 | AlkLEₓᵢᵢᵢEᵥᵢᵢEᵢᵢ | E_{iib}EᵥᵢEₓ | Eₑ | E₁ |
| 736 | AlkLEₓᵢᵢᵢEᵥᵢᵢEᵢᵢE_{iib} | E_{iib}EᵥᵢEₓ | Eₑ | E₁ |
| 737 | AlkLEₓᵢᵢᵢEᵥᵢᵢEᵢᵢᵢ | E_{iib}EᵥᵢEₓ | Eₑ | E₁ |
| 738 | AlkLEₓᵢᵢᵢEᵥᵢᵢEᵢᵥ | E_{iib}EᵥᵢEₓ | Eₑ | E₁ |
| 739 | AlkLEₓᵢᵢᵢEᵥᵢᵢ | E_{iib}EᵥᵢEₓ | E_{f} | E₁ |
| 740 | AlkLEₓᵢᵢᵢEᵥᵢᵢEᵢ | E_{iib}EᵥᵢEₓ | E_{f} | E₁ |
| 741 | AlkLEₓᵢᵢᵢEᵥᵢᵢEᵢᵢ | E_{iib}EᵥᵢEₓ | E_{f} | E₁ |
| 742 | AlkLEₓᵢᵢᵢEᵥᵢᵢEᵢᵢE_{iib} | E_{iib}EᵥᵢEₓ | E_{f} | E₁ |
| 743 | AlkLEₓᵢᵢᵢEᵥᵢᵢEᵢᵢᵢ | E_{iib}EᵥᵢEₓ | E_{f} | E₁ |
| 744 | AlkLEₓᵢᵢᵢEᵥᵢᵢEᵢᵥ | E_{iib}EᵥᵢEₓ | E_{f} | E₁ |

Especially preferred alternative embodiments of microorganisms according to the invention are explained hereinafter:

For preparing carboxylic acids with 6 to 18 carbon atoms, in particular fatty acids, microorganisms according to the invention which are particularly preferably suitable are characterized in that the first genetic modification is an activity which is increased in comparison with the enzymatic activity of the wild type of the microorganism of at least one of the enzymes Eᵢ which comprises sequences selected from among: AAC49180.1 (encoded by SEQ ID No.: 10), AAC49269.1 (encoded by SEQ ID No.: 8), Q39513.1 (encoded by SEQ ID No.: 9), AAC49001.1 (encoded by SEQ ID No.: 37), AEM72521.1 (encoded by SEQ ID No.: 35)

and
proteins having a polypeptide sequence in which up to 60%, preferably up to 25%, particularly preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢ is generally understood in particular as meaning the hydrolysis of dodecanoyl-ACP thioester,
and the alkL gene product is selected from among those that are encoded by the alkL gene of Pseudomonas putida GPo1, which is rendered by SEQ ID No. 1, and proteins with polypeptide sequence SEQ ID No. 2, SEQ ID No. 31, SEQ ID No. 32 or SEQ ID No. 33 or with a polypeptide sequence in which up to 60%, preferably up to 25%, particularly preferably up to 15%, in particular up to 10, 9, 8, 7, 6, 5, 4, 3, 2, 1% of the amino acid residues are modified in comparison with SEQ ID No. 2, SEQ ID No. 31, SEQ ID No. 32 or SEQ ID No. 33 by deletion, insertion, substitution or a combination thereof and which still have at least 50%, preferably 65%, particularly preferably 80%, in particular more than 90%, of the activity of the protein with the respective reference sequence SEQ ID No. 2, SEQ ID No. 31, SEQ ID No. 32 or SEQ ID No. 33, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, more particularly in a system as is described in the exemplary embodiments, in which glucose is reacted to form palmitoleic acid in an E. coli cell.

In this context it can be advantageous when the microorganism contains a fifth genetic modification which has an activity which is reduced compared with the enzymatic activity of the wild type of the microorganism of at least one of the enzymes E_{b}, where E_{b} is selected from among enzymes which comprise sequences selected from YP_{—}488518.1 (encoded by SEQ ID No. 14, formerly AP_{—}000876.1) and

proteins having a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% of the activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to 2-dodecenoyl-CoA thioester.

For producing esters of carboxylic acids having 6 to 18 carbon atoms in the carboxylic acid portion in which the alcohol component is derived from methanol or ethanol, especially preferably, microorganisms according to the invention are suitable which are characterized in that the first genetic modification is an activity which is increased in comparison with the enzymatic activity of the wild type of the microorganism of at least one of the enzymes Eᵢ, which comprises sequences selected from among: AAC49180.1 (encoded by SEQ ID No.: 10), AAC49269.1 (encoded by SEQ ID No.: 8), Q39513.1 (encoded by SEQ ID No.: 9), AAC49001.1 (encoded by SEQ ID No.: 37), AEM72521.1 (encoded by SEQ ID No.: 35)

and
proteins having a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢ is generally understood in particular as meaning the hydrolysis of dodecanoyl-ACP thioester,
and
the alkL gene product is selected from those which are encoded by the alkL gene of Pseudomonas putida GPo1, which is rendered by SEQ ID No. 1, and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with SEQ ID No. 2 by deletion, insertion, substitution or a combination thereof and which still have at least 50%, preferably 65%, especially preferably 80%, in particular more than 90% of the activity of the protein with the reference sequence SEQ ID No. 2, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, namely in a system as described in the exemplary embodiments in which glucose is reacted to form palmitoleic acid in an E. coli cell,
and
in that it has a third genetic modification which has an activity of the enzymes Eᵥ and Eᵥᵢ, which is increased in comparison with the enzymatic activity of the wild type of the microorganism,
where Eᵥ is selected from among YP_{—}694462.1 (encoded by SEQ ID No. 67) and YP_{—}045555.1 (encoded by SEQ ID No. 19), and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥ is generally understood in particular as meaning the conversion of dodecanoyl-CoA thioester with methanol to form dodecanoyl methyl ester,
and Eᵥᵢ is selected from among YP_{—}001724804.1 (encoded by SEQ ID No.: 18), and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥᵢ is generally understood in particular as meaning the synthesis of dodecanoyl-CoA thioester.

In this context, it can also be advantageous if the microorganism contains a fifth genetic modification which has an activity which is reduced compared with the enzymatic activity of the wild type of the microorganism of at least one of the enzymes E_{b}, where E_{b} is selected from enzymes which comprises sequences that are selected from among YP_{—}488518.1 (encoded by SEQ ID No. 14, formerly AP_{—}000876.1), and

proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to form 2-dodecenoyl-CoA thioester.

In an alternative embodiment for producing esters of carboxylic acid with 6 to 18 carbon atoms in the carboxylic acid portion, in which the alcohol component is derived from methanol or ethanol, microorganisms according to the invention suitable which are particularly preferably are characterized in that the first genetic modification is an activity which is increased in comparison with the enzymatic activity of the wild type of at least one of the enzymes Eᵢ which comprises sequences that are selected from among: AAC49269.1 (encoded by SEQ ID No.: 8), Q39513.1 (encoded by SEQ ID No.: 9), AAC49001.1 (encoded by SEQ ID No.: 37), AEM72521.1 (encoded by SEQ ID No.: 35)

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢ is generally understood in particular as meaning the hydrolysis of dodecanoyl-ACP thioester,
and
the alkL gene product is selected from those which are encoded by the alkL gene of Pseudomonas putida GPo1, which is shown by SEQ ID No. 1, and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with SEQ ID No. 2 by deletion, insertion, substitution or a combination thereof and which still have at least 50%, preferably 65%, especially preferably 80%, in particular more than 90%, of the activity of the protein with the reference sequence SEQ ID No. 2, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, more precisely in a system as described in the exemplary embodiments in which glucose is reacted to form palmitoleic acid in an E. coli cell,
and
in that it has a third genetic modification which has an activity which is increased in comparison with the enzymatic activity of the wild type of the microorganism of the enzyme Eᵥₐ,
where Eᵥₐ is selected from among YP_{—}888622.1 (encoded by SEQ ID No. 114) and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥₐ is generally understood in particular as meaning the conversion of lauric acid and S-adenosylmethionine to form lauric acid methyl ester and S-adenosylhomocysteine.

In this context it can also be advantageous if the microorganism contains a fifth genetic modification which has an activity which is reduced compared with the enzymatic activity of the wild type of the microorganism of at least one of the enzymes E_{b}, where E_{b} is selected from enzymes which have sequences that are selected from among YP_{—}488518.1 (encoded by SEQ ID No. 14, formerly AP_{—}000876.1), and

proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to give 2-dodecenoyl-CoA thioester.

For production of monohydric alcohols with 6 to 18 carbon atoms, microorganisms according to the invention which are especially preferably suitable are characterized in that the first genetic modification is an activity that is increased in comparison with the enzymatic activity of the wild type of the microorganism of at least one of the enzymes Eᵢ which comprises sequences selected from among: AAC49269.1 (encoded by SEQ ID No: 8), AEM72521.1 (encoded by SEQ ID No: 35)

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢ is generally understood in particular as meaning the hydrolysis of dodecanoyl-ACP thioester,
and
the alkL gene product is selected from among those encoded by the alkL gene of Pseudomonas putida GPo1, which is shown by SEQ ID No. 1, and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with SEQ ID No. 2 by deletion, insertion, substitution or a combination thereof and which still have at least 50%, preferably 65%, especially preferably 80%, in particular more than 90%, of the activity of the protein with the reference sequence SEQ ID No. 2, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, namely in a system as described in the exemplary embodiments in which glucose is reacted to form palmitoleic acid in an E. coli cell, and in that it has a third genetic modification which has an activity which is increased in comparison with the enzymatic activity of the wild type of the microorganism of the enzyme Eᵥᵢ,
where Eᵥᵢ is selected from among YP_{—}001724804.1 (encoded by SEQ ID No: 18)
and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵥᵢ is generally understood in particular as meaning the synthesis of dodecanoyl-CoA thioester, and
in that it has a fourth genetic modification which has an activity which is increased in comparison with the enzymatic activity of the wild type of the microorganism of the enzyme Eₓ, where Eₓ is selected from among BAB85476.1 (encoded by SEQ ID No. 77), YP_{—}047869.1 (encoded by SEQ ID No. 79 or 81), YP_{—}959486.1 (encoded by SEQ ID No. 83), YP_{—}959769.1 (encoded by SEQ ID No. 139), B9TSP7.1 (encoded by SEQ ID No. 141), and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), in comparison with the activity of the biocatalyst without the presence of the reference protein, where activity in this context and in the context of the determination of the activity of the enzyme Eₓ is generally understood in particular as meaning the synthesis of lauryl alcohol and NAD(P)⁺ from lauryl-ACP, NAD(P)H and H⁺. In this context it can also be advantageous if the microorganism contains a fifth genetic modification which has an activity which is reduced compared with the enzymatic activity of the wild type of the microorganism of at least one of the enzymes E_{b}, wherein E_{b} is selected from enzymes that have sequences which are selected from among YP_{—}488518.1 (encoded by SEQ ID No. 14, formerly AP_{—}000876.1), and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution, or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to form 2-dodecenoyl-CoA thioester.

For producing monohydric alcohols and aldehydes with 6 to 18 carbon atoms, microorganisms according to the invention which are especially preferably suitable are characterized in that the first genetic modification is an activity that is increased in comparison with the enzymatic activity of the wild type of the microorganism of at least one of the enzymes Eᵢ which comprises sequences selected from among: AAC49269.1 (encoded by SEQ ID No: 8), Q39513.1 (encoded by SEQ ID No: 9), AAC49001.1 (encoded by SEQ ID No: 37), AEM72521.1 (encoded by SEQ ID No: 35)

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢ is generally understood in particular as meaning the hydrolysis of dodecanoyl-ACP thioester,
and
the alkL gene product is selected from those encoded by the alkL gene of Pseudomonas putida GPo1, which is shown by SEQ ID No. 1, and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with SEQ ID No. 2 by deletion, insertion, substitution or a combination thereof and which still have at least 50%, preferably 65%, especially preferably 80%, in particular more than 90%, of the activity of the protein with the reference sequence SEQ ID No. 2, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, namely in a system as described in the exemplary embodiments, in which glucose is reacted to form palmitoleic acid in an E. coli cell,
and
in that it has a fourth genetic modification which has an activity which is increased in comparison with the enzymatic activity of the wild type of the microorganism of the enzyme Eᵢₓ, where Eᵢₓ is selected from among YP_{—}887275.1 (encoded by SEQ ID No. 117), ABI83656.1 (encoded by SEQ ID No. 122), and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢₓ is generally understood in particular as meaning the synthesis of lauryl aldehyde, NADP, AMP and 2 Pᵢ from lauric acid, ATP, NADPH and H⁺.

In this context it can also be advantageous if the microorganism contains a fifth genetic modification which has an activity which is reduced in comparison with the enzymatic activity of the wild type of the microorganism of at least one of the enzymes E_{b}, where E_{b} is selected from enzymes which comprise sequences selected from among YP_{—}488518.1 (encoded by SEQ ID No. 14, formerly AP_{—}000876.1), and

proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to form 2-dodecenoyl-CoA thioester.

For production of alkylamines with 8 to 16 carbon atoms, microorganisms according to the invention which are especially preferably suitable are characterized in that the first genetic modification is an activity that is increased in comparison with the enzymatic activity of the wild type of the microorganism of at least one of the enzymes Eᵢ, which comprises sequences that are selected from among: AAC49269.1 (encoded by SEQ ID No: 8), Q39513.1 (encoded by SEQ ID No: 9), AAC49001.1 (encoded by SEQ ID No: 37),

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), in comparison with the activity of the biocatalyst without the presence of the reference protein, where activity in this context and in the context of the determination of the activity of the enzyme Eᵢ is generally understood in particular as meaning the hydrolysis of dodecanoyl-ACP thioester,
and
the alkL gene product is selected from those encoded by the alkL gene of Pseudomonas putida GPo1, which is shown by SEQ ID No. 1, and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with SEQ ID No. 2 by deletion, insertion, substitution or a combination thereof and which still have at least 50%, preferably 65%, especially preferably 80%, in particular more than 90%, of the activity of the protein with the reference sequence SEQ ID No. 2, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, more precisely in a system as described in the exemplary embodiments in which glucose is reacted to form palmitoleic acid in an E. coli cell,
and
it has a fourth genetic modification which has an activity which is increased in comparison with the enzymatic activity of the wild type of the microorganism of at least one enzyme Eₓᵢᵢᵢ, where this is selected from among NP_{—}901695.1 (encoded by SEQ ID No. 132) and
proteins having a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eₓᵢᵢᵢ is generally understood in particular as meaning the reaction of ω-oxolauric acid and/or ω-oxolauric acid methyl ester to form ω-aminolauric acid and/or ω-aminolauric acid methyl ester.

It can also be advantageous in this context if the microorganism contains a fifth genetic modification which has an activity which is reduced in comparison with the enzymatic activity of the wild type of the microorganism of at least one of the enzymes E_{b}, where E_{b} is selected from enzymes which have sequences selected from among YP_{—}488518.1 (encoded by SEQ ID No. 14, formerly AP_{—}000876.1), and

proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to form 2-dodecenoyl-CoA thioester.

For production of alkenes with 6 to 18 carbon atoms, microorganisms according to the invention which are particularly preferably suitable are characterized in that the first genetic modification is an activity which is increased in comparison with the enzymatic activity of the wild type of the microorganism of at least one of the enzymes Eᵢ, which comprises sequences selected from among: AAC49269.1 (encoded by SEQ ID No: 8), Q39513.1 (encoded by SEQ ID No: 9), AAC49001.1 (encoded by SEQ ID No: 37), AEM72521.1 (encoded by SEQ ID No: 35)

and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eᵢ is generally understood in particular as meaning the hydrolysis of dodecanoyl-ACP thioester,
and
the alkL gene product is selected from among those encoded by the alkL gene of Pseudomonas putida GPo1, which is shown by SEQ ID No. 1, and proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with SEQ ID No. 2, by deletion, insertion, substitution or a combination thereof and which still have at least 50%, preferably 65%, especially preferably 80%, in particular more than 90%, of the activity of the protein with the reference sequence SEQ ID No. 2, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, namely in a system as is described in the exemplary embodiments, in which glucose is reacted to form palmitoleic acid in an E. coli cell,
and
it has a fourth genetic modification which has an activity which is increased in comparison with the enzymatic activity of the wild type of the microorganism of at least one enzyme Eₓᵢ, where this is selected from among ADW41779.1 (encoded by SEQ ID No. 168) and
proteins with a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequence by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme Eₓᵢᵢᵢ is generally understood in particular as meaning the reaction of sodium palmitate with hydrogen peroxide to form pentadecene, CO₂ and water.

In this context it can also be advantageous if the microorganism contains a fifth genetic modification which has an activity which is reduced in comparison with the enzymatic activity of the wild type of the microorganism of at least one of the enzymes E_{b}, where E_{b} is selected from among enzymes which comprise sequences selected from among YP_{—}488518.1 (encoded by SEQ ID No. 14),

and
proteins having a polypeptide sequence in which up to 60%, preferably up to 25%, especially preferably up to 15%, in particular up to 10, 9, 8, 7, 6, 5, 4, 3, 2, 1% of the amino acid residues are modified in comparison with the abovementioned reference sequences by deletion, insertion, substitution or a combination thereof and which still have at least 50%, preferably 65%, especially preferably 80%, in particular more than 90%, of the activity of the protein with the corresponding abovementioned reference sequence, where 100% activity of the reference protein is understood as meaning the increase of the activity of the cells used as biocatalyst, that is to say the amount of substance reacted per unit time, based on the cell weight used (units per gram of cell dry weight [U/g CDW]), compared with the activity of the biocatalyst without the presence of the reference protein, where activity in this context and in the context of the determination of the activity of the enzyme E_{b} is generally understood in particular as meaning the oxidation of dodecanoyl-CoA thioester to 2-dodecenoyl-CoA thioester.

### Use of the Microorganisms According to the Invention

A further subject matter of the present invention relates to the use of the abovementioned microorganisms for the production of organic substances, in particular of fatty acids, fatty acid esters, alkan-1-als, alkan-1-ols and alkan-1-amines, alken-1-als, alken-1-ols, alken-1-amines, alkanes and alkenes, in particular 1-alkenes, which may optionally include a further double bond.

Organic substances and microorganisms which have been emphasized as being preferred in the context of the microorganisms according to the invention are also preferred in the context of the use according to the invention.

The organisms according to the invention which are preferably used for specific organic substances have already been emphasized in the context of the microorganisms according to the invention.

Process for the Production of an Organic Substance from a Simple Carbon Source

A further subject matter of the present invention relates to a process for the production of an organic substance, in particular of fatty acids, fatty acid esters, alkan-1-als, alkan-1-ols and alkan-1-amines, alken-1-als, alken-1-ols, alken-1-amines, alkanes and alkenes, in particular 1-alkenes, which may optionally include a further double bond, from a simple carbon source comprising the process steps

I) bringing a microorganism according to the invention into contact with a medium comprising the simple carbon source,
II) culturing the microorganism under conditions which make it possible for the microorganism to form the organic substance from the simple carbon source, and
III) if appropriate, isolation of the organic substance formed.

In the process according to the invention, the microorganisms according to the invention may, for the purpose of producing the organic substance, be brought into contact with the nutrient medium and thus cultured continuously or discontinuously in the batch method or in the fed-batch method or in the repeated fed-batch method. Also feasible is a semicontinuous process as described in GB-A-1009370. A summary of known culture methods is described in the textbook by Chmiel (“Bioprozesstechnik 1. Einfuhrung in die Bioverfahrenstechnik”, Gustav Fischer Verlag, Stuttgart, 1991) or in the textbook by Storhas (“Bioreaktoren and periphere Einrichtungen”, Vieweg Verlag, Braunschweig/Wiesbaden, 1994).

The culture medium to be used must suitably satisfy the requirements of the respective strains. Descriptions of culture media of various microorganisms are contained in the handbook “Manual of Methods for General Bacteriology” of the American Society for Bacteriology (Washington D.C., USA, 1981).

In the process according to the invention, it is preferred to employ preferred microorganisms according to the invention.

The simple carbon source which is employed in the process according to the invention are those mentioned above as being preferred.

Nitrogen sources which can be employed are organic nitrogenous compounds such as peptones, yeast extract, meat extract, malt extract, cornsteep liquor, soyabean meal and urea or inorganic compounds such as ammonium sulphate, ammonium chloride, ammonium phosphate, ammonium carbonate and ammonium nitrate, ammonia, ammonium hydroxide or ammonia water. The nitrogen sources may be employed individually or as a mixture. Phosphorus sources which can be used are phosphoric acid, potassium dihydrogen phosphate or dipotassium hydrogen phosphate or the corresponding sodium-containing salts. The culture medium must furthermore contain salts of metals such as, for example, magnesium sulphate or iron sulphate, which are necessary for growth. Finally, essential growth factors such as amino acids and vitamins may be employed in addition to the abovementioned substances. Moreover, suitable precursors may be added to the culture medium. The feed substances mentioned may be added to the culture as a single batch or may be fed in a suitable manner during culturing. The pH of the culture is controlled by employing basic compounds such as sodium hydroxide, potassium hydroxide, ammonia or ammonia water, or acidic compounds such as phosphoric acid or sulphuric acid in a suitable manner. Foaming may be controlled by using antifoams such as, for example, fatty acid polyglycol esters. To maintain stability of plasmids, suitable selective substances such as antibiotics, for example, may be added to the medium. Oxygen or oxygen-containing gas mixtures such as, for example, air are introduced into the culture so as to maintain aerobic conditions.

According to one embodiment of the process according to the invention, said process is carried out in a two-phase system comprising

A) an aqueous phase and
B) an organic phase,
where the organic substance is formed by the microorganism in process step II) in the aqueous phase and the organic substance formed accumulates in the organic phase. In this manner, it is possible for the organic substance formed to be extracted in situ.

Preferred organic substances which are produced by the process according to the invention are the substances mentioned hereinabove as being preferred, in particular the fatty acids and fatty acid derivatives.

In the examples mentioned hereinbelow, the present invention will be described with the aid of examples without it being intended to limit the invention, whose scope of use is revealed in the entire description and the claims, to the embodiments mentioned in the examples.

The organisms according to the invention which are preferably employed for specific organic substances in preferred processes according to the invention have already been emphasized in the context of the microorganisms according to the invention.

### EXAMPLES

### Example 1

### Preparation of an E. coli Expression Vector for the Overexpression of the alkL Gene from P. putida GPo1

To prepare an E. coli expression vector for the overexpression of the Pseudomonas putida alkL gene (SEQ ID No.: 01), this gene was prepared synthetically and then amplified like the P_{lacuv5} promoter (SEQ ID No.: 34) from a pJ294 derivative, with the introduction of homologous regions for recombination cloning. At the same time, a cleavage site was introduced upstream of the promoter and a cleavage site was introduced downstream of the alkL stop codon via the oligonucleotides used.

The following oligonucleotides were employed for the amplification of the alkL gene and the P_{lacuv5} promoter from the respective pJ294 derivatives as the template:

Promoter region:
(SEQ ID No.: 03)

| | |
|---|---|
| fw-Prom + H1: | 5′-ACC ACA GCC AGG ATC CTT CAA TAT |
| | TAT TGA AGC-3′ |

(SEQ ID No.: 04)

| | |
|---|---|
| rv-Prom: | 5′-ATG CCA CTC TCC TTG-3′ |

(SEQ ID No.: 05)

| | |
|---|---|
| fw-alkL + H2: | 5′-CAA GGA GAG TGG CAT GTG AGT TTT |
| | TCT AAT TAT -3′ |

(SEQ ID No.: 06)

| | |
|---|---|
| rv-alkL + H3: | 5′-TTA CCA GAC TCG AGG GTA CCT TAG |
| | AAA ACA TAT GAC-3′ |

The following parameters were employed for the PCR: 1×: initial denaturation, 98° C., 0:30 min; 35×: denaturation, 98° C., 0:30 min, annealing, 50.5° C., 0:45 min; elongation, 72° C., 0:15 min; 1×: terminal elongation, 72° C., 5 min. The Phusion™ High-Fidelity Master Mix from New England Biolabs (Frankfurt) was used for the amplification, following the manufacturer's recommendations. Thereafter, in each case 100 μl of the PCR reactions were separated on a 2% agarose gel. The procedure of the PCR, the agarose gel electrophoresis, the ethidium bromide staining of the DNA and the determination of the PCR fragment sizes were carried out in a manner known to the skilled worker.

In both cases, PCR fragments of the expected size were successfully amplified. The size was 654 base pairs for the promoter region and 728 base pairs for the alkL construct.

To isolate the DNA from an agarose gel, the target DNA was excised from the gel using a surgical blade and purified using the “Quick Gel Extraction Kit” from Qiagen (Hilden). This was done following the manufacturer's instructions. In the next step, the PCR products together with the BamHI-KpnI-cut pCDFDuet-1 (71340-3, Merck, Darmstadt) underwent recombination by means of in vitro cloning using the “In-Fusion Advantage PCR Cloning Kit” from Clontech (Saint-Germain-en-Laye), giving rise to the resulting vector. The use corresponded to the manufacturer's instructions.

pCDFDuet-1 is an E. coli vector which confers spectinomycin/streptomycin resistance to the organism and which contains a CoIDF13 replication origin. The transformation of chemically competent E. coli DH5α cells (New England Biolabs, Frankfurt) was performed in the manner known to the skilled worker.

The correctness of the plasmid was checked by restriction analysis with XbaI. The authenticity of the inserted fragments was verified by DNA sequencing. The finished E. coli expression vector was named pCDF[alkL] (SEQ ID No.:07).

### Example 2

### Preparation of Expression Vectors for the fatB2 and fatB1 Genes from Cuphea hookeriana and fatB2 from Cuphea palustris

To prepare expression vectors for the fatB2 and fatB1 genes from Cuphea hookeriana (SEQ ID No. 08 and SEQ ID No. 09, respectively) and fatB2 from Cuphea palustris (SEQ ID No. 10), these genes were codon-optimized for the expression in Escherichia coli. The genes were synthesized together with a tac promoter (SEQ ID No. 39) and, simultaneously, a cleavage site was introduced upstream of the promoter and a cleavage site was introduced downstream of the terminator. The synthesized DNA fragments P_{tac}-ChFatB2, P_{tac}-ChFatB1 and P_{tac}-CpFatB2 were digested with the restriction endonucleases BamHI and NotI and ligated into the correspondingly cut vector pJ294 (DNA 2.0 Inc.; Menlo Park, Calif., USA). The finished E. coli expression vectors were named pJ294[Ptac-ChFATB2_optEc] (SEQ ID No. 11), pJ294[Ptac-CpFATB2_optEc] (SEQ ID No. 13) and pJ294[Ptac-ChFATB1_optEc] (SEQ ID No. 12), respectively.

### Example 3

### Chromatographic Quantification of Products

Fatty acids were quantified following derivatization as fatty acid methyl esters, using gas chromatography. After the addition of 1 ml of acetone and 2 ml of water, 50 μl of heptadecanoic acid (10 g/l dissolved in ethanol) were added as internal reference substance to the samples, consisting of 2 ml of culture broth. The samples were acidified with 200 μl of acetic acid and treated with 10 ml of a 1:1 (v/v) chloroform/methanol mixture. The samples were mixed thoroughly for at least 1 min. Thereafter, the chloroform phase was removed and evaporated. The dry residue was taken up in 1 ml of 1.25 M methanolic hydrochloric acid and incubated at 50° C. overnight to esterify the fatty acids present. The reaction was stopped by addition of 5 ml of saturated sodium carbonate solution (all substances from Sigma-Aldrich, Steinheim). The fatty acid methyl esters were extracted by addition of 1 ml of n-heptane and mixing vigorously for 15 seconds. The heptane phase was measured by means of gas chromatography. To separate fatty acid methyl esters, the capillary column SP™-2560 of dimensions 100 m×0.25 mm and a film thickness of 0.2 μm (Supelco, Sigma-Aldrich, Steinheim) was employed as the stationary phase. The carrier gas employed was helium. The separation was carried out within 45 min at an injector temperature of 260° C., a detector temperature of 260° C. and a column temperature of 140° C. at the beginning, held for 5 min, and increased to 240° C. at a rate of 4° C./min and held for 15 min. The injection volume was 1 μl, the split rate was 1:20 and the flow rate of the carrier gas 1 ml/min. Detection was by means of a flame ionization detector (GC Perkin Elmer Clarus 500, Perkin Elmer, Rodgau). Heptadecanoic acid (Sigma-Aldrich, Steinheim) was used as the internal reference substance for quantifying the fatty acid methyl esters. The reference substances C8:0-Me caprylic acid methyl ester, C10:0-Me capric acid methyl ester, C12:0-Me lauric acid methyl ester, C14:0-Me myristic acid methyl ester, C16:0-Me palmitic acid methyl ester, C16:1-Me palmitoleic acid methyl ester, C18:0-Me stearic acid methyl ester, C18:1-Me oleic acid methyl ester (GLC Standard Mix GLC-20 1892-1AMP, GLC-30 1893-1AMP, GLC-50 1894-1AMP, Sigma-Aldrich, Steinheim) were used for the calibration. The lower detection limits were a concentration of 10 mg/l for all fatty acid methyl esters.

### Example 4

### Production of Fatty Acids by E. coli Strains with Deletion in the fadE Gene, which Overexpress the alkL Genes from Pseudomonas putida GPo1 in Various Modifications and fatB2 from Cuphea hookeriana

The first step was to construct an E. coli strain with deletion in the fadE gene (SEQ ID No. 14). To make the gene deletion, a plasmid which carries the DNA sequence ΔfadE (SEQ ID No. 15) was constructed. This sequence was synthesized and is composed of homologous regions 500 base pairs upstream and downstream of the fadE gene and the recognition sequence for the restriction endonuclease NotI at the 5′ and the 3′ end. The sequence ΔfadE was digested with the restriction endonuclease NotI and ligated into the analogously cut vector pKO3. The strain E. coli W3110 ΔfadE was constructed using the pKO3-ΔfadE construct (SEQ ID No. 16) using methods known to the skilled worker (see Link A J, Phillips D, Church G M. J. Bacteriol. 1997. 179(20).). The DNA sequence after the deletion is shown in SEQ ID No. 17.

To generate E. coli strains with the expression vector for the alkL gene from Pseudomonas putida GPo1 in combination with the expression vector for the fatB2 gene from Cuphea hookeriana, electrocompetent cells of E. coli W3110 ΔfadE were prepared. This was done in a manner known to the skilled worker. The cells were transformed with the plasmids pCDFDuet-1 or pCDF[alkL] in combination with pJ294[Ptac-ChFATB2_optEc] and plated onto LB plates supplemented with spectinomycin (100 μg/ml) and ampicillin (100 μg/ml). Transformants were checked for the presence of the correct plasmids by plasmid preparation and analytical restriction analysis.

The following E. coli strains were generated in this manner:
E. coli W3110 ΔfadE pCDFDuet-1/pJ294[Ptac-ChFATB2_optEc]
E. coli W3110 ΔfadE pCDF[alkL]/pJ294[Ptac-ChFATB2_optEc]

These strains were used to study their ability to produce fatty acids. The following procedure was employed:

The strains were subjected to a multi-stage aerobic culturing process. The strains to be studied were first grown from in each case one single colony in Luria-Bertani broth as described by Miller (Merck, Darmstadt) as a 5 ml preculture. The next culturing step was performed in M9 medium. The medium, composed of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all chemicals from Merck, Darmstadt) and 0.1% (v/v) trace element solution, was brought to pH 7.4 using 25% strength ammonium hydroxide solution. The added trace element solution, composed of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate, dissolved in 37% hydrochloric acid solution (all chemicals from Merck, Darmstadt), was filter-sterilized before being added to the M9 medium. 10 ml of M9 medium together with 100 μg/ml of spectinomycin and 100 μg/ml of ampicillin were introduced into 100 ml baffled Erlenmeyer flasks and inoculated with 0.5 ml of the preculture. Culturing was done at 37° C. and 200 rpm in a shaker-incubator. After a culturing time of 8 hours, 50 ml of M9 medium supplemented with 100 μg/ml of spectinomycin and 100 μg/ml of ampicillin were introduced into a 250 ml baffled Erlenmeyer flask and inoculated with the 10 ml culture so that an optical density (600 nm) of 0.2 was obtained. Culturing was done at 30° C. and 200 rpm in a shaker-incubator. When an optical density (600 nm) of from 0.4 to 0.5 had been reached, the gene expression was induced by adding 1 mM of IPTG (time t₀). The strains were cultured for at least another 24 hours under identical conditions. During the culture period, 2 ml samples were taken, and the concentration of fatty acids with different carbon chain length was quantified analogously to Example 3. The results are shown in the table which follows.

TABLE 1
Production of fatty acids using E. coli W3110 ΔfadE, which overexpresses fatB2 from
C. hookeriana and alkL from P. putida GPo1. The data shown are the concentrations of fatty
acids with different carbon chain lengths after incubation for 29 hours.

| | C_{Caprylic acid} | C_{Capric acid} | C_{Myristic acid} | C_{Palmitic acid} | C_{Palmitoleic acid} | C_{Stearic acid} | C_{Oleic acid} |
|---|---|---|---|---|---|---|---|
| Strain | [mg/l * OD] | [mg/l * OD] | [mg/l * OD] | [mg/l * OD] | [mg/l * OD] | [mg/l * OD] | [mg/l * OD] |
| E. coli W3110 ΔfadE pCDFDuet- | 30.1 | 2.4 | 1.9 | 13.6 | 17.3 | 3.6 | 1.9 |
| 1/pJ294[Ptac-ChFATB2_optEc] | | | | | | | |
| E. coli W3110 ΔfadE | 82.6 | 10.6 | 4.1 | 23.7 | 74.0 | 9.1 | 4.1 |
| pCDF[alkL]/pJ294[Ptac- | | | | | | | |
| ChFATB2_optEc] | | | | | | | |

This demonstrated that the strains with alkL form considerably more caprylic acid, capric acid, myristic acid, palmitic acid, palmitoleic acid, stearic acid and oleic acid than the strains without alkL. This demonstrates that enhancing alkL promotes the production of fatty acids of different chain lengths and degrees of saturation from nonrelated carbon sources.

### Example 5

### Preparation of an E. coli Expression Vector for the Genes fadD from Escherichia coli and atfA from Acinetobacter sp. ADP1

To prepare the E. coli expression vector for the genes fadD (SEQ ID No.: 18) from Escherichia coli and atfA with terminator (SEQ ID No.: 19) from Acinetobacter sp. ADP1 under the control of a tac promoter, these genes were amplified by PCR from chromosomal DNA of E. coli W3110 and Acinetobacter calcoaceticus ADP1, respectively, with the introduction of homologous regions for recombination cloning. The synthetic tac promoter (SEQ ID No.: 20) was amplified with ribosome binding site from a pJ294 derivative, with introduction of homologous regions. Chromosomal DNA was prepared from E. coli W3110 and Acinetobacter calcoaceticus ADP1, respectively, by means of the DNeasy Blood & Tissue Kit (Qiagen, Hilden) following the manufacturer's instructions. The following oligonucleotides were employed in the amplification of the genes fadD from E. coli and atfA from Acinetobacter sp. ADP1 with chromosomal DNA of E. coli W3110 and Acinetobacter calcoaceticus ADP1, respectively, as the template and in the amplification of the synthetic P_{tac} promoter from a pJ294 derivative:

P_{tac}:
(SEQ ID No.: 21)

| | |
|---|---|
| 11-001_fw: | 5′-TTATGCGACTCCTGCGTTTAGGGAAAGAGCATTT |
| | G-3′ |

(SEQ ID No.: 22)

| | |
|---|---|
| Ptac-rv: | 5′-GTTAACATATGTTTTACCTCCTGTTAAACAAA-3′ |

fadD [E. coli]:
(SEQ ID No.: 23)

| | |
|---|---|
| fadD-fw: | 5′-TAAAACATATGTTAACGGCATGTATATCATTT-3′ |

(SEQ ID No.: 24)

| | |
|---|---|
| fadD-rv: | 5′-TCTCCTCAGACTTAACGCTCAGGCTTTATTGT-3′ |

atfA [Acinetobacter sp. ADP1]:
(SEQ ID No.: 25)

| | |
|---|---|
| atfA-fw: | 5′-GTTAAGTCTGAGGAGATCCACGCTATGCGCCC-3′ |

(SEQ ID No.: 26)

| | |
|---|---|
| 11-00_rv: | 5′-CAATTGAGATCTGCCACGACTGCAATGGTTCATC-3′ |

The following parameters were employed for the PCR: 1×: initial denaturation, 103° C., 3:00 min; 35×: denaturation, 98° C., 0:10 min, annealing, 65° C., 0:15 min; elongation, 72° C., 0:45 min; 1×: terminal elongation, 72° C., 10 min. The Phusion™ High-Fidelity Master Mix from New England Biolabs (Frankfurt) was used for the amplification, following the manufacturer's recommendations. Thereafter, in each case 50 μl of the PCR reactions were separated on a 1% TAE agarose gel. The procedure of the PCR, the agarose gel electrophoresis, the ethidium bromide staining of the DNA and the determination of the PCR fragment sizes were carried out in a manner known to the skilled worker.

In all cases, PCR fragments of the expected size were successfully amplified. The size was 607 bp for the P_{tac} promoter region, 1778 by for fadD and 1540 by for atfA.

To isolate the DNA from an agarose gel, the target DNA was isolated from the gel using a surgical blade and purified using the “Quick Gel Extraction Kit” from Qiagen (Hilden) following the manufacturer's instructions. The purified PCR products underwent recombination with the EcoNI/NdeI-cut vector pCDFDuet™-1 (71340-3, Merck, Darmstadt) by means of in-vitro cloning using the Geneart Seamless Cloning and Assembly Kit from Invitrogen (Darmstadt). The use corresponded to the manufacturer's instructions. pCDFDuet-1 is an E. coli vector which confers spectinomycin/streptomycin resistance to the organism and which contains a CoIDF13 replication origin. The transformation of chemically competent E. coli DH5α cells (New England Biolabs, Frankfurt) was performed in the manner known to the skilled worker. The correctness of the plasmid was checked by restriction analysis with XbaI. The authenticity of the inserted fragments was verified by DNA sequencing. The finished E. coli expression vector was named pCDF[fadD-atfA] (SEQ ID No.:27).

### Example 6

### Preparation of an E. coli Expression Vector for the Genes fadD from Escherichia coli atfA from Acinetobacter sp. ADP1 and alkL from Pseudomonas putida GPo1

To prepare an E. coli expression vector for the genes fadD from Escherichia coli, atfA from Acinetobacter sp. ADP1 and alkL from Pseudomonas putida GPo1, the plasmid pCDF[alkL] (SEQ ID No.: 07) is digested with FseI and XhoI, and the fragment which carries the alkL gene from Pseudomonas putida GPo1 under the control of the P_{lacuv5} promoter (see Example 1) is subsequently isolated.

To this end, the digested plasmid is separated on a 1% TAE agarose gel. The procedure of the restriction digestion, the agarose gel electrophoresis, the ethidium bromide staining of the DNA and the determination of the restriction fragment sizes are performed in a manner known to the skilled worker. To isolate the DNA from an agarose gel, the target DNA is isolated from the gel using a surgical blade and purified using the Quick Gel Extraktion Kit from Qiagen (Hilden) following the manufacturer's instructions.

Thereafter, the purified restriction fragment is ligated with the likewise FseI- and XhoI-cut vector fragment (7290 bp) of pCDF[fadD-atfA] (SEQ ID No.: 27). Ligation of the DNA fragment and transformation of chemically competent E. coli DH5α cells (New England Biolabs, Frankfurt) are performed in the manner known to the skilled worker.

The correctness of the plasmids produced is checked by restriction analysis with FseI and XhoI. The authenticity of the inserted fragments is verified by DNA sequencing. The finished E. coli/expression vector is named pCDF[fadD-atfA]-[alkL] (SEQ ID No.:28).

### Example 7

### Chromatographic Quantification of Products

The quantification of fatty acid esters is performed using gas chromatography. 100 μl of methyl heptadecanoate solution (5 g/l dissolved in acetone) are added to the samples, consisting of 1 ml of culture broth, and then 1.1 ml of n-heptane are added and the samples are vortexed vigorously for 15 seconds. The heptane phase is measured by means of gas chromatography. To separate fatty acid esters, the capillary column SP™-2560 of dimensions 100 m×0.25 mm and a film thickness of 0.2 μm (Supelco, Sigma-Aldrich, Steinheim) is employed as the stationary phase. The carrier gas employed is helium. The separation is carried out within 45 min at an injector temperature of 260° C., a detector temperature of 260° C. and a column temperature of 140° C. at the beginning, held for 5 min, and increased to 240° C. at a rate of 4° C./min and held for 15 min. The injection volume is 1 μl, the split rate is 1:20 and the flow rate of the carrier gas 1 ml/min. Detection is by means of a flame ionization detector (GC Perkin Elmer Glarus 500, Perkin Elmer, Rodgau). Methyl heptanoate (Sigma-Aldrich, Steinheim) is used as the internal reference substance for quantifying the fatty acid esters. The reference substances C8:0-Me caprylic acid methyl ester, C10:0-Me capric acid methyl ester, C12:0-Me lauric acid methyl ester, C14:0-Me myristic acid methyl ester, C16:0-Me palmitic acid methyl ester, C16:1-Me palmitoleic acid methyl ester, C18:0-Me stearic acid methyl ester, C18:1-Me oleic acid methyl ester (GLC Standard Mix GLC-20 1892-1AMP, GLC-30 1893-1AMP, GLC-50 1894-1AMP, Sigma-Aldrich, Steinheim), C8:0-Et caprylic acid ethyl ester, C10:0-Et capric acid ethyl ester, C12:0-Et lauric acid ethyl ester, C14:0-Et myristic acid ethyl ester, C16:0-Et palmitic acid ethyl ester, C18:0-Et stearic acid ethyl ester, C18:1-Et oleic acid ethyl ester (all from Sigma-Aldrich, Steinheim) and C16:1-Et palmitoleic acid ethyl ester (Biomol, Hamburg) are used for the calibration. The lower detection limits are a concentration of 10 mg/l for all fatty acid esters.

### Example 8

### Production of Fatty Acid Esters by E. coli Strains which have a Deletion in the fadE Gene and which Overexpress the alkL Genes from Pseudomonas putida GPo1, ChfatB1 and ChfatB2 from Cuphea hookeriana, and Cpfat2″ from Cuphea palustris, fadD from E. coli and atfA from Acinetobacter sp. ADP1

To generate E. coli strains with the expression vector for the alkL genes from Pseudomonas putida GPo1, fadD from Escherichia coli and atfA from Acinetobacter sp. ADP1 from Pseudomonas putida GPo1 in combination with the expression vector for the fatB2 gene from Cuphea hookeriana, electrocompetent cells of E. coli W3110 ΔfadE (see Example 4) are prepared. This was done in a manner known to the skilled worker. They are transformed with the plasmids pCDF[fadD-atfA] (SEQ ID No.: 27) and pCDF[fadD-atfA]-[alkL] (SEQ ID No.: 28), respectively, in combination with pJ294[Ptac-ChFATB2_optEc] (SEQ ID No. 11), pJ294[Ptac-CpFATB2_optEc] (SEQ ID No.: 13) and pJ294[Ptac-ChFATB1_optEc] (SEQ ID No.: 12), respectively, in and plated onto LB plates supplemented with spectinomycin (100 μg/ml) and ampicillin (100 μg/ml). Transformants are checked for the presence of the correct plasmids by plasmid preparation and analytical restriction analysis.

The following E. coli strains are generated in this manner:
E. coli W3110 ΔfadE pCDF[fadD-atfA]pJ294[Ptac-ChFATB2_optEc]
E. coli W3110 ΔfadE pCDF[fadD-atfA]alkl-4-pJ294[Ptac-ChFATB2_optEc]
E. coli W3110 ΔfadE pCDF[fadD-atfA]/pJ294[Ptac-CpFATB2_optEc]
E. coli W3110 ΔfadE pCDF[fadD-atfA]alkLypJ294[Ptac-CpFATB2_optEc]
E. coli W3110 ΔfadE pCDF[fadD-atfA]/pJ294[Ptac-ChFATB1_optEc]
E. coli W3110 ΔfadE pCDF[fadD-atfA]alkl-4-pJ294[Ptac-ChFATB1_optEc]

These strains are used to study their ability to produce fatty acid esters. The following procedure is employed:

The strains are subjected to a multi-stage aerobic culturing process. The strains to be studied are first grown from in each case one single colony in Luria-Bertani broth as described by Miller (Merck, Darmstadt) as a 5 ml preculture. The next culturing step is performed in M9 medium. The medium, composed of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all chemicals from Merck, Darmstadt) and 0.1% (v/v) trace element solution, is brought to pH 7.4 using 25% strength ammonium hydroxide solution. The added trace element solution, composed of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate, dissolved in 37% hydrochloric acid solution (all chemicals from Merck, Darmstadt), is filter-sterilized before being added to the M9 medium. 10 ml of M9 medium together with 100 μg/ml of spectinomycin and 100 μg/ml of ampicillin are introduced into 100 ml baffled Erlenmeyer flasks and inoculated with 0.5 ml of the preculture. Culturing is done at 37° C. and 200 rpm in a shaker-incubator. After a culturing time of 8 hours, 50 ml of M9 medium supplemented with 100 μg/ml of spectinomycin and 100 μg/ml of ampicillin are introduced into a 250 ml baffled Erlenmeyer flask and inoculated with the 10 ml culture so that an optical density (600 nm) of 0.2 is obtained. Culturing is done at 30° C. and 200 rpm in a shaker-incubator. When an optical density (600 nm) of from 0.4 to 0.5 has been reached, the gene expression is induced by adding 1 mM of IPTG (time t₀). The strains are cultured for at least another 24 hours under identical conditions. During the culture period, 2 ml samples are taken, and the concentration of fatty acid methyl esters or fatty acid ethyl esters with different carbon chain lengths is quantified analogously to Example 7. This demonstrates that the strains E. coli W3110 ΔfadE pCDF[fadD-atfA]-/pJ294[Ptac-ChFATB2_optEc] and E. coli W3110 ΔfadE pCDF[fadD-atfA]-[alkL]/pJ294[Ptac-ChFATB2_optEc] are predominantly capable of forming C8:0-Me caprylic acid methyl ester, C10:0-Me capric acid methyl ester, C16:0-Me palmitic acid methyl ester, C16:1-Me palmitoleic acid methyl ester and C18:1-Me oleic acid methyl ester (when methanol is added) and C8:0-Et caprylic acid ethyl ester, C10:0-Et capric acid ethyl ester, C16:0-Et palmitic acid ethyl ester, C16:1-Et palmitoleic acid ethyl ester and C18:1-Et oleic acid ethyl ester (when ethanol is added), respectively.

Furthermore, it is demonstrated that the strains E. coli W3110 ΔfadE pCDF[fadD-atfA]-/pJ294[Ptac-CpFATB2_optEc] and E. coli W3110 ΔfadE pCDF[fadD-atfA]-[alkL]/pJ294[Ptac-CpFATB2_optEc] are capable of predominantly forming C12:0-Me lauric acid methy ester, C14:0-Me myristic acid methyl ester, C16:0-Me palmitic acid methyl ester, C16:1-Me palmitoleic acid methyl ester, C18:0-Me stearic acid methyl ester and C18:1-Me oleic acid methyl ester (when methanol is added) and C12:0-Et lauric acid ethyl ester, C14:0-Et myristic acid ethyl ester, C16:0-Et palmitic acid ethyl ester, C16:1-Et palmitoleic acid ethyl ester and C18:1-Et oleic acid ethyl ester (when ethanol is added), respectively.

Furthermore, it is demonstrated that the strains E. coli W3110 ΔfadE pCDF[fadD-atfA]-/pJ294[Ptac-ChFATB1_optEc] and E. coli W3110 ΔfadE pCDF[fadD-atfA]-[alkL]/pJ294[Ptac-ChFATB1_optEc] are capable of predominantly forming C14:0-Me methyl myristate, C16:0-Me methyl palmitate, C16:1-Me methyl palmitoleate, C18:0-Me methyl stearate and C18:1-Me methyl oleate (when methanol is added) and C14:0-Et ethyl myristate, C16:0-Et ethyl palmitate, C16:1-Et ethyl palmitoleate and C18:1-Et ethyl oleate (when ethanol is added), respectively. Finally, it is demonstrated that the strains E. coli W3110 ΔfadE pCDF[fadD-atfA]-[alkL]/pJ294[Ptac-ChFATB2_optEc], E. coli W3110 ΔfadE pCDF[fadD-atfA]-[alkL]/pJ294[Ptac-CpFATB2_optEc] and E. coli W3110 ΔfadE pCDF[fadD-atfA]-[alkL]/pJ294[Ptac-ChFATB1_optEc]ₜ, which are named in this example, form substantially more of the respective fatty acid methyl esters (when methanol is added) and fatty acid ethyl esters (when ethanol is added), respectively, than the strains E. coli W3110 ΔfadE pCDF[fadD-atfA]/pJ294[Ptac-ChFATB2_optEc], E. coli W3110 ΔfadE pCDF[fadD-atfA]/pJ294[Ptac-CpFATB2_optEc] and E. coli W3110 ΔfadE pCDF[fadD-atfA]/pJ294[Ptac-ChFATB1_optEc]. This demonstrates that the enhancement of the alkL gene product promotes the production of fatty acid esters with various chain lengths of the alkyl chain both of the fatty acid residue and the alcohol residue of the fatty acid esters and with a different degree of saturation of the alkyl chain of the fatty acid, respectively, from unrelated carbon sources.

### Example 9

### Preparation of Expression Vectors for the Genes CnfatB3 from Cocos nucifera and synUcTE from Umbellularia californica

To prepare expression vectors for the genes fatB3 (SEQ ID No. 35) from Cocos nucifera and synUcTE (SEQ ID No. 37) from Umbellularia californica (each encoding one enzyme Eᵢ), these genes were codon-optimized for expression in Escherichia coli. The genes were synthesized in each case together with a tac promoter (SEQ ID No. 39) and at the same time a cleavage site was introduced upstream of the promoter and a cleavage site was introduced downstream of the terminator. The synthesized DNA fragments P_{tac}-CnFATB3 and P_{tac} synUcTE were digested with the restriction endonucleases BamHI and NotI and ligated into the correspondingly cut vector pJ294 (DNA2.0 Inc., Menlo Park, Calif., USA). The completed E. coli expression vectors were termed pJ294{Ptac}[CnFATB3(co_Ec)] (SEQ ID No. 40), pJ294[Ptac-synUcTE] (SEQ ID No. 41). The vector pJ294 is an E. coli vector which imparts ampicillin resistance and also carries a p15A replication origin and therefore has a low copy number (10-15 copies per cell).

### Example 10

### Preparation of Expression Vectors for the Genes alkL_Oa from Oceanocaulis alexandrii, alkL_Ma from Marinobacter aquaeolei, alkL_CspK31 from Caulobacter sp. K31

To prepare expression vectors for the genes alkL_Oa (SEQ ID No. 42) from Oceanocaulis alexandrii HTCC2633, alkL_Ma (SEQ ID No. 44) from Marinobacter aquaeolei VT8, alkL_CspK31 (SEQ ID No. 46) from Caulobacter sp. K31 (in each case encoding one AlkL gene product), these genes were synthesized together with a lacuv5 promoter (SEQ ID No. 34). The synthesized DNA fragments P_{lacuv5} alkL_Oa, P_{lacuv5} alkL_Ma and P_{lacuv5} alkL_CspK31 were amplified with introduction of homologous regions for recombination cloning.

The following oligonucleotides were used for amplification of the target genes.

(SEQ ID No. 48)

| | |
|---|---|
| alkL_H1_fw: | 5′-GCTTACTGAATTTGCCTGAACCATGGGGCAGTGA |
| | G-3′ |

(SEQ ID No. 49)

| | |
|---|---|
| alkL_H2_rv: | 5′-TTCTGAAGTGGGGGCGGCCGCCCTTTTGACGGGTAC |
| | C-3′ |

The following parameters were used for the PCR: 1×: initial denaturation, 98° C., 1 min; 35×: denaturation, 98° C., 0:15 min, annealing, 60° C., 0:45 min; elongation, 72° C., 1:30 min; 1×: terminal elongation, 72° C., 10 min. For the amplification, the Phusion™ High-Fidelity Master Mix from New England Biolabs (Frankfurt) was used in accordance with the manufacturer's recommendations. In each case 50 μl of the PCR reactions were then separated on a 1% strength TAE agarose gel. The PCR, agarose gel electrophoresis, ethidium bromide staining of the DNA and determination of the PCR fragment sizes were carried out in a manner known to those skilled in the art. In all cases PCR fragments of the expected size were able to be amplified. These were 906 base pairs for P_{lacuv5} alkL_Oa, 960 base pairs for P_{lacuv5} alkL_Ma and 903 base pairs for P_{lacuv5} alkL_CspK31. To isolate the DNA from the TAE agarose gel, the target DNA was cut out of the gel with a scalpel and purified using the QiaQuick Gel extraction kit according to the manufacturer's instructions (Qiagen, Hilden). The purified PCR products were cloned together with the NotI-cut vector pJ294[Ptac-ChFATB2_optEc] (SEQ ID No. 11) by recombination, using the Geneart® Seamless Cloning and Assembly Kit according to the manufacturer's instructions (Life Technologies, Carlsbad, Calif., USA). From the resultant pJ294 derivatives pJ294{Ptac}[ChFATB2(co_Ec){Placuv5}[alkL_Oa] (SEQ ID No. 50), pJ294{Ptac}[ChFATB2(co_Ec){Placuv5}[alkL_Ma] (SEQ ID No. 51) and pJ294{Ptac}[ChFATB2(co_Ec){Placuv5}[alkL_CspK31] (SEQ ID No. 52) the fragments P_{lacuv5} alkL_Oa, P_{lacuv5} alkL_Ma and P_{lacuv5} alkL_CspK31 were cut out using a restriction digest with the restriction endonucleases NcoI and NotI and ligated into the corresponding cut vector pCDFDuet-1 (71340-3, Merck, Darmstadt; SEQ ID No. 53). Chemically competent E. coli DH5α (New England Biolabs, Frankfurt) was transformed in a manner known to those skilled in the art. Correct insertion of the target genes was examined by restriction analysis, and authenticity of the introduced genes was validated by DNA sequencing. The resultant expression vectors were named pCDF[alkL_Oa] (SEQ ID No. 54), pCDF[alkL_Ma] (SEQ ID No. 55) and pCDF[alkL_CspK31] (SEQ ID No. 56).

### Example 11

### HPLC/ESI-Based Quantification of Fatty Acids

Octanoic acid, 3-hydroxydecanoic acid, decanoic acid, lauric acid, 3-hydroxymyristic acid, myristic acid, palmitoleic acid, palmitic acid, oleic acid and stearic acid in fermentation samples were quantified by means of HPLC-ESI/MS on the basis of internal calibration for all analytes and using the internal standards D3-lauric acid (methyl-D3, 99%) for octanoic acid, 3-hydroxydecanoic acid, decanoic acid, lauric acid, 3-hydroxymyristic acid, myristic acid, palmitoleic acid and D3-stearic acid (methyl-D3, 98%) for palmitic acid, oleic acid, stearic acid.

The following apparatuses were used:
HPLC system: Surveyor (Thermo Fisher Scientific, Waltham, Mass., USA), consisting of Surveyor MS Pump, Surveyor Autosampler plus and Surveyor PDA Surveyor
Mass spectrometer: TSQ Vantage with HESI II—source (Thermo Fisher Scientific, Waltham, Mass., USA)
HPLC columns: XBridge BEH C8, 100×2.1 mm, particle size: 2.5 μm, pore size 130 Å (Waters, Milford Mass., USA)

The samples were prepared in that 1200 μl of acetone and 300 μl of sample were mixed for approximately 10 seconds and then centrifuged at approximately 13 000 rpm for 5 min. The clear supernatant was taken off and analysed after appropriate dilution with acetone. To each 900 μl of the diluted sample were added 100 μl of ISTD by pipette.

HPLC separation proceeded using the abovementioned HPLC column. The injection volume was 2 μl, the column temperature was 25° C., and the flow rate was 0.3 ml/min. The mobile phase consisted of eluent A (water+10 mmol of ammonium acetate adjusted with ammonia to pH=9) and eluent B (acetonitrile/eluent A 95/5). The following gradient profile was used

| Time [min] | Eluent A [%] | Eluent B [%] |
|---|---|---|
| 0 | 95 | 5 |
| 1 | 95 | 5 |
| 1.1 | 70 | 30 |
| 7 | 5 | 95 |
| 8 | 5 | 95 |

The ESI-MS analysis proceeded with negative ionization using the following parameters of the ESI source:
Spray voltage: 3000 V
Vaporizer temperature: 380° C.
Sheath gas pressure: 40
Aux gas pressure: 15
Capillary temperature: 380° C.

The individual compounds were detected and quantified using “single ion monitoring” (SIM) using the following parameters:

| | Ion | Scan | Scan | Peak |
|---|---|---|---|---|
| | [M − H]⁻ | width | time | width |
| Analyte | [m/z] | [m/z] | [ms] | Q₃ |
| Octanoic acid | 143.13 | 0.002 | 100 | 0.7 |
| 3-Hydroxydecanoic acid | 187.13 | 0.002 | 50 | 0.7 |
| Decanoic acid | 171.13 | 0.002 | 100 | 0.7 |
| Lauric acid | 199.16 | 0.002 | 50 | 0.7 |
| 3-Hydroxymyristic acid | 243.18 | 0.002 | 50 | 0.7 |
| Myristic acid | 227.19 | 0.002 | 50 | 0.7 |
| Palmitoleic acid | 253.18 | 0.002 | 50 | 0.7 |
| Palmitic acid | 255.22 | 0.002 | 30 | 0.7 |
| Oleic acid | 281.23 | 0.002 | 30 | 0.7 |
| Stearic acid | 283.25 | 0.002 | 30 | 0.7 |
| D3-lauric acid | 202.16 | 0.002 | 50 | 0.7 |
| D3-stearic acid | 286.25 | 0.002 | 30 | 0.7 |

### Example 12

### Production of Fatty Acids by E. coli Strains with a Deletion in the fadE Gene, which Overexpresses the Genes alkL from Pseudomonas putida GPo1, alkL from Oceanocaulis alexandrii HTCC2633 or alkL from Caulobacter sp. K31 and fatB1 from Cuphea hookeriana, fatB2 from Cuphea hookeriana, synUcTE from Umbellularia californica or fatB3 from Cocos nucifera

To generate E. coli strains having the expression vector for the gene alkL from Pseudomonas putida GPo1, alkL from Oceanocaulis alexandrii HTCC2633 or alkL from Caulobacter sp. K31 in combination with the expression vector for the fatB1 gene from Cuphea hookeriana, fatB2 from Cuphea hookeriana, synUcTE from Umbellularia californica or fatB3 from Cocos nucifera electrocompetent cells of E. coli W3110 ΔfadE and E. coli JW5020-1 Kan^{S} were prepared. This was carried out in a manner known to those skilled in the art. E. coli JW5020-1 Kan^{S} is a derivative of E. coli JW5020-1 (CGSC, The coli genetic stock center, Yale University, New Haven, USA), and this in turn is an E. coli BW25113 derivative, which carries a deletion of the fadE gene. The fadE gene was replaced by a kanamycin cassette. This was removed before equipping the strain with the expression vectors using a helper plasmid which encodes flp recombinase, in a manner known to those skilled in the art (see Datsenko K. A. and Wanner B. L. (2000) PNAS 97(12):6640-6645) resulting in strain E. coli JW5020-1 Kan^{S}. E. coli JW5020-1 Kan^{S} was transformed with the plasmids pJ294[Ptac-ChFATB1_optEc] (SEQ ID No. 12), pJ294[Ptac-ChFATB2_optEc] (SEQ ID No. 11) or pJ294{Ptac}[CnFATB3(co_Ec)] (SEQ ID No. 40) in combination with pCDFDuet-1, pCDF[alkL] (SEQ ID No. 7) or pCDF[alkL_Oa] (SEQ ID No. 54) or pCDF[alkL_CspK31] (SEQ ID No. 56), and E. coli W3110 ΔfadE was transformed with the plasmids pJ294[Ptac-synUcTE] (SEQ ID No. 41) in combination with pCDFDuet-1 (SEQ ID No. 53) or pCDF[alkL] (SEQ ID No. 7) and plated onto LB-agar plates with spectinomycin (100 μg/ml) and ampicillin (100 μg/ml). Transformants were checked for presence of the correct plasmids by plasmid preparation and analytical restriction analysis.

The following E. coli strains were generated in this manner:
E. coli JW5020-1 Kan^{S} pJ294[Ptac-ChFATB1_optEc]/pCDFDuet-1
E. coli JW5020-1 Kan^{S} pJ294[Ptac-ChFATB1_optEc]/pCDF[alkL]
E. coli JW5020-1 Kan^{S} pJ294[Ptac-ChFATB2_optEc]/pCDFDuet-1
E. coli JW5020-1 Kan^{S} pJ294[Ptac-ChFATB2_optEc]/pCDF[alkL_Oa]
E. coli JW5020-1 Kan^{S} pJ294[Ptac-ChFATB2_optEc]/pCDF[alkL_CspK31]
E. coli JW5020-1 Kan^{S} pJ294{Ptac}[CnFATB3(co_Ec)]/pCDFDuet-1
E. coli JW5020-1 Kan^{S} pJ294{Ptac}[CnFATB3(co_Ec)]/pCDF[alkL]
E. coli W3110 ΔfadE pJ294[Ptac-synUcTE]/pCDFDuet-1
E. coli W3110 ΔfadE pJ294[Ptac-synUcTE]/pCDF[alkL]

These strains were employed to investigate their ability to produce fatty acids. The following procedure was used:

The strains were subjected to a multistage aerobic culturing process. The strains under investigation were first initially grown in Luria-Bertani Bouillon according to Miller (Merck, Darmstadt) with 100 μg/ml ampicillin and 100 μg/ml spectinomycin as 5 ml preliminary culture from one single colony each. The next culturing step proceeded in M9 medium. The medium, consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution, was adjusted to a pH of 7.4 using 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1 M hydrochloric acid (all substances from Merck, Darmstadt) was sterile-filtered before addition to the M9 medium. 10 ml of M9 medium were charged with 100 μg/ml spectinomycin and 100 μg/ml ampicillin into 100 ml conical flasks with chicane and inoculated with 0.5 ml from the preliminary culture. The culturing proceeded at 37° C. and 200 rpm in an incubating shaker. After a cultivating time of 8 hours, 50 ml of M9 medium containing 100 μg/ml of spectinomycin and 100 μg/ml of ampicillin are charged into a 250 ml conical flask with chicane and inoculated with the 10 ml culture, in such a manner that an optical density (600 nm) of 0.2 is achieved. The cuturing proceeded at 37° C. and 200 rpm in an incubating shaker. When an optical density (600 nm) of 0.6 to 0.8 is achieved, gene expression is induced by adding 1 mM IPTG. The strains were cultured for a further 24 hours at 30° C. and 200 rpm. During the culturing, samples of 300 μl are taken off and the concentration of fatty acids of differing carbon chain lengths is quantified as described in Example 10. The results are shown in the tables hereinafter.

Production of fatty acids using E. coli JW5020-1 Kan^{S}, which overexpresses fatB2 from C. hookeriana and alkL from Oceanocaulis alexandrii HTCC2633 and alkL from Caulobacter sp.

K31. The concentrations of fatty acids of differing carbon chain length are reported after culturing for 24 hours (n.d.=not detectable):

| | c_{Caprylic acid} | c_{Capric acid} | c_{Palmitoleic acid} | C_{Vaccenic acid} |
|---|---|---|---|---|
| Strain | [mg/l/OD] | [mg/l/OD] | [mg/l/OD] | [mg/l/OD] |
| E. coli JW5020-1 Kan^{S} pJ294[Ptac- | 21.0 | 1.8 | 0.1 | 1.3 |
| ChFATB2_optEc]/pCDFDuet-1 | | | | |
| E. coli JW5020-1 Kan^{S} pJ294[Ptac- | 27.5 | 6.3 | 5.9 | 2.3 |
| ChFATB2_optEc]/pCDF[alkL_Oa] | | | | |
| E. coli JW5020-1 Kan^{S} pJ294[Ptac- | 38.6 | 4.7 | | |
| ChFATB2_optEc]/pCDF[alkL_CspK31] | | | | |

Production of fatty acids with E. coli JW5020-1 Kan^{S}, which overexpresses fatB3 from C. nucifera and alkL from Pseudomonas putida GPo1. The concentrations of fatty acids of differing carbon chain length are reported after 48 hours of culturing (n.d.=not detectable):

| | C_{Caprylic acid} | C_{Lauric acid} | C_{Myristic acid} | C_{Palmitoleic acid} | C_{Palmitic acid} | C_{Vaccenic acid} |
|---|---|---|---|---|---|---|
| Strain | [mg/l/OD] | [mg/l/OD] | [mg/l/OD] | [mg/l/OD] | [mg/l/OD] | [mg/l/OD] |
| E. coli JW5020-1 Kan^{S} | 0.1 | 3.2 | 4.5 | 13.4 | 1.7 | 4.0 |
| pJ294{Ptac}[CnFATB3(co_Ec)]/pCDFDuet-1 | | | | | | |
| E. coli JW5020-1 Kan^{S} | 0.5 | 6.8 | 9.9 | 21.2 | 3.1 | 5.6 |
| pJ294{Ptac}[CnFATB3(co_Ec)]/pCDF[alkL] | | | | | | |

Production of fatty acids with E. coli JW5020-1 Kan^{S}, which overexpresses fatB1 from C. hookeriana and alkL from Pseudomonas putida GPo1. The concentrations of fatty acids of differing carbon chain lengths are reported after 24 hours of culturing (n.d.=not detectable):

| | c_{Myristic acid} | c_{Palmitoleic acid} |
|---|---|---|
| Strain | [mg/l/OD] | [mg/l/OD] |
| E. coli JW5020-1 Kan^{S} pJ294[Ptac- | 38.2 | 0.1 |
| ChFATB1_optEc]/pCDFDuet-1 | | |
| E. coli JW5020-1 Kan^{S} pJ294[Ptac- | 42.8 | 62.0 |
| ChFATB1_optEc]/pCDF[alkL] | | |

Production of fatty acids using E. coli JW5020-1 Kan^{S}, which overexpresses synUcTE from U. californica and alkL from Pseudomonas putida GPo1. The concentrations of fatty acids of differing carbon chain lengths are reported after 24 hours of culturing (n.d.=not detectable):

| | c_{Lauric acid} | c_{Myristic acid} |
|---|---|---|
| Strain | [mg/l/OD] | [mg/l/OD] |
| E. coli W3110 ΔfadE pJ294[Ptac- | 0.1 | 0.1 |
| synUcTE]/pCDFDuet-1 | | |
| E. coli W3110 ΔfadE pJ294[Ptac- | 20.1 | 1.9 |
| synUcTE]/pCDF[alkL] | | |

Therefore it was found that strains which overexpress alkL from P. putida, O. alexandrii or Caulobacter sp. are able to form more caprylic acid, capric acid, lauric acid, myristic acid, palmitic acid, palmitoleic acid or vaccenic acid, depending on the specificity of the overexpressed acyl-ACP thioesterase. This shows that reinforcement of alkL is required for the preparation of fatty acids of differing chain lengths and degrees of saturation from unrelated carbon sources.

### Example 13

### Preparation of Vectors for the Coexpression of the Genes fatB2 from Cuphea hookeriana, fatB3 from Cocos nucifera, synUcTE from Umbellularia californica with alkL from Pseudomonas putida

For preparation of vectors for the coexpression of the gene fatB2 (SEQ ID No. 8) from Cuphea hookeriana, fatB3 (SEQ ID No. 35) from Cocos nucifera, synUcTE (SEQ ID No. 37) from Umbellularia californica with an alkL gene from Pseudomonas putida, the gene alkL (SEQ ID No. 1) was amplified together with the lacuv5 promotor and terminator from the vector pCDF[alkL] (SEQ ID No. 7).

For amplification of the fragment P-alkL-T (SEQ ID No. 58) for coexpression with fatB2 and synUcTE, the following oligonucleotides were used.

(SEQ ID No. 59)

| | |
|---|---|
| NP-FA-P19: | 5′-ATCCGCTCACAATTGCAAATGCCTGAGGTTTCAG |
| | C-3′ |

(SEQ ID No. 60)

| | |
|---|---|
| NP-FA-P20: | 5′-CTTCCCTTCATTTTGGTCTCGGTCGATCATTCAG |
| | C-3′ |

For amplification of the fragment P-alkL-T (SEQ ID No. 58) for coexpression with fatB3, the following oligonucleotides were used.

(SEQ ID No. 59)

| | |
|---|---|
| NP-FA-P19: | 5′-ATCCGCTCACAATTGCAAATGCCTGAGGTTTCAG |
| | C-3′ |

(SEQ ID No. 61)

| | |
|---|---|
| NP-FA-P21: | 5′-ACTTAGTCGCTGAAGGTCTCGGTCGATCATTCAG |
| | C-3′ |

The following parameters were used for the PCR: 1×: initial denaturation, 98° C., 1 min; 35×: denaturation, 98° C., 0:15 min, annealing, 65° C., 0:45 min; elongation, 72° C., 1:30 min; 1×: terminal elongation, 72° C., 10 min. For the amplification, the Phusion™ High-Fidelity Master Mix from New England Biolabs (Frankfurt) was used in accordance with the recommendations of the manufacturer. In each case 50 μl of the PCR reactions were then separated on a 1% strength TAE agarose gel. The PCR, agarose gel electrophoresis, ethidium bromide staining of the DNA and determination of the PCR fragment sizes were carried out in a manner known to those skilled in the art. The PCR fragments with an expected size of 1095 base pairs were able to be amplified. To isolate the DNA from the TAE agarose gel, the target DNA was cut out from the gel with a scalpel and purified by the QiaQuick Gel extraction Kit according to the manufacturer's instructions (Qiagen, Hilden). The purified PCR products were cloned together with the above-described vectors pJ294[Ptac-ChFATB2_optEc] (SEQ ID No. 11), pJ294{Ptac}[CnFATB3(co_Ec)] (SEQ ID No. 40), pJ294[Ptac-synUcTE] (SEQ ID No. 41), which were linearized with BamHI, by means of recombination, using the Geneart® Seamless Cloning and Assembly Kit according to the manufacturer's instructions (Life Technologies, Carlsbad, Calif., USA). The resultant expression vectors were named pJ294{Placuv5}[alkL]{Ptac}[ChFATB2(co_Ec)] (SEQ ID No. 62), pJ294{Placuv5}[alkL]{Ptac}[CnFATB3(co_Ec)] (SEQ ID No. 63) and pJ294{Placuv5}[alkL]{Ptac}[synUcTE(co_Ec)] (SEQ ID No. 64). The transformation of chemically competent E. coli DH5α proceeded in a manner known to those skilled in the art. Correct insertion of the target genes was checked by restriction analysis and the authenticity of the insert was verified by DNA sequencing.

### Example 14

### Preparation of Expression Vectors for the Genes fadD from Escherichia coli and Wax-dgaT (atfA) from Acinetobacter Sp. ADP1 and atfA1 from Alcanivorax borkumensis

To produce expression vectors for the genes fadD (SEQ ID No. 57) from Escherichia coli (encoding an enzyme Eᵥᵢ) and wax-dgaT (atfA in Example 5) (SEQ ID No. 65) from Acinetobacter sp. ADP1 and atfA1 (SEQ ID No. 67) from Alcanivorax borkumensis SK2 (in each case encoding an enzyme Eᵥ), the genes wax-dgaT and atfA1 were codon-optimized for expression in Escherichia coli and synthesized in combination with the gene fadD from E. coli. The synthesized DNA fragments wax-dgaT_AsADP1-fadD_Ec (SEQ ID No. 69) and atfA1_Ab-fadD_Ec (SEQ ID No. 70) were amplified with introduction of homologous regions for recombination cloning.

To amplify the fragment wax-dgaT_AsADP1-fadD_Ec, the following oligonucleotides were used:

(SEQ ID No. 71)

| | |
|---|---|
| wax-dgaT_H1_fw: | 5′-ACAGGAGGTAAAACATATGCGTCCTCTGCACC |
| | CG-3′ |

(SEQ ID No. 72)

| | |
|---|---|
| fadD_H2_rv: | 5′-GTTTCTTTACCAGACTCGAGATTGTTTTCTCT |
| | TTAGTGGGCGTC-3′ |

To amplify the fragment atfA1_Ab-fadD_Ec, the following oligonucleotides were used:

(SEQ ID No. 73)

| | |
|---|---|
| atfA_Ab_fw_kurz: | 5′-ACAGGAGGTAAAACATATGAAAGCGCT |
| | GTCCC-3′ |

(SEQ ID No. 74)

| | |
|---|---|
| fadD_H2_rv_N: | 5′-GTTTCTTTACCAGACTCGAGATTGTTT |
| | TCTCTTTAGTGGGC-3′ |

The following parameters were used for the PCR: 1×: initial denaturation, 98° C., 0:30 min; 35×: denaturation, 98° C., 0:10 min, annealing, 70° C., 0:20 min; elongation, 72° C., 1 min; 1×: terminal elongation, 72° C., 10 min. For the amplification, the Phusion™ High-Fidelity Master Mix from New England Biolabs (Frankfurt) was used in accordance with the manufacturer's recommendations. Each 50 μl of the PCR reactions was then separated on a 1% strength TAE agarose gel. The PCR, agarose gel electrophoresis, ethidium bromide staining of the DNA and determination of the PCR fragment sizes was carried out in a manner known to those skilled in the art. In both cases, PCR fragments of the expected size were able to be amplified. These were for wax-dgaT_AsADP1-fadD_Ec 3192 base pairs and atfA1_Ab-fadD_Ec 3189 base pairs. To isolate the DNA from the agarose gel, the target DNA was cut out of the gel using a scalpel and purified with the QiaQuick Gel extraction kit according to the manufacturer's instructions (Qiagen, Hilden). The purified PCR products were cloned into a NdeI- and XhoI-cut pCDF derivative which already contains a synthetic tac promotor (SEQ ID No. 39), by recombination, using the Geneart® Seamless Cloning and Assembly Kit according to the manufacturer's instructions (Life Technologies, Carlsbad, Calif., USA). Chemically competent E. coli DH5α (New England Biolabs, Frankfurt) was transformed in a manner known to those skilled in the art. Correct insertion of the target genes was checked by restriction analysis and the authenticity of the introduced genes was verified by DNA sequencing. The resultant expression vectors were named pCDF[wax-dgaT_AsAPD1(co_Ec)-fadD_Ec] (SEQ ID No. 75) and pCDF[atfA1_Ab(co_Ec)-fadD_Ec] (SEQ ID No. 76).

### Example 15

### Gas-Chromatographic Quantification of Fatty Acid Methyl Esters

Fatty acid methyl esters were quantified in the culture broth by means of gas chromatography. 500 mg/l of heptadecanoic acid methyl ester were added to the culture broth as internal reference substance. The culture broth was shaken in an equivalent volume of n-heptane for 15 min at 12 Hz to extract the fatty acid methyl esters. For phase separation, the sample was centrifuged for 10 min at 16 000×g and the organic phase was measured by gas chromatography. To separate fatty acid methyl esters, the capillary column SP™-2560 with the dimensions 100 m×0.25 mm and a film thickness of 0.2 μm (Supelco, Sigma-Aldrich, Steinheim) was used as stationary phase. The carrier gas used was helium. The separation proceeded in the course of 45 min with an injector temperature of 260° C., detector temperature of 260° C. and column temperature of 140° C. at the start, held for 5 min and increased to 240° C. at a rate of 4° C./min and held for 15 min. The injection volume was 1 μl, the split rate 1:20 and the flow rate of the carrier gas 1 ml/min. The detection was carried out by means of a flame-ionization detector (GC Perkin Elmer Clarus 500, Perkin Elmer, Rodgau). Heptadecanoic acid methyl ester (Sigma-Aldrich, Steinheim) was used as internal reference substance for quantifying the fatty acid methyl esters. The reference substances C8:0-Me caprylic acid methyl ester, C10:0-Me capric acid methyl ester, C12:0-Me lauric acid methyl ester, C14:0-Me myristic acid methyl ester, C16:0-Me palmitic acid methyl ester, C16:1-Me palmitoleic acid methyl ester, C18:0-Me stearic acid methyl ester, C18:1-Me oleic acid methyl ester (GLC Standard Mix GLC-20 1892-1AMP, GLC-30 1893-1AMP, GLC-50 1894-1AMP, Sigma-Aldrich, Steinheim) were used for calibration. The determination limits for all fatty acid methyl esters were at a concentration of 10 mg/l.

### Example 16

### Production of Fatty Acid Methyl Esters by E. coli Strains with Deletion in the fadE Gene which Overexpresses the Genes alkL from Pseudomonas putida GPo1 and a Plant Acyl-ACP Thioesterase and an Acyl-CoA Synthetase and a Wax-Ester Synthase

To generate E. coli strains having the expression vector for the genes alkL from Pseudomonas putida GPo1 and fatB2 from Cuphea hookeriana, fatB3 from Cocos nucifera and synUcTE from Umbellularia californica in combination with the expression vector for the genes fadD from Escherichia coli and wax-dgaT from Acinetobacter sp. ADP1 and atfA1 from Alcanivorax borkumensis SK2, electrocompetent cells of E. coli W3110 ΔfadE and E. coli JW5020-1 Kan^{S} were produced. This took place in a manner known to those skilled in the art. E. coli JW5020-1 Kan^{S} was transformed with the vectors pJ294{Placuv5}[alkL]{Ptac}[ChFATB2(co_Ec)] (SEQ ID No. 62) or pJ294[Placuv5}[alkL]{Ptac}[CnFATB3(co_Ec)] (SEQ ID No. 63) and pJ294[Ptac-ChFATB2_optEc] (SEQ ID No. 11) or pJ294{Ptac}[CnFATB3(co_Ec)] (SEQ ID No. 40) in combination with pCDF[atfA1_Ab(co_Ec)-fadD_Ec] (SEQ ID No. 76) and E. coli W3110 ΔfadE with the vectors pJ294{Placuv5}[alkL]{Ptac}[synUcTE] (SEQ ID No. 62) and pJ294[Ptac-synUcTE] (SEQ ID No. 41) in combination with pCDF[wax-dgaT_AsAPD1(co_Ec)-fadD_Ec] (SEQ ID No. 75) and were plated onto LB-agar plates containing spectinomycin (100 μg/ml) and ampicillin (100 μg/ml). Transformants were checked for the presence of the correct plasmids by plasmid preparation and analytical restriction analysis.

In this manner the E. coli strains hereinafter were generated:
E. coli JW5020-1 Kan^{S} pJ294[Ptac-ChFATB2_optEc]/pCDF[atfA1_Ab(co_Ec)-fadD_Ec]
E. coli JW5020-1 Kan^{S} pJ294{Placuv5}[alkL]{Ptac}[ChFATB2(co_Ec)]/pCDF[atfA1_Ab(co_Ec)-fadD_Ec]
E. coli JW5020-1 Kan^{S} pJ294{Ptac}[CnFATB3(co_Ec)]/pCDF[atfA1_Ab(co_Ec)-fadD_Ec]
E. coli JW5020-1 Kan^{S} pJ294{Placuv5}[alkL]{Ptac}[CnFATB3(co_Ec)]/pCDF[atfA1_Ab(co_Ec)-fadD_Ec]
E. coli W3110 ΔfadE pJ294[Ptac-synUcTE]/pCDF[wax-dgaT_AsAPD1(co_Ec)-fadD_Ec]
E. coli W3110 ΔfadE pJ294{Placuv5}[alkL]{Ptac}[synUcTE]/pCDF[wax-dgaT_AsAPD1(co_Ec)-fadD_Ec]

These strains were employed to investigate their ability to produce fatty acid methyl esters. In this process the following procedure was used:

The strains are subjected to a multistage aerobic culturing process. The strains under test were initially grown in Luria-Bertani Bouillon according to Miller (Merck, Darmstadt) containing 100 μg/ml of ampicillin and 100 μg/ml of spectinomycin as 5 ml preliminary culture from a single colony each time. The next culture step proceeded in M9 medium. The medium, consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution was adjusted to pH 7.4 using 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1 M hydrochloric acid (all substances from Merck, Darmstadt) was sterile-filtered before addition to the M9 medium. 10 ml of M9 medium were charged with 100 μg/ml of spectinomycin and 100 μg/ml of ampicillin into 100 ml conical flasks with chicane and inoculated with 0.5 ml from the preliminary culture. The culturing proceeded at 37° C. and 200 rpm in an incubating shaker. After a culturing time of 8 hours, 50 ml of M9 medium containing 100 μg/ml of spectinomycin and 100 μg/ml of ampicillin were charged in a 250 ml conical flask with chicane and inoculated with the 10 ml culture, in such a manner that an optical density (600 nm) of 0.2 was achieved. The culturing proceeded at 37° C. and 200 rpm in an incubating shaker. When an optical density (600 nm) of 0.6 to 0.8 is achieved, the gene expression is induced by adding 1 mM IPTG. The strains were cultured for a further 24 hours at 30° C. and 200 rpm. One hour after induction of gene expression, 1% (v/v) methanol is added to the culture broth. During the culturing, samples are withdrawn and the concentration of fatty acid methyl ester of different carbon chain lengths are quantified as described in Example 15. The results are shown in the tables hereinafter.

Production of fatty acid methyl esters with E. coli JW5020-1 Kan^{S} and E. coli W3110 ΔfadE, which overexpress one acyl-ACP thioesterase, fadD from E. coli and a wax-ester synthase. Strains with and without overexpression of alkL from P. putida GPo1 are shown. The concentrations of fatty acid methyl ester of differing carbon chain length are reported after 24 hours of culturing (n.d.=not detectable):

| | c_{Caprylic acid} | c_{Capric acid} | c_{Lauric acid} | c_{Myristic acid} |
|---|---|---|---|---|
| | _{methyl ester} | _{methyl ester} | _{methyl ester} | _{methyl ester} |
| Strain | [mg/L/OD] | [mg/L/OD] | [mg/L/OD] | [mg/L/OD] |
| E. coli W3110 ΔfadE pJ294[Ptac-synUcTE]/pCDF[wax- | n.d. | n.d. | 0.1 | 1.5 |
| dgaT_AsADP1(co_Ec)-fadD_Ec] | | | | |
| E. coli W3110 ΔfadE pJ294[alkL][Ptac-synUcTE]/ | n.d. | n.d. | 11.1 | 3.7 |
| pCDF[wax-dgaT_AsADP1(co_Ec)-fadD_Ec] | | | | |
| E. coli JW5020-1 Kan^{S} pJ294[Ptac-ChFATB2_optEc]/ | 8.0 | 4.0 | | |
| pCDF[atfA1_Ab(co_Ec)-fadD_Ec] | | | | |
| E. coli JW5020-1 Kan^{S} | 9.5 | 4.8 | | |
| pJ294{Placuv5}[alkL]{Ptac}[ChFATB2(co_Ec)]/ | | | | |
| pCDF[atfA1_Ab(co_Ec)-fadD_Ec] | | | | |
| E. coli JW5020-1 Kan^{S} pJ294{Ptac}[CnFATB3(co_Ec)]/ | n.d. | n.d. | 3.1 | 6.3 |
| pCDF[atfA1_Ab(co_Ec)-fadD_Ec] | | | | |
| E. coli JW5020-1 Kan^{S} | n.d. | n.d. | 4.3 | 8.5 |
| pJ294{Placuv5}[alkL]{Ptac}[CnFATB3(co_Ec)]/ | | | | |
| pCDF[atfA1_Ab(co_Ec)-fadD_Ec] | | | | |

It was thus shown that strains which overexpress alkL from P. putida are able to form more caprylic acid methyl ester, capric acid methyl ester, lauric acid methyl ester and myristic acid methyl ester, depending on the specificity of the overexpressed acyl-ACP thioesterase. This shows that a reinforcement of alkL is necessary for the preparation of fatty acid methyl esters of differing carbon chain length from unrelated carbon sources.

### Example 17

### Preparation of Expression Vectors for the Coexpression of Acyl-ACP Reductase Genes with the Acyl-CoA Synthetase Gene fadD from Escherichia coli and alkL from Pseudomonas putida

To produce expression vectors for the coexpression of fadD (SEQ ID No. 57) from Escherichia coli (encoding an enzyme Eᵥᵢ) and acrM (SEQ ID No. 77) from Acinetobacter sp. M-1, acr1b (SEQ ID No 79) from Acinetobacter sp. ADP1, acr1a (SEQ ID No. 81) from Acinetobacter sp. ADP1 and Maqu_{—}2220 (SEQ ID No. 83) from Marinobacter aquaeolei VT8 (encoding an enzyme Eₓ) and alkL from Pseudomonas putida (SEQ ID No. 1, encoding an AlkL gene product), the genes acr1a from Acinetobacter sp. ADP1 and Maqu_{—}2220 were codon-optimized for expression in Escherichia coli and these genes and the gene acrM from Acinetobacter sp. M-1 were synthesized (DNA2.0 Inc., Menlo Park, Calif., USA). These genes were amplified by PCR proceeding from the synthetic DNA and also the gene acr1b from Acinetobacter sp. ADP1 proceeding from chromosomal DNA as a matrix. Via the oligonucleotides used, the amplified DNA fragments were provided with homologous regions to the respective neighbouring fragment and to the PspXl-linearized target vector pCDF[alkL] (SEQ ID No. 7) for recombination cloning. At the same time, the gene fadD from Escherichia coli was amplified by PCR together with a synthetic tac promotor (SEQ ID No. 39) proceeding from a pCDF derivative as a matrix and likewise provided with homologous regions via the oligonucleotides used.

To produce the expression vector for the genes luxC, luxD and luxE from the lux operon of Photorhabdus luminescens and alkL from Pseudomonas putida GPo1, the luxCDE operon (SEQ ID No. 85) was codon-optimized for expression in Escherichia coli and synthesized (DNA2.0 Inc., Menlo Park, Calif., USA). The operon was amplified by PCR proceeding from the synthesized DNA as matrix, and the tac promotor, proceeding from a pCDF derivative which contains this promotor (SEQ ID No. 39). Both DNA fragments were provided via the oligonucleotides used with homologous regions for the target vector to the respective neighbouring fragment and to the linearized target vector for the recombination cloning.

The following oligonucleotides were employed in amplification of the tac promotor, the acyl-CoA synthetase gene and the acyl-ACP reductase genes for the coexpression with alkL:

P_{tac} and fadD for the coexpression with acr1a
[Acinetobacter sp. ADP1]:
(SEQ ID No. 86)

| | |
|---|---|
| NP-FA-P1: | 5′-TTTTCTAAGGTACCCGATAACAATTACGAGCTT |
| | CATG-3′ |

(SEQ ID No. 87)

| | |
|---|---|
| NP-FA-P2: | 5′-CTCCTTCAGCTCAGGCTTTATTGTCCAC-3′ |

P_{tac} and fadD for the coexpression with acrM
[Acinetobacter sp. M-1]:
(SEQ ID No. 86)

| | |
|---|---|
| NP-FA-P1: | 5′-TTTTCTAAGGTACCCGATAACAATTACGAGCTT |
| | CATG-3′ |

(SEQ ID No. 88)

| | |
|---|---|
| NP-FA-P5: | 5′-CTCCTTCAGCTCAGGCTTTATTGTC-3′ |

P_{tac} and fadD for the coexpression with Maqu_2220
[Marinobacterium aquaeolei VT8]:
(SEQ ID No. 86)

| | |
|---|---|
| NP-FA-P1: | 5′-TTTTCTAAGGTACCCGATAACAATTACGAGCTT |
| | CATG-3′ |

(SEQ ID No. 89)

| | |
|---|---|
| NP-FA-P8: | 5′-TCCTTCTCGCTCAGGCTTTATTGTCC-3′ |

P_{tac} and fadD for the coexpression with acr1b
[Acinetobacter sp. ADP1]:
(SEQ ID No. 86)

| | |
|---|---|
| NP-FA-P1: | 5′-TTTTCTAAGGTACCCGATAACAATTACGAGCTT |
| | CATG-3′ |

(SEQ ID No. 90)

| | |
|---|---|
| NP-FA-P14: | 5′-CCTGATTGGCTCAGGCTTTATTGTC-3′ |

P_{tac} for the expression of luxCDE
[Photorhabdus luminescens]:
(SEQ ID No. 86)

| | |
|---|---|
| NP-FA-P1: | 5′-TTTTCTAAGGTACCCGATAACAATTACGAGCTT |
| | CATG-3′ |

(SEQ ID No. 91)

| | |
|---|---|
| NP-FA-P11: | 5′-ACCTCCTAGTTTTACCTCCTGTTAAACAA-3′ |

acr1a [Acinetobacter sp. ADP1]:
(SEQ ID No. 92)

| | |
|---|---|
| NP-FA-P3: | 5′-CCTGAGCTGAAGGAGTTACAGTTTGATC-3′ |

(SEQ ID No. 93)

| | |
|---|---|
| NP-FA-P4: | 5′-GTTTCTTTACCAGACTTATCACCAGTGCTCACC |
| | -3′ |

acrM [Acinetobacter sp. M1]:
(SEQ ID No. 94)

| | |
|---|---|
| NP-FA-P6: | 5′-CCTGAGCTGAAGGAGTTACAGTATGAATG-3′ |

(SEQ ID No. 95)

| | |
|---|---|
| NP-FA-P7: | 5′-GTTTCTTTACCAGACTTATTACCAGTGTTCG- |
| | 3′ |

Maqu_2220 [Marinobacterium aquaeolei VT8]:
(SEQ ID No. 96)

| | |
|---|---|
| NP-FA-P9: | 5′-CCTGAGCGAGAAGGAGTTCTATCATGG-3′ |

(SEQ ID No. 97)

| | |
|---|---|
| NP-FA-P10: | 5′-GTTTCTTTACCAGACTCATTACGCGGCCTTTT |
| | TGC-3′ |

acr1b [Acinetobacter sp. ADP1]:
(SEQ ID No. 98)

| | |
|---|---|
| NP-FA-P15: | 5′-CCTGAGCCAATCAGGGAAAAACGCGTG-3′ |

(SEQ ID No. 99)

| | |
|---|---|
| NP-FA-P16: | 5′-GTTTCTTTACCAGACCTCTCGGTATGAGAGGC |
| | TTC-3′ |

luxCDE [Photorhabdus luminescens]:
(SEQ ID No. 100)

| | |
|---|---|
| NP-FA-P12: | 5′-GTAAAACTAGGAGGTAAAAAAAATGACG-3′ |

(SEQ ID No. 101)

| | |
|---|---|
| NP-FA-P13: | 5′-GTTTCTTTACCAGACTTAGCTATCGAACGAACG |
| | CCTCG-3′ |

The following parameters were used for the PCR: 1×: initial denaturation, 98° C., 0:30 min; 35×: denaturation, 98° C., 0:10 min, annealing, 60° C., 0:45 min; elongation, 72° C., 1:30 min; 1×: terminal elongation, 72° C., 10 min. For the amplification, the Phusion™ High-Fidelity Master Mix from New England Biolabs (Frankfurt) was used in accordance with the manufacturer's recommendations. In each case 50 μl of the PCR reactions were then separated on a 1% strength TAE-agarose gel. The PCR, the agarose-gel electrophoresis, ethidium bromide staining of the DNA and determination of the PCR fragment sizes were carried out in a manner known to those skilled in the art.

In all cases, PCR fragments of the expected size were able to be amplified. These were, for the tac promotor 171 base pairs, for the tac promotor and fadD for coexpression with acr1a [A.sp. ADP1] and Maqu2220 1927 base pairs, for coexpression with acrM 1919 base pairs and for coexpression with acr1b [A.sp. ADP1] 1933 base pairs. The PCR fragments for acr1a [A.sp. ADP1] were 952 base pairs, for acrM 906 base pairs, for Maqu2220 1561 base pairs, for acr1b [A.sp.ADP1] 903 base pairs, and for luxCDE 3621 base pairs. For isolation of the DNA from the agarose gel, the target DNA was cut out from the gel using a scalpel and purified using the QiaQuick Gel extraction kit according to the manufacturer's instructions (Qiagen, Hilden). The purified PCR products were cloned by recombination, using the Geneart® Seamless Cloning and Assembly Kit according to the manufacturer's instructions (Life Technologies, Carlsbad, Calif., USA) into the PspX/-linearized vector pCDF[alkL] (SEQ ID No. 7). Chemically competent E. coli DH5α (New England Biolabs, Frankfurt) was transformed in a manner known to those skilled in the art. The correct insertion of the target genes was checked by restriction analysis and the authenticity of the introduced genes was validated by DNA sequencing.

In this manner the following expression vectors resulted:

(SEQ ID No. 102)
• pCDF{Placuv5}[alkL]{Ptac}[fadD_Ec-acr1b_AsADP1]
(SEQ ID No. 103)
• pCDF{Placuv5}[alkL]{Ptac}
[fadD_Ec-acr1a_AsADP1(co_Ec)]
(SEQ ID No. 104)
• pCDF{Placuv5}[alkL]{Ptac}[fadD_Ec-acrM_AsM1]
(SEQ ID No. 105)
• pCDF{Placuv5}[alkL]{Ptac}
[fadD_Ec-Maqu2220(co_Ec)]
(SEQ ID No. 106)
• pCDF{Placuv5}[alkL]{Ptac}[luxCDE_Pl(co_Ec)]

For preparation of vectors for the coexpression of fadD from Escherichia coli and acrM from Acinetobacter sp. M-1, acr1b from Acinetobacter sp. ADP1, acr1a from Acinetobacter sp. ADP1 (codon-optimized) and Maqu_{—}2220 from Marinobacter aquaeolei VT8 (codon-optimized) and the expression of luxC, luxD and luxE from Photorhabdus luminescens (codon-optimized) without the coexpression of alkL, these genes were amplified by PCR starting from the previously generated expression vectors pCDF{Placuv5}[alkL]{Ptac}[fadD_Ec-acr1b_AsADP1], pCDF{Placuv5}[alkL]{Ptac}[fadD_Ec-acr1a_AsADP1(co_Ec)], pCDF{Placuv5}[alkL]{Ptac}[fadD_Ec-acrM_AsM1], pCDF{Placuv5}[alkL]{Ptac}[fadD_Ec-Maqu2220(co_Ec)] and pCDF{Placuv5}[alkL]{Ptac}[luxCDE_Pl(co_Ec)] with introduction of homologous regions to PspXI/NcoI-cut target vector pCDF[alkL] (SEQ ID No. 7).

The following oligonucleotides were employed here:

(SEQ ID No. 107)

| | |
|---|---|
| NP-FA-P17: | 5′-AATAAGGAGATATACGATAACAATTACGAGCTTCAT |
| | G-3′ |

(SEQ ID No. 108)

| | |
|---|---|
| NP-FA-P18: | 5′-GTTTCTTTACCAGACGCGTTCAAATTTCGCAGCA |
| | G-3′ |

The following parameters were employed for the PCR: 1×: initial denaturation, 98° C., 0:30 min; 35×: denaturation, 98° C., 0:15 min, annealing, 60° C., 0:45 min; elongation, 72° C., 1:30 min; 1×: terminal elongation, 72° C., 10 min. The Phusion™ High-Fidelity Master Mix from New England Biolabs (Frankfurt) was used for the amplification, following the manufacturer's recommendations. 50 μl of the PCR reactions in each case were then separated on a 1% strength TAE-agarose gel. The PCR, the agarose gel electrophoresis, ethidium bromide staining of the DNA and determination of the PCR fragment sizes were carried out in a manner known to those skilled in the art.

In all cases, PCR fragments of the expected size were able to be amplified. These were 2901 base pairs for P_{tac}-fadD_Ec-acr1a_AsADP1, 2877 base pairs for P_{tac}-fadD_Ec-acrM_AsM1, 3532 base pairs for P_{tac}-fadD_Ec-Maqu_{—}2220, 2907 base pairs for P_{tac}-fadD_Ec-acr1b_AsADP1, and 3810 base pairs for P_{tac}-luxCDE.

To isolate the DNA from the agarose gel, the target gel was cut out of the gel using a scalpel and purified using the QiaQuick Gel extraction kit according to the manufacturer's instructions (Qiagen, Hilden). The purified PCR products were cloned into the vector pCDF[alkL] (SEQ ID No. 7) digested with PspXI and NcoI by means of recombination, using the Geneart® Seamless Cloning and Assembly Kit according to the manufacturer's instructions (Life Technologies, Carlsbad, Calif., USA). Owing to the restriction of the vector, the alkL gene is removed therefrom. The transformation of chemically competent E. coli DH5α (New England Biolabs, Frankfurt) was performed in a manner known to those skilled in the art. The correct insertion of the target genes was checked by restriction analysis and the authenticity of the introduced genes was verified by DNA sequencing.

The expression vectors hereinafter resulted in this manner:

(SEQ ID No. 109)
• pCDF{Ptac}[fadD_Ec-acr1b_AsADP1]
(SEQ ID No. 110)
• pCDF{Ptac}[fadD_Ec-acr1a_AsADP1(co_Ec)]
(SEQ ID No. 111)
• pCDF{Ptac}[fadD_Ec-acrM_AsM1]
(SEQ ID No. 112)
• pCDF{Ptac}[fadD_Ec-Maqu2220(co_Ec)]
(SEQ ID No. 113)
• pCDF{Ptac}[luxCDE_Pl(co_Ec)]

### Example 18

### Chromatographic Quantification of Fatty Alcohols and Fatty Aldehydes

Fatty alcohols and fatty aldehydes are quantified by gas chromatography with mass-spectrometric coupling (GC/MS).

To extract the samples consisting of 1 ml of culture broth they are admixed with 500 μl of ethyl acetate (Chromasolv®Plus 99.9%, Sigma No. 650528-1L), shaken for 10 min at 12 Hz and sedimented for 5 min at 13 200 rpm in a bench centrifuge (Eppendorf, Hamburg). The organic phase (ethyl acetate) is transferred to HPLC vials with an insert and analysed for fatty alcohols and fatty aldehydes of differing chain length (C8-C18) by GC/MS coupling.

To separate fatty alcohols and fatty aldehydes, the ZB-50 capillary column having the dimensions 30 m×320 μm and a film thickness of 0.5 μm (Phenomenex, Aschaffenburg) is used as stationary phase. The carrier gas used is helium at a constant flow rate of 1.5 ml/min. The separation proceeds in the course of 45 min at an injection temperature of 250° C. and a detector temperature of 250° C. The column temperature at the start is 40° C. and is held for 2 min. Thereafter the column temperature is increased at 7° C./min to 150° C., then at 15° C./min to 320° C. and held for 10 min. The injection volume is 1 μl splitless. Detection is performed by means of MS (DSQ II) detector (Thermo Fisher Scientific) with a mass range of 12-800 m/z. The reference substance employed is a standard mixture consisting of in each case 10 μg/ml 1-octanal (99%, Sigma-Aldrich), 1-octanol (Sigma-Aldrich), 1-decanal (>98%, Sigma-Aldrich), 1-decanol (>99%, Sigma-Aldrich), 1-dodecanal (>92%, Sigma-Aldrich), 1-dodecanol (>98%, Sigma-Aldrich), 1-tetradecanal, 1-tetradecanol (>99%, Fluka), 1-hexadecanal and 1-hexadecanol (99%, Sigma-Aldrich) for calibration. Relative quantification of the samples is performed via the peak areas.

### Example 19

### Production of Fatty Alcohols by E. coli Strains Having a Deletion in the fadE Gene which Overexpresses the alkL Genes from Pseudomonas putida GPo1 and fatB2 from Cuphea hookeriana or fatB3 from Cocos nucifera and the fadD Gene from Escherichia coli and an Acyl-ACP Reductase Gene

To generate E. coli strains having the expression vector for the alkL gene from Pseudomonas putida GPo1 and the fadD gene from E. coli, and the genes acr1a from Acinetobacter sp. ADP1 or acr1b from Acinetobacter sp. ADP1 or acrM from Acinetobacter sp. M-1 or Maqu2220 from Marinobacterium aquaeolei VT8 or luxCDE from Photorhabdus luminescens in combination with the expression vector for the fatB2 gene from Cuphea hookeriana and/or fatB3 from Cocos nucifera, electrocompetent cells of E. coli W3110 ΔfadE and E. coli JW5020-1 Kan^{S} were prepared. This took place in a manner known to those skilled in the art. E. coli JW5020-1 Kan^{S} is a derivative of E. coli JW5020-1 (CGSC, The coli genetic stock center, Yale University, New Haven, USA), this in turn is a E. coli BW25113 derivative which bears a deletion of the fadE gene. The fadE gene was replaced by a kanamycin cassette. This was removed in a manner known to those skilled in the art before the strain was equipped with the expression vectors using a helper plasmid which encodes fip recombinase (see Datsenko K. A. and Wanner B. L. (2000) PNAS 97(12):6640-6645) resulting in strain E. coli JW5020-1 Kan^{S}. E. coli JW5020-1

Kan^{S} was transformed with the plasmids pJ294[Ptac-ChFATB2_optEc] (SEQ ID No. 10) in combination with pCDF{Ptac}[fadD_Ec-acr1a_AsADP1(co_Ec)] (SEQ ID No. 110), pCDF{Placuv5}[alkL]{Ptac}[fadD_Ec-acr1a_AsADP1(co_Ec)] (SEQ ID No. 103), pCDF{Ptac}[fadD_Ec-Maqu2220(co_Ec)] (SEQ ID No. 112) or pCDF{Placuv5}[alkL]{Ptac}[fadD_Ec-Maqu2220(co_Ec)] (SEQ ID No. 105) and E. coli W3110 ΔfadE with the plasmids pJ294{Ptac}[CnFATB3(co_Ec)] (SEQ ID No. 40) in combination with pCDF{Ptac}[fadD_Ec-acr1b_AsADP1] (SEQ ID No. 109), pCDF{Placuv5}[alkL]{Ptac}[fadD_Ec-acr1b_AsADP1] (SEQ ID No. 102), pCDF{Ptac}[fadD_Ec-acrM_AsM1] (SEQ ID No. 111), pCDF{Placuv5}[alkL]{Ptac}[fadD_Ec-acrM_AsM1] (SEQ ID No. 104), pCDF{Ptac}[luxCDE_Pl(co_Ec)] (SEQ ID No. 113) or pCDF{Placuv5}[alkL]{Ptac}[luxCDE_Pl(co_Ec)] (SEQ ID No. 106) and plated out on LB-agar plates containing spectinomycin (100 μg/ml) and ampicillin (100 μg/ml). Transformants were checked for the presence of the correct plasmids by plasmid preparation and analytical restriction analysis.

In this manner the E. coli strains hereinafter were generated:
E. coli JW5020-1 Kan^{S} pJ294[Ptac-ChFATB2_optEc]/pCDF{Ptac}[fadD_Ec-acr1a_AsADP1(co_Ec)]
E. coli JW5020-1 Kan^{S} pJ294[Ptac-ChFATB2_optEc]/pCDF{Placuv5}[alkL]{Ptac}[fadD_Ec-acr1a_AsADP1(co_Ec)]
E. coli JW5020-1 Kan^{S} pJ294[Ptac-ChFATB2_optEc]/pCDF{Ptac}[fadD_Ec-Maqu2220(co_Ec)]
E. coli JW5020-1 Kan^{S} pJ294[Ptac-ChFATB2_optEc]/pCDF{Placuv5}[alkL]{Ptac}[fadD_Ec-Maqu2220(co_Ec)]
E. coli W3110 ΔfadE pJ294{Ptac}[CnFATB3(co_Ec)]/pCDF{Ptac}[fadD_Ec-acrM_AsM1]
E. coli W3110 ΔfadE pJ294{Ptac}[CnFATB3(co_Ec)]/pCDF{Ptac}[alkL][fadD_Ec-acrM_AsM1]
E. coli W3110 ΔfadE pJ294{Ptac}[CnFATB3(co_Ec)]/pCDF{Ptac}[fadD_Ec-acr1b_AsADP1]
E. coli W3110 ΔfadE pJ294{Ptac}[CnFATB3(co_Ec)]/pCDF{Placuv5}[alkL]{Ptac}[fadD_Ec-acr1b_AsADP1]
E. coli W3110 ΔfadE pJ294{Ptac}[CnFATB3(co_Ec)]/pCDF{Ptac}[luxCDE_Pl(co_Ec)]
E. coli W3110 ΔfadE pJ294{Ptac}[CnFATB3(co_Ec)]/pCDF{Placuv5}[alkL]{Ptac}[luxCDE_Pl(co_Ec)]

These strains were used to study their ability to produce fatty alcohols. The following procedure was adopted here:

The strains are subjected to a multistage aerobic culturing process. The strains under investigation were first initially grown in Luria-Bertani Bouillon according to Miller (Merck, Darmstadt) containing 100 μg/ml of ampicillin and 100 μg/ml of spectinomycin as a 5 ml preliminary culture in each case from a single colony. The next culturing step proceeded in M9 medium. The medium consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution were adjusted to a pH of 7.4 with 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1 M hydrochloric acid (all substances from Merck, Darmstadt) was sterile-filtered before addition to the M9 medium. 10 ml of M9 medium were charged into a 100 ml conical flask with chicane with 100 μg/ml of spectinomycin and 100 μg/ml of ampicillin and inoculated with 0.5 ml from the preliminary culture. The culturing proceeded at 37° C. and 200 rpm in an incubating shaker. After a culturing time of 8 hours, 50 ml of M9 medium containing 100 μg/ml of spectinomycin and 100 μg/ml of ampicillin were charged into a 250 ml conical flask with chicane and inoculated with the 10 ml culture, in such a manner that an optical density (600 nm) of 0.2 is achieved. Culturing proceeded at 37° C. and 200 rpm in an incubating shaker. When an optical density (600 nm) of 0.6 to 0.8 is achieved, the gene expression is induced by adding 1 mM IPTG. The strains were cultured for a further 24 hours at 30° C. and 200 rpm. During culturing, samples are withdrawn and the concentration of fatty alcohols of differing carbon chain lengths is quantified as described in Example 18. The results are shown in the table hereinafter.

Production of fatty alcohols using E. coli JW5020-1 Kan^{S} and E. coli W3110 ΔfadE which overexpress a plant acyl-ACP thioesterase, fadD from E. coli and also a fatty acyl-CoA reductase. Strains with and without overexpression of alkL from P. putida GPo1 are shown. The concentrations of fatty alcohols of differing carbon chain length are reported after culturing for 24 hours (n.d.=not detectable):

| | Decanol | Dodecanol | Tetradecanol | Hexadecanol |
|---|---|---|---|---|
| | [Peak area/OD] | [Peak area/OD] | [Peak area/OD] | [Peak area/OD] |
| E. coli W3110 ΔfadE pJ294{Ptac}[CnFATB3(co_Ec)]/ | n.d. | 2.53E+06 | 9.74E+06 | 1.39E+07 |
| pCDF{Ptac}[fadD_Ec-acrM_AsM1(co_Ec)] | | | | |
| E. coli W3110 ΔfadE pJ294{Ptac}[CnFATB3(co_Ec)]/ | n.d. | 1.15E+07 | 2.90E+07 | 3.01E+07 |
| pCDF{Placuv5}[alkL]{Ptac}[fadD_Ec- | | | | |
| acrM_AsM1(co_Ec)] | | | | |
| E. coli W3110 ΔfadE pJ294{Ptac}[CnFATB3(co_Ec)]/ | n.d. | 1.42E+06 | 6.91E+06 | n.d. |
| pCDF{Ptac}[luxCDE_PI(co_Ec)] | | | | |
| E. coli W3110 ΔfadE pJ294{Ptac}[CnFATB3(co_Ec)]/ | n.d. | 3.78E+06 | 1.77E+07 | n.d. |
| pCDF{Placuv5}[alkL]{Ptac}[luxCDE_PI(co_Ec)] | | | | |
| E. coli W3110 ΔfadE pJ294{Ptac}[CnFATB3(co_Ec)]/ | n.d. | 2.43E+04 | 1.59E+06 | n.d. |
| pCDF{Ptac}[fadD_Ec-acr1b_AsADP1)] | | | | |
| E. coli W3110 ΔfadE pJ294{Ptac}[CnFATB3(co_Ec)]/ | n.d. | 8.65E+05 | 2.44E+06 | n.d. |
| pCDF{Placuv5}[alkL]{Ptac}[fadD_Ec-acr1b_AsADP1)] | | | | |
| E. coli JW5020-1 Kan^{S} pJ294{Ptac}[Ptac- | 4.65E+06 | n.d. | 3.49E+06 | 5.63E+06 |
| ChFATB2_optEc]/pCDF{Ptac}[fadD_Ec- | | | | |
| acr1a_AsADP1(co_Ec)] | | | | |
| E. coli JW5020-1 Kan^{S} pJ294[Ptac-ChFATB2_optEc]/ | 1.61E+07 | n.d. | 4.23E+07 | 3.45E+07 |
| pCDF{Placuv5}[alkL]{Ptac}[fadD_Ec- | | | | |
| acr1a_AsADP1(co_Ec)] | | | | |
| E. coli JW5020-1 Kan^{S} pJ294[Ptac-ChFATB2_optEc]/ | n.d. | n.d. | 5.66E+04. | 7.12E+04 |
| pCDF{Ptac}[fadD_Ec-Maqu2220_Ma(co_Ec)] | | | | |
| E. coli JW5020-1 Kan^{S} pJ294[Ptac-ChFATB2_optEc]/ | n.d. | n.d. | 6.74E+07 | 2.48E+08 |
| pCDF{Placuv5}[alkL]{Ptac}[fadD_Ec- | | | | |
| Maqu2220_Ma(co_Ec)] | | | | |

It was thus shown that strains which overexpress alkL from P. putida are able to form more decanol, dodecanol, tetradecanol and hexadecanol than strains without alkL. This shows that reinforcement of alkL is necessary for producing fatty alcohols of various chain lengths from unrelated carbon sources.

### Example 20

### Preparation of an Expression Vector for the Mmar_{—}3356 Gene from Mycobacterium marinum

To produce an expression vector for the Mmar_{—}3356 gene (SEQ ID No. 114) from Mycobacterium marinum, the gene was codon-optimized for expression in E. coli. The synthesized gene for the SAM-dependent methyltransferase (Eᵥₐ) was amplified with introduction of an NdeI cleavage site upstream and an XbaI cleavage site downstream. The restriction cleavage sites were introduced via the oligonucleotides used.

(SEQ ID No.119)

| | |
|---|---|
| mt_fw_Ndel: | 5′-TATATACATATGCCAAGAGAGATTAGATTACC-3′ |

(SEQ ID No. 120)

| | |
|---|---|
| mt_rv_Xbal: | 5′-TATATATCTAGACTGAGTTAGGCACGTTTCG-3′ |

The following parameters were used for the PCR: 1×: initial denaturation, 98° C., 0:30 min; 35×: denaturation, 98° C., 0:10 min, annealing, 62° C., 0:20 min; elongation, 72° C., 0:30 min; 1×: terminal elongation, 72° C., 10 min. The Phusion™ High-Fidelity Master Mix from New England Biolabs (Frankfurt) was used for the amplification in accordance with the manufacturer's recommendations. 50 μl of the PCR reactions were then separated in each case on a 1.5% strength TAE-agarose gel. The PCR, the agarose gel electrophoresis, ethidium bromide staining of the DNA and determination of the PCR fragment sizes were carried out in a manner known to those skilled in the art.

The PCR fragment having the expected size of 1133 base pairs was able to be amplified. To isolate the DNA from the agarose gel, the target DNA was cut out of the gel using a scalpel and purified using the QiaQuick Gel extraction kit in accordance with the manufacturer's instructions (Qiagen, Hilden). The purified PCR product was digested using the restriction endonucleases NdeI and XbaI and was ligated into an appropriately cut pJ281 derivative (SEQ ID No. 121) which contains a lacuv5 promotor. The transformation of chemically competent E. coli DH5α (New England Biolabs, Frankfurt) proceeded according to a manner known to those skilled in the art. Correct insertion of the target genes was checked by restriction analysis and the authenticity of the introduced genes was validated by DNA sequencing. The completed E. coli expression vector was termed pJ281{Placuv5}[Mmar_{—}3356(co_Ec)] (SEQ ID No. 116).

### Example 21

### Production of Fatty Acid Esters by E. coli Strains with a Deletion in the fadE Gene which Overexpress a Plant Acyl-ACP Thioesterase Gene, the alkL Genes from Pseudomonas putida GPo1 and Mmar_{—}3356 from Mycobacterium marinum

To generate an E. coli strain having expression vectors for the genes fatB1 from Cuphea hookeriana, fatB2 from Cuphea hookeriana, fatB3 from Cocos nucifera or synUcTE from Umbellularia californica in combination with an expression vector for the Mmar_{—}3356 gene from Mycobacterium marinum and an expression vector for the alkL gene from Pseudomonas putida GPo1, electrocompetent cells of E. coli JW5020-1 Kan^{S} and E. coli W3110 ΔfadE are produced. This proceeded in a manner are known to those skilled in the art. The strains are transformed sequentially with the vectors pJ294[Ptac-ChFATB1_optEc] (SEQ ID No. 12), pJ294[Ptac-ChFATB2_optEc] (SEQ ID No. 10), pJ294{Ptac}[CnFATB3(co_Ec)] (SEQ ID No. 40) and/or pJ294[Ptac-synUcTE] (SEQ ID No. 41) and pJ281{Placuv5}[Mmar_{—}3356(co_Ec)] (SEQ ID No. 116) and pCDF[alkL] (SEQ ID No. 7) and/or pCDFDuet-1 (71340-3, Merck, Darmstadt) and plated onto LB-agar plates containing ampicillin (100 μg/ml), kanamycin (50 μg/ml) and spectinomycin (100 μg/ml). Transformants are checked by plasmid preparation and analytical restriction analysis with respect to the presence of the correct plasmids. In this manner the strains hereinafter were constructed:
E. coli JW5020-1 Kan^{S} pJ294[Ptac-ChFATB1_optEc]/pJ281{Placuv5}[Mmar_{—}3356(co_Ec)]/pCDF[alkL]
E. coli JW5020-1 Kan^{S} pJ294[Ptac-ChFATB1_optEc]/pJ281{Placuv5}[Mmar_{—}3356(co_Ec)]/pCDFDuet-1
E. coli JW5020-1 Kan^{S} pJ294[Ptac-ChFATB2_optEc]/pJ281{Placuv5}[Mmar_{—}3356(co_Ec)]/pCDF[alkL]
E. coli JW5020-1 Kan^{S} pJ294[Ptac-ChFATB2_optEc]/pJ281{Placuv5}[Mmar_{—}3356(co_Ec)]/pCDFDuet-1
E. coli JW5020-1 Kan^{S} pJ294{Ptac}[CnFATB3(co_Ec)]/pJ281{Placuv5}[Mmar_{—}3356(co_Ec)]/pCDF[alkL]
E. coli JW5020-1 Kan^{S} pJ294{Ptac}[CnFATB3(co_Ec)]/pJ281{Placuv5}[Mmar_{—}3356(co_Ec)]/pCDFDuet-1
E. coli W3110 ΔfadE pJ294[Ptac-synUcTE]/pJ281{Placuv5}[Mmar_{—}3356(co_Ec)]/pCDF[alkL]
E. coli W3110 ΔfadE pJ294[Ptac-synUcTE]/pJ281{Placuv5}[Mmar_{—}3356(co_Ec)]/pCDFDuet-1

These strains are used to study their ability to produce fatty acid methyl esters from glucose. The following procedure is adopted here:

The strains are subjected to a multistage aerobic culturing process. The strains under investigation are first initially grown in Luria-Bertani Bouillon according to Miller (Merck, Darmstadt) containing 50 μg/ml of kanamycin, 100 μg/ml of spectinomycin and 100 μg/ml of ampicillin as 5 ml preliminary culture from a single colony in each case. The next culture step proceeds in M9 medium. The medium, consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution, is adjusted to a pH of 7.4 using 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1 M hydrochloric acid (all substances from Merck, Darmstadt) is sterile-filtered before addition to the M9 medium. 10 ml of M9 medium are charged together with 50 μg/ml kanamycin, 100 μg/ml spectinomycin and 100 μg/ml ampicillin into a 100 ml conical flask with chicane and inoculated with 0.5 ml of the preliminary culture. The culturing proceeds at 37° C. and 200 rpm in an incubating shaker. After a culturing time of 8 hours, 50 ml of M9 medium containing 50 μg/ml of kanamycin, 100 μg/ml of spectinomycin and 100 μg/ml of ampicillin are charged in a 250 ml conical flask with chicane and inoculated with the 10 ml culture in such a manner that an optical density (600 nm) of 0.2 is achieved. The culturing proceeds at 37° C. and 200 rpm in an incubating shaker. When an optical density (600 nm) of 0.6 to 0.8 is reached, the gene expression is induced by adding 1 mM IPTG. The strains are cultured for at least a further 24 hours at 30° C. and 200 rpm. During the culturing, samples are withdrawn and the concentration of fatty acid methyl esters of differing carbon chain lengths is quantified as described in Example 15. It is shown that the strains E. coli JW5020-1 Kan^{S} pJ294[Ptac-ChFATB1_optEc]/pJ281{Placuv5}[Mmar_{—}3356(co_Ec)]/pCDF[alkL], E. coli JW5020-1 Kan^{S} pJ294[Ptac-ChFATB2_optEc]/pJ281{Placuv5}[Mmar_{—}3356(co_Ec)]/pCDF[alkL], E. coli JW5020-1 Kan^{S} pJ294{Ptac}[CnFATB3(co_Ec)]/pJ281{Placuv5}[Mmar_{—}3356(co_Ec)]/pCDF[alkL] and E. coli W3110 ΔfadE pJ294[Ptac-synUcTE]/pJ281{Placuv5}[Mmar_{—}3356(co_Ec)]/pCDF[alkL] are able, depending on the specificity of the overexpressed acyl-CoA thioesterase gene, to form fatty acid methyl esters of different carbon chain length and degree of saturation compared with the corresponding strains which do not overexpress the alkL gene. In particular, E. coli JW5020-1 Kan^{S} pJ294[Ptac-ChFATB1_optEc]/pJ281{Placuv5}[Mmar_{—}3356(co_Ec)]/pCDF[alkL] can produce more fatty acid methyl esters of chain length C14:0, C16:0 and C16:1, E. coli JW5020-1 Kan^{S} pJ294[Ptac-ChFATB2_optEc]/pJ281{Placuv5}[Mmar_{—}3356(co_Ec)]/pCDF[alkL] can produce more fatty acid methyl esters of chain length C8:0 and C10:0, E. coli JW5020-1 Kan^{S} pJ294{Ptac}[CnFATB3(co_Ec)]/pJ281{Placuv5}[Mmar_{—}3356(co_Ec)]/pCDF[alkL] can produce more fatty acid methyl esters of chain length C12:0, C14:0 and C16:1 and E. coli W3110 ΔfadE pJ294[Ptac-synUcTE]/pJ281{Placuv5}[Mmar_{—}3356(co_Ec)]/pCDF[alkL] can produce more fatty acid methyl esters of chain length C12:0 and C14:0 from glucose than the corresponding strains which lack the alkL gene from Pseudomonas putida GPo1.

### Example 22

### Preparation of an Expression Vector for the Coexpression of the Genes MSMEG_{—}2956 from Mycobacterium smegmatis, npt from Nocardia sp. with alkL from Pseudomonas putida

To prepare a E. coli expression vector for the genes MSMEG_{—}2956 (SEQ ID No. 117) from Mycobacterium smegmatis, npt (SEQ ID No: 122) from Nocardia sp. and alkL (SEQ ID No. 1) from Pseudomonas putida GPo1, the genes MSMEG_{—}2956 and npt are codon-optimized for expression in Escherichia coli and synthesized. The synthesized genes are cloned as an operon following a lacuv5 promoter using recombination cloning. MSMEG 2956 and npt are derivative with introduction of homologous regions for recombination cloning. The oligonucleotides hereinafter are used here:

Promoter region P_{lacuv5}:
(SEQ ID No. 126)

| | |
|---|---|
| NP-FA-P22: | 5′-CCGGTAGTCAATAAAATCGCACCTGGTGTTTAAAC |
| | G-3′ |

(SEQ ID No. 127)

| | |
|---|---|
| NP-FA-P23: | 5′-TGTCATATGCCACTCTCCTTGGTTCC-3′ |

MSMEG_2956(co_Ec) and npt_Noc(co_Ec):
(SEQ ID No. 128)

| | |
|---|---|
| NP-FA-P24: | 5′-GAGTGGCATATGACAATTGAAACGCGCGAAG-3′ |

(SEQ ID No. 129)

| | |
|---|---|
| NP-FA-P25: | 5′-TCTATTGCTGGTTTACCTAGGTTATCATTATCATG |
| | C-3′ |

The following parameters are used for the PCR: 1×: initial denaturation, 98° C., 0:30 min; 35×: denaturation, 98° C., 0:15 min, annealing, 60° C., 0:30 min; elongation, 72° C., 0:20 min; 1×: terminal elongation, 72° C., 10 min. The Phusion™ High-Fidelity Master Mix from New England Biolabs (Frankfurt) is used for the amplification in accordance with the manufacturer's recommendations. 50 μl of the PCR reactions in each case are then separated on a 1% strength TAE agarose gel and cut out from the agarose gel and purified. The PCR, agarose gel electrophoresis, ethidium bromide staining of the DNA and determination of the PCR fragment sizes and purification of the DNA fragments are carried out in a manner known to those skilled in the art. PCR fragments of 210 base pairs for the lacuv5 promoter region and 4241 base pairs for the DNA fragment MSMEG_{—}2956(co_Ec)-npt_Noc(co_Ec) are expected. The purified PCR fragments are cloned into the restriction endonuclease-Age/-digested vector pCDF[alkL] (SEQ ID No. 7) and pCDFDuet-1 (71340-3, Merck, Darmstadt) by means of recombination and using the Geneart® Seamless Cloning and Assembly Kit in accordance with the manufacturer's instructions (life Technologies, Carlsbad, Calif., USA). This generates the vectors pCDF[alkL][MSMEG_{—}2956(co_Ec)-npt_Noc(co_Ec)] (SEQ ID No. 124) and pCDF[MSMEG_{—}2956(co_Ec)-npt_Noc(co_Ec)] (SEQ ID No. 125). Chemically competent E. coli DH10β is transformed in a manner known to those skilled in the art. Correct insertion of the target genes is checked by restriction analysis and the authenticity of the insert is validated by DNA sequencing.

### Example 23

### Production of Fatty Aldehydes and Fatty Alcohols by E. coli Strains with a Deletion in the fadE Gene which Overexpress a Plant Acyl-ACP Thioesterase Gene, the Genes alkL from Pseudomonas putida GPo1 and MSMEG_{—}2956 from Mycobacterium smegmatis and npt from Nocardia sp

To generate E. coli strains having expression vectors for the genes fatB1 from Cuphea hookeriana, fatB2 from Cuphea hookeriana, fatB3 from Cocos nucifera or synUcTE from Umbellularia californica in combination with an expression vector for the genes MSMEG_{—}2956 from Mycobacterium smegmatis, npt from Nocardia sp. and alkL from Pseudomonas putida GPo1 electrocompetent cells of E. coli JW5020-1 Kan^{S} and E. coli W3110 ΔfadE are prepared. This is performed in a manner known to those skilled in the art. The strains are transformed with the vectors pJ294[Ptac-ChFATB1_optEc] (SEQ ID No. 12), pJ294[Ptac-ChFATB2_optEc] (SEQ ID No. 11), pJ294{Ptac}[CnFATB3(co_Ec)] (SEQ ID No. 40) and/or pJ294[Ptac-synUcTE] (SEQ ID No. 41) and pCDF[alkL][MSMEG_{—}2956(co_Ec)-npt_Noc(co_Ec)] (SEQ ID No. 124) and/or pCDF[MSMEG_{—}2956(co_Ec)-npt_Noc(co_Ec)] (SEQ ID No. 128) and plated onto LB-agar plates containing ampicillin (100 μg/ml) and spectinomycin (100 μg/ml). Transformants are checked with respect to the presence of the correct plasmids by plasmid preparation and analytical restriction analysis. The strains hereinafter are constructed in this manner:
E. coli JW5020-1 Kan^{S} pJ294[Ptac-ChFATB1_optEc]/pCDF[alkL][MSMEG_{—}2956(co_Ec)-npt_Noc(co_Ec)]
E. coli JW5020-1 Kan^{S} pJ294[Ptac-ChFATB1_optEc]/pCDF[MSMEG_{—}2956(co_Ec)-npt_Noc(co_Ec)]
E. coli JW5020-1 Kan^{S} pJ294[Ptac-ChFATB2_optEc]/pCDF[alkL][MSMEG_{—}2956(co_Ec)-npt_Noc(co_Ec)]
E. coli JW5020-1 Kan^{S} pJ294[Ptac-ChFATB2_optEc]/pCDF[MSMEG_{—}2956(co_Ec)-npt_Noc(co_Ec)]
E. coli JW5020-1 Kan^{S} pJ294{Ptac}[CnFATB3(co_Ec)]/pCDF[alkL][MSMEG_{—}2956(co_Ec)-npt_Noc(co_Ec)]
E. coli JW5020-1 Kan^{S} pJ294{Ptac}[CnFATB3(co_Ec)]/pCDF[MSMEG_{—}2956(co_Ec)-npt_Noc(co_Ec)]
E. coli W3110 ΔfadE pJ294{Ptac}[CnFATB3(co_Ec)]/pCDF[alkL][MSMEG_{—}2956(co_Ec)-npt_Noc(co_Ec)]
E. coli W3110 ΔfadE pJ294{Ptac}[CnFATB3(co_Ec)]/pCDF[MSMEG_{—}2956(co_Ec)-npt_Noc(co_Ec)]
E. coli W3110 ΔfadE pJ294[Ptac-synUcTE]/pCDF[alkL][MSMEG_{—}2956(co_Ec)-npt_Noc(co_Ec)]
E. coli W3110 ΔfadE pJ294[Ptac-synUcTE]/pCDF[MSMEG_{—}2956(co_Ec)-npt_Noc(co_Ec)]

These strains are employed to study their ability to produce fatty alcohols and fatty aldehydes from glucose. The following procedure is adopted here:

The strains are subjected to a multistage aerobic culturing process. The strains under investigation are initially grown in Luria-Bertani Bouillon according to Miller (Merck, Darmstadt) containing 100 μg/ml of ampicillin and 100 μg/ml of spectinomycin as a 5 ml preliminary culture from a single colony in each case. The next culturing step proceeds in M9 medium. The medium, consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution, is adjusted to a pH of 7.4 using 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1 M hydrochloric acid (all substances from Merck, Darmstadt) is sterile-filtered before addition to the M9 medium. 10 ml of M9 medium are charged with 100 μg/ml spectinomycin and 100 μg/ml ampicillin into 100 ml conical flasks with chicane and inoculated with 0.5 ml of the preliminary culture. The culturing proceeds at 37° C. and 200 rpm in an incubating shaker. After a culturing time of 8 hours, 50 ml of M9 medium are charged with 100 μg/ml of spectinomycin and 100 μg/ml of ampicillin in a 250 ml conical flask with chicane and inoculated with the 10 ml culture in such a manner that an optical density (600 nm) of 0.2 is achieved. The culturing proceeds at 37° C. and 200 rpm in an incubating shaker. When an optical density (600 nm) of 0.6 to 0.8 is achieved, the gene expression is induced by adding 1 mM IPTG. The strains are cultured for at least a further 24 hours at 30° C. and 200 rpm. During the culturing, samples are withdrawn and the concentration of fatty alcohols and fatty aldehydes of different carbon chain lengths is quantified as described in Example 18. It is shown that the strains E. coli JW5020-1 Kan^{S} pJ294[Ptac-ChFATB1_optEc]/pCDF[alkL][MSMEG_{—}2956(co_Ec)-npt_Noc(co_Ec)], E. coli JW5020-1 Kan^{S} pJ294[Ptac-ChFATB2_optEc]/pCDF[alkL][MSMEG_{—}2956(co_Ec)-npt_Noc(co_Ec)], E. coli JW5020-1 Kan^{S} pJ294{Ptac}[CnFATB3(co_Ec)]/pCDF[alkL][MSMEG_{—}2956(co_Ec)-npt_Noc(co_Ec)], E. coli W3110 ΔfadE pJ294{Ptac}[CnFATB3(co_Ec)]/pCDF[alkL][MSMEG_{—}2956(co_Ec)-npt_Noc(co_Ec)] and E. coli W3110 ΔfadE pJ294[Ptac-synUcTE]/pCDF[alkL][MSMEG_{—}2956(co_Ec)-npt_Noc(co_Ec)] are able, depending on the specificity of the overexpressed acyl-CoA thioesterase gene, to form fatty alcohols and fatty aldehydes of differing carbon chain length and differing degree of saturation compared to the corresponding strains which do not overexpress the gene alkL. In particular, E. coli JW5020-1 Kan^{S} pJ294[Ptac-ChFATB1_optEc]/pCDF[alkL][MSMEG_{—}2956(co_Ec)-npt_Noc(co_Ec)] can produce from glucose more fatty alcohols and fatty aldehydes of chain length C14:0, C16:0 and C16:1, E. coli JW5020-1 Kan^{S} pJ294[Ptac-ChFATB2_optEc]/pCDF[alkL][MSMEG_{—}2956(co_Ec)-npt_Noc(co_Ec)] can produce from glucose more fatty alcohols and fatty aldehydes of chain length C8:0 and C10:0, E. coli JW5020-1 Kan^{S} pJ294{Ptac}[CnFATB3(co_Ec)]/pCDF[alkL][MSMEG_{—}2956(co_Ec)-npt_Noc(co_Ec)] can produce from glucose more fatty alcohols and fatty aldehydes of chain length C12:0, C14:0 and C16:1, E. coli W3110 ΔfadE pJ294{Ptac}[CnFATB3(co_Ec)]/pCDF[alkL][MSMEG_{—}2956(co_Ec)-npt_Noc(co_Ec)] can produce from glucose more fatty alcohols and fatty aldehydes of chain length C12:0, C14:0 and C16:1 and E. coli W3110 ΔfadE pJ294[Ptac-synUcTE]/pCDF[alkL][MSMEG_{—}2956(co_Ec)-npt_Noc(co_Ec)] can produce from glucose more fatty alcohols and fatty aldehydes of chain length C12:0 and C14:0, than the corresponding strains which lack the gene alkL from Pseudomonas putida GPo1.

### Example 24

### Preparation of Expression Vectors for the Coexpression of an Acyl-ACP Thioesterase Gene, Ald from Bacillus subtilis and Cv 2025 from Chromobacterium violaceum

To produce E. coli expression vectors for the genes fatB1 (SEQ ID No. 9) from Cuphea hookeriana, fatB2 (SEQ ID No. 8) from Cuphea hookeriana and synUcTE (SEQ ID No. 37) from Umbellularia californica (in each case encoding an enzyme Eᵢ) and ald (SEQ ID No. 130) from Bacillus subtilis (encoding an enzyme Eₓᵢᵥ) and Cv_{—}2025 (SEQ ID No. 132) from Chromobacterium violaceum (encoding an enzyme Eₓᵢᵢᵢ), the genes fatB1, fatB2 and synUcTE are codon-optimized for expression in Escherichia coli and synthesized together with a tac promotor (SEQ ID No. 39). During the synthesis, a cleavage site is introduced upstream of the promoter and a cleavage site is introduced downstream of the terminator. The synthesized DNA fragments are digested with the restriction endonucleases BamHI and NotI and ligated into the correspondingly cut vector pJ294_alaDH_B.s._TA_C.v.(Ct) (SEQ ID No. 121). The expression vector used here has already been described in German patent application DE102011110946 and recorded there under SEQ ID No. 17. The completed vectors are named pJ294[alaDH_B.s._TA_C.v.(Ct)_Ptac-ChFATB1_optEc] (SEQ ID No. 134), pJ294[alaDH_B.s._TA_C.v.(Ct)_Ptac-ChFATB2_optEc] (SEQ ID No. 135) and pJ294[alaDH_B.s._TA_C.v.(Ct)_Ptac-synUcTE] (SEQ ID No. 136).

### Example 25

### Production of Alkylamines by E. coli Strains Having a Deletion in the fadE Gene which Overexpress an Acyl-CoA Thioesterase Gene, the Genes Cv_{—}2025 from Chromobacterium violaceum and ald from Bacillus subtilis, alkL from Pseudomonas putida GPo1, carA from Mycobacterium smegmatis and npt from Nocardia sp

To generate E. coli strains having expression vectors for the genes ald from Bacillus subtilis, Cv_{—}2025 from Chromobacterium violaceum and fatB1 from Cuphea hookeriana, fatB2 from Cuphea hookeriana, synUcTE from Umbellularia californica in combination with an expression vector for the genes MSMEG_{—}2956 from Mycobacterium smegmatis, npt from Nocardia sp. and alkL from Pseudomonas putida GPo1, electrocompetent cells of E. coli JW5020-1 Kan^{S} and E. coli W3110 ΔfadE are prepared. This takes place in a manner known to those skilled in the art. The strains are transformed with the vectors pJ294[alaDH_B.s._TA_C.v.(Ct)_Ptac-ChFATB1_optEc] (SEQ ID No. 134), pJ294[alaDH_B.s._TA_C.v.(Ct)_Ptac-ChFATB2_optEc](SEQ ID No. 135) and/or pJ294[alaDH_B.s._TA_C.v.(Ct)_Ptac-synUcTE] (SEQ ID No. 136) in combination with pCDF[alkL][MSMEG_{—}2956(co_Ec)-npt_Noc(co_Ec)] (SEQ ID No. 124) and/or pCDF[MSMEG_{—}2956(co_Ec)-npt_Noc(co_Ec)] (SEQ ID No. 125) and plated onto LB-agar plates containing ampicillin (100 μg/ml) and spectinomycin (100 μg/ml). Transformants are checked with regard to the presence of the correct plasmids via plasmid preparation and analytical restriction analysis. The strains hereinafter are constructed in this manner:
E. coli JW5020-1 Kan^{S} pJ294[alaDH_B.s._TA_C.v.(Ct)_Ptac-ChFATB1_optEc]/pCDF[alkL][MSMEG_{—}2956(co_Ec)-npt_Noc(co_Ec)]
E. coli JW5020-1 Kan^{S} pJ294[alaDH_B.s._TA_C.v.(Ct)_Ptac-ChFATB1_optEc]/pCDF[MSMEG_{—}2956(co_Ec)-npt_Noc(co_Ec)]
E. coli JW5020-1 Kan^{S} pJ294[alaDH_B.s._TA_C.v.(Ct)_Ptac-ChFATB2_optEc]/pCDF[alkL][MSMEG_{—}2956(co_Ec)-npt_Noc(co_Ec)]
E. coli JW5020-1 Kan^{S} pJ294[alaDH_B.s._TA_C.v.(Ct)_Ptac-ChFATB2_optEc]/pCDF[MSMEG_{—}2956(co_Ec)-npt_Noc(co_Ec)]
E. coli W3110 ΔfadE pJ294[alaDH_B.s._TA_C.v.(Ct)_Ptac-synUcTE]/pCDF[alkL][MSMEG_{—}2956(co_Ec)-npt_Noc(co_Ec)]
E. coli W3110 ΔfadE pJ294[alaDH_B.s._TA_C.v.(Ct)_Ptac-synUcTE]/pCDF[MSMEG_{—}2956(co_Ec)-npt_Noc(co_Ec)]

These strains are employed to study their ability to produce alkylamines and glucose. The following procedure is adopted here:

The strains are subjected to a multistage aerobic culturing process. The strains under investigation are initially grown in Luria-Bertani Bouillon according to Miller (Merck, Darmstadt) containing 100 μg/ml of ampicillin and 100 μg/ml of spectinomycin as 5 ml preliminary culture each from an individual colony. The next culturing step proceeds in M9 medium. The medium consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution is adjusted to a pH of 7.4 using 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1 M hydrochloric acid (all substances from Merck, Darmstadt) is sterile-filtered before addition to the M9 medium. 10 ml of M9 medium are charged with 100 μg/ml of spectinomycin and 100 μg/ml of ampicillin in 100 ml conical flask with chicane and inoculated with 0.5 ml of the preliminary culture. Culturing proceeds at 37° C. and 200 rpm in an incubating shaker. After a culturing time of 8 hours, 50 ml of M9 medium containing 100 μg/ml of spectinomycin and 100 μg/ml of ampicillin are charged in a 250 ml conical flask with chicane and inoculated with the 10 ml culture, in such a manner that an optical density (600 nm) of 0.2 is achieved. The culturing proceeds at 37° C. and 200 rpm in an incubating shaker. When an optical density (600 nm) of 0.6 to 0.8 is achieved, the gene expression is induced by addition of 1 mM IPTG. The strains are cultured for at least a further 24 hours at 30° C. and 200 rpm. During the culturing, samples are withdrawn and the concentration of fatty aldehydes of differing carbon chain lengths is quantified. It is shown that the strains E. coli JW5020-1 Kan^{S} pJ294[alaDH_B.s._TA_C.v.(Ct)_Ptac-ChFATB1_optEc]/pCDF[alkL][MSMEG_{—}2956(co_Ec)-npt_Noc(co_Ec)], E. coli JW5020-1 Kan^{S} pJ294[alaDH_B.s._TA_C.v.(Ct)_Ptac-ChFATB2_optEc]/pCDF[alkL][MSMEG_{—}2956(co_Ec)-npt_Noc(co_Ec)] and E. coli W3110 ΔfadE pJ294[alaDH_B.s._TA_C.v.(Ct)_Ptac-synUcTE]/pCDF[alkL][MSMEG_{—}2956(co_Ec)-npt_Noc(co_Ec)] are able, depending on the specificity of the overexpressed alkyl-CoA thioesterase gene, to form alkylamines of differing carbon chain length and differing degree of saturation in comparison with the corresponding strains which do not overexpress the alkL gene. In particular, E. coli JW5020-1 Kan^{S} pJ294[alaDH_B.s._TA_C.v.(Ct)_Ptac-ChFATB1_optEc]/pCDF[alkL][MSMEG_{—}2956(co_Ec)-npt_Noc(co_Ec)] can produce from glucose more alkylamines of chain length C14:0, C16:0 and C16:1, E. coli JW5020-1 Kan^{S} pJ294[alaDH_B.s._TA_C.v.(Ct)_Ptac-ChFATB2_optEc]/pCDF[alkL][MSMEG_{—}2956(co_Ec)-npt_Noc(co_Ec)] can produce from glucose more alkylamines of chain length C8:0 and C10:0 and E. coli W3110 ΔfadE pJ294[alaDH_B.s._TA_C.v.(Ct)_Ptac-synUcTE]/pCDF[alkL][MSMEG_{—}2956(co_Ec)-npt_Noc(co_Ec)] can produce from glucose more alkylamines of chain length C12:0 and C14:0 than the corresponding strains which lack the gene alkL from Pseudomonas putida GPo1.

### Example 26

### Preparation of E. coli Expression Vectors for the Expression of Various Acyl-CoA Reductases for Preparation of Fatty Alcohols and Fatty Aldehydes

The gene Maqu_{—}2220 (SEQ ID No. 137) from Marinobacter aquaeolei VT8 or Maqu_{—}2507 (SEQ ID No. 139) from Marinobacter aquaeolei VT8 or AtFAR6 (SEQ ID No. 141) from Arabidopsis thaliana or AcrM (SEQ ID No. 143) from Acinetobacter sp. M-1 or Acr1a (SEQ ID No. 145) from Acinetobacter sp. ADP1 or Acr1b (SEQ ID No. 147) from Acinetobacter sp. ADP1 (in each case encoding an enzyme Eₓ) was cloned into a pJ294 derivative (DNA2.0 Inc., Menlo Park, Calif., USA) following the P_{lac} promotor (SEQ ID No. 149) via the cleavage sites NdeI and NotI. The genes Maqu_{—}2220, Maqu_{—}2507, AtFAR6, AcrM and Acr1a are codon-optimized sequences for E-coli. The Acr1b gene is the wild type sequence. All codon-optimizations were carried out by DNA2.0 (DNA2.0 Inc., Menlo Park, Calif., USA). The DNA sequences were held in a vector specific to DNA2.0.

The genes Maqu_{—}2220, Maqu_{—}2507, AtFAR6 and AcrM were amplified using the polymerase chain reaction (PCR), while introducing the restriction cleavage sites NdeI (at the 5′ end of the respective gene) and NotI (at the 3′ end of the respective gene) as described hereinafter. The matrices used were the vectors from DNA2.0 (DNA2.0 Inc., Menlo Park, Calif., USA) pJ221[Maqu_{—}2220(co_ec)], pJ207[Maqu_{—}2507(co_Ec)], pJ201[AtFAR6(co_Ec)] and pJ221[AcrM(AsM1)].

The oliaonucleotides hereinafter were used in the PCR solutions:

| | | | Seq |
|---|---|---|---|
| | | | ID |
| Gene | Primer | Sequence (5′ => 3′) | NO. |
| Maqu_2220 | Pr-DesFA- | TATATACATATGGCAATTCAGCAGGT | 150 |
| | 1-FW | ACATCACG | |
| | Pr-DesFA- | TATATAGCGGCCGCTCATTACGCGGC | 151 |
| | 1-RV | CTTTTTGC | |
| Maqu_2507 | Pr-DesFA- | TATATACATATGAACTATTTTCTTAC | 152 |
| | 2-FW | AGGCGGTACAGG | |
| | Pr-DesFA- | TATATAGCGGCCGCTTATTACCAGTA | 153 |
| | 2-RV | AATACCACGCATAATTGC | |
| AtFAR6 | Pr-DesFA- | TATATACATATGGCGACGACGAATGT | 154 |
| | 3-FW | ACTGGC | |
| | Pr-DesFA- | TATATAGCGGCCGCTTATTACTCGGT | 155 |
| | 3-RV | TTTCTTCTTGCTCAGG | |
| AcrM | Pr-DesFA- | TATATACATATGAATGCAAAACTCAA | 156 |
| | 5-FW | AAAACTTTTTCAGC | |
| | Pr-DesFA- | TATATAGCGGCCGCTTATTACCAGTG | 157 |
| | 5-RV | TTCGCCTGGG | |

The following parameters were used for the PCRs: 1×: initial denaturation, 98° C., 0:30 min; 35 x: denaturation, 98° C., 0:30 min, annealing, 50° C. (Maqu_{—}2220) 160° C. (Maqu_{—}2507, AcrM, AtFAR6), 0:20 min; elongation, 72° C., 0:35 min; 1×: terminal elongation, 72° C., 5 min. The Phusion™ High-Fidelity Master Mix from New England Biolabs (Frankfurt) was used for the amplification in accordance with the manufacturer's recommendations. 50 μl of the PCR reactions in each case were then separated on a 1% strength agarose gel. The PCR, the agarose gel electrophoresis, ethidium bromide staining of the DNA and determination of the PCR fragment sizes was carried out in the manner known to those skilled in the art. The genes Acr1a and Acr1b from Acinetobacter sp. ADP1 were cloned via in-vitro cloning using the “GeneArt® Seamless Cloning and Assembly Kit” (Cat. No. A13288, Life Technologies GmbH, Darmstadt) in accordance with the manufacturer's instructions. For this purpose, both genes were amplified by PCR, while introducing homologous regions for recombination cloning. The matrices used were the DNA2.0 vector pJ221[Acr1a_AsADP1(co_Ec)] and the vector pCDF{Ptac}[fadD_Ec-acr1b_AsADP1] (SEQ ID No. 109).

The oligonucleotides hereinafter were used in the PCR solutions:

| Gene | Primer | Sequence (5′ => 3′) | Seq |
|---|---|---|---|
| | | | ID NO. |
| Acr1a | Pr-FA_4.1- | AACAGGAGGTAAAACATTGATCTC | 158 |
| | FW | GATCCGTGAAAAACGT | |
| | Pr-FA_4.1- | TGAAGTGGGGGCGGCCTTATCACC | 159 |
| | RV | AGTGCTCACCCGGGAA | |
| Acr1b | Pr- | AACAGGAGGTAAAACAGTGAACAA | 160 |
| | DesFA_4.2- | AAAACTTGAAGCTCTC | |
| | FW | | |
| | Pr- | TGAAGTGGGGGCGGCCTTATTACC | 161 |
| | DesFA_4.2- | AGTGTTCGCCTGGGAA | |
| | RV | | |

The following parameters were used for the PCRs: 1×: initial denaturation, 98° C., 0:30 min; 35×: denaturation, 98° C., 0:30 min, annealing, 64° C., 0:20 min; elongation, 72° C., 0:15 min; 1×: terminal elongation, 72° C., 5 min. The Phusion™ High-Fidelity Master Mix from New England Biolabs (Frankfurt) was used for the amplification in accordance with the manufacturer's recommendations. 50 μl of the PCR reactions in each case were then separated on a 1% strength agarose gel. The PCR, the agarose gel electrophoresis, ethidium bromide staining of the DNA and determination of the PCR fragment sizes were carried out in the manner known to those skilled in the art.

In all cases, PCR fragments of the expected size were able to be amplified. These were 1568 base pairs (bp) for Maqu_{—}2220, 2012 by for Maqu_{—}2507, 1673 by for AtFAR6, 914 by for AcrM, 947 by for Acr1a and 923 base pairs for Acr1b 923.

To isolate the DNA from an agarose gel, the target DNA was cut out of the gel using a scalpel and purified using the “Quick Gel Extraction Kit” from Qiagen (Hilden). The procedure was carried out according to the manufacturer's instructions.

In the next step, the PCR products of Maqu_{—}2220, Maqu_{—}2507, AtFAR6 and AcrM, just like the pJ294 derivate (DNA2.0 Inc., Menlo Park, Calif., USA), were cut using the restriction enzymes NdeI and NotI (New England Biolabs, Frankfurt) in accordance with the manufacturer's instructions. The cut vector was then applied to a 1% strength agarose gel. The agarose gel electrophoresis, ethidium bromide staining of the DNA and determination of the fragment sizes were carried out in the manner known to those skilled in the art. To isolate the DNA from an agarose gel, the target DNA was cut out from the gel using a scalpel and purified with the “Quick Gel Extraction Kit” from Qiagen (Hilden). The procedure was carried out according to the manufacturer's instructions. The NdeI-NotI-cut PCR amplificates Maqu_{—}2220, Maqu_{—}2507, AtFAR6 and AcrM were then ligated in each case with the NdeI-NotI-cut vector via the T4 DNA ligase (New England Biolabs, Frankfurt) in accordance with the manufacturer's instructions, obtaining the resultant vectors.

The PCR products of Acr1a and Acr1b from Acinetobacter sp. ADP1 were recombined together with the NdeII-NotI-cut pJ294 derivative using in-vitro cloning, using the “GeneArt® Seamless Cloning and Assembly Kit” (Cat. No. A13288, Life Technologies GmbH, Darmstadt), obtaining the resulting vectors. The use corresponded to the manufacturer's recommendations. The vector pJ294 is a E. coli expression vector which imparts an ampicillin resistance to the organism, and bears a p15A replication origin. Upstream of the cleavage site NdeI there is a P_{lac} promotor. The transformation of chemically competent E. coli DH5α cells (New England Biolabs, Frankfurt) proceeded in the manner known to those skilled in the art.

The correctness of the respective plasmid was controlled by restriction analysis using NruI. The authenticity of the inserted fragments was checked by DNA sequencing.

The completed E. coli expression vectors were named as follows:

| Vector name | Vector | Gene | SEQ ID No. |
|---|---|---|---|
| pHg-12-58 | pJ294 | Maqu_2220 | 162 |
| pHg-12-59 | pJ294 | Maqu_2507 | 163 |
| pHg-12-60 | pJ294 | AtFAR6 | 164 |
| pHg-12-61 | pJ294 | AcrM | 165 |
| pHg-12-62 | pJ294 | Acr1a | 166 |
| pHg-12-63 | pJ294 | Acr1b | 167 |

### Example 27

### Production of Fatty Alcohols and Fatty Aldehydes by E. coli Strains Having a Deletion in the fadE Gene and Expression Vectors for the Genes Maqu_{—}2220 from Marinobacter aquaeolei VT8, Maqu_{—}2507 from Marinobacter aquaeolei VT8, AtFAR6 from Arabidopsis thaliana, AcrM from Acinetobacter sp. M-1, Acr1 from Acinetobacter sp. ADP1 or Acr1 from Acinetobacter calcoaceticus in Combination with an Expression Vector for the alkL Gene from Pseudomonas putida

First, a E. coli W3110 strain having a deletion in the fadE gene is produced as described in Example 4.

To generate E. coli strains having the expression vector for the alkL gene from Pseudomonas putida GPo1 in combination with the expression vector for the Maqu_{—}2220 gene from Marinobacter aquaeolei VT8 or Maqu_{—}2507 from Marinobacter aquaeolei VT8 or AtFAR6 from Arabidopsis thaliana or AcrM from Acinetobacter sp. M-1 or Acr1 from Acinetobacter sp. ADP1 or Acr1 from Acinetobacter calcoaceticus, electrocompetent cells of E. coli W3110 ΔfadE and E. coli JW5020-1 Kan^{S} were prepared. This proceeds in a manner known to those skilled in the art. The host strain E. coli JW5020-1 Kan^{S} is a descendant of the E. coli JW5020-1 (CSGC, The coli genetic stock center, Yale University, New Haven, USA) and is a E. coli BW25113 derivative which carries a deletion of the fadE gene. The fadE gene was replaced by a kanamycin cassette. It was removed in a manner known to those skilled in the art (see Datsenko K. A. and Wanner B. L. (2000) PNAS 97(12):6640-6645) before the strain is equipped with the expression vectors using a helper plasmid which encodes the Hp recombinase, resulting in strain E. coli JW5020-1 Kan^{S}.

The competent cells were transformed with the plasmids pCDFDuet-1 or pCDF[alkL] in combination with pHg-12-58 or pHg-12-59 or pHg-12-60 or pHg-12-61 or pHg-12-62 or pHg-12-63 and plated out on LB plates containing spectinomycin (100 μg/ml) and ampicillin (100 μg/ml). Transformants were examined with respect to the presence of the correct plasmids via plasmid preparation and analytical restriction analysis.

In this manner the E. coli strains hereinafter were generated:
E. coli W3110 ΔfadE pCDF[alkL]/pHg-12-58
E. coli W3110 ΔfadE pCDF[alkL]/pHg-12-59
E. coli W3110 ΔfadE pCDF[alkL]/pHg-12-60
E. coli W3110 ΔfadE pCDF[alkL]/pHg-12-61
E. coli W3110 ΔfadE pCDF[alkL]/pHg-12-62
E. coli W3110 ΔfadE pCDF[alkL]/pHg-12-63
E. coli W3110 ΔfadE pCDFDuet-1/pHg-12-58
E. coli W3110 ΔfadE pCDFDuet-1/pHg-12-59
E. coli W3110 ΔfadE pCDFDuet-1/pHg-12-60
E. coli W3110 ΔfadE pCDFDuet-1/pHg-12-61
E. coli W3110 ΔfadE pCDFDuet-1/pHg-12-62
E. coli W3110 ΔfadE pCDFDuet-1/pHg-12-63
E. coli JW5020-1 Kan^{s} ΔfadE pCDF[alkL]/pHg-12-58
E. coli JW5020-1 Kan^{s} ΔfadE pCDF[alkL]/pHg-12-59
E. coli JW5020-1 Kan^{s} ΔfadE pCDF[alkL]/pHg-12-60
E. coli JW5020-1 Kan^{s} ΔfadE pCDF[alkL]/pHg-12-61
E. coli JW5020-1 Kan^{s} ΔfadE pCDF[alkL]/pHg-12-62
E. coli JW5020-1 Kan^{s} ΔfadE pCDF[alkL]/pHg-12-63
E. coli JW5020-1 Kan^{s} ΔfadE pCDFDuet-1/pHg-12-58
E. coli JW5020-1 Kan^{s} ΔfadE pCDFDuet-1/pHg-12-59
E. coli JW5020-1 Kan^{s} ΔfadE pCDFDuet-1/pHg-12-60
E. coli JW5020-1 Kan^{s} ΔfadE pCDFDuet-1/pHg-12-61
E. coli JW5020-1 Kan^{s} ΔfadE pCDFDuet-1/pHg-12-62
E. coil JW5020-1 Kan^{s} ΔfadE pCDFDuet-1/pHg-12-63

These strains are employed to investigate their ability to produce fatty alcohols and fatty aldehydes from glucose. The following procedure is followed here:

The strains are subjected to a multistage aerobic culturing process. The strains under examination are initially grown in Luria-Bertani Bouillon according to Miller (Merck, Darmstadt) as a 5 ml preliminary culture from an individual colony in each case. The next culturing step proceeds in M9 medium. The medium consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution is adjusted to a pH of 7.4 using 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1 M hydrochloric acid (all substances from Merck, Darmstadt) is sterile-filtered before addition to the M9 medium. 10 ml of M9 medium are charged with 100 μg/ml of spectinomycin and 100 μg/ml of ampicillin into 100 ml conical flasks with chicane and inoculated with 0.5 ml of the preliminary culture. The culturing proceeds at 37° C. and 200 rpm in an incubating shaker. After a culturing time of 8 hours, 50 ml of M9 medium containing 100 μg/ml of spectinomycin and 100 μg/ml of ampicillin are charged in a 250 ml conical flask with chicane and inoculated with the 10 ml culture in such a manner that an optical density (600 nm) of 0.2 is achieved. The culturing proceeds at 37° C. and 200 rpm in an incubating shaker. When an optical density (600 nm) of 0.6 to 0.8 is achieved, the gene expression is induced by adding 1 mM of IPTG. The strains are cultured for a further 48 hours at 30° C. and 200 rpm in an incubating shaker. During the culturing, samples of 1 ml are withdrawn and the concentration of fatty alcohols and fatty aldehydes of differing carbon chain lengths is quantified using the method described in Example 18. It is shown that the strains E. coli W3110 ΔfadE pCDF[alkL]/pHg-12-58, E. coli W3110 ΔfadE pCDF[alkL]/pHg-12-59, E. coli W3110 ΔfadE pCDF[alkL]/pHg-12-60, E. coli W3110 ΔfadE pCDF[alkL]/pHg-12-61, E. coli W3110 ΔfadE pCDF[alkL]/pHg-12-62, E. coli W3110 ΔfadE pCDF[alkL]/pHg-12-63 and the strains E. coli JW5020-1 Kan^{s} ΔfadE pCDF[alkL]/pHg-12-58, E. coli JW5020-1 Kan^{s} ΔfadE pCDF[alkL]/pHg-12-59, E. coli JW5020-1 Kan^{s} ΔfadE pCDF[alkL]/pHg-12-60, E. coli JW5020-1 Kan^{s} ΔfadE pCDF[alkL]/pHg-12-61, E. coli JW5020-1 Kan^{s} ΔfadE pCDF[alkL]/pHg-12-62 and E. coli JW5020-1 Kan^{s} ΔfadE pCDF[alkL]/pHg-12-63 can produce a higher titre of fatty alcohols and fatty aldehydes of differing chain length from glucose than the strains which lack the gene alkL from Pseudomonas putida GPo1. In particular, E. coli W3110 ΔfadE pCDF[alkL]/pHg-12-58 can produce more fatty alcohols and fatty aldehydes of chain length C14:0, C16:0 and C16:1, E. coli W3110 ΔfadE pCDF[alkL]/pHg-12-59 can produce more fatty alcohols and fatty aldehydes of chain length C14:0, C16:0 and C16:1, E. coli W3110 ΔfadE pCDF[alkL]/pHg-12-60 can produce more fatty alcohols and fatty aldehydes of chain length C16:0 and C16:1, E. coli W3110 ΔfadE pCDF[alkL]/pHg-12-61 can produce more fatty alcohols and fatty aldehydes of chain length C8:0 and 010:0, E. coli W3110 ΔfadE pCDF[alkL]/pHg-12-62 can produce more fatty alcohols and fatty aldehydes of chain length C12:0 and C14:0, E. coli W3110 ΔfadE pCDF[alkL]/pHg-12-63 can produce more fatty alcohols and fatty aldehydes of chain length C12:0 and C14:0, E. coli JW5020-1 Kan^{s} ΔfadE pCDF[alkL]/pHg-12-58 can produce more fatty alcohols and fatty aldehydes of chain length C14:0, C16:0 and C16:1, E. coli JW5020-1 Kan^{s} ΔfadE pCDF[alkL]/pHg-12-59 can produce more fatty alcohols and fatty aldehydes of chain length C14:0, C16:0 and C16:1, E. coli JW5020-1 Kan^{s} ΔfadE pCDF[alkL]/pHg-12-60 can produce more fatty alcohols and fatty aldehydes of chain length C16:0 and C16:1, E. coli JW5020-1 Kan^{s} ΔfadE pCDF[alkL]/pHg-12-61 can produce more fatty alcohols and fatty aldehydes of chain length C8:0 and C10:0, E. coli JW5020-1 Kan^{s} ΔfadE pCDF[alkL]/pHg-12-62 can produce more fatty alcohols and fatty aldehydes of chain length C12:0 and C14:0 and E. coli JW5020-1 Kan^{s ΔfadE pCDF[alkL]/pHg-}12-63 can produce more fatty alcohols and fatty aldehydes of chain length C12:0 and C14:0 from glucose than the corresponding strains which lack the gene alkL from Pseudomonas putida GPo1.

### Example 28

### Preparation of E. coli Expression Vectors for the Gene oleT_{JE} from Jeotgalicoccus sp. ATCC 8456 for Preparation of Alkenes

For the preparation of expression vectors, the sequence of the gene oleT_{JE} (SEQ ID No. 168) from Jeotgalicoccus sp. ATCC 8456 (encoding an enzyme Eₓᵢ) was codon-optimized for expression in E. coli with DNA2.0 (DNA2.0 Inc., Menlo Park, Calif., USA) and synthesized in combination with the P_{lac} promotor (SEQ ID No. 149) or the P_{lac} promotor and the alkL gene (SEQ ID No. 1). The cloning of the constructs P_{lac}-oleT_{JE} (SEQ ID No. 170) and P_{lac}-oleT_{JE}-alkL (SEQ ID No. 171) proceeded in vectors specific to DNA2.0. Both constructs are terminated by a terminator sequence (SEQ ID No. 172). In addition, a cleavage site (EcoNI or NotI) was introduced upstream of the P_{lac} promotor and downstream of the terminator in each case. The synthesized DNA fragments P_{lac}-oleT_{JE} and P_{lac}-oleT_{JE}-alkL and the vector pCDFDuet-1 (Merck, Darmstadt) (SEQ ID No 53) were cut with the restriction endonucleases EcoNI and NotI (New England Biolabs, Frankfurt) in accordance with the manufacturer's instructions. The NdeI-NotI-cut constructs and the cut vector were then applied to a 1% strength agarose gel. The agarose gel electrophoresis, ethidium bromide staining of the DNA and determination of the fragment sizes was carried out in the manner known to those skilled in the art. For isolation of the DNA from an agarose gel, the target DNA was cut out from the gel with a scalpel and purified using the “Quick Gel Extraction Kit” from Qiagen (Hilden). The procedure was in accordance with the manufacturer's instructions.

Subsequently, the fragment P_{lac}-oleT_{JE} or P_{lac}-oleT_{JE}-alkL carried out was ligated into the vector pCDFDuet-1 vector via the T4 DNA ligase (New England Biolabs, Frankfurt) in accordance with the manufacturer's instructions, obtaining the resultant vectors.

The vector pCDFDuet-1 is an E. coli vector which imparts a spectinomycin/streptomycin resistance to the organism, and also carries a CoIDF13 replication origin. The transformations of chemically competent E. coli DH5α cells (New England Biolabs, Frankfurt) proceeded in the manner known to those skilled in the art.

The correctness of the respective plasmid was controlled by restriction analysis with EcoRV.

The authenticity of the inserted fragments was checked by DNA sequencing.

The completed E. coli expression vectors were named pHg-12-66 (pCDF[P_{lac}-oleT_{JE}]; SEQ ID No 173) and pHg-12-67 (pCDF[P_{lac}-oleT_{JE}-alkL]; SEQ ID No 174).

### Example 29

### Production of Alkenes by E. coli Strains Having a Deletion in the fadE Gene and Expression Vectors for the Genes fatB1 from Cuphea Hookeriana, fatB2 from Cuphea hookeriana, fatB3 from Cocos nucifera, synUcTE from Umbellularia californica in Combination with Expression Vectors for the Gene oleT_{JE} from Jeotgalicoccus Sp. ATCC 8456 and alkL from Pseudomonas putida

First, an E. coli W3110 strain having a deletion in the fadE gene is prepared as described in Example 4.

To generate E. coli strains having the expression vectors for the genes fatB1 from Cuphea palustris or fatB2 from Cuphea palustris or fatB3 from Cocos nucifera or synUcTE from Umbellularia californica in combination with the expression vectors for the gene oleT_{JE} from Jeotgalicoccus sp. ATCC 8456 or the genes oleT_{JE} from Jeotgalicoccus sp. ATCC 8456 and alkL from Pseudomonas putida, electrocompetent cells of E. coli W3110 ΔfadE and E. coli JW5020-1 Kan^{S} are prepared. This proceeds in a manner known to those skilled in the art. The host strain E. coli JW5020-1 Kan^{s} is a descendant of E. coli JW5020-1 (CSGC, The coli genetic stock center, Yale University, New Haven, USA) and is an E. coli BW25113 derivative which carries a deletion of the fadE gene. The fadE gene was replaced by a kanamycin cassette. This was removed in a manner known to those skilled in the art (see Datsenko K. A. and Wanner B. L. (2000) PNAS 97(12):6640-6645) before equipping the strain with the expression vectors using a helper plasmid which encodes the Flp recombinase, resulting in strain E. coli JW5020-1 Kan^{S}. The competent cells were transformed using the plasmids pJ294[Ptac-ChFATB1_optEc] or pJ294[Ptac-ChFATB2_optEc] or pJ294{Ptac}[CnFATB3(co_Ec)] or pJ294[Ptac-synUcTE] in combination with pHg-12-66 or pHg-12-67 and plated onto LB plates containing ampicillin (100 μg/ml) and spectinomycin (100 μg/ml). Transformants were checked with respect to the presence of the correct plasmids by plasmid preparation and analytical restriction analysis.

In this manner the E. coli strains hereinafter were generated:
E. coli W3110 ΔfadE pJ294[Ptac-ChFATB1_optEc]/pHg-12-66
E. coli W3110 ΔfadE pJ294[Ptac-ChFATB1_optEc]/pHg-12-67
E. coli JW5020-1 Kan^{s} pJ294[Ptac-ChFATB1_optEc]/pHg-12-66
E. coli JW5020-1 Kan^{s} pJ294[Ptac-ChFATB1_optEc]/pHg-12-67
E. coli JW5020-1 Kan^{s} pJ294[Ptac-ChFATB2_optEc]/pHg-12-66
E. coli JW5020-1 Kan^{s} pJ294[Ptac-ChFATB2_optEc]/pHg-12-67
E. coli JW5020-1 Kan^{s} pJ294[Ptac-CnFATB3_optEc]/pHg-12-66
E. coli JW5020-1 Kan^{s} pJ294[Ptac-CnFATB3_optEc]/pHg-12-67
E. coli W3110 ΔfadE pJ294[Ptac-synUcTE]/pHg-12-66
E. coli W3110 ΔfadE pJ294[Ptac-synUcTE]/pHg-12-67

These strains are employed in order to investigate their ability to produce alkenes via the production of fatty acids from glucose. The following procedure is used:

The strains are subjected to a multistage aerobic culturing process. The strains under examination are initially grown in Luria-Bertani Bouillon according to Miller (Merck, Darmstadt) as a 5 ml preliminary culture each from a single colony. The next culturing step proceeds in M9 medium. The medium consisting of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all substances from Merck, Darmstadt) and 0.1% (v/v) trace element solution is adjusted to a pH of 7.4 using 25% strength ammonium hydroxide solution. The added trace element solution consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 1M hydrochloric acid (all substances from Merck, Darmstadt) is sterile-filtered before addition to the M9 medium. 10 ml of M9 medium are charged with 100 μg/ml of spectinomycin and 100 μg/ml of ampicillin into 100 ml conical flasks having chicane and inoculated with 0.5 ml from the preliminary culture. The culturing proceeds at 37° C. and 200 rpm in an incubating shaker. After a culturing time of 8 hours, 50 ml of M9 medium containing 100 μg/ml of spectinomycin and 100 μg/ml of ampicillin are charged in a 250 ml conical flask with chicane and inoculated with the 10 ml culture in such a manner that an optical density (600 nm) of 0.2 is achieved. The culturing proceeds at 37° C. and 200 rpm in an incubating shaker. On reaching an optical density (600 nm) of 0.6 to 0.8, the gene expression is induced by addition of 1 mM IPTG. The strains are cultured for a further 48 hours at 30° C. and 200 rpm in an incubating shaker. During the culturing, samples of 1 ml are withdrawn and the concentration of free fatty acids and alkenes of differing carbon chain lengths are quantified using the method described in Example 30. It is shown that the strains E. coli W3110 ΔfadE pJ294[Ptac-ChFATB1_optEc]/pHg-12-67, E. coli JW5020-1 Kan^{s} pJ294[Ptac-ChFATB1_optEc]/pHg-12-67, E. coli JW5020-1 Kan^{s} pJ294[Ptac-ChFATB2_optEc]/pHg-12-67, E. coli JW5020-1 Kan^{s} pJ294[Ptac-CnFATB3_optEc]/pHg-12-67 and E. coil W3110 ΔfadE pJ294[Ptac-synUcTE]/pHg-12-67 can produce higher titres of alkenes of different chain length from glucose than the strains which lack the gene alkL from Pseudomonas putida GPo1. In particular E. coli W3110 ΔfadE pJ294[Ptac-ChFATB1_optEc]/pHg-12-67 can produce more 1-alkenes of chain length C13 and C15 and also 1,8-dienes of chain length C15, E. coli JW5020-1 Kan^{s} pJ294[Ptac-ChFATB1_optEc]/pHg-12-67 can produce more 1-alkenes of chain length C13 and C15 and also 1,8-dienes of chain length C15, E. coli JW5020-1 Kan^{s} pJ294[Ptac-ChFATB2_optEc]/pHg-12-67 can produce more 1-alkenes of chain length C7 and C9, E. coil JW5020-1 Kan^{s} pJ294[Ptac-CnFATB3_optEc]/pHg-12-67 can produce more 1-alkenes of chain length C11 and C13 and also 1,8-dienes of chain length C15 and E. coli W3110 ΔfadE pJ294[Ptac-synUcTE]/pHg-12-67 can produce more 1-alkenes of chain length C11 and C13 from glucose than the corresponding strains which lack the gene alkL from Pseudomonas putida GPo1.

### Example 30

### Chromatographic Quantification of Alkenes

Alkenes are quantified by means of gas chromatography with coupled mass spectrometry (GC/MS).

To extract the samples, consisting of 1 ml of culture broth, they are admixed with 500 μl of ethyl acetate (Chromasolv®Plus 99.9%, Sigma No. 650528-1L), shaken for 10 min at 12 Hz and sedimented for 5 min at 13 200 rpm in a bench centrifuge (Eppendorf, Hamburg). The organic phase (ethyl acetate) is transferred to HPLC vials with an insert and analysed by means of coupled GC/MS for alkenes of differing chain length (C8-C18).

For separation of alkenes, the capillary column ZB-50 having the dimensions 30 m×320 μm and a film thickness of 0.5 μm (Phenomenex, Aschaffenburg) is used as stationary phase. The carrier gas used is helium at a constant flow rate of 1.5 ml/min. The separation proceeds in the course of 45 min at an injector temperature of 250° C. and a detector temperature of 250° C. The column temperature at the start is 40° C. and is held for 2 min. Subsequently, the column temperature is raised at 7° C./min to 150° C., then raised at 15° C./min to 320° C. and held for 10 min. The injection volume is 1 μl splitless. The detection proceeds by means of an MS (DSQ II) detector (Thermo Fisher Scientific) with a mass range of 12-800 m/z (0-8 min SIM at m/z 55.97). The reference substance employed for the alkenes is a standard mixture consisting of in each case 10 μg/ml 1-octene (Sigma-Aldrich), 1-decene (94%, Sigma-Aldrich), 1-dodecene (>99%, Sigma-Aldrich), 1-tetradecene (>97%, Sigma-Aldrich), 1-hexadecene (99.9%, Sigma-Aldrich), 1-octadecene (Sigma-Aldrich), for calibration. Relative quantification of the samples is performed via the peak areas.

1. A microorganism comprising:
a first genetic modification so that the microorganism is capable of forming more of an organic substance from at least one simple carbon source in comparison to a wild type version of the microorganism, and
a second genetic modification so that the microorganism forms more of an alkL gene product in comparison to a wild type version of the microorganism.

2. The microorganism of claim 1, wherein the organic substance is selected from the group consisting of:
an optionally substituted carboxylic acid,
an optionally substituted carboxylic acid ester,
an optionally substituted alkane having 3 to 34 carbon atoms,
an optionally substituted alkene having 3 to 34 carbon atoms,
an optionally substituted monohydric alcohol having 3 to 34 carbon atoms,
an optionally substituted aldehydes having 3 to 34 carbon atoms, and
an optionally substituted monovalent amine having 3 to 34 carbon atoms.

3. The microorganism of claim 1, wherein the organic substance is selected from the group consisting of a fatty acid, a fatty acid ester, an alkan-1-al, and alkan-1-ol, an alkan-1-amine, and alkane and an alkene.

4. The microorganism of claim 1, wherein the alkL gene product is encoded by an alkL gene from a Gram-negative bacterium.

5. The microorganism of claim 1, wherein the alkL gene product is selected from the group consisting of:
a protein encoded by SEQ ID NO: 1;
a protein encoded by SEQ ID NO: 3;
a protein comprising a polypeptide sequence selected from the group consisting of SEQ ID NO: 2, SEQ ID NO: 30, SEQ ID NO: 31, SEQ ID NO: 32, and SEQ ID NO: 33; and
a protein comprising a polypeptide sequence in which up to 60% of the amino acid residues are modified compared to SEQ ID NO: 2, SEQ ID NO: 30, SEQ ID NO: 31, SEQ ID NO: 32, or SEQ ID NO: 33 by deletion, insertion, substitution or a combination thereof, wherein the protein has at least 50% of an activity compared to SEQ ID NO: 2, SEQ ID NO: 30, SEQ ID NO: 31, SEQ ID NO: 32, or SEQ ID NO: 33, respectively.

6. The microorganism of claim 1, which is a Gram-negative bacterium.

7. The microorganism of claim 1, wherein the first genetic modification affects an activity of at least one enzyme selected from the group consisting of:
Eᵢ acyl-ACP thioesterase,
Eᵢᵢ acyl-CoA thioesterase,
E_{iib} acyl-CoA:ACP transacylase,
Eᵢᵢᵢ polyketide synthase that catalyzes a reaction involved in the synthesis of carboxylic acids and carboxylic acid esters, and
Eᵢᵥ hexanoic acid synthase,
wherein the activity is increased in comparison to an enzymatic activity of the wild type version of the microorganism.

8. The microorganism of claim 1, further comprising a third genetic modification that affects an activity of at least one enzyme selected from the group consisting of:
E_{iib} acyl-CoA:ACP transacylase,
Eᵥ wax ester synthase or alcohol O-acyl transferase,
Eᵥₐ fatty acid-O-methyltransferase that catalyzes the synthesis of a fatty acid methyl ester from a fatty acid and S-adenosylmethionine,
Eᵥᵢ acyl-CoA synthetase, and
Eᵥᵢᵢ acyl thioesterase,
wherein the activity is increased in comparison to an enzymatic activity of the wild type version of the microorganism.

9. The microorganism of claim 1, further comprising a fourth genetic modification that affects an activity of at least one enzyme selected from the group consisting of:
E_{iib} acyl-CoA:ACP transacylase,
Eᵥᵢ acyl-CoA synthetase,
Eᵥᵢᵢᵢ acyl-CoA reductase,
Eᵢₓ fatty acid reductase,
Eₓ acyl-ACP reductase,
Eₓᵢ cytochrome P450 fatty acid decarboxylase that catalyzes the conversion of an alkanoic acid with n carbon atoms into a corresponding terminal olefin with n−1 carbon atoms,
Eₓᵢᵢ alkan-1-al decarbonylase that catalyzes the conversion of an alkan-1-al (n carbon atoms) into a corresponding alkane (n−1 carbon atoms) or terminal olefin (n−1 carbon atoms), and
Eₓᵢᵢᵢ alkan-1-al transaminase that catalyzes the conversion of an alkan-1-al into a corresponding alkan-1-amine,
wherein the activity is increased in comparison to an enzymatic activity of the wild type version of the microorganism.

10. The microorganism of claim 1, further comprising a fifth genetic modification that affects an activity of at least one enzyme selected from the group consisting of:
Eₐ acyl-CoA synthetase (EC 6.2.1.3) that catalyzes the synthesis of an acyl-coenzyme A thioester,
E_{b} acyl-CoA dehydrogenase (EC 1.3.99.-, EC 1.3.99.3 or EC 1.3.99.13) that catalyzes the oxidation of an acyl-coenzyme A thioester to give a corresponding enoyl-coenzyme A thioester,
E_{c} acyl-CoA oxidase (EC 1.3.3.6) that catalyzes the oxidation of an acyl-coenzyme A thioester to give a corresponding enoyl-coenzyme A thioester,
E_{d} enoyl-CoA hydratase (EC 4.2.1.17 or EC 4.2.1.74) that catalyzes the hydratization of an enoyl-coenzyme A thioester to give a corresponding 3-hydroxyacyl-coenzyme A thioester,
E_{f} 3-hydroxyacyl-CoA dehydrogenase (EC 1.1.1.35 or EC 1.1.1.211) that catalyzes the oxidation of a 3-hydroxyacyl-coenzyme A thioester to give a corresponding 3-oxoacyl-coenzyme A thioester, and
E_{g} acetyl-CoA acyltransferase (EC 2.3.1.16) that catalyzes the transfer of an acetyl residue from a 3-oxoacyl-coenzyme A thioester to coenzymes A and thus generates an acyl-coenzyme A thioester that is shortened by two carbon atoms,
wherein the activity is reduced in comparison to an enzymatic activity of the wild type version of the microorganism.

11. The microorganism of claim 1, further comprising a seventh genetic modification that affects an activity of at least one enzyme selected from the group consisting of:
E₁ P450 alkane hydroxylases,
E_{1b} AlkB alkane hydroxylases of EC 1.14.15.3,
E_{1c} fatty alcohol oxidases of EC 1.1.3.20,
E_{1d} AlkJ alcohol dehydrogenases of EC 1.1.99,
E₁ₑ alcohol dehydrogenase of EC 1.1.1.1 or EC 1.1.1.2 and
E_{1f} aldehyde dehydrogenases of EC 1.2.1.3, EC 1.2.1.4 or EC 1.2.1.5
wherein the activity is reduced in comparison to the wild type version of the microorganism.

12. (canceled)

13. A process for producing an organic substance from a simple carbon source, the process comprising
I) contacting the microorganism of claim 1 with a medium comprising the simple carbon source,
II) culturing the microorganism under conditions which make it possible for the microorganism to form the organic substance from the simple carbon source, and
III) optionally isolating the organic substance formed.
